SEQUENCE LISTING (24 sequences)

SEQ ID NO: 1 - DNA, 126 bp, Artificial Sequence
  chemically synthesized
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 2 - DNA, 336 bp, Artificial Sequence
  chemically synthesized
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 3 - protein, 42 aa, Artificial Sequence
  chemically synthesized
KRGRKKLLYIFKQPFMRPVQTTQEEDGCSCRFPEEEEGGCEL

SEQ ID NO: 4 - protein, 112 aa, Artificial Sequence
  chemically synthesized
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 1016 aa, Homo sapiens
MKMASTRCKLARYLEDLEDVDLKKFKMHLEDYPPQKGCIPLPRGQTEKADHVDLATLMIDFNGEEKAWAMAVWIFAAINRRDLYEKAKRDEPKWGSDNARVSNPTVICQEDSIEEEWMGLLEYLSRISICKMKKDYRKKYRKYVRSRFQCIEDRNARLGESVSLNKRYTRLRLIKEHRSQQEREQELLAIGKTKTCESPVSPIKMELLFDPDDEHSEPVHTVVFQGAAGIGKTILARKMMLDWASGTLYQDRFDYLFYIHCREVSLVTQRSLGDLIMSCCPDPNPPIHKIVRKPSRILFLMDGFDELQGAFDEHIGPLCTDWQKAERGDILLSSLIRKKLLPEASLLITTRPVALEKLQHLLDHPRHVEILGFSEAKRKEYFFKYFSDEAQARAAFSLIQENEVLFTMCFIPLVCWIVCTGLKQQMESGKSLAQTSKTTTAVYVFFLSSLLQPRGGSQEHGLCAHLWGLCSLAADGIWNQKILFEESDLRNHGLQKADVSAFLRMNLFQKEVDCEKFYSFIHMTFQEFFAAMYYLLEEEKEGRTNVPGSRLKLPSRDVTVLLENYGKFEKGYLIFVVRFLFGLVNQERTSYLEKKLSCKISQQIRLELLKWIEVKAKAKKLQIQPSQLELFYCLYEMQEEDFVQRAMDYFPKIEINLSTRMDHMVSSFCIENCHRVESLSLGFLHNMPKEEEEEEKEGRHLDMVQCVLPSSSHAACSHGLVNSHLTSSFCRGLFSVLSTSQSLTELDLSDNSLGDPGMRVLCETLQHPGCNIRRLCNQKLVELDLSDNALGDFGIRLLCVGLKHLLCNLKKLWLVSCCLTSACCQDLASVLSTSHSLTRLYVGENALGDSGVAILCEKAKNPQCNLQKLGLVNSGLTSVCCSALSSVLSTNQNLTHLYLRGNTLGDKGIKLLCEGLLHPDCKLQVLELDNCNLTSHCCWDLSTLLTSSQSLRKLSLGNNDLGDLGVMMFCEVLKQQSCLLQNLGLSEMYFNYETKSALETLQEEKPELTVVFEPSW

SEQ ID NO: 6 - DNA, 3207 bp, Homo sapiens
tgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacactgtggtgttccagggggcggcagggattgggaaaacaatcctggccaggaagatgatgttggactgggcgtcggggacactctaccaagacaggtttgactatctgttctatatccactgtcgggaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattaggagattgtgcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaa

SEQ ID NO: 7 - protein, 1031 aa, Bos Taurus
MRMVSVRCKLARYLEDLEDIDFKKFKMHLEDYPSQKGCTSIPRGQTEKADHVDLATLMIDFNGEEKAWAMAKWIFAAINRRDLYEKAKREEPEWENANISVLSQEESLEEEWMGLLGYLSRISICRKKKDYCKKYRKYVRSKFQCIKDRNARLGESVNLNKRFTRLRLIKEHRSQQEREHELLAIGRTWAKIQDSPVSSVNLELLFDPEDQHSEPVHTVVFQGAAGIGKTILARKIMLDWASEKLYQDRFDYLFYIHCREVSLGTQRSLGDLIASCCPGPNPPIGKIVSKPSRILFLMDGFDELQGAFDEHTEALCTNWRKVERGDILLSSLIRKRLLPEASLLITTRPVALEKLQHLLGQARHVEILGFSEARRKEYFLKYFSDEQQAREAFRLIQENEILFTMCFIPLVCWIVCTGLKQQMDSGKSLARTSKTTTAVYIFFLSSLLQSQGGSQENHNSATLWGLCSLAADGIWNQKILFQECDLRNHGLQKADVSAFLRMNLFQKEVDCEKFYSFIHMTFQEFFAAMYYLLEEDNHGEMRNTPQACSKLPNRDVKVLLENYGKFEKGYLIFVVRFLFGLINQERTSYLEKKLSCKISQKIRLELLKWIEAKANAKTLQIEPSQLELFYCLYEMQEEDFVQRAMSHFPKIEIKLSTRMDHVVSSFCIENCRHVESLSLRLLHNSPKEEEEEEEVRHSHMDRSVLSDFEVAYSQGLVNYLTSSICRGIFSVLSNNWNLTELNLSGNTLGDPGMNVLCETLQQPGCNIRRLWLGQCCLSHQCCFNISSVLSNNQKLVELDLSHNALGDFGIRLLCVGLRHLFCNLKKLWLVSCCLTSASCEDLASVLSTNHSLTRLYLGENALGDSGVGILCEKVKNPHCNLQKLGLVNSGLTSGCCPALSSVLSTNQNLTHLYLQGNALGDMGVKLLCEGLLHRNCKLQVLELDNCSLTSHCCWDLSTLLTSNQSLRKLCLGNNDLGDLGVMLLCEVLKQQGCLLKSLRLCEMYFNYDTKRALETLQEEKPELTIVFEPSR

SEQ ID NO: 8 - DNA, 3715 bp, Bos taurus
ggctgacatctgggggcacctttcttccatggctcaggacacacacctgggttgagccaatagaaagactttctggactctgaccgggtgtgcagatgagaatggtaagtgtccgctgcaagctggctcgttacctggaggacctggaagacatagactttaagaaattcaagatgcatttagaagactatcccagtcagaagggctgcacctcaattcctcggggtcagacagaaaaagcagatcacgtggatctagccactctgatgattgatttcaacggggaagagaaggcatgggccatggccaagtggatttttgctgcaatcaacaggagagacctttatgagaaagctaagagggaagagccagaatgggagaatgcaaatatttctgtgctaagtcaggaggaaagccttgaagaagaatggatgggtttactgggatacctttccagaatctctatttgtaggaaaaaaaaagattactgtaagaagtacagaaaatatgtgagaagcaaattccagtgtattaaagacaggaatgcccgtctgggtgagagcgtgaacctcaacaaacgcttcaccaggctgcgtctcatcaaggaacacaggagccaacaggagagggagcatgagctcctggccattggtaggacctgggccaagatacaggatagccctgtgagttctgtgaacttggaattgctgtttgatcctgaggaccagcactctgagcctgtgcacacagtggtattccagggagcagcgggcattgggaaaacaatactggctaggaagatcatgttggactgggcatcagagaaactttaccaggatagatttgactatttgttttacattcactgccgggaggtgagccttgggacgcagaggagtctgggggacctgatcgccagctgctgccctggcccaaacccacccataggcaagattgtaagcaagccttccaggatcctcttcctcatggacggctttgatgagctgcaaggtgcctttgatgagcacacagaagcactctgcacaaactggcggaaggtggagcggggagacattctcctgagcagcctcatcagaaaaagactgcttcctgaggcctccctcctcatcaccacaagacccgtggccctggagaaacttcagcacttgctgggccaggctcgtcatgtggagatcctgggtttctcagaggccaggaggaaggaatatttcttaaagtatttctcagatgagcagcaagcaagggaagccttcagactgattcaggagaatgagatcctcttcaccatgtgctttattcctctggtctgctggattgtgtgcactgggctgaaacagcagatggatagtggtaagagtcttgctcggacatccaagaccaccactgcagtgtatatcttcttcctctccagtttgttgcaatctcagggagggagccaggagaaccacaactctgctaccctctggggtctctgctcactggctgcagatggaatctggaaccagaaaatcctatttcaggagtgtgatctcagaaatcatggcctgcagaaggcagatgtgtctgctttcttgaggatgaacctgttccaaaaggaagtggactgcgagaaattctacagcttcatccacatgactttccaggagttctttgctgccatgtactacctgctggaagaagataatcatggggagatgaggaacacgcctcaggcttgttcaaagcttcccaaccgagatgtgaaggtccttctcgaaaactacggcaaattcgaaaagggatatctgatttttgttgtccgtttcctctttggccttataaaccaggagagaacctcctacttagagaaaaaactgagttgtaagatctctcagaaaatcaggctggagctgctgaaatggattgaagcaaaagccaatgccaagacactacagattgagcccagccagctggaattgttctattgtttgtatgagatgcaggaggaggactttgtgcaaagggccatgagccatttccccaaaattgagatcaagctgtccaccagaatggaccatgtggtttcttctttttgtattgagaactgtcgccatgtggaatccctttccttgaggttgctccataactcacccaaggaggaggaagaagaggaggaagttagacactctcatatggaccgttctgttctctctgattttgaggtggcatattctcagggattggtgaactatctgacctccagcatttgcaggggcatcttctcagtcctgagcaataactggaatctcactgaattgaacctcagtggcaataccctgggagacccaggcatgaacgtgttatgtgaaacactccaacaacctggctgtaacattcgtagattgtggttgggacagtgctgcctttcccatcagtgctgcttcaacatctcctcagtcttgagcaacaaccagaagctggtggagttggacctgagccacaatgccctgggagacttcgggatcagacttctgtgtgtgggactgaggcatctattctgcaatctgaagaagctctggttggtcagttgctgtctcacatcggcaagctgtgaggaccttgcatctgtcctgagcaccaaccattccctgaccagactctacttgggggaaaatgctctgggagactcaggagttgggattttgtgtgaaaaagtaaagaatccacactgtaacttgcaaaaactggggttggtgaattctggccttacatcaggttgttgtccagctctatcctcagtgctcagtacaaatcagaatctcacccacctttacctacagggcaatgctcttggagacatgggggtcaagctactctgtgagggactcttgcaccgcaactgcaagcttcaggtgttggagttagacaactgcagcctcacatcacactgctgctgggatctttccacacttctaacttctaaccagagcctgcgaaagctgtgcctaggcaacaacgacttgggtgatctgggggtcatgcttctctgtgaagtgctcaaacagcagggctgcctcctgaaaagcctgaggttgtgtgaaatgtatttcaactatgacacaaaacgtgccctagaaacacttcaagaagaaaagcctgaattaaccattgtctttgagccttctcggtagtggtggaagcactctgccattcaggtgtggacaagtggtagtgagcattcctccttcagcattaagatcacctctctgcaattcttctggtccagtgaggaagagtgaaaacttgctgatggtccttctgcttggtgaatgtgagaatctcctttacccctaggttgaagacaccagtgcagtaaaatcatcaaatgatacagttcttaccgaagtggctcagtcagaggatgtgttcctcttggtgacctcatgtatctaactcattcaggaaaacactttttctttctttttctctttttactctaactctcctttcctttgtacctctccccactttttttctttgactatatcatctttcctgccatctttctcttaactgaccataacagaatgagttaactatatattatgctgcaattttgtggcagcaacttatttatttttaattttttaactgttatatattctaataaaataactgatttttattgtaaaaaaaaaaaaaaaaaaaaaaaaaa

SEQ ID NO: 9 - protein, 1033 aa, Mus musculus
MTSVRCKLAQYLEDLEDVDLKKFKMHLEDYPPEKGCIPVPRGQMEKADHLDLATLMIDFNGEEKAWAMAVWIFAAINRRDLWEKAKKDQPEWNDTCTSHSSMVCQEDSLEEEWMGLLGYLSRISICKKKKDYCKMYRRHVRSRFYSIKDRNARLGESVDLNSRYTQLQLVKEHPSKQEREHELLTIGRTKMRDSPMSSLKLELLFEPEDGHSEPVHTVVFQGAAGIGKTILARKIMLDWALGKLFKDKFDYLFFIHCREVSLRTPRSLADLIVSCWPDPNPPVCKILRKPSRILFLMDGFDELQGAFDEHIGEVCTDWQKAVRGDILLSSLIRKKLLPKASLLITTRPVALEKLQHLLDHPRHVEILGFSEAKRKEYFFKYFSNELQAREAFRLIQENEVLFTMCFIPLVCWIVCTGLKQQMETGKSLAQTSKTTTAVYVFFLSSLLQSRGGIEEHLFSDYLQGLCSLAADGIWNQKILFEECDLRKHGLQKTDVSAFLRMNVFQKEVDCERFYSFSHMTFQEFFAAMYYLLEEEAEGETVRKGPGGCSDLLNRDVKVLLENYGKFEKGYLIFVVRFLFGLVNQERTSYLEKKLSCKISQQVRLELLKWIEVKAKAKKLQWQPSQLELFYCLYEMQEEDFVQSAMDHFPKIEINLSTRMDHVVSSFCIKNCHRVKTLSLGFFHNSPKEEEEERRGGRPLDQVQCVFPDTHVACSSRLVNCCLTSSFCRGLFSSLSTNRSLTELDLSDNTLGDPGMRVLCEALQHPGCNIQRLWLGRCGLSHQCCFDISSVLSSSQKLVELDLSDNALGDFGIRLLCVGLKHLLCNLQKLWLVSCCLTSACCQDLALVLSSNHSLTRLYIGENALGDSGVQVLCEKMKDPQCNLQKLGLVNSGLTSICCSALTSVLKTNQNFTHLYLRSNALGDTGLRLLCEGLLHPDCKLQMLELDNCSLTSHSCWNLSTILTHNHSLRKLNLGNNDLGDLCVVTLCEVLKQQGCLLQSLQLGEMYLNRETKRALEALQEEKPELTIVFEISW

SEQ ID NO: 10 - DNA, 4021 bp, Mus musculus
ccaggtcctagcctcgtcaccatgggttctggtcagacacgagtcctggtgactttgtatatgcgtgttctctgtataccacatctgattgtgttaatggctttcttatttttatctctacagaggaacttttcttccatggctcaggacatacgtctggatcaagctaagagaactttctgtgtggacctaagccccgagaccctcgaaagggctgctgctgaagatgacgagtgtccgttgcaagctggctcagtatctagaggaccttgaagatgtggacctcaagaaattcaaaatgcatttggaagattacccgcccgagaaaggctgtatcccagtccccaggggccagatggagaaggcagatcacttggatctagccacactcatgattgacttcaatggcgaggagaaggcctgggccatggctgtgtggatctttgctgcgatcaacaggcgagacctctgggaaaaagctaagaaggaccagccagagtggaatgacacgtgtacatcacattcctctatggtatgccaggaggacagccttgaagaagagtggatgggtttgctgggatatctctcccgcatctccatttgtaaaaagaagaaagattactgtaagatgtacagacgacatgtgagaagcaggttctactctatcaaggacaggaacgcgcgtctaggtgagagtgtggacctcaacagtcgctacacgcagctccaactggtcaaggagcatccaagcaagcaggagcgggagcatgaactcctgaccatcggccggactaaaatgcgggacagccccatgagttcccttaagctggagctgctgtttgagcccgaggacgggcactcggagcctgtgcacacagtggtgttccagggagcagcaggcatcgggaaaaccatcctagccaggaagattatgttggactgggcactgggaaagctcttcaaagacaaatttgactatttgttctttatccactgccgagaggtgagcctcaggacgccaaggagtctagcagacctgattgtcagctgctggcctgacccaaacccaccagtgtgcaagatcctgcgcaagccttccaggatcctcttcctcatggatggctttgatgagctacaaggggcctttgacgagcacattggggaggtctgcacagactggcaaaaggctgtgcggggagacattctgctaagcagcctcatccgaaagaaactgctgcccaaggcctctctgctcataacgacgaggccggtagccttggagaaactgcagcatctcctggaccacccccgccatgtggagatcctaggtttctctgaggccaaaaggaaggagtatttctttaagtatttctccaacgagctgcaggcccgggaggccttcaggctgatccaagagaatgaggtcctctttaccatgtgcttcatccccctggtctgctggattgtgtgcacggggctaaagcaacagatggagaccgggaagagcctggcccagacctccaagaccactacggccgtctacgtcttcttcctttccagcctgctgcaatcccgggggggcattgaggagcatctcttctctgactacctacaggggctctgttcactggctgcggatggaatttggaaccagaaaatcctatttgaggagtgtgatctgcggaagcacggcctgcagaagactgacgtctccgctttcctgaggatgaacgtgttccagaaggaagtggactgcgagagattctacagcttcagccacatgactttccaggagttcttcgctgctatgtactatttgctggaagaggaggcagagggggagaccgtgaggaaaggaccaggaggttgttcagatcttctgaaccgagacgtgaaggtcctactagaaaattacggcaagtttgaaaaaggctatctgatttttgttgtccgattcctctttggccttgtaaaccaggagagaacctcttatttggagaagaaactaagttgcaagatctctcagcaagtcagactggaactactgaagtggattgaagtgaaagccaaggccaagaagctgcagtggcagcccagccaactggaactgttctactgcctgtacgagatgcaggaggaagactttgtgcagagtgccatggaccactttcccaaaattgagatcaacctctctaccagaatggaccacgtggtttcctccttttgtattaagaactgtcatagggtcaaaacgctttccctgggtttttttcacaactcgcccaaggaggaagaagaagagaggagaggaggtcgacccttggaccaggttcagtgtgttttcccagacactcatgttgcctgttcttccagactggtgaactgctgcctcacttctagcttctgccgtggtctcttctcaagtctaagcaccaaccggagcctcactgaactggacctcagtgacaatactctgggagacccgggcatgagggtgctgtgtgaggcactccagcacccaggctgtaacattcagagactgtggttggggcgctgcggactgtcccatcaatgctgcttcgacatctcctctgtcctgagcagcagccagaagctggtggagctggacctcagtgacaatgccctgggggactttggaatcagattgctgtgtgtgggactgaagcacctgctctgcaacctccagaaactgtggttggtgagctgctgtctcacatccgcgtgttgtcaggatctcgcattggttctgagctccaaccattctctgaccagactgtacattggagaaaatgccttgggagactcaggagtccaagttttgtgtgaaaagatgaaggacccacagtgtaacttgcagaagctggggttggtgaattccggccttacttcaatctgttgttcagctctgacctctgtgctcaaaaccaaccagaacttcacacacctctatctacgaagcaatgcccttggagacacaggactcaggctcctctgtgaggggcttctgcacccggactgtaaactacagatgctggaattagacaactgcagcctcacctcacacagctgctggaatctctccacaattctgacccacaaccacagccttcggaagctgaacctgggcaacaatgatcttggcgatctgtgcgtggtgaccctctgtgaggtgctgaaacagcagggctgcctcctgcagagcctacagttgggtgaaatgtacttaaatcgtgaaacaaaacgtgccttagaagcgctccaggaagaaaagcctgagctgactatagtcttcgagatttcctggtaggcgtggaagcaggaccaccaggtgcctcggtcctgccccaagtcctgccccaagccccagtgcgcactgctcttcactgctatcaagccctccttcaccatcaggatcacagccgaggctcttctggtatagggtctggagcaaaggcttgtgtgggaccaaatatttttcctcacatcgataacgtgaaactgccagaggctgcccttcccatcatatcctcagtgggcaaggtgttccctcttggtgacttcatggaaacagcttcaagaaaacgccttttctgtcctcccccgccctcctcttactcctgcccctcctcctcctcctcctcccctccccccccctcctcctccgcttctccccccacctgtctttctctctctgggcctctggttttttgacctttgcccataccttcagtcttgtcttcctgttaactgaccatcccgcataaggagctgcccgtgggctagatggaaggtttgtggcagcctctcagctacattgtttgtttttattttttaatagttatgatttctctttagctacctgaaaactcagagatttataaaacccatttttgtatttattgtatgtttgtactgctttcttaatttaaaaatgtatctagaattcttttaagttatttatccaaactactaaaaataaatcagtttacacatttaaaat

SEQ ID NO: 11 - protein, 1035 aa, Rattus norvegicus
MKMMSVRCKLAQYLEDLEDVDLKKFKMHLEDYPPEKGCVPIPRGQMEKADHLDLATLMIDFNGEEKAWGMAVWIFAAINRRDLWEKAKKDQPEWNDACTSNLSMVCQEDSLEEEWIGLLGYLSRISICKKKKDYCKIYRRHVRSRFYSIKDRNARLGESVDLNRRYTQLQLVKEHPSKQEREHELLTIGRTKMWDRPMSSLKLELLFEPEDEHLEPVHTVVFQGAAGIGKTILARKIMLDWALGKLFKDKFDYLFFIHCREVSLRAPKSLADLIISCWPDPNPPVCKILCKPSRILFLMDGFDELQGAFDEHIEEVCTDWQKAVRGDILLSSLIRKKLLPKASLLITTRPVALEKLQHLLDHPRHVEILGFSEAKRKEYFFKYFSNELQAREAFRLIQENEILFTMCFIPLVCWIVCTGLKQQMETGKSLAQTSKTTTAVYVFFLSSLLQSRGGIEEHLFSAYLPGLCSLAADGIWNQKILFEECDLRKHGLQKTDVSAFLRMNVFQKEVDCERFYSFSHMTFQEFFAAMYYLLEEEEEGVTVRKGPEGCSDLLNRDVKVLLENYGKFEKGYLIFVVRFLFGLVNQERTSYLEKKLSCKISQQVRLELLKWIEVKAKAKKLQRQPSQLELFYCLYEMQEEDFVQSAMGHFPKIEINLSTRMDHVVSSFCIKNCHRVKTLSLGFLHNSPKEEEEEKRGSQPLDQVQCVFPDPHVACSSRLVNCCLTSSFCRGLFSSLSTNQSLTELDLSDNTLGDPGMRVLCEALQHPGCNIQRLWLGRCGLTHQCCFNISSVLSSSQKLVELDLSDNALGDFGVRLLCVGLKHLLCNLQKLWLVSCCLTSACCQDLALVLSSNHSLTRLYIGENALGDSGVQVLCEKMKDPQCNLQKLGLVNSGLTSLCCSALTSVLKTNQNLTHLYLRSNALGDMGLKLLCEGLLHPDCKLQMLELDNCSLTSHSCWDLSTILTHNQSLRKLNLSNNDLGDLCVVTLCEVLKQQGCLLQSLQLGEMYLNCETKRTLEALQEEKPELTVVFEISW

SEQ ID NO: 12 - DNA, 3857 bp, Rattus norvegicus
cacgggtcccggtgaccttgtgtgtgcttgttctctgcatgccgtatctggttgtgttaatgcctttcttctttttaatttctacagaggaactttgcttccatggctcaggacatgatcctggatcaagctaagagaactttctaggaagcctttagcttgctcagctcttcctaagtcagagatgacgattcacagaatcaagtcgtagtttttttcctttgagtttttcgagttaagaaactttaaaaactgtgccagtctatggggtaccctgagatgtttcaatattagcatctagtttcctggatttctgctaacgccgttttccggaagtggagacttttgactctttttttactcacacggagttttcattcctgctctgccagcgtggatctaagcccaaagaccctcgcaagaggtgctgatgaagatgatgagtgttcgctgcaagctggcccagtatctagaggacctggaagatgtggacctcaagaaatttaaaatgcatttggaagattacccacccgagaaaggctgtgttccgatccccaggggccagatggagaaggcagatcacttggatctagccacactcatgattgacttcaatggggaggagaaggcgtggggcatggctgtgtggatctttgcagcgatcaacaggcgagacctctgggaaaaagctaagaaggaccagccagagtggaatgatgcatgcacgtctaatctctccatggtgtgccaggaagacagccttgaagaggagtggataggtttgctgggatatctctcccgcatctcgatttgtaaaaaaaagaaagattactgtaagatatacagaaggcatgtgagaagcaggttctactccatcaaagacaggaatgcacgtctaggtgagagcgtggacctcaacagacgctacacccagctccaactggtcaaggagcatccaagcaagcaggaaagggagcatgaactcttgaccattggcaggactaagatgtgggacaggcccatgagctcccttaagctggagctgctctttgagcctgaggatgaacacttggagcccgtgcacacagtggtgttccagggagcagcaggcatcggaaaaacaatcctagccaggaagattatgttggactgggccttggggaagctcttcaaagacaaatttgattatttgttctttatccactgtcgagaggtgagcctcagggcaccaaagagcctagcagatctcattatcagctgctggcctgacccaaacccaccagtctgcaagatcctgtgcaagccttccaggatcctcttcctcatggacggctttgatgagctgcagggggcctttgatgagcacatcgaggaggtctgcacagactggcagaaggctgtgaggggagacattctgctaagcagcctcatccgaaagaagttgctgcctaaggcctctctgcttattacgacgaggccagtagccttggagaagctgcagcatctcctggaccacccccgccacgtggagatcctaggtttctctgaggccaaacggaaggagtatttctttaagtatttctcaaacgagctgcaggccagggaggccttcaggctgatccaagagaatgagatcctgtttactatgtgcttcatccccctggtctgctggattgtgtgcacagggctaaagcagcagatggagactggaaagagcttggcccagacctccaagaccacgactgccgtctatgtctttttcctttccagcctgctgcagtcccgggggggcattgaggagcacctcttctctgcctacctgccagggctctgttcattggctgcggatggaatttggaaccagaagatcctatttgaagagtgtgatctgcggaaacacggcctgcagaagaccgacgtctctgctttcctgagaatgaacgtgttccagaaggaggtggactgcgagagattttacagcttcagccacatgactttccaggagttctttgcggctatgtactatctgctagaagaggaggaagagggggtgaccgtgaggaagggaccagaaggctgttcagaccttctgaaccgagacgtgaaggtcctattagaaaattacggcaagttcgaaaaaggctatctgatttttgttgtacgcttcctctttggccttgtaaaccaggagagaacttcttatttggagaagaagctaagttgcaagatctctcagcaagtcagactggagctgctgaagtggatcgaagtgaaagccaaggccaagaagctgcagaggcagcccagccagctggaactgttctactgcttgtacgagatgcaggaggaagactttgtgcagagcgccatgggccacttccccaaaattgagatcaacctctctaccagaatggaccacgtggtttcttccttttgcattaagaattgccatagggtaaaaacactttccctgggatttctccacaactcacccaaggaggaagaagaagagaaaagaggaagtcaacccttggaccaggtccagtgtgttttcccagaccctcatgttgcctgttcttccagactggtgaactgctgcctcacctctagcttctgccgaggtctcttctcaagtctcagcaccaaccagagcctcactgaactggacctcagtgacaatactctaggggatccaggcatgagggtgctgtgtgaggcactccagcacccaggctgtaacattcagagactgtggttggggcgctgcggactgacccatcaatgctgtttcaacatctcctctgtcctgagcagcagccagaagctggtggagctggacctcagtgacaatgccctgggggactttggagtcagactgctgtgcgtgggactgaagcatctgctctgcaacctgcagaaactctggttggtcagctgctgtctcacatctgcgtgttgtcaggatctcgcattggttctgagctccaaccattctctgaccagactgtacattggagaaaatgccttgggagactcaggagttcaagttttgtgtgaaaaaatgaaggacccacagtgtaacttgcagaagctggggttggtgaattccggccttacttcactgtgctgctcagctctgacctctgtgctcaaaaccaaccagaacctcacacacctgtacctacgaagcaatgcccttggagacatgggactcaagctcctctgtgagggacttctgcaccccgactgtaagctacagatgctggagttagacaactgcagcctcacctcacactcctgctgggatctctccacaattctgacccataaccagagcctccggaagctgaacttgagcaacaacgacctgggcgatctgtgtgtggtgacgctctgtgaggttctgaaacagcagggctgcctcctgcagagcctacagttgggtgaaatgtatttaaattgtgaaacaaaacgtaccttagaagctctccaggaggaaaagcctgagctgactgttgtttttgagatttcttggtaggtgtggaagcaggactgccaggtgccccggtcccaccccaagccctactgttcttcagtgctgcaagccctccttcaccagcaggaccaagccagggcgcttttagtgtagggtctggagcaaaggcctgtgtgggaacaagtatgctttctccagggaagacactgataatacgaaactactagaggacgcccttcctgtcaggtcctcagtcagcaagttgttccctcttggtgacctgatgggaccattttctagaaaatgcattttctgtccttttcctcctcttcctcctcctcttctcccccacttctctctt

SEQ ID NO: 13 - protein, 159 aa, Homo sapiens
SAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 14 - DNA, 2943 bp, Homo sapiens
accaggcaacaccattgaaggctcatatgtaaaaatccatgccttcctttctcccaatctccattcccaaacttagccactggcttctggctgaggccttacgcatacctcccggggcttgcacacaccttcttctacagaagacacaccttgggcatatcctacagaagaccaggcttctctctggtccttggtagagggctactttactgtaacagggccagggtggagagttctctcctgaagctccatcccctctataggaaatgtgttgacaatattcagaagagtaagaggatcaagacttctttgtgctcaaataccactgttctcttctctaccctgccctaaccaggagcttgtcaccccaaactctgaggtgatttatgccttaatcaagcaaacttccctcttcagaaaagatggctcattttccctcaaaagttgccaggagctgccaagtattctgccaattcaccctggagcacaatcaacaaattcagccagaacacaactacagctactattagaactattattattaataaattcctctccaaatctagccccttgacttcggatttcacgatttctcccttcctcctagaaacttgataagtttcccgcgcttccctttttctaagactacatgtttgtcatcttataaagcaaaggggtgaataaatgaaccaaatcaataacttctggaatatctgcaaacaacaataatatcagctatgccatctttcactattttagccagtatcgagttgaatgaacatagaaaaatacaaaactgaattcttccctgtaaattccccgttttgacgacgcacttgtagccacgtagccacgcctacttaagacaattacaaaaggcgaagaagactgactcaggcttaagctgccagccagagagggagtcatttcattggcgtttgagtcagcaaagaagtcaagatggccaaagttccagacatgtttgaagacctgaagaactgttacagtgaaaatgaagaagacagttcctccattgatcatctgtctctgaatcagaaatccttctatcatgtaagctatggcccactccatgaaggctgcatggatcaatctgtgtctctgagtatctctgaaacctctaaaacatccaagcttaccttcaaggagagcatggtggtagtagcaaccaacgggaaggttctgaagaagagacggttgagtttaagccaatccatcactgatgatgacctggaggccatcgccaatgactcagaggaagaaatcatcaagcctaggtcagcaccttttagcttcctgagcaatgtgaaatacaactttatgaggatcatcaaatacgaattcatcctgaatgacgccctcaatcaaagtataattcgagccaatgatcagtacctcacggctgctgcattacataatctggatgaagcagtgaaatttgacatgggtgcttataagtcatcaaaggatgatgctaaaattaccgtgattctaagaatctcaaaaactcaattgtatgtgactgcccaagatgaagaccaaccagtgctgctgaaggagatgcctgagatacccaaaaccatcacaggtagtgagaccaacctcctcttcttctgggaaactcacggcactaagaactatttcacatcagttgcccatccaaacttgtttattgccacaaagcaagactactgggtgtgcttggcaggggggccaccctctatcactgactttcagatactggaaaaccaggcgtaggtctggagtctcacttgtctcacttgtgcagtgttgacagttcatatgtaccatgtacatgaagaagctaaatcctttactgttagtcatttgctgagcatgtactgagccttgtaattctaaatgaatgtttacactctttgtaagagtggaaccaacactaacatataatgttgttatttaaagaacaccctatattttgcatagtaccaatcattttaattattattcttcataacaattttaggaggaccagagctactgactatggctaccaaaaagactctacccatattacagatgggcaaattaaggcataagaaaactaagaaatatgcacaatagcagttgaaacaagaagccacagacctaggatttcatgatttcatttcaactgtttgccttctacttttaagttgctgatgaactcttaatcaaatagcataagtttctgggacctcagttttatcattttcaaaatggagggaataatacctaagccttcctgccgcaacagttttttatgctaatcagggaggtcattttggtaaaatacttcttgaagccgagcctcaagatgaaggcaaagcacgaaatgttattttttaattattatttatatatgtatttataaatatatttaagataattataatatactatatttatgggaaccccttcatcctctgagtgtgaccaggcatcctccacaatagcagacagtgttttctgggataagtaagtttgatttcattaatacagggcattttggtccaagttgtgcttatcccatagccaggaaactctgcattctagtacttgggagacctgtaatcatataataaatgtacattaattaccttgagccagtaattggtccgatctttgactcttttgccattaaacttacctgggcattcttgtttcaattccacctgcaatcaagtcctacaagctaaaattagatgaactcaactttgacaaccatgagaccactgttatcaaaactttcttttctggaatgtaatcaatgtttcttctaggttctaaaaattgtgatcagaccataatgttacattattatcaacaatagtgattgatagagtgttatcagtcataactaaataaagcttgcaacaaaattctctgacaaaaaaaaaaaaaaaa

SEQ ID NO: 15 - protein, 156 aa, Mus musculus
SAPYTYQSDLRYKLMKLVRQKFVMNDSLNQTIYQDVDKHYLSTTWLNDLQQEVKFDMYAYSSGGDDSKYPVTLKISDSQLFVSAQGEDQPVLLKELPETPKLITGSETDLIFFWKSINSKNYFTSAAYPELFIATKEQSRVHLARGLPSMTDFQIS

SEQ ID NO: 16 - DNA, 1974 bp, Mus musculus
aagtctccagggcagagagggagtcaactcattggcgcttgagtcggcaaagaaatcaagatggccaaagttcctgacttgtttgaagacctaaagaactgttacagtgaaaacgaagactacagttctgccattgaccatctctctctgaatcagaaatccttctatgatgcaagctatggctcacttcatgagacttgcacagatcagtttgtatctctgagaacctctgaaacgtcaaagatgtccaacttcaccttcaaggagagccgggtgacagtatcagcaacgtcaagcaacgggaagattctgaagaagagacggctgagtttcagtgagaccttcactgaagatgacctgcagtccataacccatgatctggaagagaccatccaacccagatcagcaccttacacctaccagagtgatttgagatacaaactgatgaagctcgtcaggcagaagtttgtcatgaatgattccctcaaccaaactatatatcaggatgtggacaaacactatctcagcaccacttggttaaatgacctgcaacaggaagtaaaatttgacatgtatgcctactcgtcgggaggagacgactctaaatatcctgttactctaaaaatctcagattcacaactgttcgtgagcgctcaaggagaagaccagcccgtgttgctgaaggagttgccagaaacaccaaaactcatcacaggtagtgagaccgacctcattttcttctggaaaagtatcaactctaagaactacttcacatcagctgcttatccagagctgtttattgccaccaaagaacaaagtcgggtgcacctggcacggggactgccctctatgacagacttccagatatcataaaagcagccttatttcgggagtctattcacttgggaagtgctgacagtctgtatgtaccatgtacaggaaccttcctcaccctgagtcacttgcacagcatgtgctgagtctctgtaattctaaatgaatgtttaccctctttgtaagagaagagcaaaccctagtggagccaccccgacatatgatactatctgttattttaaagagtaccctatagtttgctcagtactaatcattttaattactattctgcatggcattcttaggaggatcaaaaagactctacacatattacagatgggttaacaaagggataaaacaactgaaaagcacactcaatgcatttggaatataaattcacagaccaatctcactgtgcaccttcggcttcaaaatgccagttgagtaggataaaggtataagaacttaatgctgtcattttcaaaaggaaggggacaatagctacatctttcctacctcagtgggttttactccagtgagatcatttggatgaaatcctcctgtaacagacctcaagaaggagacagactgttgaatgttatttttaagttattttatctatgtatttataaatatatttatgataattatattatttatggaacatccttaaatcctctgagcttgacggcaccctcgcagcagggttttctaggtggtcagttagatgtagtctcctctagagctccatgctacagacttttacactttttccacagccacgaagctctccgtacattcctgcacttgggagccctttcatcatgatcttaatctgtgctgtttactttgtgcatctaaaatgataattgagtcagtctttctccctcccgtccttaaagctgtctgggtattcttacatcattcagtctcacctgtaactaacaccaaccatctaaagatggaaagagcttaactgtgacaaccacatcactgatacctgaagtttcttttctagaatgtaatcagtgtttcccctggattccaatttttttttcaaaccacagtgtcatgtaactatcaacaataacaatcaactcattattattaatcataattaaataaaacaggtttgagctg

SEQ ID NO: 17 - protein, 156 aa, Rat norvegicus
SAPHSFQNNLRYKLIRIVKQEFIMNDSLNQNIYVDMDRIHLKAASLNDLQLEVKFDMYAYSSGGDDSKYPVTLKVSNTQLFVSAQGEDKPVLLKEIPETPKLITGSETDLIFFWEKINSKNYFTSAAFPELLIATKEQSQVHLARGLPSMIDFQIS

SEQ ID NO: 18 - DNA, 1992 bp, Rat norvegicus
aaagtctccagggcacagagggagtcaactcattggcgcttgagtcggcaaagaaatcaagatggccaaagttcctgacttgtttgaagacctaaagaactgctatagtgaaaatgaagaatacagttctgccattgaccatctgtctctgaatcagaaatccttctatgatgcaagctatggctcacttcatgagaactgcacagataaatttgtatctctgagaacctctgaaacatcaaagatgtccaccttcaccttcaaggagagccgggtggtggtgtcagcaacatcaaacaaagggaagattctgaagaagagacggctaagtttcaatcagccctttactgaagatgacctggaggccatagcccatgatttagaagagaccatccaacccagatcagcacctcacagcttccagaataatttgagatacaaattgataaggatcgtcaagcaggagttcatcatgaatgattccctcaaccaaaatatatatgtggatatggacagaatacatctcaaagctgcttcgttaaatgacctgcagcttgaagtaaaatttgacatgtatgcctactcatcgggaggagacgactctaaatatcctgtgactctcaaagtctcaaatactcagctctttgtgagtgctcagggagaagacaagcctgtgttgctgaaggagattccggaaacaccaaaactcatcacaggtagtgagaccgacctcattttcttctgggaaaaaatcaactctaagaactacttcacatccgcagctttcccagagctgttaattgccacaaaagaacaaagtcaggtgcacctggcacggggactgccctccatgatagatttccagatatcataaaaacagccttatttaggagtctactttcttgagaagtgctgacaatctgtatgtaccatgtacaggaaatgttcctcgtcctaagtcactcgcatggcatgtgctgagtctttgtaattctaaatgaatgtttaccctctttgtaagagaagagcaaagcctagtggaaccagcccgacatatgatactgtttgttatttaaagagtaccctatagtttgctcactactaatcattttaattattattcttcatggtattcttaggagggtcaaaaagactctgctcatattacagatgggctaactaagggataagacaactgaaaagtacactcaattgcatctggaatgggaattcacagacccatgaaacttatctcactgtgcaccttctgcttctaaagtgccggatgggtaggataaagttgtaagaacgtaatgctatcattttcaaaaggaaggggacaatagctacatctttcctacctcagtgggttttattccagtaagagcatttggataaaatccttctgtaacagacctcaagaaggagacagactgttgaatgttatttttaagttattttatatatgtatttataaatatatttatgataattatattatttatggcacatccttaaatcctctgagcttgccaggcatcctcagcagcagaattttctaggtggtcagttagatgcagtgtcctctgaaacaccatgctatagacagacgtacactgtttccacatccgtggagctctctttacattcttgtccttaggagccctgcagtcatgatcaccaatctgtactgttcacttcgttcatttaaaatgataactgggtcagtcttttgccctcctgtccttaaagctgtctgggtcttcttacatcattcaactcacctgtaattaaagctaaccatctaaagacgggaagaacctaactgtgacaaccacatccctgttacctgaagtttcttttctaggatgtaatcagtgtttcctctgggttccaaatttgttctcaaaccagggtgtcacaaaaatatcaacaataataatcaactcatcattactattgatcataattaaataaagcaagtttgagctg

SEQ ID NO: 19 - protein, 153 aa, Homo sapiens
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 20 - DNA, 1498 bp, Homo sapiens
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 21 - protein, 152 aa, Mus musculus
VPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSS

SEQ ID NO: 22 - DNA, 1348 bp, Mus musculus
aacaaaccctgcagtggttcgaggcctaataggctcatctgggatcctctccagccaagcttccttgtgcaagtgtctgaagcagctatggcaactgttcctgaactcaactgtgaaatgccaccttttgacagtgatgagaatgacctgttctttgaagttgacggaccccaaaagatgaagggctgcttccaaacctttgacctgggctgtcctgatgagagcatccagcttcaaatctcgcagcagcacatcaacaagagcttcaggcaggcagtatcactcattgtggctgtggagaagctgtggcagctacctgtgtctttcccgtggaccttccaggatgaggacatgagcaccttcttttccttcatctttgaagaagagcccatcctctgtgactcatgggatgatgatgataacctgctggtgtgtgacgttcccattagacaactgcactacaggctccgagatgaacaacaaaaaagcctcgtgctgtcggacccatatgagctgaaagctctccacctcaatggacagaatatcaaccaacaagtgatattctccatgagctttgtacaaggagaaccaagcaacgacaaaatacctgtggccttgggcctcaaaggaaagaatctatacctgtcctgtgtaatgaaagacggcacacccaccctgcagctggagagtgtggatcccaagcaatacccaaagaagaagatggaaaaacggtttgtcttcaacaagatagaagtcaagagcaaagtggagtttgagtctgcagagttccccaactggtacatcagcacctcacaagcagagcacaagcctgtcttcctgggaaacaacagtggtcaggacataattgacttcaccatggaatccgtgtcttcctaaagtatgggctggactgtttctaatgccttccccagggcatgttaaggagctcccttttcgtgaatgagcagacagctcaatctccaggggactccttagtcctcggccaagacaggtcgctcagggtcacaagaaaccatggcacattctgttcaaagagagcctgtgttttcctccttgcctctgatgggcaaccacttacctatttatttatgtatttattgattggttgatctatttaagttgattcaaggggacattaggcagcactctctagaacagaacctagctgtcaacgtgtgggggatgaattggtcatagcccgcactgaggtctttcattgaagctgagaataaataggttcctataatatggatgagactttttatgaatgaagcaccagcacattgctttgatgagtatgaaataaatttcattaaaacaaacaaac

SEQ ID NO: 23 - protein, 152 aa, Rattus norvegicus
VPIRQLHCRLRDEQQKCLVLSDPCELKALHLNGQNISQQVVFSMSFVQGETSNDKIPVALGLKGKNLYLSCVMKDGTPTLQLESVDPKQYPKKKMEKRFVFNKIEVKTKVEFESAQFPNWYISTSQAEHRPVFLGNSNGRDIVDFTMEPVSS

SEQ ID NO: 24 - DNA, 1339 bp, Rattus norvegicus
ggctcatctgggatcctctccagtcaggcttccttgtgcaagtgtctgaagcagctatggcaactgtccctgaactcaactgtgaaatagcagctttcgacagtgaggagaatgacctgttctttgaggctgacagaccccaaaagattaaggattgcttccaagcccttgacttgggctgtccagatgagagcatccagcttcaaatctcacagcagcatctcgacaagagcttcaggaaggcagtgtcactcattgtggctgtggagaagctgtggcagctacctatgtcttgcccgtggagcttccaggatgaggacccaagcaccttcttttccttcatctttgaagaagagcccgtcctctgtgactcgtgggatgatgacgacctgctagtgtgtgatgttcccattagacagctgcactgcaggcttcgagatgaacaacaaaaatgcctcgtgctgtctgacccatgtgagctgaaagctctccacctcaatggacagaacataagccaacaagtggtattctccatgagctttgtacaaggagagacaagcaacgacaaaatccctgtggccttgggcctcaaggggaagaatctatacctgtcctgtgtgatgaaagacggcacacccaccctgcagctggagagtgtggatcccaaacaatacccaaagaagaagatggaaaagcggtttgtcttcaacaagatagaagtcaagaccaaagtggagtttgagtctgcacagttccccaactggtacatcagcacctctcaagcagagcacagacctgtcttcctaggaaacagcaatggtcgggacatagttgacttcaccatggaacccgtgtcttcctaaagatggctgcactattcctaatgccttccccaggacatgctagggagcccccttgtcgagaatgggcagtctccaggggaagcctttgtcctctgccaagtcaggtctctcagagccataagaaaaccgtggcacattctggtcaaagaaaacgtgtgtttccctccctgcctctgacaggcaaccacttacctatttatttatgtatttattgattggttgatctatttaagttgattcaggggggtcacgaggcagcattgtcgacagaagaatctagttgtccgtgtgtatgggatgaattgaatttggaccagtgcacagccagcactgagttctttcattgatgctgaaaatgaagagtttcatattgtgtggatgagagtgtttatgaatgaagcacaagcacatcattttgatgagtatgaaataaatgtcactaaaacaaaaaaaaaaaaaaaaaaaaaaaaaaaaaa

(19) United States
(12) Patent Application Publication
(10) Pub. No.: US 2017/0056448 A1
(43) Pub. Date: Mar. 2, 2017

(54) IMMUNE CELLS HAVING INCREASED IMMUNITY OR RESISTANCE TO AN IMMUNOSUPPRESSIVE CYTOKINE AND USE OF THE SAME

(71) Applicant: IFM THERAPEUTICS, INC, Cambridge, MA (US)

(72) Inventors: Gary D. Glick, Ann Arbor, MI (US); Luigi Franchi, Ann Arbor, MI (US)

(73) Assignee: IFM THERAPEUTICS, INC, Cambridge, MA (US)

(21) Appl. No.: 15/253,215

(22) Filed: Aug. 31, 2016

Related U.S. Application Data
(60) Provisional application No. 62/212,747, filed on Sep. 1, 2015.

(51) Int. Cl. A61K 35/17; G01N 33/50; C12N 5/0783; C07K 16/18; C07K 14/725

(52) U.S. Cl. CPC A61K 35/17; C07K 16/18; C07K 14/7051; C12N 5/0636; G01N 33/505; A61K 2035/124

(57) ABSTRACT

Provided herein are immune cells having increased immunity or resistance to an immunosuppressive cytokine and use of the same.

### CROSS-REFERENCE TO RELATED APPLICATION

This application claims priority to U.S. Provisional Patent Application Ser. No. 62/212,747, filed Sep. 1, 2015; the entire contents of which are herein incorporated by reference.

### TECHNICAL FIELD

This invention relates to methods of immunology and the adoptive cell therapy.

### BACKGROUND

T cells of the immune system are known to recognize and interact with specific molecules through one or more receptors (e.g., a T cell receptor in complex with a CD3 dimer) which, upon recognition or interaction with these molecules, result in the activation of the T cell to perform various immune activities. Innate immune cells are cells of the immune system that are known to be activated by one or more agents (e.g., allergens, chemicals produced upon injury (e.g., opioids and alcohols), polymyxins, crosslinked IgE, crosslinked complement proteins, cytokines produced by T cells or other immune cells (e.g., interferon-γ), DAMPs, or PAMPs) that activate downstream signaling pathway(s) in the innate immune cell and result in the activation of one or more immune activites of the innate immune cell.

Both T cells and innate immune cells play a role in a mammal's immune defense. For example, the immune activities of an innate immune cell provides protects a mammal against a variety of different infectious diseases. The immune activities of a T cell protect a mammal against, e.g., different cancers and against different infectious diseases.

Adoptive cell therapy is a method of treatment that includes harvesting one or more different types of immune cells from a mammal, culturing and/or manipulating the harvested immune cells ex vivo, and administering the cultured and/or manipulated immune cells back to the mammal. The manipulating of a harvested immune cell ex vivo can include introducing a recombinant nucleic acid into the immune cell.

### SUMMARY

The present invention is based, at least in part, on the discovery that contacting T cells with an NLRP3 activator causes the T cells to have increased resistance to an immunosuppressive cytokine and also increases the anti-tumor activity of the T-cell. In view of this discovery, provided herein are methods of increasing resistance of a T cell or an innate immune cell to at least one immunosuppressive cytokine that include culturing the T cell or the innate immune cell in a culture medium including an amount of an NLRP3 activator sufficient to induce resistance of the T cell or the innate immune cell to the at least one immunosuppressive. Also provided are methods of increasing the anti-tumor activity of a T cell that include culturing the T cell in a culture medium sufficient to induce and/or increase anaplerosis and/or including a NLRP3 activator in an amount sufficient to improve anti-tumor activity of the T cell, thereby resulting in an increase in the anti-tumor activity of the T cell. Also provided are T cells or innate immune cells produced by these methods, pharmaceutical compositions containing these T cells or innate immune cells, or kits including these pharmaceutical compositions. Also provided are methods of increasing anti-tumor activity in a mammal having a cancer, methods of increasing time of remission of a cancer in a mammal, methods of treating a mammal having a cancer or an infectious disease, methods of increasing the time of survival of a mammal having a cancer, methods of decreasing the size of a solid tumor in a mammal having a cancer, methods of improving the prognosis of a mammal having a cancer, methods of decreasing the risk of developing a metastasis or an additional metastasis in a mammal having a cancer, and methods of increasing the level of at least one anti-tumor lymphokine and/or at least one anti-tumor cytokine in a mammal having a cancer that include (in part): (i) harvesting a T cell from the mammal, and culturing the T cell in a culture medium sufficient to induce and/or increase anaplerosis and/or including a NLRP3 activator in an amount sufficient to improve anti-tumor activity of a T cell, and/or under conditions sufficient to induce and/or increase anaplersosis, and administering the T cell to the mammal or (ii) administering a therapeutically effective amount of a NLRP3 activator to the mammal. Also provided are methods for maintaining a population of recombinant T cells in a mammal for at least one month (e.g., at least two months or at least one month after administering the recombinant T cell to the mammal that include: harvesting a T cell from the mammal, introducing a recombinant nucleic acid into the T cell to generate a recombinant T cell, culturing the recombinant T cell in a culture medium sufficient to induce and/or increase anaplerosis and/or including a NLRP3 activator in an amount sufficient to increase anti-tumor activity, and administering the recombinant T cell to the mammal. Also provided are methods of identifying a candidate agent for increasing resistance of a T cell to at least one immunosuppressive cytokine.

In one aspect, a method of increasing resistance of a T cell or an innate immune cell to at least one immunosuppressive cytokine is provided. Such a method typically includes (a) providing a T cell or an innate immune cell, and (b) culturing the T cell or the innate immune cell in a culture medium including an amount of an NLRP3 activator sufficient to increase resistance of a T cell or an innate immune cell to the at least one immunosuppressive cytokine.

In some embodiments, the culture medium further includes one or more agents that activate the T cell or the innate immune cell. Representative agents that activate the T cell or the innate immune cell includes, without limitation, an isolated tumor antigen, a tumor lysate, necrotic tumor cells, tumor apoptotic bodies, an isolated antigen from a pathogenic organism or virus, or a tumor vaccine. In some embodiments, the innate immune cell is a dendritic cell, a macrophage, or a monocyte.

In some embodiments, culturing the T cell or the innate immune cell in step (b) results in an increase of NLRP3 downstream signaling in the T cell or the innate immune cell. In some embodiments, culturing the T cell or the innate immune cell in step (b) results in an increase in the level of NLRP3 protein in the T cell or the innate immune cell.

In some embodiments, the method includes providing an innate immune cell and culturing the innate immune cell in a culture medium including an amount of an NLRP3 activator sufficient to increase resistance of an innate immune cell to the at least one immunosuppressive cytokine. In some embodiments, the method includes providing a T cell and culturing the T cell in a culture medium including an amount of an NLRP3 activator sufficient to increase resistance of a T cell to the at least one immunosuppressive cytokine.

In some embodiments, the T cell is a CD4⁺ T cell or a CD8⁺ cell. Representative T cells include, without limitation, a lymphoid progenitor cell, an immature thymocyte, a peripheral blood lymphocyte, a naïve T cell, a pluripotent T_{H} cell precursor, a T_{reg} cell, a memory T cell, a T_{H}17 cell, a T_{H}22 cell, a T_{H}9 cell, a T_{H}2 cell, a T_{H}1 cell, a T_{H}3 cell, γδ T cell, an αβ T cell, and a tumor-infiltrating T cell. In some embodiments, the T cell is a chimeric antigen receptor (CAR)-T cell. In some embodiments, the CAR-T cell includes a nucleic acid encoding a CAR protein including an antigen-binding domain, a transmembrane domain, and a cytoplasmic signaling domain. Representative antigen-binding domain include, without limitation, an antibody, an antigen-binding fragment of an antibody, a Fab fragment, and a scFv. In some embodiments, the antibody is a human or humanized antibody, or the antigen-binding fragment of an antibody or the Fab is a fragment of a human or humanized antibody. In some embodiments, the cytoplasmic signaling domain includes a CD3ζ cytoplasmic sequence. In some embodiments, the cytoplasmic signaling domain further includes a cytoplasmic sequence of one or more of the following proteins: CD27, CD28, 4-1BB, OX40, CD30, CD40, PD-1, ICOS, LFA-1, CD2, CD7, LIGHT, NKG2C, B7-H3, and a ligand that specifically binds to CD83. In some embodiments, the transmembrane domain includes a transmembrane sequence from CD3ζ, CD8, or CD28. In some embodiments, the CAR protein further includes a linker sequence between the antigen-binding domain and the transmembrane domain. In some embodiments, the T cell is harvested from a mammal. In some embodiments, the innate immune cell is harvested from a mammal.

In some embodiments, the mammal has been identified as having a cancer or an infectious disease. Representative cancers include, without limitation, acute lymphoblastic leukemia, acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastric cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, gastric cancer, testicular cancer, throat cancer, thyroid cancer, urethral cancer, uterine cancer, vaginal cancer, and vulvar cancer.

Representative infectious diseases include, without limitation, Acinobacter infection, actinomycosis, African sleeping sickness, acquired immunodeficiency syndrome, amebiasis, anaplasmosis, anthrax, Arcanobacterium haemolyticum infection, Argentine hemorrhagic fever, ascariasis, aspergillosis, astrovirus infection, babesiosis, Bacillus cereus infection, bacterial pneumonia, bacterial vaginosis, Bacteroides infection, balantidiasis, Baylisascaris infection, BK virus infection, black piedra, Blastocystic hominis infection, blastomycosis, Bolivian hemorrhagic fever, botulism, Brazilian hemorrhagic fever, brucellosis, bubonic plaque, Burkholderi infection, Buruli ulcer, Calicivirus infection, camptobacteriosis, candidiasis, cat-scratch disease, cellulitis, Chagas disease, chancroid, chickenpox, chikungunya, chlamydia, Chlamydophila pneumoniae infection, cholera, chromoblastomycosis, clonorchiasis, Clostridium difficile infection, coccidioidomycosis, Colorado tick fever, common cold, Creutzfeldt-Jakob disease, Crimean-Congo hemorrhagic fever, crytococcosis, cryptosporidiosis, cutaneous larva migrans, cyclosporiasis, cysticercosis, cytomegalovirus infection, dengue fever, Desmodesmus infection, deintamoebiasis, diphtheria, diphyllobothriasis, dracunculiasis, ebola hemorrhagic fever, echinococcosis, ehrlichiosis, enterobiasis, Enterococcus infection, Enterovirus infection, epidemic typhus, erythema infection, exanthema subitum, fasciolopsiasis, fasciolosis, fatal familial insomnia, filariasis, food poisoning by Clostridium myonecrosis, free-living amebic infection, Fusobacterium infection, gas gangrene, geotrichosis, Gerstmann-Sträussler-Scheinker syndrome, giardiasis, glanders, gnathostomiasis, gonorrhea, granuloma inguinale, Group A streptococcal infection, Group B streptococcal infection, Haemophilus influenzae infection, hand foot and mouth disease, hantavirus pulmonary syndrome, Heartland virus disease, Heliobacter pylori infection, hemolytic-uremic syndrome, hemorrhagic fever with renal syndrome, hepatitis A, hepatitis B, hepatitis C, hepatitis D, hepatitis E, herpes simplex, histoplasmosis, hookworm infection, human bocavirus infection, human ewingii ehrlichiosis, human granulocyte anaplasmosis, human metapneuomovirus infection, human monocytic ehrlichiosis, human papillomavirus infection, human parainfluenza virus infection, hymenolepiasis, Epstein-Barr virus infectious mononucleosis, influenza, isosporiasis, Kawasaki disease, keratitis, Kingella kingae infection, kuru, lassa fever, Legionnaires' disease, Pontiac fever, leishmaniasis, leprosy, leptospirosis, listeriosis, lyme disease, lymphatic filariasis, lymphocytic choriomeningitis, malaria, Marburg hemorrhagic fever, measles, Middle East respiratory syndrome, melioidosis, meningitis, meningococcal disease, metagonimiasis, microsporidiosis, molluscum contagiosum, monkeypox, mumps, murine typhus, mycoplasma pneumonia, mycetoma, myiasis, neonatal conjunctivitis, variant Creutzfeldt-Jakob disease, nocardiosis, onchocerciasis, paracoccidioidomycosis, paragonimiasis, pasteurellosis, pediculosis capitis, pediculosis corporis, pediculosis pubis, pelvic inflammatory disease, pertussis, plague, pneumonia, poliomyelitis, Prevotella infection, primary amoebic meningoencephalitis, progressive multifocal leukoencephalopathy, psittacosis, Q fever, rabies, relapsing fever, respiratory syncytial virus infection, rhinosporidiosis, rhinovirus infection, rickettsial infection, rickettsialpox, Rift Valley Fever, Rocky Mountain spotted fever, rotavirus infection, rubella, salmonellosis, severe acute respiratory syndrome, scabies, schistosomiasis, sepsis, shigellosis, shingles, smallpox, sporothrichosis, staphylococcal food poisoning, staphylococcal infection, staphylococcal infection, strongyloidiasis, subacute sclerosing panencephalitis, syphilis, taeniasis, tetanus, tinea barabe, tinea capitis, tinea corporis, tinea cruris, tinea manum, tinea nigra, tinea pedis, tinea unguium, tinea versicolor, toxocariasis, trachoma, toxoplasmosis, trichinosis, trichomoniasis, trichuriasis, tuberculosis, tularemia, typhoid fever, Ureaplasma urealyticum infection, valley fever, Venezuelan hemorrhagic fever, viral pneumonia, West Nile fever, white piedra, Yersinia psuedotuberculosis infection, yersiniosis, yellow fever, and zygomycosis.

In some embodiments, a method as described herein further includes harvesting the T cell from the mammal. In some embodiments, a method as described herein further includes harvesting the innate immune cell from the mammal. Representative immunosuppressive cytokines include, without limitation, IL-10, TGF-β, IL-1Ra, IL-18Ra, IL-2, IL-3, IL-4, IL-5, IL-6, IL-7, IL-8, IL-9, IL-11, IL-12, IL-13, IL-14, IL-15, IL-16, IL-17, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, PGE2, SCF, G-CSF, CSF-1R, M-CSF, GM-CSF, IFN-α, IFN-β, IFN-γ, IFN-λ, bFGF, CCL2, CXCL1, CXCL8, CXCL12, CX3CL1, CXCR4, and VEGF. In some embodiments, the at least one immunosuppressive cytokine is TGF-β. In some embodiments, the NLRP3 activator is IL-1α or IL-1β. In some embodiments, the NLRP3 activator is a molecule having a molecular weight of less than 5 kDa. Representative molecules having a molecular weight of less than 5 kDa include, for example, imiquimod or resiquimod, or a pharmaceutically acceptable salt thereof.

In some embodiments, the NLRP3 activator is:

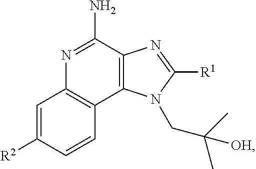

wherein R¹ is H, and R² is H; R¹ is a butyl group and R² is H; R¹ is H and R² is —CO₂CH₃; or R¹ is a butyl group and R² is —CO₂CH₃. Representative NLRP3 activators include, without limitation, an imadazoquinoline; an imidazonaphthyridine; a pyrazolopyridine; an aryl-substituted imidazoquinoline; a compound having a 1-alkoxy 1H-imidazo ring system; an oxazolo [4,5-c]-quinolin-4-amine; an thiazolo [4,5-c]-quinolin-4-amine; a selenazolo [4,5-c]-quinolin-4-amine; an imidazonaphthyridine; an imidazoquinolinamine; a 1-substituted, 2-substituted 1H-imidazo[4,5-C]quinolin-4-amine; a fused cycloalkylimidazopyridine; a 1H-imidazo[4,5-c]quinolin-4-amine; a 1-substituted 1H-imidazo-[4,5-c]quinolin-4-amine; an imidazo-[4,5-C]quinolin-4-amine; a 2-ethyl 1H-imidazo[4,5-ciquinolin-4-amine; an olfenic 1H-imidazo[4,5-c]quinolin-4-amine; a 6,7-dihydro-8-(imidazol-1-yl)-5-methyl-1-oxo-1H,5H-benzo[ij]quinolizine-2-carboxylic acid; a pyridoquinoxaline-6-carboxylic acid; a 6,7-dihydro-8-(imidazol-1-yl)-5-methyl-1-oxo-1H,5H-benzo [ij]quinolizine-2-carboxylic acid; a substituted naphtho[ij]quinolizine; a substituted pyridoquinoxaline-6-carboxylic acid; a 7-hydroxy-benzo[ij]quinolizine-2-carboxylic acid derivative; a substituted benzo[ij]quinolizine-2-carboxylic acid; a 7-hydroxy-benzo[ij]quinolizine-2-carboxylic acid; a substituted pyrido[1,2,3,-de]-1,4-benzoxazine; and a N-methylene malonate of tetrahydroquinoline.

Also provided are T cells having increased resistance to at least one immunosuppressive cytokine produced by any of the methods described herein. Further provided is a pharmaceutical composition that includes such a T cell. Also provided is a kit that includes such a pharmaceutical composition.

Further provided is an innate immune cell having increased resistance to at least one immunosuppressive cytokine produced by any of the methods described herein. Also provided is a pharmaceutical composition that includes such an innate immune cell. Further provided is a kit that includes such a pharmaceutical composition.

In another aspect, a method of improving the anti-tumor activity of a T cell is provided. Such a method typically includes (a) providing a T cell; and (b) culturing the T cell: in a culture medium sufficient to induce and/or increase anaplerosis and/or including a NLRP3 activator in an amount sufficient to improve anti-tumor activity of a T cell; and/or under conditions sufficient to induce and/or increase anaplerosis. Such a method generally results in an improvement in the anti-tumor activity of the T cell.

In some embodiments, such a method includes culturing the T cell in a culture medium sufficient to induce and/or increase anaplerosis. In some embodiments, such a method includes culturing the T cell in a culture medium including a NLRP3 activator in an amount sufficient to improve anti-tumor activity of a T cell. In some embodiments, such a method includes culturing the T cell in a culture medium sufficient to induce and/or increase anaplerosis and including a NLRP3 activator in an amount sufficient to improve anti-tumor activity of a T cell. In some embodiments, the method further includes culturing the T cell under conditions sufficient to induce and/or increase anaplerosis. In some embodiments, the method includes culturing the T cell under conditions sufficient to induce and/or increase anaplerosis.

In some embodiments, the culture medium further includes one or more agents that activate the T cell. Representative agents that activate the T cell include, without limitation, an isolated tumor antigen, tumor lysate, necrotic tumor cells, tumor apoptotic bodies, an isolated antigen from a pathogenic organism or virus, or a tumor vaccine. In some embodiments, the culture medium sufficient to induce and/or increase anaplerosis includes one or more of: (i) galactose without glucose supplementation; (ii) culture media that contains galactose and glucose added in any amount that does not prohibit an induce and/or increase in anaplerosis above rates in control cells; and/or (iii) one or more of 3-bromopyruvate, 2-deoxyglucose, pentavalent arsenic (H₃AsO₄), lonidamine, imatinib, oxythiamine, pyruvate, odd-chain fatty acids, 5-carbon ketone bodies, and triheptanoin.

In some embodiments, the conditions sufficient to induce and/or increase anaplerosis are conditions that result in one or more of the following in the T cell: a decrease in the rate of lactate production from glycolysis, a decrease in the rate of lactate production from glycolysis relative to the rate of oxidative phosphorylation, an increase in the rate of glutamine uptake by the T cell, an increase in the fraction of lipids and/or amino acids synthesized using glutamine as a substrate, an increase in the rate at which pyruvate is converted to oxaloacetate by pyruvate carboxylase, an increase in the rate at which adenylosuccinate synthetase produces fumurate, an increase in the rate at which aspartate aminotransferase produces oxaloacetate, and an increase in the rate at which propionyl-CoA carboxylase produces succinyl-CoA.

In some embodiments, the NLRP3 activator results in an increase in the level of an NLRP3 protein in the T cell. In some embodiments, the NLRP3 activator results in an increase NLRP3 downstream signaling in the T cell. Representative NLRP3 activators include, without limitation, interleukin-1α or interleukin-1β. In some embodiments, the NLRP3 activator is a molecule having a molecular weight of less than 5 kDa. Representative NLRP3 activators having a molecular weight of less than 5 kDa is imiquimod or resiquimod, or a pharmaceutically acceptable salt thereof. In some embodiments, the NLRP3 activator is:

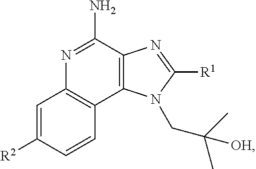

wherein R¹ is H, and R² is H; le is a butyl group and R² is H; le is H and R² is —CO₂CH₃; or R¹ is a butyl group and R² is —CO₂CH₃.

Representative NLRP3 activators include, without limitation, an imadazoquinoline; an imidazonaphthyridine; a pyrazolopyridine; an aryl-substituted imidazoquinoline; a compound having a 1-alkoxy 1H-imidazo ring system; an oxazolo [4,5-c]-quinolin-4-amine; an thiazolo [4,5-c]-quinolin-4-amine; a selenazolo [4,5-c]-quinolin-4-amine; an imidazonaphthyridine, an imidazoquinolinamine; a 1-substituted, 2-substituted 1H-imidazo[4,5-C]quinolin-4-amine; a fused cycloalkylimidazopyridine; a 1H-imidazo[4,5-c]quinolin-4-amine; a 1-substituted 1H-imidazo-[4,5-c]quinolin-4-amine; an imidazo-[4,5-C]quinolin-4-amine; a 2-ethyl 1H-imidazo[4,5-ciquinolin-4-amine; an olfenic 1H-imidazo[4,5-c]quinolin-4-amine; a 6,7-dihydro-8-(imidazol-1-yl)-5-methyl-1-oxo-1H,5H-benzo[ij]quinolizine-2-carboxylic acid; a pyridoquinoxaline-6-carboxylic acid; a 6,7-dihydro-8-(imidazol-1-yl)-5-methyl-1-oxo-1H,5H-benzo [ij]quinolizine-2-carboxylic acid; a substituted naphtho[ij]quinolizine; a substituted pyridoquinoxaline-6-carboxylic acid; a 7-hydroxy-benzo[ij]quinolizine-2-carboxylic acid derivative; a substituted benzo[ij]quinolizine-2-carboxylic acid; a7-hydroxy-benzo[ij]quinolizine-2-carboxylic acid; a substituted pyrido[1,2,3,-de]-1,4-benzoxazine; and a N-methylene malonate of tetrahydroquinoline.

In some embodiments, the T cell is a CD4⁺ T cell or a CD8⁺ cell. Representative T cells include, without limitation, a lymphoid progenitor cell, an immature thymocyte, a peripheral blood lymphocyte, a naïve T cell, a pluripotent T_{H} cell precursor, a T_{reg} cell, a memory T cell, a T_{H}17 cell, a T_{H}22 cell, a T_{H}9 cell, a T_{H}2 cell, a T_{H}1 cell, a T_{H}3 cell, γδ T cell, an αβ T cell, and a tumor-infiltrating T cell.

In some embodiments, the T cell is a chimeric antigen receptor (CAR)-T cell. In some embodiments, the CAR-T cell includes a nucleic acid encoding a CAR protein including: an antigen-binding domain, a transmembrane domain, and a cytoplasmic signaling domain. Representative antigen-binding domains include, without limitation, an antibody, an antigen-binding fragment of an antibody, a Fab fragment, and a scFv. In some embodiments, the antibody is a human or humanized antibody, or the antigen-binding fragment of an antibody or the Fab is a fragment of a human or humanized antibody. Representative cytoplasmic signaling domains include a CD3ζ cytoplasmic sequence. In some embodiments, the cytoplasmic signaling domain further includes a cytoplasmic sequence of one or more of the following proteins: CD27, CD28, 4-1BB, OX40, CD30, CD40, PD-1, ICOS, LFA-1, CD2, CD7, LIGHT, NKG2C, B7-H3, and a ligand that specifically binds to CD83. In some embodiments, the transmembrane domain includes a transmembrane sequence from CD3ζ, CD8, or CD28. In some embodiments, the CAR protein further includes a linker sequence between the antigen-binding domain and the transmembrane domain.

In some embodiments, the T cell is harvested from a mammal. In some embodiments, the mammal has been identified as having a cancer or an infectious disease. Representative cancers include, without limitation, acute lymphoblastic leukemia, acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastric cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, gastric cancer, testicular cancer, throat cancer, thyroid cancer, urethral cancer, uterine cancer, vaginal cancer, and vulvar cancer.

Representative infectious diseases include, without limitation, Acinobacter infection, actinomycosis, African sleeping sickness, acquired immunodeficiency syndrome, amebiasis, anaplasmosis, anthrax, Arcanobacterium haemolyticum infection, Argentine hemorrhagic fever, ascariasis, aspergillosis, astrovirus infection, babesiosis, Bacillus cereus infection, bacterial pneumonia, bacterial vaginosis, Bacteroides infection, balantidiasis, Baylisascaris infection, BK virus infection, black piedra, Blastocystic hominis infection, blastomycosis, Bolivian hemorrhagic fever, botulism, Brazilian hemorrhagic fever, brucellosis, bubonic plaque, Burkholderi infection, Buruli ulcer, Calicivirus infection, camptobacteriosis, candidiasis, cat-scratch disease, cellulitis, Chagas disease, chancroid, chickenpox, chikungunya, chlamydia, Chlamydophila pneumoniae infection, cholera, chromoblastomycosis, clonorchiasis, Clostridium difficile infection, coccidioidomycosis, Colorado tick fever, common cold, Creutzfeldt-Jakob disease, Crimean-Congo hemorrhagic fever, crytococcosis, cryptosporidiosis, cutaneous larva migrans, cyclosporiasis, cysticercosis, cytomegalovirus infection, dengue fever, Desmodesmus infection, deintamoebiasis, diphtheria, diphyllobothriasis, dracunculiasis, ebola hemorrhagic fever, echinococcosis, ehrlichiosis, enterobiasis, Enterococcus infection, Enterovirus infection, epidemic typhus, erythema infection, exanthema subitum, fasciolopsiasis, fasciolosis, fatal familial insomnia, filariasis, food poisoning by Clostridium myonecrosis, free-living amebic infection, Fusobacterium infection, gas gangrene, geotrichosis, Gerstmann-Sträussler-Scheinker syndrome, giardiasis, glanders, gnathostomiasis, gonorrhea, granuloma inguinale, Group A streptococcal infection, Group B streptococcal infection, Haemophilus influenzae infection, hand foot and mouth disease, hantavirus pulmonary syndrome, Heartland virus disease, Heliobacter pylori infection, hemolytic-uremic syndrome, hemorrhagic fever with renal syndrome, hepatitis A, hepatitis B, hepatitis C, hepatitis D, hepatitis E, herpes simplex, histoplasmosis, hookworm infection, human bocavirus infection, human ewingii ehrlichiosis, human granulocyte anaplasmosis, human metapneuomovirus infection, human monocytic ehrlichiosis, human papillomavirus infection, human parainfluenza virus infection, hymenolepiasis, Epstein-Barr virus infectious mononucleosis, influenza, isosporiasis, Kawasaki disease, keratitis, Kingella kingae infection, kuru, lassa fever, Legionnaires' disease, Pontiac fever, leishmaniasis, leprosy, leptospirosis, listeriosis, lyme disease, lymphatic filariasis, lymphocytic choriomeningitis, malaria, Marburg hemorrhagic fever, measles, Middle East respiratory syndrome, melioidosis, meningitis, meningococcal disease, metagonimiasis, microsporidiosis, molluscum contagiosum, monkeypox, mumps, murine typhus, mycoplasma pneumonia, mycetoma, myiasis, neonatal conjunctivitis, variant Creutzfeldt-Jakob disease, nocardiosis, onchocerciasis, paracoccidioidomycosis, paragonimiasis, pasteurellosis, pediculosis capitis, pediculosis corporis, pediculosis pubis, pelvic inflammatory disease, pertussis, plague, pneumonia, poliomyelitis, Prevotella infection, primary amoebic meningoencephalitis, progressive multifocal leukoencephalopathy, psittacosis, Q fever, rabies, relapsing fever, respiratory syncytial virus infection, rhinosporidiosis, rhinovirus infection, rickettsial infection, rickettsialpox, Rift Valley Fever, Rocky Mountain spotted fever, rotavirus infection, rubella, salmonellosis, severe acute respiratory syndrome, scabies, schistosomiasis, sepsis, shigellosis, shingles, smallpox, sporothrichosis, staphylococcal food poisoning, staphylococcal infection, staphylococcal infection, strongyloidiasis, subacute sclerosing panencephalitis, syphilis, taeniasis, tetanus, tinea barabe, tinea capitis, tinea corporis, tinea cruris, tinea manum, tinea nigra, tinea pedis, tinea unguium, tinea versicolor, toxocariasis, trachoma, toxoplasmosis, trichinosis, trichomoniasis, trichuriasis, tuberculosis, tularemia, typhoid fever, Ureaplasma urealyticum infection, valley fever, Venezuelan hemorrhagic fever, viral pneumonia, West Nile fever, white piedra, Yersinia psuedotuberculosis infection, yersiniosis, yellow fever, and zygomycosis.

In some embodiments, a method as described herein further includes harvesting the T cell from the mammal.

Also provided is a T cell having improved resistance to at least one immunosuppressive cytokine produced by any of the methods described herein. Further provided is a pharmaceutical composition that includes such a T cell. Also provided is a kit that includes such a pharmaceutical composition.

In still another aspect, a method of increasing anti-tumor immunity in a mammal having a cancer is provided. Such a method typically includes (a) identifying a mammal having a cancer; (b) harvesting a T cell from the identified mammal; (c) culturing the T cell: in a culture medium sufficient to induce and/or increase anaplerosis and/or including a NLRP3 activator in an amount sufficient to improve anti-tumor activity of a T cell; and/or under conditions sufficient to induce and/or increase anaplerosis, thereby resulting in an improvement in the anti-tumor activity of the T cell; and (d) administering the T cell to a mammal. Such a method generally results in increasing anti-tumor immunity in the mammal.

In yet another aspect, a method of increasing the time of remission of a cancer in a mammal is provided. Such a method typically includes (a) identifying a mammal having a cancer; (b) harvesting a T cell from the identified mammal; (c) culturing the T cell: in a culture medium sufficient to induce and/or increase anaplerosis and/or including a NLRP3 activator in an amount sufficient to improve anti-tumor activity of a T cell; and/or under conditions sufficient to induce and/or increase anaplerosis, thereby resulting in an improvement in the anti-tumor activity of the T cell; and (c) administering the T cell to the mammal. Such a method generally results in an increase in the time of remission of a cancer in the mammal.

In another aspect, a method of treating a mammal having a cancer or an infectious disease is provided. Such a method typically includes (a) identifying a mammal having a cancer or an infectious disease; (b) harvesting a T cell from the identified mammal; (c) culturing the T cell: in a culture medium sufficient to induce and/or increase anaplerosis and/or including a NLRP3 activator in an amount sufficient to improve anti-tumor or anti-infectious disease activity of a T cell; and/or under conditions sufficient to induce and/or increase anaplerosis, to thereby result in an improvement in the anti-tumor or anti-infectious disease activity of the T cell; and administering the T cell to the mammal.

In still another aspect, a method of increasing the time of survival of a mammal having a cancer is provided. Such a method typically includes (a) identifying a mammal having a cancer; (b) harvesting a T cell from the identified mammal; (c) culturing the T cell: in a culture medium sufficient to induce and/or increase anaplerosis and/or including a NLRP3 activator in an amount sufficient to improve anti-tumor activity of a T cell; and/or under conditions sufficient to induce and/or increase anaplerosis, to thereby result in an improvement in the anti-tumor activity of the T cell; and (d) administering the T cell to the mammal.

In another aspect, a method of decreasing the size of a solid tumor in a mammal having a cancer is provided. Such a method typically includes (a) identifying a mammal having a cancer and a solid tumor; (b) harvesting a T cell from the identified mammal; (c) culturing the T cell: in a culture medium sufficient to induce and/or increase anaplerosis and/or including a NLRP3 activator in an amount sufficient to improve anti-tumor activity of a T cell; and/or under conditions sufficient to induce and/or increase anaplerosis, thereby resulting in an improvement in the anti-tumor activity of the T cell; and (d) administering the T cell to the mammal.

In one aspect, a method of improving the prognosis of a mammal having a cancer is provided. Such a method typically includes (a) identifying a mammal having a cancer; (b) harvesting a T cell from the identified mammal; (c) culturing the T cell: in a culture medium sufficient to induce and/or increase anaplerosis and/or including a NLRP3 activator in an amount sufficient to improve anti-tumor activity of a T cell; and/or under conditions sufficient to induce and/or increase anaplerosis, thereby resulting in an improvement in the anti-tumor activity of the T cell; and (d) administering the T cell to the mammal.

In still another aspect, a method of decreasing the risk of developing a metastasis or an additional metastasis in a mammal having a cancer is provided. Such a method typically includes (a) identifying a mammal having a cancer; (b) harvesting a T cell from the identified mammal; (c) culturing the T cell: in a culture medium sufficient to induce and/or increase anaplerosis and/or including a NLRP3 activator in an amount sufficient to improve anti-tumor activity of a T cell; and/or under conditions sufficient to induce and/or increase anaplerosis, thereby resulting in an improvement in the anti-tumor activity of the T cell; and (d) administering the T cell to the mammal.

In yet another aspect, a method of increasing the level of at least one anti-tumor lymphokine and/or at least one anti-tumor cytokine in a mammal having a cancer is provided. Such a method typically includes (a) identifying a mammal having a cancer; (b) harvesting a T cell from the identified mammal; (c) culturing the T cell: in a culture medium sufficient to induce and/or increase anaplerosis and/or including a NLRP3 activator in an amount sufficient to improve anti-tumor activity of a T cell; and/or under conditions sufficient to induce and/or increase anaplerosis, to thereby result in an improvement in the anti-tumor activity of the T cell; and (d) administering the T cell to the mammal.

In another aspect, a method for maintaining a population of recombinant T cells in a mammal for at least one month after administering the recombinant T cell to the mammal is provided. Such a method typically includes (a) identifying a mammal having a cancer; (b) harvesting a T cell from the identified mammal; (c) introducing a recombinant nucleic acid into the T cell to generate a recombinant T cell; (d) culturing the recombinant T cell: in a culture medium sufficient to induce and/or increase anaplerosis and/or including a NLRP3 activator in an amount sufficient to improve anti-tumor activity of a T cell; and/or under conditions sufficient to induce and/or increase anaplerosis, thereby resulting in an improvement in the anti-tumor activity of the recombinant T cell; and (d) administering the recombinant T cell to the mammal, whereby a population of the recombinant T cells persists in the mammal for at least one month after the administering of the recombinant T cell to the mammal.

In some embodiments, the method includes culturing the T cell in a culture medium sufficient to induce and/or increase anaplerosis. In some embodiments, the method includes culturing the recombinant T cell in a culture medium sufficient to induce and/or increase anaplerosis. In some embodiments, the method includes culturing the T cell in a culture medium including a NLRP3 activator in an amount sufficient to improve anti-tumor activity of a T cell. In some embodiments, the method includes culturing the recombinant T cell in a culture medium including a NLRP3 activator in an amount sufficient to improve anti-tumor activity of a T cell. In some embodiments, the method includes culturing the T cell in a culture medium sufficient to induce and/or increase anaplerosis and including a NLRP3 activator in an amount sufficient to improve anti-tumor activity of a T cell.

In some embodiments, the method includes culturing the recombinant T cell in a culture medium sufficient to induce and/or increase anaplerosis and including a NLRP3 activator in an amount sufficient to improve anti-tumor activity of a T cell. In some embodiments, the method further includes culturing the T cell under conditions sufficient to induce and/or increase anaplerosis. In some embodiments, the method further includes culturing the recombinant T cell under conditions sufficient to induce and/or increase anaplerosis. In some embodiments, the method includes culturing the T cell under conditions sufficient to induce and/or increase anaplerosis. In some embodiments, the method includes culturing the recombinant T cell under conditions sufficient to induce and/or increase anaplerosis.

In some embodiments, the culture medium further includes one or more agents that activate the T cell. In some embodiments, the culture medium includes one or more agents that activate the recombinant T cell. Representative agents that activate the T cell include, without limitation, an isolated tumor antigen, an isolated antigen from a pathogenic organism or virus, or a tumor vaccine. Representative agents that activate the recombinant T cell include, without limitation, an isolated tumor antigen, an isolated antigen from a pathogenic organism or virus, or a tumor vaccine.

In some embodiments, the culture medium sufficient to induce and/or increase anaplerosis includes one or more of: (i) galactose without glucose supplementation; (ii) culture media that contains galactose and glucose added in any amount that does not prohibit an increase in anaplerosis above rates in control cells; and/or (iii) one or more of 3-bromopyruvate, 2-deoxyglucose, pentavalent arsenic (H₃AsO₄), lonidamine, imatinib, oxythiamine, pyruvate, odd-chain fatty acids, 5-carbon ketone bodies, and triheptanoin.

In some embodiments, the conditions sufficient to induce and/or increase anaplerosis are conditions that result in one or more of the following in the T cell: a decrease in the rate of lactate production from glycolysis, a decrease in the rate of lactate production from glycolysis relative to the rate of oxidative phosphorylation, an increase in the rate of glutamine uptake by the T cell, an increase in the fraction of lipids and/or amino acids synthesized using glutamine as a substrate, an increase in the rate at which pyruvate is converted to oxaloacetate by pyruvate carboxylase, an increase in the rate at which adenylosuccinate synthetase produces fumurate, an increase in the rate at which aspartate aminotransferase produces oxaloacetate, and an increase in the rate at which propionyl-CoA carboxylase produces succinyl-CoA.

In some embodiments, the NLRP3 activator results in an increase in the level of an NLRP3 protein in the T cell. In some embodiments, the NLRP3 activator results in an increase in the level of an NLRP3 protein in the recombinant T cell. In some embodiments, the NLRP3 activator results in an increase NLRP3 downstream signaling in the T cell. In some embodiments, the NLRP3 activator results in an increase in NLRP3 downstream signaling in the recombinant T cell. In some embodiments, the NLRP3 activator is interleukin-1α or interleukin-1β. In some embodiments, the NLRP3 activator is a molecule having a molecular weight of less than 5 kDa. In some embodiments, the NLRP3 activator is imiquimod or resiquimod, or a pharmaceutically acceptable salt thereof. In some embodiments, the NLRP3 activator is:

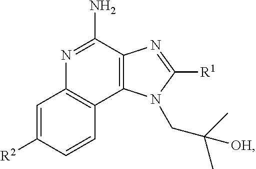

wherein R¹ is H, and R² is H; le is a butyl group and R² is H; R¹ is H and R² is —CO₂CH₃; or R¹ is a butyl group and R² is —CO₂CH₃.

Representative NLRP3 activators included, without limitation, an imadazoquinoline; an imidazonaphthyridine; a pyrazolopyridine; an aryl-substituted imidazoquinoline; a compound having a 1-alkoxy 1H-imidazo ring system; an oxazolo [4,5-c]-quinolin-4-amine; an thiazolo [4,5-c]-quinolin-4-amine; a selenazolo [4,5-c]-quinolin-4-amine; an imidazonaphthyridine, an imidazoquinolinamine, a 1-substituted, 2-substituted 1H-imidazo[4,5-C]quinolin-4-amine; a fused cycloalkylimidazopyridine; a 1H-imidazo[4,5-c]quinolin-4-amine; a 1-substituted 1H-imidazo-[4,5-c]quinolin-4-amine; an imidazo-[4,5-C]quinolin-4-amine; a 2-ethyl 1H-imidazo[4,5-ciquinolin-4-amine; an olfenic 1H-imidazo[4,5-c]quinolin-4-amine; a 6,7-dihydro-8-(imidazol-1-yl)-5-methyl-1-oxo-1H,5H-benzo[ij]quinolizine-2-carboxylic acid; a pyridoquinoxaline-6-carboxylic acid; a 6,7-dihydro-8-(imidazol-1-yl)-5-methyl-1-oxo-1H,5H-benzo [ij]quinolizine-2-carb oxylic acid; a substituted naphtho[ij]quinolizine; a substituted pyridoquinoxaline-6-carboxylic acid; a 7-hydroxy-benzo[ij]quinolizine-2-carboxylic acid derivative; a substituted benzo[ij]quinolizine-2-carboxylic acid; a7-hydroxy-benzo[ij]quinolizine-2-carboxylic acid; a substituted pyrido[1,2,3,-de]-1,4-benzoxazine; and a N-methylene malonate of tetrahydroquinoline.

In some embodiments, the T cell is a CD4⁺ T cell or a CD8⁺ cell. Representative T cells include, without limitation, a lymphoid progenitor cell, an immature thymocyte, a peripheral blood lymphocyte, a naïve T cell, a pluripotent T_{H} cell precursor, a T_{reg} cell, a memory T cell, a T_{H}17 cell, a T_{H}22 cell, a T_{H}9 cell, a T_{H}2 cell, a T_{H}1 cell, a T_{H}3 cell, γδ T cell, an αβ T cell, and a tumor-infiltrating T cell. In some embodiments, the T cell is a chimeric antigen receptor (CAR)-T cell. In some embodiments, the recombinant T cell is a CAR-T cell. In some embodiments, the CAR-T cell includes a nucleic acid encoding a CAR protein including: an antigen-binding domain, a transmembrane domain, and a cytoplasmic signaling domain. Representative antigen-binding domains include, without limitation, an antibody, an antigen-binding fragment of an antibody, a Fab fragment, and a scFv. In some embodiments, the antibody is a human or humanized antibody, or the antigen-binding fragment of an antibody or the Fab is a fragment of a human or humanized antibody. In some embodiments, the cytoplasmic signaling domain includes a CD3ζ cytoplasmic sequence. In some embodiments, the cytoplasmic signaling domain further includes a cytoplasmic sequence of one or more of the following proteins: CD27, CD28, 4-1BB, OX40, CD30, CD40, PD-1, ICOS, LFA-1, CD2, CD7, LIGHT, NKG2C, B7-H3, and a ligand that specifically binds to CD83. In some embodiments, the transmembrane domain includes a transmembrane sequence from CD3ζ, CD8, or CD28. In some embodiments, the CAR protein further includes a linker sequence between the antigen-binding domain and the transmembrane domain.

In some embodiments, the mammal has been identified as having a cancer. Representative cancers include, without limitation, acute lymphoblastic leukemia, acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastric cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, gastric cancer, testicular cancer, throat cancer, thyroid cancer, urethral cancer, uterine cancer, vaginal cancer, and vulvar cancer.

In some embodiments, the mammal has been identified as having an infectious disease. Representative infectious diseases include, without limitation, Acinobacter infection, actinomycosis, African sleeping sickness, acquired immunodeficiency syndrome, amebiasis, anaplasmosis, anthrax, Arcanobacterium haemolyticum infection, Argentine hemorrhagic fever, ascariasis, aspergillosis, astrovirus infection, babesiosis, Bacillus cereus infection, bacterial pneumonia, bacterial vaginosis, Bacteroides infection, balantidiasis, Baylisascaris infection, BK virus infection, black piedra, Blastocystic hominis infection, blastomycosis, Bolivian hemorrhagic fever, botulism, Brazilian hemorrhagic fever, brucellosis, bubonic plaque, Burkholderi infection, Buruli ulcer, Calicivirus infection, camptobacteriosis, candidiasis, cat-scratch disease, cellulitis, Chagas disease, chancroid, chickenpox, chikungunya, chlamydia, Chlamydophila pneumoniae infection, cholera, chromoblastomycosis, clonorchiasis, Clostridium difficile infection, coccidioidomycosis, Colorado tick fever, common cold, Creutzfeldt-Jakob disease, Crimean-Congo hemorrhagic fever, crytococcosis, cryptosporidiosis, cutaneous larva migrans, cyclosporiasis, cysticercosis, cytomegalovirus infection, dengue fever, Desmodesmus infection, deintamoebiasis, diphtheria, diphyllobothriasis, dracunculiasis, ebola hemorrhagic fever, echinococcosis, ehrlichiosis, enterobiasis, Enterococcus infection, Enterovirus infection, epidemic typhus, erythema infection, exanthema subitum, fasciolopsiasis, fasciolosis, fatal familial insomnia, filariasis, food poisoning by Clostridium myonecrosis, free-living amebic infection, Fusobacterium infection, gas gangrene, geotrichosis, Gerstmann-Sträussler-Scheinker syndrome, giardiasis, glanders, gnathostomiasis, gonorrhea, granuloma inguinale, Group A streptococcal infection, Group B streptococcal infection, Haemophilus influenzae infection, hand foot and mouth disease, hantavirus pulmonary syndrome, Heartland virus disease, Heliobacter pylori infection, hemolytic-uremic syndrome, hemorrhagic fever with renal syndrome, hepatitis A, hepatitis B, hepatitis C, hepatitis D, hepatitis E, herpes simplex, histoplasmosis, hookworm infection, human bocavirus infection, human ewingii ehrlichiosis, human granulocyte anaplasmosis, human metapneuomovirus infection, human monocytic ehrlichiosis, human papillomavirus infection, human parainfluenza virus infection, hymenolepiasis, Epstein-Barr virus infectious mononucleosis, influenza, isosporiasis, Kawasaki disease, keratitis, Kingella kingae infection, kuru, lassa fever, Legionnaires' disease, Pontiac fever, leishmaniasis, leprosy, leptospirosis, listeriosis, lyme disease, lymphatic filariasis, lymphocytic choriomeningitis, malaria, Marburg hemorrhagic fever, measles, Middle East respiratory syndrome, melioidosis, meningitis, meningococcal disease, metagonimiasis, microsporidiosis, molluscum contagiosum, monkeypox, mumps, murine typhus, mycoplasma pneumonia, mycetoma, myiasis, neonatal conjunctivitis, variant Creutzfeldt-Jakob disease, nocardiosis, onchocerciasis, paracoccidioidomycosis, paragonimiasis, pasteurellosis, pediculosis capitis, pediculosis corporis, pediculosis pubis, pelvic inflammatory disease, pertussis, plague, pneumonia, poliomyelitis, Prevotella infection, primary amoebic meningoencephalitis, progressive multifocal leukoencephalopathy, psittacosis, Q fever, rabies, relapsing fever, respiratory syncytial virus infection, rhinosporidiosis, rhinovirus infection, rickettsial infection, rickettsialpox, Rift Valley Fever, Rocky Mountain spotted fever, rotavirus infection, rubella, salmonellosis, severe acute respiratory syndrome, scabies, schistosomiasis, sepsis, shigellosis, shingles, smallpox, sporothrichosis, staphylococcal food poisoning, staphylococcal infection, staphylococcal infection, strongyloidiasis, subacute sclerosing panencephalitis, syphilis, taeniasis, tetanus, tinea barabe, tinea capitis, tinea corporis, tinea cruris, tinea manum, tinea nigra, tinea pedis, tinea unguium, tinea versicolor, toxocariasis, trachoma, toxoplasmosis, trichinosis, trichomoniasis, trichuriasis, tuberculosis, tularemia, typhoid fever, Ureaplasma urealyticum infection, valley fever, Venezuelan hemorrhagic fever, viral pneumonia, West Nile fever, white piedra, Yersinia psuedotuberculosis infection, yersiniosis, yellow fever, and zygomycosis.

In some embodiments, the T cell is administered by intravenously, intraarterial, or intra-tumor administration. In some embodiments, the recombinant T cell is administered by intravenous, intraarterial, or intra-tumor administration.

In still another aspect, a method of increasing anti-tumor immunity in a mammal having cancer is provided. Such a method typically includes (a) identifying a mammal as having a cancer; and (b) administering a therapeutically effective amount of a NLRP3 activator to the identified mammal.

In yet another aspect, a method of increasing the time of remission of a cancer in a mammal is provided. Such a method typically includes (a) identifying a mammal as having a cancer; and (b) administering a therapeutically effective amount of a NLRP3 activator to the identified mammal.

In another aspect, a method of treating a mammal having a cancer or an infectious disease is provided. Such a method typically includes (a) identifying a mammal having a cancer or an infectious disease; and (b) administering a therapeutically effective amount of a NLRP3 activator to the identified mammal.

In one aspect, a method of increasing the time of survival of a mammal having a cancer is provided. Such a method typically includes (a) identifying a mammal having a cancer; and (b) administering a therapeutically effective amount of a NLRP3 activator to the identified mammal.

In another aspect, a method of decreasing the size of a solid tumor in a mammal having a cancer is provided. Such a method typically includes (a) identifying a mammal having a cancer and a solid tumor; and (b) administering a therapeutically effective amount of a NLRP3 activator to the identified mammal.

In still another aspect, a method of improving the prognosis of a mammal having a cancer is provided. Such a method typically includes (a) identifying a mammal having a cancer; and (b) administering a therapeutically effective amount of a NLRP3 activator to the identified mammal.

In another aspect, a method of decreasing the risk of developing a metastasis or an additional metastasis in a mammal having a cancer is provided. Such a method typically includes (a) identifying a mammal having a cancer; and (b) administering a therapeutically effective amount of a NLRP3 activator to the identified mammal.

In yet another aspect, a method of increasing the level of at least one anti-tumor lymphokine and/or at least one anti-tumor cytokine in a mammal having a cancer is provided. Such a method typically includes (a) identifying a mammal having a cancer; and (b) administering a therapeutically effective amount of a NLRP3 activator to the identified mammal.

In some embodiments, the mammal is identified as having a cancer. Representative cancers include, without limitation, acute lymphoblastic leukemia, acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastric cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, gastric cancer, testicular cancer, throat cancer, thyroid cancer, urethral cancer, uterine cancer, vaginal cancer, and vulvar cancer.

In some embodiments, the mammal is identified as having an infectious disease. Representative infectious diseases include, without limitation, Acinobacter infection, actinomycosis, African sleeping sickness, acquired immunodeficiency syndrome, amebiasis, anaplasmosis, anthrax, Arcanobacterium haemolyticum infection, Argentine hemorrhagic fever, ascariasis, aspergillosis, astrovirus infection, babesiosis, Bacillus cereus infection, bacterial pneumonia, bacterial vaginosis, Bacteroides infection, balantidiasis, Baylisascaris infection, BK virus infection, black piedra, Blastocystic hominis infection, blastomycosis, Bolivian hemorrhagic fever, botulism, Brazilian hemorrhagic fever, brucellosis, bubonic plaque, Burkholderi infection, Buruli ulcer, Calicivirus infection, camptobacteriosis, candidiasis, cat-scratch disease, cellulitis, Chagas disease, chancroid, chickenpox, chikungunya, chlamydia, Chlamydophila pneumoniae infection, cholera, chromoblastomycosis, clonorchiasis, Clostridium difficile infection, coccidioidomycosis, Colorado tick fever, common cold, Creutzfeldt-Jakob disease, Crimean-Congo hemorrhagic fever, crytococcosis, cryptosporidiosis, cutaneous larva migrans, cyclosporiasis, cysticercosis, cytomegalovirus infection, dengue fever, Desmodesmus infection, deintamoebiasis, diphtheria, diphyllobothriasis, dracunculiasis, ebola hemorrhagic fever, echinococcosis, ehrlichiosis, enterobiasis, Enterococcus infection, Enterovirus infection, epidemic typhus, erythema infection, exanthema subitum, fasciolopsiasis, fasciolosis, fatal familial insomnia, filariasis, food poisoning by Clostridium myonecrosis, free-living amebic infection, Fusobacterium infection, gas gangrene, geotrichosis, Gerstmann-Sträussler-Scheinker syndrome, giardiasis, glanders, gnathostomiasis, gonorrhea, granuloma inguinale, Group A streptococcal infection, Group B streptococcal infection, Haemophilus influenzae infection, hand foot and mouth disease, hantavirus pulmonary syndrome, Heartland virus disease, Heliobacter pylori infection, hemolytic-uremic syndrome, hemorrhagic fever with renal syndrome, hepatitis A, hepatitis B, hepatitis C, hepatitis D, hepatitis E, herpes simplex, histoplasmosis, hookworm infection, human bocavirus infection, human ewingii ehrlichiosis, human granulocyte anaplasmosis, human metapneuomovirus infection, human monocytic ehrlichiosis, human papillomavirus infection, human parainfluenza virus infection, hymenolepiasis, Epstein-Barr virus infectious mononucleosis, influenza, isosporiasis, Kawasaki disease, keratitis, Kingella kingae infection, kuru, lassa fever, Legionnaires' disease, Pontiac fever, leishmaniasis, leprosy, leptospirosis, listeriosis, lyme disease, lymphatic filariasis, lymphocytic choriomeningitis, malaria, Marburg hemorrhagic fever, measles, Middle East respiratory syndrome, melioidosis, meningitis, meningococcal disease, metagonimiasis, microsporidiosis, molluscum contagiosum, monkeypox, mumps, murine typhus, mycoplasma pneumonia, mycetoma, myiasis, neonatal conjunctivitis, variant Creutzfeldt-Jakob disease, nocardiosis, onchocerciasis, paracoccidioidomycosis, paragonimiasis, pasteurellosis, pediculosis capitis, pediculosis corporis, pediculosis pubis, pelvic inflammatory disease, pertussis, plague, pneumonia, poliomyelitis, Prevotella infection, primary amoebic meningoencephalitis, progressive multifocal leukoencephalopathy, psittacosis, Q fever, rabies, relapsing fever, respiratory syncytial virus infection, rhinosporidiosis, rhinovirus infection, rickettsial infection, rickettsialpox, Rift Valley Fever, Rocky Mountain spotted fever, rotavirus infection, rubella, salmonellosis, severe acute respiratory syndrome, scabies, schistosomiasis, sepsis, shigellosis, shingles, smallpox, sporothrichosis, staphylococcal food poisoning, staphylococcal infection, staphylococcal infection, strongyloidiasis, subacute sclerosing panencephalitis, syphilis, taeniasis, tetanus, tinea barabe, tinea capitis, tinea corporis, tinea cruris, tinea manum, tinea nigra, tinea pedis, tinea unguium, tinea versicolor, toxocariasis, trachoma, toxoplasmosis, trichinosis, trichomoniasis, trichuriasis, tuberculosis, tularemia, typhoid fever, Ureaplasma urealyticum infection, valley fever, Venezuelan hemorrhagic fever, viral pneumonia, West Nile fever, white piedra, Yersinia psuedotuberculosis infection, yersiniosis, yellow fever, and zygomycosis.

Representative NLRP3 activators include, without limitation, interleukin-1α or interleukin-1β. Representative NLRP3 activators include, for example, a molecule having a molecular weight of less than 5 kDa. Representative NLRP3 activators include, for example, imiquimod or resiquimod, or a pharmaceutically acceptable salt thereof. Representative NLRP3 activator is:

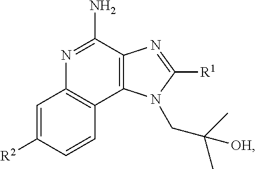

wherein R¹ is H, and R² is H; R¹ is a butyl group and R² is H; is H and R² is —CO₂CH₃; or R¹ is a butyl group and R² is —CO₂CH₃.

Representative NLRP3 activators include, without limitation, an imadazoquinoline; an imidazonaphthyridine; a pyrazolopyridine; an aryl-substituted imidazoquinoline; a compound having a 1-alkoxy 1H-imidazo ring system; an oxazolo [4,5-c]-quinolin-4-amine; an thiazolo [4,5-c]-quinolin-4-amine; a selenazolo [4,5-c]-quinolin-4-amine; an imidazonaphthyridine, an imidazoquinolinamine; a 1-substituted, 2-substituted 1H-imidazo[4,5-C]quinolin-4-amine; a fused cycloalkylimidazopyridine; a 1H-imidazo[4,5-c]quinolin-4-amine; a 1-substituted 1H-imidazo-[4,5-c]quinolin-4-amine; an imidazo-[4,5-C]quinolin-4-amine; a 2-ethyl 1H-imidazo[4,5-ciquinolin-4-amine; an olfenic 1H-imidazo[4,5-c]quinolin-4-amine; a 6,7-dihydro-8-(imidazol-1-yl)-5-methyl-1-oxo-1H,5H-benzo[ij]quinolizine-2-carboxylic acid; a pyridoquinoxaline-6-carboxylic acid; a 6,7-dihydro-8-(imidazol-1-yl)-5-methyl-1-oxo-1H,5H-benzo [ij]quinolizine-2-carboxylic acid; a substituted naphtho[ij]quinolizine; a substituted pyridoquinoxaline-6-carboxylic acid; a 7-hydroxy-benzo[ij]quinolizine-2-carboxylic acid derivative; a substituted benzo[ij]quinolizine-2-carboxylic acid; a7-hydroxy-benzo[ij]quinolizine-2-carboxylic acid; a substituted pyrido[1,2,3,-de]-1,4-benzoxazine; and a N-methylene malonate of tetrahydroquinoline.

In some embodiments, the NLRP3 activator is administered by intravenously, intraarterial, or intra-tumor administration.

In one aspect, a method of identifying a candidate agent for increasing resistance of a T cell to at least one immunosuppressive cytokine is provided. Such a method typically includes (a) providing a mammalian cell that expresses NLRP3 protein; (b) contacting the mammalian cell with an agent; (c) detecting the level of NLRP3 activity in the cell in step (b); and (d) identifying an agent that increases an increased level of NLRP3 activity in the cell as compared to a control level of NLRP3 activity as a candidate agent for increasing resistance of a T cell to at least one immunosuppressive cytokine.

In some embodiments, the control level of NLRP3 activity is the level of NLRP3 activity in the cell in the absence of the agent. In some embodiments, the NLRP3 activity is NLRP3 downstream signaling activity. In some embodiments, the mammalian cell is a T cell. In some embodiments, such a method further includes (e) contacting a T cell with the at least one immunosuppressive cytokine and the candidate agent; and (f) determining the ability of the candidate agent to block the immunosuppressive activity of the at least one immunosuppressive cytokine on the T cell.

As used herein, the word “a” before a noun represents one or more of the particular noun. For example, the phrase “a T cell” represents “one or more T cells.”

The term “T cell” is known in the art and means a type of immune cell that expresses a T-cell receptor (TCR) protein on its cell surface. A variety of different TCRs are known in the art, including, e.g., a TCR including an α and β chain, a TCR including a γ and a δ chain. Non-limiting examples of T cells are descripted herein. Additional examples of T cells are known in the art.

The term “recombinant T cell” means a mammalian T cell that includes a nucleic acid that includes an exogenously-introduced sequence (e.g., a sequence that is not normally present in the mammalian T cell or is not found in a mammalian T cell in its natural state).

The term “innate immune cell” is known in the art and means cells that do not recognize pathogenic material (e.g., cancer cells, bacteria, viruses, and yeast) by expressing an antibody or a TCR on its cell surface, but rather expresses receptors (e.g., receptors on its cell surface) or proteins that bind to the Fc region of other antibodies that are bound to a pathogen and/or receptors that bind to pattern-associated molecular patterns (PAMPs) that are associated with pathogens and/or danger-associated molecular patterns (DAMPs) that are associated with damaged or transformed cells. Non-limiting examples of innate immune cells include mast cells, macrophages, neutrophils, dendritic cells, basophils, eosinophils, and natural killer cells. Additional examples of innate immune cells are known in the art.

The term “mammalian cell” means any cell from or derived from any mammal (e.g., a human, a hamster, a mouse, a green monkey, a rat, a pig, a cow, or a rabbit). For example, a mammalian cell can be an immortalized cell. In some embodiments, the mammalian cell is a differentiated cell. Non-limiting examples of mammalian cells are described herein. Additional examples of mammalian cells are known in the art.

The term “culturing” or “cell culturing” means the maintenance or proliferation of a mammalian cell under a controlled set of physical conditions.

The term “liquid culture medium” means a fluid that contains sufficient nutrients to allow a cell (e.g., a mammalian cell) to grow or proliferate in vitro. For example, a liquid culture medium can contain one or more of: amino acids (e.g., 20 amino acids), a purine (e.g., hypoxanthine), a pyrimidine (e.g., thymidine), choline, inositol, thiamine, folic acid, biotin, calcium, niacinamide, pyridoxine, riboflavin, thymidine, cyanocobalamin, pyruvate, lipoic acid, magnesium, glucose, sodium, potassium, iron, copper, zinc, and sodium bicarbonate. In some embodiments, a liquid culture medium can contain serum from a mammal. In some embodiments, a liquid culture medium does not contain serum or another extract from a mammal (a defined liquid culture medium). In some embodiments, a liquid culture medium can contain trace metals, a mammalian growth hormone, and/or a mammalian growth factor. Another example of liquid culture medium is minimal medium (e.g., a medium containing only inorganic salts, a carbon source, and water). Non-limiting examples of liquid culture medium are described herein. Additional examples of liquid culture medium are known in the art and are commercially available. A liquid culture medium can contain any density of mammalian cells. For example, as used herein, a volume of liquid culture medium removed from a bioreactor can be substantially free of mammalian cells.

The term “animal-derived component free liquid culture medium” means a liquid culture medium that does not contain any components (e.g., proteins or serum) derived from a mammal.

The term “serum-free liquid culture medium” means a liquid culture medium that does not contain a mammalian serum.

The term “serum-containing liquid culture medium” means a liquid culture medium that contains a mammalian serum.

The term “chemically-defined liquid culture medium” is a term of art and means a liquid culture medium in which all of the chemical components are known. For example, a chemically-defined liquid culture medium does not contain fetal bovine serum, bovine serum albumin, or human serum albumin, as these preparations typically contain a complex mix of albumins and lipids.

The term “protein-free liquid culture medium” means a liquid culture medium that does not contain any protein (e.g., any detectable protein).

The term “immunosuppressive cytokine” means a cytokine or lymphokine that leads to suppression of at least one immune function of one or more of a lymphocyte (e.g., including, but not limited to, T_{reg} and innate immune cells), a myeloid suppressor cell, a neutrophil, an eosinophil, a basophil, a cancer-associated fibroblast, an endothelial cell, a cancer cell, and an epithelial cell (e.g., an epithelial cell proximal to a solid tumor). Non-limiting examples of immune functions for lymphocytes, myeloid suppressor cells, neutrophils, eosinophils, basophils, cancer-associated fibroblasts, endothelial cells, cancer cells, and epithelial cells (e.g., epithelial cells proximal to a solid tumor) are described herein. Additional examples of immune functions for lymphocytes, myeloid suppressor cells, neutrophils, eosinophils, basophils, cancer-associated fibroblasts, endothelial cells, cancer cells, and epithelial cells (e.g., epithelial cells proximal to a solid tumor) are known in the art.

For example, suppression of at least one immune function can include increased expression and/or function of at least one (e.g., two, three, four, five, six, seven, eight, nine, ten, eleven, twelve, thirteen, fourteen, fifteen, sixteen, seventeen, eighteen, or nineteen) of LAG3, CTLA4, PDL1, PDL2, PD1, CD80, CD244, TIM3, BTLA4, CD160, B7-H3, B7-H4, BTNL2, VISTA, CD48, HVEM, Galectin, FAS, and FASL, and/or decreased expression and/or function of at least one (e.g., two, three, four, five, six, seven, eight, nine, ten, eleven, twelve, thirteen, fourteen, fifteen, sixteen, seventeen, eighteen, or nineteen) of CD40L, CD40, TL1A, TNFSF25, GITRL, GITR, 41BBL, 4-1BB, OX40L, OX40, CD27, CD70, HHLA2, TMIGD2, ICOS, ICOSL, SIGLEC family, CD28, and LIGHT.

For example, suppression of at least one immune function can included increased expression and/or function at least one (e.g., two, three, four, five, or six) IDO, TDO, ARG1, ARG2, iNOS, and PDES, and/or decreased expression and/or function of P2X7R, and/or increased expression at least one (e.g., two, three, four, five, six, seven, eight, nine, ten, eleven, twelve, thirteen, fourteen, or fifteen) of P2Y₁₁, A2A receptor, CD39, CD73, COX2, EP2 receptor, EP4 receptor, TLR4, TLR7, TLR8, TLR9, RIG-I, MDA-5, CGAS, and STING.

For example, suppression include of at least one immune function can include increased expression and/or function of at least one (e.g., two, three, four, five, or six) ALK5, BRAF^{V600E}, RON, CFS1, PI3Kδ, and PI3Kγ.

For example, suppression of at least one immune function can include increased production/and or function of at least one (e.g., two, three, four, five, six, seven, eight, nine, ten, eleven, twelve, thirteen, fourteen, fifteen, sixteen, seventeen, eighteen, nineteen, or twenty) of IL-10, TGF-β, CSF-1R, GM-CSF, CXCR1, CXCR2, CXCR4, CXCR5, CCR2, CCR5, include IL-10, TGF-β, IL-1Ra, IL-18Ra, IL-2, IL-3, IL-4, IL-5, IL-6, IL-7, IL-8, IL-9, IL-11, IL-12, IL-13, IL-14, IL-15, IL-16, IL-17, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, PGE2, SCF, G-CSF, CSF-1R, M-CSF, GM-CSF, IFN-α, IFN-β, IFN-γ, bFGF, CCL2, CXCL1, CXCL8, CXCL12, CX3CL1, CXCR4, and VEGF, and/or or decreased production and/or function of at least one (e.g., two, three, four, five, six, seven, eight, nine, ten, eleven, twelve, thirteen, or fourteen) of TNF, IL-1α, IL-1β, IL-7, IL-12, IL-15, IL-17, IL-18, IL-21, GM-CSF, IFNα, IFNβ, IFNγ, and IFNλ.

Non-limiting examples of immunosuppressive cytokines include IL-10, TGF-β, IL-1Ra, IL-18Ra, IL-2, IL-3, IL-4, IL-5, IL-6, IL-7, IL-8, IL-9, IL-11, IL-12, IL-13, IL-14, IL-15, IL-16, IL-17, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, PGE2, SCF, G-CSF, CSF-1R, M-CSF, GM-CSF, IFN-α, IFN-β, IFN-γ, IFN-λ, bFGF, CCL2, CXCL1, CXCL8, CXCL12, CX3CL1, CXCR4, and VEGF. Additional examples of immunosuppressive cytokines are known in the art.

The phrase “resistance to at least one immunosuppressive cytokine” means a detectable decrease in the level of suppression of at least one immune function (e.g., at least one of any of the immune functions described herein) in a T cell or an innate immune cell induced by at least one immunosuppressive cytokine. For example, resistance to at least one immunosuppressive cytokine can be a decrease in the level of suppression of at least one immune function (e.g., at least one of any of the immune functions described herein) in a T cell or an innate immune cell induced by at least one immunosuppressive cytokine in the presence of an agent (e.g., a candidate agent or a NLRP3 activator) as compared to the level of suppression of at least one immune function induced by at least one immunosuppressive cytokine in the absence of the agent.

The phrase “anti-tumor activity of a T cell” means an activity of a T cell that contributes to the death of a cancer cell in a mammal. Non-limiting examples of T cell activities that contribute to the death of a cancer cell in a mammal include: differentiation into a T cells, division and proliferation of a T cell, extravasation of a T cell from the blood into a tissue, ability of a T cell to infiltrate a solid tumor, activation of a T cell, ability of T cell to kill tumor cell, ability of T cell to recruit other cell of the immune system to kill tumor cell, ability of T cell to activate factors that kill tumor cell.

The phrase “anti-infectious disease activity of a T cell” means an activity of a T cell that contributes to the death of a microbe or a mammalian cell including a microbe. Non-limiting examples of T cell activities that contribute to the death of a microbe or a mammalian cell including a microbe in a mammal include: differentiation into a T cells, division and proliferation of a T cell, extravasation of a T cell from the blood into a tissue, activation of a T cell, the fability of T cell to recruit other cell of the immune system to kill microbes or mammalian cells including a microbe, and the ability of T cell to activate factors that kill microbes or mammalian cells including a microbe.

The term “anti-tumor cytokine” means a mammalian cytokine that contributes to the death of a cancer cell in a mammal. Non-limiting examples of anti-tumor cytokines are described herein. Additional examples of anti-tumor cytokines are known in the art.

The term “anti-tumor lymphokine” means a mammalian lymphokine that contributes to the death of a cancer cell in a mammal. Non-limiting examples of anti-tumor lymphokines are described herein. Additional examples of anti-tumor lymphokines are known in the art.

The phrase “activate a T cell” is an art known term and means the binding of one or more receptors (e.g., a TCR optionally in association with a CD3 dimer) on the surface of a T cell with one or more cognate receptors on the surface of an antigen-presenting cell. The term activate a T cell can include the binding of one or more integrins on the surface of the T cell with their respective ligands (e.g., intercellular adhesion molecule (ICAM), vascular cell adhesion molecule (VCAM), or fibronectin), or the activation of a co-stimulatory receptor on a T cell (e.g., CD28, ICSO, CD40) by binding to its ligand. For example CD28 is activated by CD80 or CD86, ICOS is activated by ICOS-L, and ICOS is activated by ICOS-L. The activation of a T cell can be performed using, e.g., an isolated tumor antigen, a tumor lysate, necrotic tumor cells, tumor apoptotic bodies, an isolated antigen from a pathogenic organism or virus, or a tumor vaccine. The activation of a T cell can be performed using in part one or more reagents that activate a T cell. Non-limiting examples of methods of activating a T cell are described herein. Additional methods of activating a T cell are known in the art.

The phrase “activate the innate immune cell” is art known and means contacting an innate immune cell with one or more agents (e.g., allergens, chemicals produced upon injury (e.g., opioids and alcohols), polymyxins, crosslinked IgE, crosslinked complement proteins, cytokines produced by T cells or other immune cells (e.g., interferon-γ), DAMPs, or PAMPs) that activate downstream signaling pathway(s) in the innate immune cell and result in the activation of one or more immune activities of the innate immune cell. In some examples, the one or more agents bind to one or more receptors (e.g., FcεR1, pattern recognition receptors (e.g., Toll-like receptors)) on the surface of the innate immune cell which thereby activates downstream signaling pathway(s) in the innate immune cell. Non-limiting examples of DAMPs include nuclear or cytosolic proteins (e.g., HMGB1 protein or S100 protein), DNA or RNA, purine metabolites (e.g., ATP, adenosine, or uric acid), and glycans or glycoconjugates (e.g., hyaluronan fragments). Non-limiting examples of PAMPs include bacterial lipopolysaccharide, flagellin, lipoteichoic acid, peptidoglycan, double-stranded RNA, and unmethylated CpG motifs.

The phrase “antigen-binding domain” is any antigen-binding molecule that can specifically bind to an antigen. Non-limiting examples of an antigen-binding domain include a monoclonal antibody (e.g., IgG1, IgG2, IgG3, IgG4, IgM, IgE, and IgD) (e.g., a fully human or a chimeric (e.g., a humanized) antibody), an antigen-binding fragment of an antibody (e.g., Fab, Fab′, or F(ab′)₂ fragments) (e.g., a fragment of a fully human or a chimeric (e.g., humanized) antibody), a diabody, a linear antibody, a multispecific antibody formed from antibody fragments, and a linear antibody.

An antigen-binding domain that “specifically binds” to a particular antigen when it binds to that antigen, but recognizes and binds to a lesser extent (e.g., does not recognize and bind) to other molecules in a sample. In some embodiments, an antigen-binding domain selectively binds to an antigen with an affinity (KD) equal to or less than 1×10⁻⁷M (e.g., equal to or less than 1×10⁻⁸M, equal to or less than 5×10⁻⁹M, equal to or less than 2×10⁻⁹ M, or equal to or less than 1×10⁻⁹M) in phosphate buffered saline.

The phrase “transmembrane domain” is a molecule that traverses the plasma membrane of a mammalian cell. As used herein, the transmembrane domain can, e.g., be linked at one end to (1) a linker and an antigen-binding domain, or (2) an antigen binding domain, and the other end of the transmembrane domain can, e.g., be linked to the cytoplasmic signaling domain (e.g., a cytoplasmic signaling domain that includes a cytoplasmic sequence of CD3ζ sufficient to provide co-stimulation activity when the antigen-binding domain binds to the antigen, and optionally, a cytoplasmic sequence of one or more of co-stimulatory proteins (e.g., a cytoplasmic sequence of one or more of CD27, CD28, 4-1BB, OX40, CD30, CD40L, CD40, PD-1, PD-L1, ICOS, LFA-1, CD2, CD7, CD160, LIGHT, BTLA, TIM3, CD244, CD80, LAG3, NKG2C, B7-H3, and a ligand that specifically binds to CD83) that provide co-stimulation activity when the antigen-binding domain binds to the antigen. Non-limiting examples and aspects of transmembrane domains are described herein. Additional examples and aspects of transmembrane domains are known in the art.

The phrase “cytoplasmic signaling domain” is a molecule that includes a primary cytoplasmic signaling sequence that stimulates a T cell when an antigen-binding domain of a CAR binds to an antigen (e.g., a sequence of CD3ζ sequence sufficient to stimulate a T cell when the antigen-binding domain binds to the antigen), and optionally, a cytoplasmic sequence of one or more of co-stimulatory proteins (e.g., a cytoplasmic sequence of one or more of CD27, CD28, 4-1BB, OX40, CD30, CD40L, CD40, PD-1, PD-L1, ICOS, LFA-1, CD2, CD7, CD160, LIGHT, BTLA, TIM3, CD244, CD80, LAG3, NKG2C, B7-H3, and a ligand that specifically binds to CD83) that provides for co-stimulation of the T cell.

The phrase “linker sequence” is a molecule that can be placed between a transmembrane domain and an antigen-binding domain and/or between a transmembrane domain and a cytoplasmic signaling domain. Non-limiting examples and aspects of linkers are described herein. Additional examples and aspects of linkers are known in the art.

The phrase “NLRP3 activator” means an agent that having one or more of: the ability to increase the level of an mRNA encoding a NLRP3 protein in a mammalian cell, the ability to increase the level of NLRP3 protein in a mammalian cell, and the ability to promote the downstream signaling of NLRP3 in a mammalian cell.

The phrase “anaplerosis” is art known and means metabolic activity that replenishes citric acid cycle intermediates that have been withdrawn for biosynthesis.

A mammalian cell using anaplerosis can demonstrate or have one or more of: detectable lactate production from glycolysis oxidative phosphorylation, glutamine uptake, a fraction of lipids and/or amino acids synthesized by the cell using glutamine as a substrate, pyruvate carboxylation to form oxaloacetate, adenylosuccinate synthetase activity that produces fumurate, aspartate aminotransferase activity that produces oxaloacetate, and propionyl-CoA carboxylase activity that produces succinyl-CoA.

The phrase “time of survival” means the length of time between the identification or diagnosis of cancer (e.g., any of the cancers described herein) in a mammal by a medical professional and the time of death of the mammal (caused by the cancer). Methods of increasing the time of survival in a mammal having a cancer are described herein.

The phrase “decreasing size of a tumor” means a decrease in the size of a solid tumor over a period of time. Exemplary methods for determining the size of a solid tumor are described herein. Additional methods for determining the size of a solid tumor are known in the art. Methods for decreasing the size of a tumor in a mammal having a cancer are described herein.

The phrase “improving prognosis of a mammal having a cancer” is art known and means a detectable reduction in the severity of a cancer in a mammal over a future period of time. For example, an improved prognosis of a mammal having a cancer can include one or more of: an increased likelihood of having an increased time of survival, a decreased likelihood of developing a metastasis, a decreased likelihood of developing additional metastasis, an increased likelihood of having at least one solid tumor having a decrease in size over time, and an increased likelihood of having an increased time of remission of the cancer in the mammal. Exemplary methods for improving the prognosis of a mammal having a cancer are described herein.

The term “metastasis” is an art known term and means the formation of an additional tumor (e.g., a solid tumor) at a site distant from a primary tumor in a mammal, where the additional tumor includes the same or similar cancer cells as the primary tumor.

The phrase “risk of developing a metastasis” means the risk that a mammal having a primary tumor will develop an additional tumor (e.g., a solid tumor) at a site distant from a primary tumor in a mammal over a set period of time, where the additional tumor includes the same or similar cancer cells as the primary tumor. Methods for reducing the risk of developing a metastasis in a mammal having a cancer are described herein.

The phrase “risk of developing additional metastases” means the risk that a mammal having a primary tumor and one or more additional tumors at sites distant from the primary tumor in a mammal (where the one or more additional tumors include the same or similar cancer cells as the primary tumor) will develop one or more further tumors distant from the primary tumor, where the further tumors include the same or similar cancer cells as the primary tumor. Methods for reducing the risk of developing additional metastasis are described herein.

The phrase “treating a cancer” means decreasing one or more of the severity of a cancer in a mammal, or decreasing the number and/or frequency of one or symptoms of a cancer in a mammal. For example, a decrease in the severity of a cancer can include a decrease in the size of one or more solid tumors in a mammal (e.g., as compared to the size of the one or more tumors prior to treatment). A decrease in the severity of a cancer in a mammal can include a decrease in the rate of growth of one or more tumors in a mammal over time (e.g., as compared to the rate of growth of the one or more tumors prior to treatment). A decrease in the severity of a cancer can also be indicated by an increased time of remission of a cancer in the mammal (e.g., as compared to the average length of remission in a mammal having a similar cancer but receiving a different treatment). A decrease in the severity of a cancer can also, e.g., mean a decrease in the rate of development of metastasis or the rate of development of additional metastasis in a mammal having a cancer (e.g., as compared to a population of mammals having a similar cancer). A decrease in the severity of a cancer can be, e.g., an increase in the time of survival of a mammal having a cancer (e.g., as compared to a population of mammals having a similar cancer). A decrease in the severity of a cancer can, e.g., result in an improved prognosis of a mammal having a cancer. Methods of treating a cancer in a mammal are described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 provides data that show that IL-1β promotes CD8⁺ antitumor activity against established cancer. To induce tumor, 1×10⁶ EG.7 tumor cells, which expressed the OVA antigen, were injected subcutaneously on the flank of Black/6 mice. In this condition, after 7 days EG.7 cells forms palpable tumors. To generate anti-tumor T cells, splenocytes from OT-1 mice, which have a transgenic TCR that recognize OVA antigen, were activated with OVA antigen (1 μg/mL) in the presence of TGF-β, or the presence of TGF-β and IL-1β. After 5 days CD8⁺ cells were purified. To determine the CD8⁺ anti-tumor activity, 5×10⁶ CD8⁺ cells of the different experimental groups (control, TGF-β, TGF-β+IL-1β) were resuspended and administered intraperitoneally in 200 μL of PBS (phosphate buffer saline) in mice with established EG.7 tumors. A control group of mice injected with vehicle alone was used to estimate tumor growth in the absence of treatment. Tumor volume was recorded 3 times a week until the end of the study.

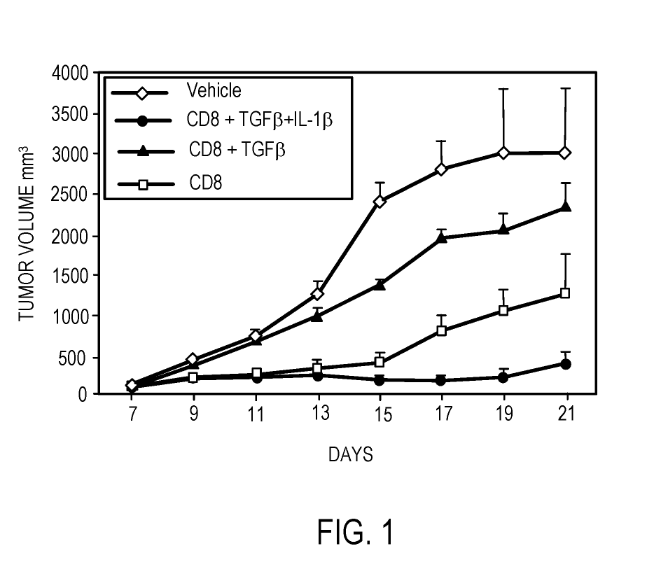

### DETAILED DESCRIPTION

Provided herein are methods of increasing resistance of a T cell or an innate immune cell to at least one immunosuppressive cytokine that include culturing the T cell or the innate immune cell in a culture medium including an amount of an NLRP3 activator sufficient to induce resistance of the T cell or the innate immune cell to the at least one immunosuppressive. Also provided are methods of increasing the anti-tumor activity of a T cell that include culturing the T cell in a culture medium sufficient to increase anaplerosis and/or including a NLRP3 activator in an amount sufficient to induce and/or improve anti-tumor activity of the T cell, and/or under conditions sufficient to induce and/or increase anaplerosis, thereby resulting in an increase in the anti-tumor activity of the T cell. Also provided are T cells or innate immune cells produced by these methods, pharmaceutical compositions containing these T cells or innate immune cells, or kits including these pharmaceutical compositions.

Also provided are methods of increasing anti-tumor activity in a mammal having a cancer, methods of increasing time of remission of a cancer in a mammal, methods of treating a mammal having a cancer or an infectious disease, methods of increasing the time of survival of a mammal having a cancer, methods of decreasing the size of a solid tumor in a mammal having a cancer, methods of improving the prognosis of a mammal having a cancer, methods of decreasing the risk of developing a metastasis or an additional metastasis in a mammal having a cancer, and methods of increasing the level of at least one anti-tumor lymphokine and/or at least one anti-tumor cytokine in a mammal having a cancer that include (in part): (i) harvesting a T cell from the mammal, and culturing the T cell in a culture medium sufficient to induce and/or increase anaplerosis and/or including a NLRP3 activator in an amount sufficient to improve anti-tumor activity of a T cell, and/or under conditions sufficient to induce and/or increase anaplersosis, and administering the T cell to the mammal or (ii) administering a therapeutically effective amount of a NLRP3 activator to the mammal. Also provided are methods for maintaining a population of recombinant T cells in a mammal for at least one month after administering the recombinant T cell to the mammal that include: harvesting a T cell from the mammal, introducing a recombinant nucleic acid into the T cell to generate a recombinant T cell, culturing the recombinant T cell in a culture medium sufficient to induce and/or increase anaplerosis and/or including a NLRP3 activator in an amount sufficient to increase anti-tumor activity, and administering the recombinant T cell to the mammal. Also provided are methods of identifying a candidate agent for increasing resistance of a T cell to at least one immunosuppressive cytokine.

Non-limiting aspects of each of these methods, cells, pharmaceutical compositions, and kits are described below. As one of skill in the art can appreciate, the various aspects described below can be used in any combination, e.g., with aspects known in the art.

### T-Cells

T cells are a type of immune cell that expresses a TCR protein on its cell surface. A variety of different TCRs are known in the art, including, e.g., a TCR including an α and β chain, or a TCR including a γ and a δ chain. Non-limiting examples of T cells include a lymphoid progenitor cell, an immature thymocyte, a peripheral blood lymphocyte, a naïve T cell, a pluripotent T_{H} cell precursor, a T_{reg} cell, a memory T cell, a cytotoxic T cell, a T_{H}17 cell, a T_{H}22 cell, a T_{H}9 cell, a T_{H}2 cell, a T_{H}1 cell, a T_{H}3 cell, γδ T cell, an αβ T cell, and a tumor-infiltrating T cell. A T cell can also be a chimeric antigen receptor (CAR) T cell. Exemplary aspects of CAR-T cells are described below. An additional description of different types of T cells is provided in, e.g., Kuby Immunology (Kindt, Kuby Immunology), Janeway's Immunobiology or Cellular and Molecular Immunology, Abbas.

In some embodiments of any of the methods described herein, the T cell is obtained from a mammal (e.g., a mammal having a cancer or a mammal having an infectious disease). T cells can be obtained from a number of sources, including peripheral blood mononuclear cells, bone marrow, lymph node tissue, cord blood, thymus tissue, tissue from a site of infection, a solid tumor, ascites, pleural effusion, and spleen tissue. In some embodiments, any number of T cell lines available in the art, may be used. In some embodiments, T cells can be obtained from a unit of blood collected from a mammal using any number of techniques known to the skilled artisan, such as Ficoll™ separation. For example, cells from the circulating blood of an individual are obtained by apheresis. An apheresis product typically contains, e.g., lymphocytes, including T cells, monocytes, granulocytes, B cells, other nucleated white blood cells, red blood cells, and platelets. In some examples, the cells collected by apheresis may be washed to remove the plasma fraction and to place the cells in an appropriate buffer or culture media for subsequent handling steps or T-cell isolation steps. For example, the cells can be washed with phosphate buffered saline (PBS) or any other isotonic solution described herein or known in the art. In some embodiments, the wash solution can lack calcium and/or magnesium, or can lack many (if not all) divalent cations. Activation of a T cell performed in the absence of calcium can lead to magnified activation. A washing step can be performed using any methods known to those in the art, such as, e.g., using a semi-automated “flow-through” centrifuge (e.g., the Cobe 2991 ceil processor, the Baxter CytoMate, or the Haemonetics Cell Saver 5) according to the manufacturer's instructions. After washing, the cells may be resuspended in a variety of biocompatible buffers, such as, e.g., Ca²⁺-free, Mg²⁺-free PBS, PlasmaLyte A, or other saline solution including or not including buffer. In some examples, the undesirable components of the apheresis product may be removed and the cells directly resuspended in an appropriate culture medium.

In some examples, T cells are isolated from peripheral blood lymphocytes (e.g., obtained from a mammal) by lysing the red blood cells and depleting the monocytes, e.g., by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺ T cells, can be further isolated by positive or negative selection techniques. In some embodiments, T cells are isolated by incubation with anti-CD3/anti-CD28 (3×28)-conjugated beads, such as DYNABEADS® M-450 CD3/CD28 T, for a time period sufficient for positive selection of the desired T cells. For example, the time period can be, e.g., about 30 minutes, about 30 minutes to 36 hours, or 36 hours or longer. For example, the time period can be, e.g., at least 1, 2, 3, 4, 5, or 6 hours. In some examples, the time period is about 10 to about 24 hours, or about 24 hours. For isolation of T cells from mammals having, identified as having, or diagnosed as having leukemia, use of longer incubation times, e.g., 24 hours or greater than 24 hours, can increase cell yield. Longer incubation times can be used to isolate T cells in any sample where there are few T cells as compared to other cell types, e.g., such as in isolating tumor infiltrating lymphocytes (TIL) from a solid tumor or from immune-compromised mammals. Longer incubation times can, e.g., increase the efficiency of capture of CD8⁺ T cells. As is known in the art, shortening or lengthening the time the T cells are allowed to bind to the CD3/CD28 beads and/or by increasing or decreasing the ratio of beads to T cells, can be used to preferentially select or select against specific subpopulations of T cells at culture inoculation or at other time points during isolation. In some examples, increasing or decreasing the ratio of anti-CD3 and/or anti-CD28 antibodies on the beads or other surface, can be used to preferentially select for or against specific subpopulations of T cells at culture inoculation or at other time point during isolation. As can be appreciated in the art, multiple rounds of selection can also be used to isolate T cells or a specific type of T cell. In some examples, it may be desirable to perform the selection procedure and use the unselected cells in the activation and expansion process. The Unselected cells can, e.g., be subjected to further rounds of selection.

Enrichment of a T cell population by negative selection can, e.g., be accomplished with a combination of antibodies directed to surface markers unique to the negatively selected cells. One exemplary method for enriching a T cell population by negative selection includes cell sorting and/or selection via negative magnetic immunoadherence or flow cytometry that uses a cocktail of one or more monoclonal antibodies directed to one or more cell surface markers present on the one or more cells negatively selected. For example, to enrich for CD4⁺ cells by negative selection, a monoclonal antibody cocktail typically includes, e.g., antibodies to CD14, CD20, CD11b, CD16, HLA-DR, and CD8. In some embodiments, it may be desirable to enrich for or positively select regulatory T cells which typically express CD4⁺, CD25⁺, CD62L1⁺, GITR⁺, and FoxP3⁺. In some examples, T regulatory cells are depleted by anti-C25 conjugated beads or another similar method of selection.

For isolation of a desired population of T cells by positive or negative selection, the concentration of cells and surface {e.g., particles, such as beads) can be varied. In some examples, it may be desirable to significantly decrease the volume in which beads and cells are mixed together {i.e., increase the concentration of cells), to ensure maximum contact of cells and beads. For example, a concentration of about 2 billion cells/mL, about 1 billion cells/mL, about 150 million cells/mL, about 125 million cells/mL, about 100 million cells/mL, about 95 million cells/mL, about 90 million cells/mL, about 85 million cells/mL, about 80 million cells/mL, about 75 million cells/mL, about 70 million cells/mL, about 65 million cells/mL, about 60 million cells/mL, about 55 million cells/mL, about 50 million cells/mL, about 45 million cells/mL, about 40 million cells/mL, about 35 million cells/mL, about 30 million cells/mL, about 25 millions cells/mL, about 20 millions cells/mL, about 15 millions cells/mL, about 10 million cells/mL, or about 5 million cells/mL is used. Using high concentrations can, e.g., result in increased cell yield, cell activation, and cell expansion. In addition, the use of high cell concentrations allows for, e.g., more efficient capture of cells that may weakly express target antigens of interest, such as CD28-negative T cells, or from samples where there are many tumor cells present (e.g., leukemic blood, a solid tissue, etc.). Such populations of cells can have therapeutic value and would be desirable to obtain. For example, using high concentration of cells allows more efficient selection of CD8⁺ T cells that normally have weaker CD28 expression.

In some examples, it may be desirable to use a lower concentrations of cells. By diluting the mixture of T cells and surface (e.g., particles, such as beads), the interactions between the particles and cells is minimized. Diluting the mixture of T cells and surface (e.g., particles, such as beads) selects for T cells that express high amounts of desired antigens to be bound to the particles. For example, CD4⁺ T cells express higher levels of CD28 and are more efficiently captured than CD8⁺ T cells in dilute concentrations. In some examples, the concentration of cells used can be 5×10⁶/mL or about 1×10⁵/mL to 1×10⁶/mL, and any integer value in between.

In some embodiments, the cells may be incubated, e.g., on a rotator, for varying lengths of time at varying speeds at, e.g., 2-10° C. or at room temperature. Some examples further include freezing the cells after the washing step, and before activation. While not being bound by any theory, the freeze and subsequent thaw step can, e.g., provide a more uniform product by removing granulocytes and to some extent monocytes from the cell population. After the washing step that removes plasma and platelets, the cells can be, e.g., suspended in a freezing solution. A variety of different freezing solutions and parameters are known in the art. An exemplary freezing solution that can be used is (1) PBS including 20% DMSO and 8% human serum albumin; (2) culture media including 10% Dextran 40, 5% Dextrose, 20% human serum albumin, and 7.5% DMSO; or (3) culture medium including 31.25% Plasmaiyte-A, 3 1.25% Dextrose 5%, 0.45% NaCl, 10% Dextran 40, 5% Dextrose, 20% Human Serum Albumin, and 7.5% DMSO; or (4) other suitable cell freezing media including for example, Hespan and PlasmaLyte A. In some examples, the cells then are frozen to −80° C. at a rate of about 1° per minute and stored in the vapor phase of a liquid nitrogen storage tank. Additional methods of controlled freezing that can be used are known in the art. In some examples, the cells can be freezing immediately (in an uncontrolled manner) at −20° C. or in liquid nitrogen.

In some examples, the cryopreserved cells are thawed and washed as described herein, and allowed to rest for about one hour at room temperature prior to activation using any of the methods described herein.

In some embodiments, the collection of blood samples or apheresis product from a mammal (e.g., a mammal having a cancer or a mammal having an infectious disease) can be performed at a time point prior to administration of any of the pharmaceutical compositions or cells provided herein. As such, the source of the cells to be handled and manipulated ex vivo can be collected from the mammal at any time point necessary, and the desired cells, such as T cells, can be isolated and frozen for later handling and manipulation ex vivo, before administration back to the mammal.

In some examples, a blood sample or an apheresis product is obtained from a generally healthy mammal (e.g., a mammal not presenting with one or more symptoms of a cancer or an infectious disease). In some embodiments, a blood sample or an apheresis product is obtained from a generally healthy mammal who is at risk of developing a disease (e.g., at risk for developing a cancer or an infectious disease), but who has not yet developed a disease (e.g., a cancer or an infectious disease), and the cells of interest are isolated and frozen for later use (e.g., later culturing, activation, and/or manipulation ex vivo). In some examples, the T cells may be expanded, frozen, and used at a later time. In some examples, the samples are collected from a mammal shortly after diagnosis of a particular disease (e.g., a diagnosis of cancer or a diagnosis of an infectious disease) as described herein but prior to administration of any treatment for the particular disease. In some embodiments, the cells are isolated from a blood sample or an apheresis product from a mammal prior to any number of relevant treatment modalities, including but not limited to treatment with one or more agents such as natalizumab, efalizumab, antiviral agents, chemotherapy, radiation, immunosuppressive agents (e.g., cyclosporin, azathioprine, methotrexate, mycophenolate, and FK506), recombinant antibodies, and other immunoablative agents (e.g., CAMPATH, anti-CD3 antibodies, Cytoxan, fludarabine, cyclosporin, FK506, rapamycin, mycophenolic acid, steroids, FR901228, and irradiation). The immunoablative agents inhibit either the calcium-dependent phosphatase calcineurin (e.g., cyclosporine and FK506) or inhibit the p70S6 kinase that is important for growth factor-induced signaling (e.g., rapamycin) (Liu et al., Cell 66:807-815, 1991); Henderson et al., Immunology 73:316-321, 1991; Bterer et al., Curr. Opin. Immun. 5:763-773, 1993). In some examples, the cells are obtained from a mammal and frozen for later use in conjunction with (e.g., before, simultaneously, or following) bone marrow or stem cell transplantation, T cell ablative therapy using either chemotherapy agents, such as, fludarabine, external-beam radiation therapy (XRT), cyclophosphamide, or antibodies, such as OT3 or CAMPATH. In some examples, the cells are isolated prior to and can be frozen for later use for treatment following B-cell ablative therapy, such as agents that react with CD20, e.g., Rituxan.

In some examples, T cells are obtained from a mammal directly following administration of a treatment. In this regard, it has been observed that following the administration of certain cancer treatments, e.g., drugs that damage the immune system shortly after treatment, during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo proliferation. Thus, some embodiments include collecting blood cells, including T cells or innate immune cells, during this recovery phase. Further, in some examples, mobilization (for example, mobilization by administering GM-CSF) and conditioning regimens can be used to create a condition in a mammal where repopulation, recirculation, regeneration, and/or proliferation of particular cell types is favored, especially during a defined window of time following administration of any of the pharmaceutical compositions or cells provided herein.

### CAR-T Cells

Genetically modifying T cells with a chimeric antigen receptor (CAR) is the most commonly used approach to generate tumor-specific T cells (Sadelain et al., Cancer Discov. 3:388-398, 2013). For example, a T cell is provided and recombinant nucleic acid encoding a chimeric antigen receptor is introduced into the T cell to generate a CAR T cell. Methods for introducing a recombinant nucleic acid into a T cell are known in the art. Non-limiting examples of introducing nucleic acid into a T cell include: calcium phosphate transfection, transfection using highly branched organic compounds, transfection using cationic polymers, transfection using liposomes (e.g., cationic liposomes), electroporation, cell squeezing, sonoporation, optical transfection, protoplast fusion, impalefection, hydrodynamic delivery, gene gun, magnetofection, particle-based transfection, viral transfection, and nucleofection.

Chimeric antigen receptors include an antigen-binding domain, a transmembrane domain, and an cytoplasmic signaling domain that includes a cytoplasmic sequence of CD3ζ sequence sufficient to stimulate a T cell when the antigen-binding domain binds to the antigen, and optionally, a cytoplasmic sequence of one or more (e.g., two, three, or four) co-stimulatory proteins (e.g., a cytoplasmic sequence of one or more of CD27, CD28, 4-1BB, OX40, CD30, CD40L, CD40, PD-1, PD-L1, ICOS, LFA-1, CD2, CD7, CD160, LIGHT, BTLA, TIM3, CD244, CD80, LAG3, NKG2C, B7-H3, and a ligand that specifically binds to CD83) that provides for co-stimulation of the T cell when the antigen-binding domain binds to the antigen. Non-limiting aspects and features of CARs are described below. Additional aspects of CARs and CAR T cells, including exemplary antigen-binding domains, linkers, transmembrane domains, and cytoplasmic signaling domains, are described in, e.g., Kakarla et al., Cancer 120:151-155, 2014; Srivastava et al., Trends Immunol. 36:494-502, 2015; Nishio et al., Oncoimmunology 4(2):e988098, 2015; Ghorashian et al., Br. J. Haematol. 169:463-478, 2015; Levine, Cancer Gene Ther. 22:79-84, 2015; Jensen et al., Curr. Opin. Immunol. 33:9-15, 2015; Singh et al., Cancer Gene Ther. 22:95-100, 2015; Li et al., Zhongguo Shi Yan Xue Ye Xue Za Zhi 22:1753-1756, 2014; Gill et al., Immunol. Rev. 263:68-89, 2015; Magee et al., Discov. Med. 18:265-271, 2014; Gargett et al., Front. Pharmacol. 5:235, 2014; Yuan et al., Zhongguo Shi Yan Xue Ye Xue Za Zhi 22:1137-1141, 2014; Pedgram et al., Cancer J. 20:127-133, 2014; Eshhar et al., Cancer J. 20:123-126, 2014; Ramos et al., Cancer J. 20:112-118, 2014; Maus et al., Blood 123:2625-2635, 2014; Jena et al., Curr. Hematol. Malig. Rep. 9:50-56, 2014; Maher et al., Curr. Gene Ther. 14:35-43, 2014; Riches et al., Discov. Med. 16:295-302, 2013; Cheadle et al., Immunol. Rev. 257:83-90, 2014; Davila et al., Int. J. Hematol. 99:361-371, 2014; Xu et al., Cancer Lett. 343:172-178, 2014; Kochenderfer et al., Nat. Rev. Clin. Oncol. 10:267-276, 2013; Hosing et al., Curr. Hematol. Malig. Rep. 8:60-70, 2013; Hombach et al., Curr. Mol. Med. 13:1079-1088, 2013; Xu et al., Leuk. Lymphoma 54:255-260, 2013; Gilham et al., Trends Mol. Med. 18:377-384, 2012; Lipowska-Bhalla et al., Cancer Immunol. Immunother. 61:953-962, 2012; Chmielewski et al., Cancer Immunol. Immunother. 61:1269-1277, 2013; Jena et al., Blood 116:1035-1044, 2010; U.S. Patent Application Publication Nos. 2015/0232880, 2015/0225480; 2015/0224143; 2015/0224142; 2015/0196599; 2015/0152181; 2015/0140023; 2015/0118202; 2015/0110760; 2015/0099299; 2015/0093822; 2015/0093401; 2015/0051266; 2015/0050729; 2015/0024482; 2015/0023937; 2015/0017141; 2015/0017136; 2015/0017120; 2014/0370045; 2014/0370017; 2014/0369977; 2014/0349402; 2014/0328812; 2014/0322275; 2014/0322216; 2014/0322212; 2014/0322183; 2014/0314795; 2014/0308259; 2014/0301993; 2014/0296492; 2014/0294784; 2014/0286973; 2014/0274909; 2014/0274801; 2014/0271635; 2014/0271582; 2014/0271581; 2014/0271579; 2014/0255363; 2014/0242701; 2014/0242049; 2014/0227272; 2014/0219975; 2014/0170114; 2014/0134720; 2014/0134142; 2014/0120622; 2014/0120136; 2014/0106449; 2014/0106449; 2014/0099340; 2014/0086828; 2014/0065629; 2014/0050708; 2014/0024809; 2013/0344039; 2013/0323214; 2013/0315884; 2013/0309258; 2013/0288368; 2013/0287752; 2013/0287748; 2013/0280221; 2013/0280220; 2013/0266551; 2013/0216528; 2013/0202622; 2013/0071414; 2012/0321667; 2012/0302466; 2012/0301448; 2012/0301447; 2012/0060230; 2011/0213288; 2011/0158957; 2011/0104128; 2011/0038836; and 2007/0036773. Additional aspects of CARs and CAR T cells, including exemplary antigen-binding domains, linkers, transmembrane domains, and cytoplasmic signaling domains, are described in WO 12/079000; 2015/0141347; 2015/0031624; 2015/0030597; 2014/0378389; 2014/0219978; 2014/0206620; 2014/0037628; 2013/0274203; 2013/0225668; 2013/0116167; 2012/0230962; 2012/0213783; 2012/0093842; 2012/0071420; 2012/0015888; 2011/0268754; 2010/0297093; 2010/0158881; 2010/0034834; 2010/0015113; 2009/0304657; 2004/0043401; 2014/0322253; 2015/0118208; 2015/0038684; 2014/0024601; 2012/0148552; 2011/0223129; 2009/0257994; 2008/0160607; 2008/0003683; 2013/0121960; 2011/0052554; and 2010/0178276.

### Antigen Binding Domains

Antigen binding domains included in the chimeric antigen receptor can specifically bind to an antigen (e.g., an antigen of cancer cell or an antigen of a parasite or a parasite infected cell). Non-limiting examples of an antigen binding domain include: a monoclonal antibody (e.g., IgG1, IgG2, IgG3, IgG4, IgM, IgE, and IgD) (e.g., a fully human or a chimeric (e.g., a humanized) antibody); an antigen binding fragment of an antibody (e.g., Fab, Fab′, or F(ab′)₂ fragments) (e.g., a fragment of a fully human or a chimeric (e.g., humanized) antibody); a diabody; a triabody; a tetrabody; a minibody; a scFv; scFv-Fc; scFab; bis-scFv; hc-IgG; a single domain antibody (e.g., a V-NAR domain or a VhH domain); IgNAR; and a multispecific (e.g., bispecific antibody) antibody. Methods of making these antigen-binding domains are known in the art.

An antigen binding domain can include at least one (e.g., one, two, three, four, five, or six) CDR (e.g., any of the three CDRs from an immunoglobulin light chain variable domain or any of the three CDRs from an immunoglobulin heavy chain variable domain) of an antibody that is capable of specifically binding to the target antigen, such as immunoglobulin molecules (e.g., light or heavy chain immunoglobulin molecules) and immunologically-active (antigen-binding) fragments of immunoglobulin molecules.

An antigen binding domain can also be a single-chain antibody (e.g., as described herein). An antigen binding domain can be a whole antibody molecule (e.g., a human, humanized, or chimeric antibody) or a multimeric antibody (e.g., a bi-specific antibody).

Antigen-binding domains also include antibody fragments and multi-specific (e.g., bi-specific) antibodies or antibody fragments. Examples of antibodies and antigen-binding fragments thereof include, but are not limited to: single-chain Fvs (sdFvs), Fab fragments, Fab′ fragments, F(ab′)₂, disulfide-linked Fvs (sdFvs), Fvs, and fragments containing either a VL or a VH domain. The term “single chain Fv” or “scFv” as used herein refers to a polypeptide comprising at least one VL domain of an antibody linked to at least one VH domain of an antibody.

Additional antigen binding domains provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG₁, IgG₂, IgG₃, IgG₄, IgA₁, and IgA₂), or subclass. In some embodiments, the antigen binding domain is an IgG₁ antibody or antigen-binding fragment thereof. In some examples, the antigen binding domain is an IgG₄ antibody or antigen-binding fragment thereof. The antigen binding domain can be an immunoglobulin having a heavy and light chain.

An isolated antigen can be used as an immunogen to generate antibodies using standard techniques for monoclonal antibody preparation. Polyclonal antibodies can be raised in animals by multiple injections (e.g., subcutaneous or intraperitoneal injections) of the antigen. In some embodiments, the antigen is injected with at least one adjuvant. In some embodiments, the antigen can be conjugated to a protein that is immunogenic in the species to be immunized, e.g., keyhole limpet hemocyanin, serum albumin, bovine thyroglobulin, or soybean trypsin inhibitor using a bifunctional or derivitizing agent, for example, malimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups. Animals can be injected with the antigen more than one time (e.g., twice, three times, or four times).

An antigen typically is used to prepare antibodies by immunizing a suitable mammal (e.g., human or transgenic animal expressing at least one human immunoglobulin locus). The preparation used in immunize the mammal can further include an adjuvant, such as Freund's complete or incomplete adjuvant, or a similar immunostimulatory agent.

Polyclonal antibodies can be prepared as described above by immunizing a suitable mammal with the antigen. The antibody titer in the immunized mammal can be monitored over time by standard techniques, such as with an enzyme-linked immunosorbent assay (ELISA) using the antigen. At an appropriate time after immunization, e.g., when the specific antibody titers are highest, antibody-producing cells can be obtained from the mammal and used to prepare monoclonal antibodies by standard techniques, such as the hybridoma technique originally described by Kohler et al., Nature 256:495-497, 1975, the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, N.Y.). Hybridoma cells producing a monoclonal antibody are detected by screening the hybridoma culture supernatants for antibodies that bind the antigen of interest, e.g., using a standard ELISA assay.

As an alternative to preparing monoclonal antibody-secreting hybridomas, a monoclonal antibody directed against an antigen can be identified and isolated by screening a recombinant combinatorial immunoglobulin library (e.g., an antibody phage display library) with the antigen of interest. Kits for generating and screening phage display libraries are commercially available (e.g., the Pharmacia Recombinant Phage Antibody System, Catalog No. 27-9400-01; and the Stratagene SurfZAP* Phage Display Kit, Catalog No. 240612). Additionally, examples of methods and reagents particularly amenable for use in generating and screening an antibody display library can be found in, for example, U.S. Pat. No. 5,223,409; WO 92/18619; WO 91/17271; WO 92/2079; WO 92/15679; WO 93/01288; WO 92/01047; WO 92/09690; WO 90/02809; Fuchs et al., Bio/Technology 9:1370-1372, 1991; Hay et al., Hum. Antibod. Hybridomas 3:81-85, 1992; Huse et al., Science 246:1275-1281, 1989; and Griffiths et al., EMBO J. 12:725-734, 1993.

In some embodiments of any of the methods described herein, the antibodies or antigen-binding fragments are human antibodies, humanized antibodies, or chimeric antibodies that contain a sequence from a human antibody (e.g., a human immunoglobulin constant domain or human immunoglobulin variable domain framework regions). Humanized antibodies are chimeric antibodies that contain a minimal sequence derived from non-human (e.g., mouse) immunoglobulin. In some embodiments, a humanized antibody is a human antibody that has been engineered to contain at least one complementary determining region (CDR) present in a non-human antibody (e.g., a mouse, rat, rabbit, or goat antibody). In some embodiments, a humanized antibody or fragment thereof can contain all three CDRs of a light chain of a non-human antibody or a human antibody that specifically binds to substantially the same antigen. In some embodiments, the humanized antibody or fragment thereof can contain all three CDRs of a heavy chain of a non-human monoclonal antibody or a human antibody that specifically binds to the same antigen. In some embodiments, the framework region residues of the human immunoglobulin are replaced by corresponding non-human (e.g., mouse) antibody residues. In some embodiments, the humanized antibodies can contain residues which are not found in the human antibody or in the non-human (e.g., mouse) antibody. Methods for making a humanized antibody from a non-human (e.g., mouse) monoclonal antibody are known in the art. Additional non-limiting examples of making a chimeric (e.g., humanized) antibody are described herein.

In some embodiments, the antibodies are chimeric antibodies that contain a light chain immunoglobulin that contains the light chain variable domain of a non-human antibody (e.g., a mouse antibody) or at least one CDR of a light chain variable domain of a non-human antibody (e.g., a mouse antibody) and the constant domain of a human immunoglobulin light chain (e.g., human K chain constant domain). In some embodiments, the antibodies are chimeric antibodies that contain a heavy chain immunoglobulin that contains the heavy chain variable domain of a non-human (e.g., a mouse antibody) or at least one CDR of a heavy chain variable domain of a non-human (e.g., a mouse antibody) and the constant domain of a human immunoglobulin heavy chain (e.g., a human IgG heavy chain constant domain). In some embodiments, the chimeric antibodies contain a portion of a constant (Fc domain) of a human immunoglobulin.

In some embodiments, the antibodies or antigen-binding fragments thereof can be multi-specific (e.g., multimeric). For example, the antibodies can take the form of antibody dimers, trimers, or higher-order multimers of monomeric immunoglobulin molecules. Dimers of whole immunoglobulin molecules or of F(ab′)₂ fragments are tetravalent, whereas dimers of Fab fragments or scFv molecules are bivalent. Individual monomers within an antibody multimer may be identical or different, i.e., they may be heteromeric or homomeric antibody multimers. For example, individual antibodies within a multimer may have the same or different binding specificities.

Multimerization of antibodies may be accomplished through natural aggregation of antibodies or through chemical or recombinant linking techniques known in the art. For example, some percentage of purified antibody preparations (e.g., purified IgG₁ molecules) spontaneously form protein aggregates containing antibody homodimers and other higher-order antibody multimers. Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) (available, for example, from Pierce Biotechnology, Inc., Rockford, Ill.) can be used to form antibody multimers. An exemplary protocol for the formation of antibody homodimers is described in Ghetie et al. (Proc. Natl. Acad. Sci. U.S.A. 94: 7509-7514, 1997). Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules. For example, the interface can contain at least a part of the C_{H}3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products, such as homodimers (see, for example, WO 96/27011).

Bi-specific antibodies include cross-linked or “heteroconjugate” antibodies. Suitable cross-linking agents are well known in the art and are disclosed in U.S. Pat. No. 4,676,980, along with a variety of cross-linking techniques.

Methods for generating bi-specific antibodies from antibody fragments are also known in the art. For example, bi-specific antibodies can be prepared using chemical linkage. Shalaby et al. (J. Exp. Med. 175:217-225, 1992) describes the production of a fully-humanized bi-specific antibody F(ab′)₂ molecule. Additional techniques for making and isolating bi-specific antibody fragments directly from recombinant cell culture have also been described. For example, bi-specific antibodies have been produced using leucine zippers (Kostelny et al., J. Immunol. 148:1547-1553, 1992). This method can also be utilized for the production of antibody homodimers.

The diabody technology described by Hollinger et al. (Proc. Natl. Acad. Sci. U.S.A. 90:6444-6448, 1993) is an additional method for making bi-specific antibody fragments. The fragments contain a heavy chain variable domain (VH) connected to a light chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another method for making bi-specific antibody fragments by the use of single-chain Fv (sFv) dimers has been described in Gruber et al. (J. Immunol. 153:5368, 1994). Alternatively, the bi-specific antibody can be a “linear” or “single-chain antibody” produced using the methods described, for example, in Zapata et al. (Protein Eng. 8:1057-1062, 1995). In some embodiments the antibodies have more than two antigen-binding sites. For example, tri-specific antibodies can be prepared as described in Tutt et al. (J. Immunol. 147:60, 1991).

Alternatively, antibodies can be made to multimerize through recombinant DNA techniques. IgM and IgA naturally form antibody multimers through the interaction with the mature J chain polypeptide. Non-IgA or non-IgM molecules, such as IgG molecules, can be engineered to contain the J chain interaction domain of IgA or IgM, thereby conferring the ability to form higher order multimers on the non-IgA or non-IgM molecules (see, for example, Chintalacharuvu et al., Clin. Immunol. 101:21-31, 2001, and Frigerio et al., Plant Physiol. 123:1483-1494, 2000). IgA dimers are naturally secreted into the lumen of mucosa-lined organs. This secretion is mediated through the interaction of the J chain with the polymeric IgA receptor (pIgR) on epithelial cells. If secretion of an IgA form of an antibody (or of an antibody engineered to contain a J chain interaction domain) is not desired, it can be greatly reduced by expressing the antibody molecule in association with a mutant J chain that does not interact well with pIgR (Johansen et al., J. Immunol., 167:5185-192, 2001). ScFv dimers can also be formed through recombinant techniques known in the art. An example of the construction of scFv dimers is given in Goel et al. (Cancer Res. 60:6964-71, 2000).

An antigen binding domain can bind to a particular with an affinity (KD) equal to or less than 1×10⁻⁷ M (e.g., equal to or less than 1×10⁻⁸ M, equal to or less than 5×10⁻⁹ M, equal to or less than 2×10⁻⁹M, or equal to or less than 1×10⁻⁹M) in phosphate buffered saline.

As can be appreciated by those in the art, the choice of the antigen binding domain to include in the CAR depends upon the type and number of ligands that define the surface of a target cancer cell, a target pathogen, or a target cell infected with a pathogen. For example, the antigen binding domain may be chosen to recognize a ligand that acts as a cell surface marker on cancer cells, pathogens, or cells infected with a pathogen. Thus examples of cell surface markers that may act as ligands for the antigen binding domain in a CAR include those associated with viral, bacterial, and parasitic infections, and cancer cells.

The antigen binding domain can bind specifically to a tumor antigen or a pathogen antigen. Tumor antigens are proteins or molecules (e.g., polysaccharides and/or lipids) that are produced by cancer cells that elicit an immune response, particularly a T cell-mediated immune response. Pathogen antigens are proteins or other molecules (e.g., polysaccharides and/or lipids) that are produced by pathogens.

The selection of the antigen binding moiety will depend on the particular type of cancer or pathogen to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulm, RAGE-1, MN-CA IX, human telomerase reverse transcriptase, RUL RU2 (AS), intestinal carboxyi esterase, mut hsp70-2, M-CSF, prostase, prostate-specific antigen (PSA), PAP, NY-ESO-1, LAGE-1a, p53, prostein, PSMA, Her2/neu, survivin, telomerase, prostate-carcinoma tumor antigen-1 (PCTA-1), MAGE, ELF2M, neutrophil elastase, ephrinB2, CD22, insulin growth factor (IGF)-1, IGF-2, IGF-1 receptor, and mesothelin.

In some examples, the tumor antigen includes one or more antigenic cancer epitopes associated with a malignant tumor. Malignant tumors express a number of proteins that can serve as target antigens. These molecules include, but are not limited to, tissue-specific antigens, such as MART-1, tyrosinase, and GP 100 in melanoma, and prostatic acid phosphatase (PAP) and prostate-specific antigen (PSA) in prostate cancer. Other target antigens belong to the group of transformation-related molecules, such as the oncogene HER-2/Neu ErbB-2. Yet another group of target antigens are onco-fetal antigens, such as carcinoembryonic antigen (CEA). In B-cell lymphoma, the tumor-specific idiotype immunoglobulin constitutes a tumor-specific immunoglobulin antigen that is unique to the individual tumor. B-cell differentiation antigens, such as CD19, CD20, and CD37, are other candidates for target antigens in B-cell lymphoma. Some of these antigens (CEA, HER-2, CD19, CD20, idiotype) have been used as targets for passive immunotherapy with monoclonal antibodies with limited success.

A tumor antigen recognized by the antigen binding domain may also be a tumor-specific antigen (TSA) or a tumor-associated antigen (TAA). A TSA is unique to tumor cells and does not occur on other cells in the body. A TAA is not unique to a tumor cell and instead is also expressed on a normal cell under conditions that fail to induce a state of immunologic tolerance to the antigen. The expression of the TAA antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells, but which are expressed at much higher levels on tumor cells.

Non-limiting examples of TSAs or TAAs include the following: differentiation antigens, such as MART-1/MelanA (MART-1), g100 (Pmel 17), tyrosinase, TRP-1, TRP-2; tumor-specific multilineage antigens, such as MAGE-1, MAGE-3, BAGE, GAGE-1, GAGE-2, and pi5; overexpressed embryonic antigens, such as CEA; overexpressed oncogenes and mutated tumor-suppressor genes such as p53, Ras, and HER-2/neu; unique tumor antigens resulting from chromosomal translocations, such as BCR-ABL, E2A-PRL, H4-RET, 1GH-IGK, and MYL-RAR. Other large, antigens include, e.g., TSP-180, MAGE-4, MAGE-5, MAGE-6, RAGE, NY-ESO, pl 85erbB2, pl 80erbB-3, c-met, nm-23H1, PSA, TAG-72, CA 19-9, CA 72-4, CAM 17.1, NuMa, K-ras, beta-Catenin, CDK4, Mum-1, p15, p16, 43-9F, 5T4, 791Tgp72, alpha-fetoprotem, beta-HCG, BCA225, BTAA, CAl25, CA15-3\CA 27.29\BCAA, CA195, CA242, CA-50, CAM43, CD68\I, CO-029, FGF-5, G250, Ga733VEpCAM, HTgp-175, M344, MA-50, MG7-Ag, MOV18, NB/70K, NY-CO-1, RCAS1, SDCCAG16, TA-90\Mac-2 binding protein A cyclophilin C-associated protein, TAAL6, TAG72, TLP, TPS, hTERT, ganglioside GD3, Hu, Yo, and GAP.

In some examples, the antigen binding domain specifically binds to an antigen that includes, but is not limited to, CD19, CD20, CD22, ROR1, mesothelin, CD33/IL3Ra, c-Met, PSMA, Glycolipid F77, EGFRvIII, GD-2, MY-ESO-1 TCR, MAGE A3 TCR, and the like. Depending on the desired antigen to be targeted, a CAR can be engineered to include the appropriate antigen binding domain that binds specifically to the desired antigen. For example, if CD19 is the desired antigen that is to be targeted, an antibody or an antigen-binding fragment of an antibody that binds specifically to CD19 can be used as the antigen binding domain in the CAR. In some examples, the antigen binding moiety portion of a CAR targets CD19. For example, the antigen binding domain in the CAR is anti-CD19 scFV.

In some examples, the antigen binding domain is of viral or bacterial origin including, but not limited to, human papilloma virus (HPV) antigens, and Epstein-Barr virus antigen, polyoma virus antigens, Kaposi's sarcoma-associated herpesvirus (KSHV) HHV-8, Hepatitis B (HBV) and C (HCV) virus antigens, herpes virus antigens, HIV antigens, Influenza (Orthomyxoviridae) antigen, Helicobacter pylori antigens, Staphylococcus aureus antigens. Examples include, but are not limited to HPV E6/E7 antigen, EBV Virus Nuclear Antigen 1, polyoma virus large T antigen, HBV core antigen, HCV nonstructural (NS) 5A, HIV gp120 antigen, HIV gp41, S. aureus surface antigen B, influenza hemagglutinin, and influenza neuraminidase. Additional examples of antigen-binding domains and antigens are described in the references cited herein (see, e.g., Van der Bruggen et al., Cancer Immunity 2013 (www.cancerimmunity.org/peptide/), and Vigneron, BioMed Res. Int. Vol. 2015 Article ID 948501, 17 pp, 2015).

In some examples, the presence of tumor antigens is defined by the ability of tumor material including lysed tumor cells, necrotic tumor cells, tumor proteins, tumor apoptotic bodies to elicit activation of an autologous T cell population without further characterization of the antigen.

### Linker

The CAR described herein can optionally include a linker (1) between the antigen binding domain and the transmembrane domain, and/or (2) between the transmembrane domain and the cytoplasmic signaling domain. The linker can be an oligonucleotide or a polypeptide linker. For example, when the linker is an oligonucleotide, the linker can be about 1 nucleotide to about 500 nucleotides, about 450 nucleotides, about 400 nucleotides, about 350 nucleotides, about 300 nucleotides, about 250 nucleotides, about 200 nucleotides, about 180 nucleotides, about 160 nucleotides, about 140 nucleotides, about 120 nucleotides, about 100 nucleotides, about 90 nucleotides, about 80 nucleotides, about 70 nucleotides, about 60 nucleotide, about 50 nucleotides, about 40 nucleotides, about 30 nucleotides, about 20 nucleotides, or about 10 nucleotides; about 10 nucleotides to about 500 nucleotides, about 450 nucleotides, about 400 nucleotides, about 350 nucleotides, about 300 nucleotides, about 250 nucleotides, about 200 nucleotides, about 180 nucleotides, about 160 nucleotides, about 140 nucleotides, about 120 nucleotides, about 100 nucleotides, about 90 nucleotides, about 80 nucleotides, about 70 nucleotides, about 60 nucleotide, about 50 nucleotides, about 40 nucleotides, about 30 nucleotides, or about 20 nucleotides; about 20 nucleotides to about 500 nucleotides, about 450 nucleotides, about 400 nucleotides, about 350 nucleotides, about 300 nucleotides, about 250 nucleotides, about 200 nucleotides, about 180 nucleotides, about 160 nucleotides, about 140 nucleotides, about 120 nucleotides, about 100 nucleotides, about 90 nucleotides, about 80 nucleotides, about 70 nucleotides, about 60 nucleotide, about 50 nucleotides, about 40 nucleotides, or about 30 nucleotides; about 30 nucleotides to about 500 nucleotides, about 450 nucleotides, about 400 nucleotides, about 350 nucleotides, about 300 nucleotides, about 250 nucleotides, about 200 nucleotides, about 180 nucleotides, about 160 nucleotides, about 140 nucleotides, about 120 nucleotides, about 100 nucleotides, about 90 nucleotides, about 80 nucleotides, about 70 nucleotides, about 60 nucleotide, about 50 nucleotides, or about 40 nucleotides; about 40 nucleotides to about 500 nucleotides, about 450 nucleotides, about 400 nucleotides, about 350 nucleotides, about 300 nucleotides, about 250 nucleotides, about 200 nucleotides, about 180 nucleotides, about 160 nucleotides, about 140 nucleotides, about 120 nucleotides, about 100 nucleotides, about 90 nucleotides, about 80 nucleotides, about 70 nucleotides, about 60 nucleotide, or about 50 nucleotides; about 50 nucleotides to about 500 nucleotides, about 450 nucleotides, about 400 nucleotides, about 350 nucleotides, about 300 nucleotides, about 250 nucleotides, about 200 nucleotides, about 180 nucleotides, about 160 nucleotides, about 140 nucleotides, about 120 nucleotides, about 100 nucleotides, about 90 nucleotides, about 80 nucleotides, about 70 nucleotides, or about 60 nucleotide; about 60 nucleotides to about 500 nucleotides, about 450 nucleotides, about 400 nucleotides, about 350 nucleotides, about 300 nucleotides, about 250 nucleotides, about 200 nucleotides, about 180 nucleotides, about 160 nucleotides, about 140 nucleotides, about 120 nucleotides, about 100 nucleotides, about 90 nucleotides, about 80 nucleotides, or about 70 nucleotides; about 70 nucleotides to about 500 nucleotides, about 450 nucleotides, about 400 nucleotides, about 350 nucleotides, about 300 nucleotides, about 250 nucleotides, about 200 nucleotides, about 180 nucleotides, about 160 nucleotides, about 140 nucleotides, about 120 nucleotides, about 100 nucleotides, about 90 nucleotides, or about 80 nucleotides; about 80 nucleotides to about 500 nucleotides, about 450 nucleotides, about 400 nucleotides, about 350 nucleotides, about 300 nucleotides, about 250 nucleotides, about 200 nucleotides, about 180 nucleotides, about 160 nucleotides, about 140 nucleotides, about 120 nucleotides, about 100 nucleotides, or about 90 nucleotides; about 90 nucleotides to about 500 nucleotides, about 450 nucleotides, about 400 nucleotides, about 350 nucleotides, about 300 nucleotides, about 250 nucleotides, about 200 nucleotides, about 180 nucleotides, about 160 nucleotides, about 140 nucleotides, about 120 nucleotides, or about 100 nucleotides; about 100 nucleotides to about 500 nucleotides, about 450 nucleotides, about 400 nucleotides, about 350 nucleotides, about 300 nucleotides, about 250 nucleotides, about 200 nucleotides, about 180 nucleotides, about 160 nucleotides, about 140 nucleotides, or about 120 nucleotides; about 120 nucleotides to about 500 nucleotides, about 450 nucleotides, about 400 nucleotides, about 350 nucleotides, about 300 nucleotides, about 250 nucleotides, about 200 nucleotides, about 180 nucleotides, about 160 nucleotides, or about 140 nucleotides; about 140 nucleotides to about 500 nucleotides, about 450 nucleotides, about 400 nucleotides, about 350 nucleotides, about 300 nucleotides, about 250 nucleotides, about 200 nucleotides, about 180 nucleotides, or about 160 nucleotides; about 160 nucleotides to about 500 nucleotides, about 450 nucleotides, about 400 nucleotides, about 350 nucleotides, about 300 nucleotides, about 250 nucleotides, about 200 nucleotides, or about 180 nucleotides; about 180 nucleotides to about 500 nucleotides, about 450 nucleotides, about 400 nucleotides, about 350 nucleotides, about 300 nucleotides, about 250 nucleotides, or about 200 nucleotides; about 200 nucleotides to about 500 nucleotides, about 450 nucleotides, about 400 nucleotides, about 350 nucleotides, about 300 nucleotides, or about 250 nucleotides; about 250 nucleotides to about 500 nucleotides, about 450 nucleotides, about 400 nucleotides, about 350 nucleotides, or about 300 nucleotides; about 300 nucleotides to about 500 nucleotides, about 450 nucleotides, about 400 nucleotides, or about 350 nucleotides; about 350 nucleotides to about 500 nucleotides, about 450 nucleotides, or about 400 nucleotides; about 400 nucleotides to about 500 nucleotides or about 450 nucleotides; or about 450 nucleotides to about 500 nucleotides.

For example, when the linker is an polypeptide, the linker can be between about 1 amino acid and about 500 amino acids, about 450 amino acids, about 400 amino acids, about 350 amino acids, about 300 amino acids, about 250 amino acids, about 200 amino acids, about 150 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, about 20 amino acids, about 18 amino acids, about 16 amino acids, about 14 amino acids, about 12 amino acids, about 10 amino acids, about 8 amino acids, about 6 amino acids, about 4 amino acids, or about 2 amino acids; about 2 amino acids to about 500 amino acids, about 450 amino acids, about 400 amino acids, about 350 amino acids, about 300 amino acids, about 250 amino acids, about 200 amino acids, about 150 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, about 20 amino acids, about 18 amino acids, about 16 amino acids, about 14 amino acids, about 12 amino acids, about 10 amino acids, about 8 amino acids, about 6 amino acids, or about 4 amino acids; about 4 amino acids to about 500 amino acids, about 450 amino acids, about 400 amino acids, about 350 amino acids, about 300 amino acids, about 250 amino acids, about 200 amino acids, about 150 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, about 20 amino acids, about 18 amino acids, about 16 amino acids, about 14 amino acids, about 12 amino acids, about 10 amino acids, about 8 amino acids, or about 6 amino acids; about 6 amino acids to about 500 amino acids, about 450 amino acids, about 400 amino acids, about 350 amino acids, about 300 amino acids, about 250 amino acids, about 200 amino acids, about 150 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, about 20 amino acids, about 18 amino acids, about 16 amino acids, about 14 amino acids, about 12 amino acids, about 10 amino acids, or about 8 amino acids; about 8 amino acids to about 500 amino acids, about 450 amino acids, about 400 amino acids, about 350 amino acids, about 300 amino acids, about 250 amino acids, about 200 amino acids, about 150 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, about 20 amino acids, about 18 amino acids, about 16 amino acids, about 14 amino acids, about 12 amino acids, or about 10 amino acids; about 10 amino acids to about 500 amino acids, about 450 amino acids, about 400 amino acids, about 350 amino acids, about 300 amino acids, about 250 amino acids, about 200 amino acids, about 150 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, about 20 amino acids, about 18 amino acids, about 16 amino acids, about 14 amino acids, or about 12 amino acids; about 12 amino acids to about 500 amino acids, about 450 amino acids, about 400 amino acids, about 350 amino acids, about 300 amino acids, about 250 amino acids, about 200 amino acids, about 150 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, about 20 amino acids, about 18 amino acids, about 16 amino acids, or about 14 amino acids; about 14 amino acids to about 500 amino acids, about 450 amino acids, about 400 amino acids, about 350 amino acids, about 300 amino acids, about 250 amino acids, about 200 amino acids, about 150 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, about 20 amino acids, about 18 amino acids, or about 16 amino acids; about 16 amino acids to about 500 amino acids, about 450 amino acids, about 400 amino acids, about 350 amino acids, about 300 amino acids, about 250 amino acids, about 200 amino acids, about 150 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, about 20 amino acids, or about 18 amino acids; about 18 amino acids to about 500 amino acids, about 450 amino acids, about 400 amino acids, about 350 amino acids, about 300 amino acids, about 250 amino acids, about 200 amino acids, about 150 amino acids, about 100 amino acids, about 90 amino acids, about 80 amino acids, about 70 amino acids, about 60 amino acids, about 50 amino acids, about 40 amino acids, about 35 amino acids, about 30 amino acids, about 25 amino acids, or about 20 amino acids; about 20 amino acids to about 500 amino acids, about 450 amino acids, about 400 amino acids, about 350 amino acids, about 300 amino acids, about 250 amino acids, about 200 amino acids, about 150 amino acids, about 100 amino acids, about 90 amino acids, about 80 amino acids, about 70 amino acids, about 60 amino acids, about 50 amino acids, about 40 amino acids, about 35 amino acids, about 30 amino acids, or about 25 amino acids; about 25 amino acids to about 500 amino acids, about 450 amino acids, about 400 amino acids, about 350 amino acids, about 300 amino acids, about 250 amino acids, about 200 amino acids, about 150 amino acids, about 100 amino acids, about 90 amino acids, about 80 amino acids, about 70 amino acids, about 60 amino acids, about 50 amino acids, about 40 amino acids, about 35 amino acids, or about 30 amino acids; about 30 amino acids to about 500 amino acids, about 450 amino acids, about 400 amino acids, about 350 amino acids, about 300 amino acids, about 250 amino acids, about 200 amino acids, about 150 amino acids, about 100 amino acids, about 90 amino acids, about 80 amino acids, about 70 amino acids, about 60 amino acids, about 50 amino acids, about 40 amino acids, or about 35 amino acids; about 35 amino acids to about 500 amino acids, about 450 amino acids, about 400 amino acids, about 350 amino acids, about 300 amino acids, about 250 amino acids, about 200 amino acids, about 150 amino acids, about 100 amino acids, about 90 amino acids, about 80 amino acids, about 70 amino acids, about 60 amino acids, about 50 amino acids, or about 40 amino acids; about 40 amino acids to about 500 amino acids, about 450 amino acids, about 400 amino acids, about 350 amino acids, about 300 amino acids, about 250 amino acids, about 200 amino acids, about 150 amino acids, about 100 amino acids, about 90 amino acids, about 80 amino acids, about 70 amino acids, about 60 amino acids, or about 50 amino acids; about 50 amino acids to about 500 amino acids, about 450 amino acids, about 400 amino acids, about 350 amino acids, about 300 amino acids, about 250 amino acids, about 200 amino acids, about 150 amino acids, about 100 amino acids, about 90 amino acids, about 80 amino acids, about 70 amino acids, or about 60 amino acids; about 60 amino acids to about 500 amino acids, about 450 amino acids, about 400 amino acids, about 350 amino acids, about 300 amino acids, about 250 amino acids, about 200 amino acids, about 150 amino acids, about 100 amino acids, about 90 amino acids, about 80 amino acids, or about 70 amino acids; about 70 amino acids to about 500 amino acids, about 450 amino acids, about 400 amino acids, about 350 amino acids, about 300 amino acids, about 250 amino acids, about 200 amino acids, about 150 amino acids, about 100 amino acids, about 90 amino acids, or about 80 amino acids; about 80 amino acids to about 500 amino acids, about 450 amino acids, about 400 amino acids, about 350 amino acids, about 300 amino acids, about 250 amino acids, about 200 amino acids, about 150 amino acids, about 100 amino acids, or about 90 amino acids; about 90 amino acids to about 500 amino acids, about 450 amino acids, about 400 amino acids, about 350 amino acids, about 300 amino acids, about 250 amino acids, about 200 amino acids, about 150 amino acids, or about 100 amino acids; about 100 amino acids to about 500 amino acids, about 450 amino acids, about 400 amino acids, about 350 amino acids, about 300 amino acids, about 250 amino acids, about 200 amino acids, or about 150 amino acids; about 150 amino acids to about 500 amino acids, about 450 amino acids, about 400 amino acids, about 350 amino acids, about 300 amino acids, about 250 amino acids, or about 200 amino acids; about 200 amino acids to about 500 amino acids, about 450 amino acids, about 400 amino acids, about 350 amino acids, about 300 amino acids, or about 250 amino acids; about 250 amino acids to about 500 amino acids, about 450 amino acids, about 400 amino acids, about 350 amino acids, or about 300 amino acids; about 300 amino acids to about 500 amino acids, about 450 amino acids, about 400 amino acids, or about 350 amino acids; about 350 amino acids to about 500 amino acids, about 450 amino acids, or about 400 amino acids; about 350 amino acids to about 500 amino acids or about 450 amino acids; about 450 amino acids to about 500 amino acids.

In some examples, the polypeptide linker includes a glycine-serine doublet. In some examples, the polypeptide linker has a secondary alpha helix structure. In some examples, the polypeptide linker has a secondary beta sheet structure. Additional examples of linkers are described in the references cited herein.

### Transmembrane Domains

The CARs described herein also include a transmembrane domain. In some examples, the transmembrane domain is naturally associated with a sequence in the cytoplasmic domain. In some examples, the transmembrane domain can be modified by one or more (e.g., two, three, four, five, six, seven, eight, nine, or ten) amino acid substitutions to avoid the binding of the domain to other transmembrane domains (e.g., the transmembrane domains of the same or different surface membrane proteins) to minimize interactions with other members of the receptor complex.

The transmembrane domain may be derived either from a natural or from a synthetic source. Where the source is natural, the domain may be derived from any membrane-bound or transmembrane protein. Non-limiting examples of transmembrane domains of particular use in this invention may be derived from (e.g., comprise at least the transmembrane sequence or a part of the transmembrane sequence of) the alpha, beta, or zeta chain of the T-cell receptor, CD28, CD3 epsilon, CD45, CD4, CD5, CDS, CD9, CD16, CD22, CD33, CD37, CD64, CD80, CD86, CD134, CD137, or CD154.

In some examples, the transmembrane domain may be synthetic, in which case it will comprise predominantly hydrophobic residues such as leucine and valine. In some examples, the synthetic transmembrane domain will include a triplet of phenylalanine, tryptophan, and valine at each end of a synthetic transmembrane domain. In some examples, the transmembrane domain of a CAR includes the CD8 hinge domain.

Additional specific examples of transmembrane domains are described in the references cited herein.

### Cytoplasmic Domains

The CARs described herein can include, e.g., a primary cytoplasmic signaling domain that includes a cytoplasmic sequence of CD3ζ sequence sufficient to stimulate a T cell when the antigen-binding domain binds to the antigen, and optionally, a cytoplasmic sequence of one or more of co-stimulatory proteins (e.g., a cytoplasmic sequence of one or more of CD27, CD28, 4-1BB, OX40, CD30, CD40L, CD40, PD-1, PD-L1, ICOS, LFA-1, CD2, CD7, CD160, LIGHT, BTLA, TIM3, CD244, CD80, LAG3, NKG2C, B7-H3, and a ligand that specifically binds to CD83) that provides for co-stimulation of the T cell. The stimulation of a CAR T cell can result in the activation of one or more anti-tumor or anti-infectious disease activities of the CAR T cell. For example, stimulation of a CAR T cell can result in an increase in the cytolytic activity or helper activity of the CAR T cell, including the secretion of cytokines. In some examples, the entire intracellular signaling domain of a co-stimulatory protein is included in the cytoplasmic signaling domain. In some embodiments, the cytoplasmic signaling domain includes a truncated portion of an intracellular signaling domain of a co-stimulatory protein, as long as the truncated portion of the intracellular signaling domain transduces an effector function signal in the CAR T cell. Non-limiting examples of intracellular signaling domains that can be included in a cytoplasmic signaling domain include the cytoplasmic sequences of the T cell receptor (TCR) and co-receptors that act in concert to initiate signal transduction following antigen receptor engagement, as well as any variant of these sequences including at least one (e.g., one, two, three, four, five, six, seven, eight, nine, or ten) substitution and having the same functional capability.

In some examples, a cytoplasmic signaling domain can include two distinct classes of cytoplasmic signaling sequences: signaling sequences that initiate antigen-dependent primary activation through the TCR (primary cytoplasmic signaling sequences) (e.g., a CD3ζ cytoplasmic signaling sequence) and a cytoplasmic sequence of one or more of co-stimulatory proteins that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs, which are known as immunoreceptor tyrosine-based activation motifs or ITAMs.

Examples of ITAM containing primary cytoplasmic signaling sequences that can be included in a cytoplasmic signaling domain include those derived from TCR zeta, FcR gamma, FcR beta, CD3 gamma, CD3 delta, CD3 epsilon, CDS, CD22, CD79a, CD79b, and CD66d. In some embodiments, the primary cytoplasmic signaling sequence in the CAR includes a cytoplasmic signaling sequence derived from CD3ζ.

In some examples, the cytoplasmic domain of a CAR can be designed to include the CD3ζ signaling domain by itself or combined with any other desired cytoplasmic signaling sequence(s) useful in the context of a CAR. For example, the cytoplasmic domain of a CAR can comprise a CD3ζ chain portion and a costimulatory cytoplasmic signaling sequence. The costimulatory cytoplasmic signaling sequence refers to a portion of a CAR including a cytoplasmic signaling sequence of a costimulatory protein. A costimulutory protein is a cell surface molecule, other than an antigen receptor or their ligands, that is required for an efficient response of lymphocytes to an antigen. Non-limiting examples of such costimulatory proteins include CD27, CD28, 4-IBB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like.

The cytoplasmic signaling sequences within the cytoplasmic signaling domain of a CAR may be linked to each other in a random or specified order. Optionally, a linker (e.g., any of the linkers described herein) may form the linkage.

In some embodiments, the cytoplasmic signaling domain is designed to include the cytoplasmic signaling sequence of CD3ζ and the cytoplasmic signaling sequence of the costimulatory protein CD28. In some examples, the cytoplasmic signaling domain is designed to include the cytoplasmic signaling sequence of CD3ζ and the cytoplasmic signaling sequence of costimulatory protein 4-IBB. In some examples, the cytoplasmic signaling domain is designed to include the cytoplasmic signaling sequence of CD3ζ and the cytoplasmic signaling sequences of costimulatory proteins CD28 and 4-1BB.

In some examples, the cytoplasmic signaling domain in a CAR can include the cytoplasmic signaling sequence of 4-IBB and the cytoplasmic signaling domain of CD3ζ, where the cytoplasmic signaling sequence of 4-1BB is encoded by the nucleic acid sequence of SEQ ID NO: 1 and the cytoplasmic signaling sequence of CD3ζ is encoded by the nucleic acid of SEQ ID NO: 2.

In some examples, the cytoplasmic signaling domain in a CAR can include the cytoplasmic signaling domain of 4-IBB and the cytoplasmic signaling domain of CD3ζ, where the cytoplasmic signaling domain of 4-IBB includes the amino acid sequence of SEQ ID NO: 3 and the cytoplasmic signaling domain of CD3ζ includes the amino acid sequence of SEQ ID NO: 4.

Agents that Activate T Cells

Some of the methods described herein further include a step of activating a T cell. In some embodiments where the T cell is a genetically modified T cell (e.g., a CAR T cell), the activation of the T cell can occur before or after the introduction of recombinant nucleic acid into the T cell. Methods for activating and culturing T cells are described in, e.g., U.S. Pat. Nos. 6,352,694; 6,534,055; 6,905,680; 6,692,964; 5,858,358; 6,887,466; 6,905,681; 7, 144,575; 7,067,318; 7, 172,869; 7,232,566; 7, 175,843; 5,883,223; 6,905,874; 6,797,514; 6,867,041; and U.S. Patent Application Publication No. 20060121005.

Generally, T cells are activated by contacting the cells a surface having attached thereto an agent that stimulates a CD3 TCR complex associated signal in the T cell and a ligand that stimulates a co-stimulatory protein on the surface of the T cell. In some examples, the T cell may be activated by contacting the T cell with an antt-CD3 antibody, or antigen-binding fragment thereof, or an anti-CD2 antibody immobilized on a surface, or by contacting the T cell with a protein kinase C activator (e.g., bryostatin) in combination with a calcium ionophore. A ligand that binds the co-stimulatory protein on the surface of a T cell can be used to active the co-stimulatory protein on the surface of a T cell. For example, a population of T cells can be contacted with an anti-CD3 antibody and an anti-CD28 antibody, under conditions appropriate for activating the T cells. An anti-CD3 antibody and an anti-CD28 antibody can be used to stimulate proliferation of either CD4⁺ T cells or CD8⁺ T cells. Non-limiting examples of an anti-CD28 antibody include 9.3, B-T3, XR-CD28 (Diacione, Besancon, France). Additional methods for activating T cells are known in the art (Berg et al., Transplant Proc. 30(8):3975-3977, 1998; Haanen et al., J. Exp. Med. 190(9):1319-1328, 1999; and Garland et al., J. Immunol. Meth. 227(1-2):53-63, 1999).

In some examples, activation of the primary cytoplasmic signaling sequence and the co-stimulatory cytoplasmic signaling sequence signal in the T cell can be provided by different protocols. For example, the agents activating each signal may be in solution or coupled to a surface. When coupled to a surface, the agents may be coupled to the same surface (i.e., in “cis” formation) or to separate surfaces (i.e., in “trans” formation), In some examples, one agent may be coupled to a surface and the other agent in solution. In some examples, the agent providing activation of the co-stimulatory cytoplasmic signaling sequence is bound to a cell surface and the agent providing activation of the primary cytoplasmic signaling sequence is in solution or coupled to a surface. In certain embodiments, both agents can be in solution. In some examples, the agents may be in soluble form, and then cross-linked to a surface, such as a cell expressing Fc receptors or an antibody or other binding agent which will bind to the agents. See, e.g., U.S. Patent Application Publication Nos. 2004/0101519 and 2006/0034810 for artificial antigen presenting cells (aAPCs) that can be used for activating T cell in the present methods.

In some examples, the two agents are immobilized on beads, either on the same bead, i.e., “cis,” or to separate beads, i.e., “trans.” By way of example, the agent that activates the primary cytoplasmic signaling sequence is an anti-CD3 antibody or an antigen-binding fragment thereof, and the agent activating the co-stimulatory cytoplasmic signaling sequence is an anti-CD28 antibody or antigen-binding fragment thereof; and both agents are co-immobilized to the same bead in equivalent molecular amounts. In some examples, a 1:1 ratio of each antibody is bound to the beads for T cell activation. In other examples, a ratio of anti-CD3:anti-CD28 antibodies is bound to the beads such that an increase in T cell expansion is observed as compared to the expansion observed using a ratio of 1:1. In some examples, the ratio of anti-CD3:anti-CD28 antibodies bound to the beads ranges from 100:1 to 1:100, and all integer values there between. In some examples, more anti-CD28 antibodies are bound to the particles than anti-CD3 antibodies (a ratio of anti-CD3:anti-CD28 antibodies of less than one). In some examples, the ratio of anti-CD28 antibodies to anti-CD3 antibodies bound to the beads is greater than 2:1. In some examples, antibodies, in a ratio of 1:100 anti-CD3:anti-CD28, are bound to the beads. In some examples, antibodies, in a ratio of 1:75 anti-CD3:anti-CD28, is bound to beads. In some examples, antibodies, in a ratio of 1:50 anti-CD3:anti-CD28, are bound to the beads. In another embodiment, antibodies, in a ratio of 1:30 anti-CD3:anti-CD28 ratio, are bound to the beads. In some examples, antibodies, in a ratio of 1:10 anti-CD3:anti-CD28, are bound to the beads. In some embodiments, antibodies, in a ratio of 1:3 anti-CD3:anti-CD28, are bound to the beads. In some examples, antibodies in a ratio of 3:1 anti-CD3:anti-CD28, are bound to the beads.

The ratio of the number of particles to the number T cells can be, e.g., about 1:500 to about 500:1, and any integer values in between. As those of ordinary skill in the art can readily appreciate, the ratio of particles to cells may depend on particle size relative to size of the target cell. For example, small sized beads could only bind a few cells, while larger beads may bind many cells. In some examples, the ratio of T cells to particles can be about 1:100 to about 100:1, and any integer values in between. In some examples the ratio of T cells to particles can be about 1:9 to about 9:1, and any integer values in between. The ratio of the number of anti-CD3- and anti-CD28-coupled particles to the number of T cells that result in T cell activation can be about 1:100, about 1:50, about 1:40, about 1:30, about 1:20, about 1:10, about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, about 10:1, or about 15:1. In some examples, a ratio of the number of particles to the number of T cells of 1:1 or less is used. In some examples, a ratio of the number of particles to the number of T cells of about 1:5 is used. In some examples, the ratio of the number of particles to the number of T cells can be varied depending on the day of activation. For example, the ratio of the number of particles to the number of T cells is about 1:1 to about 10:1 is used on the first day of activation, and additional particles are added to the T cells every day, or every other day thereafter, for up to 10 days, at final particle to T cell ratios of about 1:1 to about 1:10 (based on cell counts on each day of addition). In some examples, the ratio of particles to T cells is about 1:1 on the first day of activation and adjusted to about 1:5 on the third and fifth days of activation. In some examples, the particles are added on a daily, or every other day, basis to a final ratio of about 1:1 on the first day, and about 1:5 on the third and fifth days of activation. In some examples, the ratio of particles to cells is about 2:1 on the first day of activation and adjusted to 1:10 on the third and fifth days of activation. In some embodiments, the particles are added on a daily, or every other day, basis to a final ratio of about 1:1 on the first day, and 1:10 on the third and fifth days of activation. One of skill in the art will appreciate that a variety of other ratios may be suitable for use in these methods of activating T cells. In particular, the ratios will vary depending on particle size and the cell size and type.

In some examples, the T cells, are combined with agent-coated beads, the beads and the cells are subsequently separated, and then the cells are cultured. In an alternative, prior to culture, the agent-coated beads and cells are not separated but are cultured together. In some examples, the beads and cells are first concentrated by application of a force, such as a magnetic force, resulting in increased ligation of proteins on the T cell surface to the antibodies on the beads, thereby inducing T cell activation.

By way of example, cell surface proteins may be ligated by allowing paramagnetic beads to which anti-CD3 and anti-CD28 antibodies are attached (3×28 beads) to contact the T cells. In some examples, the T cells (e.g., 10⁴ to 10⁹ T cells) and beads (for example, DYNABEADS® M-450 CD3/CD28 T paramagnetic beads), e.g., at a ratio of about 1:1, are combined in a buffer, preferably PBS (without divalent cations, such as calcium and magnesium). Again, those of ordinary skill in the art can readily appreciate any T cell concentration may be used. For example, the target T cell may be very rare in the sample and comprise only about 0.01% of the sample or can the target T cell can comprise the entire sample (about 100%). Accordingly, any T cell number or concentration can be used in the activating step. In some examples, it may be desirable to significantly decrease the volume in which particles and T cells are mixed together (e.g., increase the concentration of T cells), to ensure maximum contact of the T cells with the particles. For example, the concentration of T cells of about 2 billion cells/mL can be used. In other examples, a T cell concentration of greater than 100 million cells/mL can be used. In other examples, a T cell concentration of about 10 million cells/mL, about 15 million cells/mL, about 20 million cells/mL, 25 million cells/mL, 30 million cells/mL, 35 million cells/mL, 40 million cells/mL, 45 million cells/mL, or 50 million cells/ml can be used. In other embodiments, a concentration of T cells of about 75 million cells/mL, 80 million cells/mL, 85 million cells/mL, 90 million cells/mL, 95 million cells/mL, or 100 million cells/mL can be used. In some examples, a T cell concentration of 125 million cells/mL or 150 million cells/mL can be used. Using high T cell concentrations can result in an increased cell yield, increased T cell activation, and increased T cell proliferation. In addition, the use of high T cell concentrations allows more efficient capture of T cells that have lower expression of target antigens of interest, such as CD28-negative T cells. Such populations of T cells may have therapeutic value and would be desirable to obtain in certain examples. For example, using a high concentration of T cells allows more efficient selection of CD8⁺ T cells that normally have lower CD28 expression.

In some examples, the mixture of T cells and particles may be cultured for about several hours (about 3 hours) to about 14 days, or any hourly integer value in between. In some examples, the mixture may be cultured for 21 days. In some embodiments, the beads and the T cells are cultured together for about eight days. In other examples, the beads and T cells are cultured together for about 2 to about 3 days. Several cycles of activation can be used, such that the T cells and particles can be cultured for about 60 days or more. The T cells and particle mixtures can be cultured using any of the culturing methods and culture media described herein.

T cells that have been exposed to varied activation times may exhibit different characteristics. For example, typical blood or apheresed peripheral blood mononuclear cell products have a helper T cell population (T_{H}, CD4⁺) that is greater than the cytotoxic or suppressor T cell population (T_{c}, CD8⁺). Ex vivo expansion of T cells by stimulating CD3 and CD28 receptors produces a population of T cells that, prior to about days 8-9 of culture, includes predominately of T_{H} cells, while after about days 8-9, the population of T cells includes an increasingly greater population of T_{c} cells. Accordingly, if T_{H} cells are desired, the cells should only be activated for up to 8 to 9 days, and if cytotoxic T cells are desired the T cells should be activated for over 9 or 10 days.

Activation of a T cell can also be performed by culturing a T cell in a culture medium that includes or further includes: an isolated tumor antigen, a tumor lysate, an isolated antigen from a pathogenic organism or virus, or a tumor vaccine.

### Innate Immune Cells

Innate immune cells are mammalian cells that do not recognize pathogenic material (e.g., cancer cells, bacteria, viruses, and yeast) by expressing an antibody or a TCR on its cell surface. Innate immune cells expresses receptors (e.g., receptors on its cell surface) or proteins that bind to the Fc region of other antibodies that are bound to a pathogen and/or receptors that bind to PAMPs that are associated with pathogens and/or DAMPs that are associated with damaged or transformed cells. Non-limiting examples of DAMPs include nuclear or cytosolic proteins (e.g., HMGB1 protein or S100 protein), DNA or RNA, purine metabolites (e.g., ATP, adenosine, or uric acid), and glycans or glycoconjugates (e.g., hyaluronan fragments). Non-limiting examples of PAMPs include bacterial lipopolysaccharide, flagellin, lipoteichoic acid, peptidoglycan, double-stranded RNA, and unmethylated CpG motifs. Additional examples of PAMPs and DAMPs are known in the art.

Non-limiting examples of innate immune cells include mast cells, macrophages, neutrophils, dendritic cells, basophils, eosinophils, and natural killer cells. Additional examples of innate immune cells are known in the art.

Methods of purifying innate immune cells from peripheral blood or tissue from a mammal are known in the art. For example, methods of purifying mast cells are described in Kulka et al., Curr. Protoc. Immunol., Chapter Unit 7.25, 2010; Radinger et al., Current Protoc. Immunol. Chapter Unit 7.37, 2010; Saito et al., Nature Protocols 1:2178-2183, 2006; Davies et al., Methods in Molecular Biology 290:105-116, 2005; Rezapour et al., J. Animal Vet. Sci. 8:11-15, 2009; Roth et al., J. Immunol. Methods 45:153-164, 1981; Thomas et al., J. Immunol. 150:1821-1834, 1993; McLellan et al., J. Immunological Methods 184:81-89, 1995; Akuthota et al., Curr. Protoc. Immunol. Chapter Unit 7.31, 2001; Fairhurst et al., FASEB J. 22:1075, 2008; Munoz et al., Nature Protocols 1:2613-2620, 2007; Falcone et al., Methods Molecular Biology 1192:35-47, 2014; Cooper et al., Current Protocols Immunology, Unit 7.34, 2004; and Pak-Wittel et al., Current Protocols Immunology, Unit 3.22, 2014.

### Culturing T-Cells or Innate Immune Cells

Methods of culturing T cells or innate immune cells are well known in the art. Non-limiting culture media, temperatures, 02 levels, and CO₂ levels that can be used to culture T cells and innate immune cells are provided below. Additional examples of culture media, temperatures, CO₂ levels, and humidity levels that can be used to culture T cells and innate immune cells are known in the art.

Non-limiting examples of culture media that can be used to culture T cells or innate immune cells are known in the art (e.g., Minimal Essential Media or RPM1 Media 1640, or X-vivo 15, (Lonza)). The culture medium used to culture T cells or innate immune cells can, e.g., include factors necessary for proliferation and viability, including, e.g., one or more of: serum (e.g., fetal bovine or human serum), interleukin-2 (IL-2), insulin, IFN-γ, IL-4, IL-7, GM-CSF, IL-10, IL-12, IL-15, a TGF, and a TNF, or any other additives for the growth of cells known to the skilled artisan. The culture media used to culture a T cell or an innate immune cell can, e.g., include a surfactant, piasmanate, and reducing agents, such as N-acetyl-cysteine and 2-mercaptoethanol. Additional examples of culture media include, e.g., RPMI 1640 medium, AIM-V®, DMEM medium, MEM medium, MEM alpha medium, F12 medium, X-VIVO™ 15 medium, X-VIVO™ 20 medium, and OpTmizer™ CTS™ T-Cell Expansion Tissue Culture Medium. Any of the exemplary media described herein can include one or more of: amino acids, sodium pyruvate, vitamins, serum or plasma, one or more hormones, and one or more cytokine(s), e.g., in amounts sufficient for the growth and expansion of T cells or innate immune cells. The culture medium can further include one or more antibiotics (e.g., any of the antibiotics described herein) and/or one or more anti-fungal agents (e.g., any of the anti-fungal agents described herein). Prior to administration to a mammal, the T cells or the innate immune cells are maintained under conditions necessary to support growth, for example, an appropriate temperature (e.g., any of the exemplary temperatures) and atmosphere (e.g., any of the CO₂ levels and/or humidity levels described herein).

In some examples, the culturing is performed in a controlled humidified atmosphere (e.g., at a humidity of greater than 20%, 30%, 40%, 50%, 60%, 70%, 75%, 80%, 85%, 90%, or 95%, or a humidity of 100%).

In some examples, the culturing step can be performed at a temperature of about 31° C. to about 40° C. Skilled practitioners will appreciate that the temperature can be changed at specific time point(s) in during the culturing step, e.g., on an hourly or daily basis. For example, the temperature can be changed or shifted (e.g., increased or decreased) at about one day, two days, three days, four days, five days, six days, seven days, eight days, nine days, ten days, eleven days, twelve days, thirteen days, or fourteen days after the start of the culturing step. For example, the temperature can be shifted upwards (e.g., a change of up to or about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, or up to or about 20° C.). For example, the temperature can be shifted downwards (e.g., a change of up to or about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, or up to or about 20° C.).

In some examples, the culturing step described can further include exposing the liquid culture medium including the cells to an atmosphere containing at most or about 15% CO₂ (e.g., at most or about 14% CO₂, 12% CO₂, 10% CO₂, 8% CO₂, 6% CO₂, 5% CO₂, 4% CO₂, 3% CO₂, 2% CO₂, or at most or about 1% CO₂).

The culturing step in any of the methods described herein can be performed for about 1 hour to about two months, about 7 weeks, about 6 weeks, about 5 weeks, about 4 weeks, about 3 weeks, about 2 weeks, about 13 days, about 12 days, about 11 days, about 10 days, about 9 days, about 8 days, about 7 days, about 6 days, about 5 days, about 4 days, about 3 days, about 2 days, about 1 day, or about 12 hours; about 12 hour to about two months, about 7 weeks, about 6 weeks, about 5 weeks, about 4 weeks, about 3 weeks, about 2 weeks, about 13 days, about 12 days, about 11 days, about 10 days, about 9 days, about 8 days, about 7 days, about 6 days, about 5 days, about 4 days, about 3 days, about 2 days, or about 1 day; about 1 day to about two months, about 7 weeks, about 6 weeks, about 5 weeks, about 4 weeks, about 3 weeks, about 2 weeks, about 13 days, about 12 days, about 11 days, about 10 days, about 9 days, about 8 days, about 7 days, about 6 days, about 5 days, about 4 days, about 3 days, or about 2 days; about 2 days to about two months, about 7 weeks, about 6 weeks, about 5 weeks, about 4 weeks, about 3 weeks, about 2 weeks, about 13 days, about 12 days, about 11 days, about 10 days, about 9 days, about 8 days, about 7 days, about 6 days, about 5 days, about 4 days, or about 3 days; about 3 days to about two months, about 7 weeks, about 6 weeks, about 5 weeks, about 4 weeks, about 3 weeks, about 2 weeks, about 13 days, about 12 days, about 11 days, about 10 days, about 9 days, about 8 days, about 7 days, about 6 days, about 5 days, or about 4 days; about 4 days to about two months, about 7 weeks, about 6 weeks, about 5 weeks, about 4 weeks, about 3 weeks, about 2 weeks, about 13 days, about 12 days, about 11 days, about 10 days, about 9 days, about 8 days, about 7 days, about 6 days, or about 5 days; about 5 days to about two months, about 7 weeks, about 6 weeks, about 5 weeks, about 4 weeks, about 3 weeks, about 2 weeks, about 13 days, about 12 days, about 11 days, about 10 days, about 9 days, about 8 days, about 7 days, or about 6 days; about 6 days to about two months, about 7 weeks, about 6 weeks, about 5 weeks, about 4 weeks, about 3 weeks, about 2 weeks, about 13 days, about 12 days, about 11 days, about 10 days, about 9 days, about 8 days, or about 7 days; about 7 days to about two months, about 7 weeks, about 6 weeks, about 5 weeks, about 4 weeks, about 3 weeks, about 2 weeks, about 13 days, about 12 days, about 11 days, about 10 days, about 9 days, or about 8 days; about 8 days to about two months, about 7 weeks, about 6 weeks, about 5 weeks, about 4 weeks, about 3 weeks, about 2 weeks, about 13 days, about 12 days, about 11 days, about 10 days, or about 9 days; about 9 days to about two months, about 7 weeks, about 6 weeks, about 5 weeks, about 4 weeks, about 3 weeks, about 2 weeks, about 13 days, about 12 days, about 11 days, or about 10 days; about 10 days to about two months, about 7 weeks, about 6 weeks, about 5 weeks, about 4 weeks, about 3 weeks, about 2 weeks, about 13 days, about 12 days, or about 11 days; about 11 days to about two months, about 7 weeks, about 6 weeks, about 5 weeks, about 4 weeks, about 3 weeks, about 2 weeks, about 13 days, or about 12 days; about 12 days to about two months, about 7 weeks, about 6 weeks, about 5 weeks, about 4 weeks, about 3 weeks, about 2 weeks, or about 13 days; about 13 days to about two months, about 7 weeks, about 6 weeks, about 5 weeks, about 4 weeks, about 3 weeks, or about 2 weeks; about 14 days to about two months, about 7 weeks, about 6 weeks, about 5 weeks, about 4 weeks, or about 3 weeks; about 3 weeks to about two months, about 7 weeks, about 6 weeks, about 5 weeks, or about 4 weeks; about 4 weeks to about two months, about 7 weeks, about 6 weeks, or about 5 weeks; about 5 weeks to about two months, about 7 weeks, or about 6 weeks; about 6 weeks to about two months or about 7 weeks; or about 7 weeks to about two months.

Exemplary methods of culturing a T cell are described in Jackson et al., J. Immunol. Methods 291:51-62, 2004; U.S. Pat. No. 5,443,983; U.S. Pat. No. 7,816,134; U.S. Patent Application Publication No. 2008/0261307; WO 02/014481; Barrett et al., Cytotherapy pp. 1-12, 2014; Keever-Taylor et al., Biol. Blood Marrow Transplantation 11:44, 2004; Monica Raulf-Heimsoth, Method Mol. Med. 138:17-30, 2008; Janas et al., Perfusion's Role in Maintenance of High-Density T-Cell Cultures,” BioProcess International website, dated Jan. 13, 2015; Fabricius et al., Immunobiology 156:364-371, 1980; and Watkins et al., J. Vis. Exp. (64):e3952, 2012.

Exemplary methods of culturing innate immune cells are described in Saito et al., Nat. Protoc. 1(4):2178-2183, 2006; Levi-Schaffer et al., Pharmacological Res. 24:307-317, 1991; Davies et al., Methods in Mol. Biology 290:105-116, 2005; Weischenfeldt et al., Cold Spring Harbor Protocols Vol 12, 2008; Zemans et al., J. Immunol. Methods 340:102-115, 2009; Celluzzi et al., J. Hematother. Stem Cell Res. 12:575-585, 2003; Morseet al., Human Cell Culture 4:171-191, 2002; Magee et al., Natural Killer Cells, Basic Science and Clinical Application, Chapter 9-Isolation, Culture and Propagation of Natural Killer Cells, Academic Press, pp. 125-135, 2010; and U.S. Patent Application Publication No. 2008/0299660.

In some embodiments, activating a T cell can include culturing a T cell (e.g., using any of the culture media, culturing conditions, or total time of culturing described herein) can include the use of a culture medium including an agent that activates a T cell (e.g., any of the agents that activate a T cell described herein).

### NLRP3 and NLRP3 Downstream Signaling

NLR Family, Pryin Domain Containing 3 (NLRP3) is a protein that forms a part of an inflammosome complex in mammalian cells. The amino acid sequence of a human NLPR3 protein can be, e.g., SEQ ID NO: 5. An exemplary cDNA sequence encoding a human NLRP3 protein is SEQ ID NO: 6. The amino acid sequence of a cow NLRP3 protein can be, e.g., SEQ ID NO: 7. An exemplary cDNA sequence encoding a cow NLRP3 protein is SEQ ID NO: 8. The amino acid sequence of a mouse NLRP3 protein can be, e.g., SEQ ID NO: 9. An exemplary cDNA sequence encoding a mouse NLRP3 protein is SEQ ID NO: 10. The amino acid sequence of a rat NLRP3 protein can be, e.g., SEQ ID NO: 11. An exemplary cDNA sequence encoding a rat NLRP3 protein is SEQ ID NO: 12.

A level of NLRP3 protein or a level of a cDNA encoding a NLRP3 protein can be detected using methods known in the art. For example, antibodies that specifically bind to a NLRP3 protein (e.g., human NLRP3 protein) can be used to determine a level of a NLRP3 protein in a cell (e.g., a T cell or an innate immune cell). Non-limiting examples of antibodies that bind specifically to human NLRP3 protein are commercially available from EMD Millipore, AdipoGen® Life Sciences, Sigma Aldrich, R&D Systems, OriGene, and Thermo Fisher Scientific.

Methods for determining the level of a cDNA encoding a NLRP3 protein include, e.g., quantitative reverse-transcription polymerase chain reaction (qRT-PCR), Northern blot, ribonuclease protection assays, DNA microarrays, differential display, Taqman RT-PCR, and serial analysis of gene expression (SAGE). For qRT-PCR, methods for designing suitable primers based on a target cDNA sequence (e.g., based on a cDNA sequence encoding a NLRP3 protein) are known in the art.

For example, downstream signaling of NLRP3 can be one or more of: an increase in the formation of an NLRP3 inflamasome complex (e.g., a complex of NLRP3 protein, Apoptosis-associated Speck-like protein containing a caspase recruitment domain (ASC) protein, and caspase-1), an increase in the activity of caspase-1, and an increase in processing of the precursor form of IL-1β and/or IL-18 in a mammalian cell (e.g., any of the T cells or innate immune cells described herein). Exemplary methods for detecting the formation of a NLRP3 inflamasome complex are described in Jhang et al., J. Agric. Food Chem. 63:7343-7352, 2015; Zhao et al., Exp. Neurol. 273:23-35, 2015; and Xiong et al., Kidney Blood Press Res. 40:344-354, 2015. Exemplary methods for detecting the formation of an NLRP3 inflamasome complex include fluorescence microscopy and immunoprecipitation or co-immunoprecitation methods. Kits for detecting an increase in the activity of caspase-1 are commercially sold by Abcam®, GeneTex, Enzo Life Sciences, MD Systems, Thermo Fisher Scientific, Abnova, and Ray Biotech. The processing of the precursor form of IL-1β can be detected using immunoblotting, an antibody that binds specifically to IL-1β protein (e.g., an antibody that binds specifically to the precursor form and the mature form of IL-1β protein, and/or an antibody that only binds to the precursor form of IL-1β protein). Non-limiting examples of antibodies that bind to IL-1β protein include those commercially available from Santa Cruz Biotech, Abcam, Novus Bio, AbD Serotec, and Cell Signaling Technology. The processing of the precursor form of IL-18 protein can be detected using immunoblotting, an antibody that binds specifically to IL-18 (e.g., an antibody that binds specifically to the precursor form and the mature form of IL-18 protein, and/or an antibody that only binds to the precursor form of IL-18 protein). Non-limiting examples of antibodies that bind to IL-18 protein include those commercially available from Santa Cruz Biotech, Abcam, AbD Serotec, Abbiotec, EMD Millipore, R&D Systems, and Thermo Fisher Scientific.

### NLRP3 Activators

In some examples of any of the methods or compositions described herein, a NLRP3 activator can be an agent that increases the level of NLRP3 protein in a mammalian cell (e.g., any of the T cells or an innate immune cells described herein) and/or increases the level of an mRNA encoding a NLRP3 protein in a mammalian cell (e.g., any of the T cells or an innate immune cells described herein). In other examples, the NLRP3 activator increases downstream signaling of NLRP3. For example, downstream signaling of NLRP3 can be one or more of: an increase in the formation of an NLRP3 inflamasome complex (e.g., a complex of NLRP3 protein, Apoptosis-associated Speck-like protein containing a caspase recruitment domain (ASC) protein, and caspase-1, an increase in the activity of caspase-1, and an increase in processing of the precursor form of IL-1β and/or IL-18 in a mammalian cell (e.g., any of the T cells or innate immune cells described herein).

In some examples, the NLRP3 activator is interleukin-1α or interleukin-1β. Human interleukin-1β is commercially available from a number of vendors including, e.g., ProSpec (East Brunswick, N.J.), Thermo Fischer Scientific Inc. (Rockford, Ill.), and ImmunoTools (Friesoythe, Germany). Human interleukin-1α is commercially available from a number of vendors including, e.g., R&D Systems, Life Technologies, and Sigma Aldrich.

For example, the sequence of human IL-1α protein can be SEQ ID NO: 13. An exemplary cDNA sequence encoding a human IL-1α protein is SEQ ID NO: 14. The sequence of mouse IL-1α protein can be, e.g., SEQ ID NO: 15. An exemplary cDNA sequence encoding a mouse IL-1α protein is SEQ ID NO: 16. The sequence of rat IL-1αprotein can be, e.g., SEQ ID NO: 17. An exemplary cDNA sequence encoding a rat IL-1α protein is SEQ ID NO: 18.

For example, the sequence of human IL-1β protein can be SEQ ID NO: 19. An exemplary cDNA sequence encoding a human IL-1β protein is SEQ ID NO: 20. The sequence of mouse IL-1β protein can be, e.g., SEQ ID NO: 21. An exemplary cDNA sequence encoding a mouse IL-1β protein is SEQ ID NO: 22. The sequence of rat IL-1β protein can be, e.g., SEQ ID NO: 23. An exemplary cDNA sequence encoding a rat IL-1β protein is SEQ ID NO: 24.

In some examples, the NLRP3 is a molecule having a molecular weight of less than 10 kDa, less than 9 kDA, less than 8 kDA, less than 7 kDa, less than 6 kDa, less than 5 kDa, less than 4 kDa, less than 3 kDa, less than 2 kDa, or less than 1 kDa. In some examples, the NLRP3 activator is imiquimod or resiquimod, or a pharmaceutically acceptable salt thereof.

In some examples, the NLRP3 activator is:

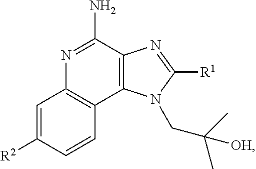

where: (a) R¹ is H, and R² is H; (b) R¹ is a butyl group and R² is H; (c) R¹ is H and R² is —CO₂CH₃; or (d) R¹ is a butyl group and R² is —CO₂CH₃. Methods of synthesizing these NLRP3 activators are described, e.g., in Shi et al., ACS Med. Chem. Lett. 3:501-504, 2012.

In some examples, the NLRP3 activator is selected from the group of:

an imadazoquinoline (see, e.g., the imadazoquinolines described in U.S. Pat. No. 8,658,666);

an imidazonaphthyridine (see, e.g., the imidazonaphthyridines described in U.S. Pat. Nos. 8,658,666 and 7,335,773);

a pyrazolopyridine (see, e.g., the pyrazolopyridines described in U.S. Pat. Nos. 7,678,918 and 7,554,697);

an aryl-substituted imidazoquinoline (see, e.g., the aryl-substituted imidazoquinolines described in U.S. Pat. Nos. 7,598,382 and 7,091,214);

a compound having a 1-alkoxy 1H-imidazo ring system (see, e.g., the compounds having a 1-alkoxy 1H-imidazo ring system described in U.S. Pat. No. 7,579,359);

an oxazolo [4,5-c]-quinolin-4-amine (see, e.g., the oxazolo [4,5-c]-quinolin-4-amines described in U.S. Pat. Nos. 7,148,232; 6,809,203; 6,703,402; 6,677,334; 6,627,640; 6,627,638; 6,440,992; 6,323,200; and 6,110,929);

an thiazolo [4,5-c]-quinolin-4-amine (see, e.g., the thiazolo [4,5-c]-quinolin-4-amines described in U.S. Pat. Nos. 7,148,232; 6,809,203; 6,703,402; 6,677,334; 6,627,640; 6,627,638; 6,440,992; 6,323,200; and 6,110,929);

a selenazolo [4,5-c]-quinolin-4-amine (see, e.g., the selenazolo [4,5-c]-quinolin-4-amines described in U.S. Pat. Nos. 7,148,232; 6,809,203; 6,703,402; 6,677,334; 6,627,640; 6,627,638; 6,440,992; 6,323,200; and 6,110,929);

an imidazonaphthyridine (see, e.g., the imidazonaphthyridines described in U.S. Pat. Nos. 7,038,051; 6,949,646; 6,894,165; 6,797,716; 6,797,716; 6,518,280; 6,514,985; and 6,194,425);

an imidazoquinolinamine (see, e.g., the imidazoquinolinamines described in U.S. Pat. Nos. 7,026,482; 6,897,314; 6,624,305; 6,613,902; 6,534,654; 6,437,131; 6,150,523; 5,998,619; and 5,741,908);

a 1-substituted, 2-substituted 1H-imidazo[4,5-C]quinolin-4-amine (see, e.g., the 1-substituted, 2-substituted 1H-imidazo[4,5-C]quinolin-4-amines described in U.S. Pat. Nos. 6,790,961; 6,686,472; 6,608,201; 6,465,654; 6,348,462; 5,977,366; 5,741,909; 5,525,612; and 5,389,640);

a fused cycloalkylimidazopyridine (see, e.g., the fused cycloaslkylimidazopyridines described in U.S. Pat. Nos. 5,886,006; 5,468,516; 5,627,281; 5,444,065; and 5,352,784);

a 1H-imidazo[4,5-c]quinolin-4-amine (see, e.g., the 1H-imidazo[4,5-c]quinolin-4-amines described in U.S. Pat. No. 5,756,747);

a 1-substituted 1H-imidazo-[4,5-c]quinolin-4-amine (see, e.g., the 1-substituted 1H-imidazo-[4,5-c]quinolin-4-amines described in U.S. Pat. Nos. 5,714,608 and 5,346,905);

an imidazo-[4,5-c]quinolin-4-amine (see, e.g., the imidazo-[4,5-c]quinolin-4-amines described in U.S. Pat. Nos. 5,367,076; 5,175,296; and 4,689,338);

a 2-ethyl 1H-imidazo[4,5-c]quinolin-4-amine (see, e.g., the 2-ethyl 1H-imidazo[4,5-c]quinolin-4-amines described in U.S. Pat. No. 5,266,575);

an olfenic 1H-imidazo[4,5-c]quinolin-4-amine (see, e.g., the olfenic 1H-imidazo[4,5-c]quinolin-4-amines described in U.S. Pat. Nos. 5,037,986 and 4,929,624);

a 1H-imidazo[4,5-c]quinoline (see, e.g., the 1H-imidazo[4,5-c]quinolines described in U.S. Pat. No. 4,698,348);

a pyridoquinoxaline-6-carboxylic acid (see, e.g., the pyridoquinoxaline-6-carboxylic acids described in U.S. Pat. No. 4,449,270);

a 6,7-dihydro-8-(imidazol-1-yl)-5-methyl-1-oxo-1H,5H-benzo [ij]quinolizine-2-carboxylic acid (see, e.g., the 6,7-dihydro-8-(imidazol-1-yl)-5-methyl-1-oxo-1H,5H benzo [ij] quinolizine-2-carboxylic acids described in U.S. Pat. Nos. 4,567,269 and 4,472,406);

a substituted naphtho[ij]quinolizine (see, e.g., the substituted naphtha[ij]quinolizines described in U.S. Pat. No. 4,456,606);

a substituted pyridoquinoxaline-6-carboxylic acid (see, e.g., the substituted pyridoquinoxaline-6-carboxylic acids in U.S. Pat. No. 4,348,521);

a 7-hydroxy-benzo[ij]quinolizine-2-carboxylic acid derivative (see, e.g., the 7-hydroxy-benzo[ij]quinolizine-2-carboxylic acid derivatives described in U.S. Pat. No. 4,051,247);

a substituted benzo[ij]quinolizine-2-carboxylic acid (see, e.g., the substituted benzo[ij]quinolizine-2-carboxylic acids described in U.S. Pat. Nos. 4,014,877; 4,001,243; and 3,985,882);

a 7-hydroxy-benzo[ij]quinolizine-2-carboxylic acid (see, e.g., the 7-hydroxy-benzo[ij]quinolizine-2-carboxylic acids described in U.S. Pat. Nos. 3,985,753 and 3,976,651);

a substituted pyrido[1,2,3,-de]-1,4-benzoxazine (see, e.g., the substituted pyrido[1,2,3,-de]-1,4-benzoxazines described in U.S. Pat. No. 3,984,548); and

a N-methylene malonate of tetrahydroquinoline (see, e.g., the N-methylene malonates of tetrahydroquinoline described in U.S. Pat. No. 3,969,463).

Additional examples of NLRP3 activators are described in U.S. Pat. Nos. 8,658,666; 7,879,849; 7,678,918; 7,598,382; 7,579,359; 7,544,697; 7,335,773; 7,148,232; 7,091,214; 7,038,051; 7,026,482; 6,949,646; 6,897,314; 6,894,165; 6,809,203; 6,797,716; 6,790,961; 6,703,402; 6,686,472; 6,677,334; 6,627,640; 6,627,638; 6,624,305; 6,613,902; 6,608,201; 6,534,654; 6,518,280; 6,514,985; 6,465,654; 6,437,131; 6,348,462; 6,323,200; 6,194,425; 6,150,523; 6,110,929; 5,998,619; 5,977,366; 5,907,083; 5,886,006; 5,756,747; 5,741,909; 5,741,908; 5,714,608; 5,468,516; 5,627,281; 5,605,899; 5,525,612; 5,444,065; 5,389,640; 5,367,076; 5,352,784; 5,346,905; 5,266,575; 5,175,296; 5,037,986; 4,929,624; 4,698,348; 4,689,338; 4,603,199; 4,567,269; 4,449,270; 4,472,406; 4,456,606; 4,443,447; 4,348,521; 4,051,247; 4,014,877; 4,001,243; 3,985,882; 3,985,753; 3,984,548; 3,976,651; and 3,969,463. Additional examples of NLRP3 activators are known in the art.

In some embodiments, a NLRP3 activator (e.g., any of the NLRP3 activators described herein) can be administered to a mammal. Skilled health professionals (e.g., physicians and physician assistants) can determined the dose of NLRP3 activator to administer to a mammal based on one or more known factors (e.g., one or more of the health of the mammal, the age of the mammal, the mass of the mammal, and the sex of the mammal).

For example, a single dose of an NLRP3 activator can include about 1 mg to about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, about 120 mg, about 100 mg, about 90 mg, about 80 mg, about 70 mg, about 60 mg, about 50 mg, about 40 mg, about 30 mg, about 20 mg, about 10 mg, or about 5 mg; about 5 mg to about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, about 120 mg, about 100 mg, about 90 mg, about 80 mg, about 70 mg, about 60 mg, about 50 mg, about 40 mg, about 30 mg, about 20 mg, or about 10 mg; about 10 mg to about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, about 120 mg, about 100 mg, about 90 mg, about 80 mg, about 70 mg, about 60 mg, about 50 mg, about 40 mg, about 30 mg, or about 20 mg; about 20 mg to about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, about 120 mg, about 100 mg, about 90 mg, about 80 mg, about 70 mg, about 60 mg, about 50 mg, about 40 mg, or about 30 mg; about 30 mg to about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, about 120 mg, about 100 mg, about 90 mg, about 80 mg, about 70 mg, about 60 mg, about 50 mg, or about 40 mg; about 40 mg to about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, about 120 mg, about 100 mg, about 90 mg, about 80 mg, about 70 mg, about 60 mg, or about 50 mg; about 50 mg to about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, about 120 mg, about 100 mg, about 90 mg, about 80 mg, about 70 mg, or about 60 mg; about 60 mg to about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, about 120 mg, about 100 mg, about 90 mg, about 80 mg, or about 70 mg; about 70 mg to about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, about 120 mg, about 100 mg, about 90 mg, or about 80 mg; about 80 mg to about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, about 120 mg, about 100 mg, or about 90 mg; about 90 mg to about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, about 120 mg, or about 100 mg; about 100 mg to about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, about 140 mg, or about 120 mg; about 120 mg to about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, about 160 mg, or about 140 mg; about 140 mg to about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 180 mg, or about 160 mg; about 160 mg to about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, or about 180 mg; about 180 mg to about 800 mg, about 750 mg, about 700 mg, about 650 mg, about 600 mg, about 550 mg, about 500 mg, about 450 mg, about 400 mg, about 350 mg, about 300 mg, about 250 mg, or about 200 mg; about 200 mg to about 800 mg, about 750 mg, about 700 mg, about 650 mg, about 600 mg, about 550 mg, about 500 mg, about 450 mg, about 400 mg, about 350 mg, about 300 mg, or about 250 mg; about 250 mg to about 800 mg, about 750 mg, about 700 mg, about 650 mg, about 600 mg, about 550 mg, about 500 mg, about 450 mg, about 400 mg, about 350 mg, or about 300 mg; about 300 mg to about 800 mg, about 750 mg, about 700 mg, about 650 mg, about 600 mg, about 550 mg, about 500 mg, about 450 mg, about 400 mg, or about 350 mg; about 350 mg to about 800 mg, about 750 mg, about 700 mg, about 650 mg, about 600 mg, about 550 mg, about 500 mg, about 450 mg, or about 400 mg; about 400 mg to about 800 mg, about 750 mg, about 700 mg, about 650 mg, about 600 mg, about 550 mg, about 500 mg, or about 450 mg; about 450 mg to about 800 mg, about 750 mg, about 700 mg, about 650 mg, about 600 mg, about 550 mg, or about 500 mg; about 500 mg to about 800 mg, about 750 mg, about 700 mg, about 650 mg, about 600 mg, or about 550 mg; about 550 mg to about 800 mg, about 750 mg, about 700 mg, about 650 mg, or about 600 mg; about 600 mg to about 800 mg, about 750 mg, about 700 mg, or about 650 mg; about 650 mg to about 800 mg, about 750 mg, or about 700 mg; about 700 mg to about 800 mg or about 750 mg; or about 750 mg to about 800 mg of an NLRP3 activator.

Anaplerosis and Conditions for Inducing and/or Increasing Anaplerosis

Anaplerosis is a metabolic activity that replenishes citric acid cycle intermediates that have been withdrawn for synthesis. Compared to control cells, a mammalian cell in which anaplerosis has been induced and/or increased can have one or more of: a decrease in the rate of lactate production from glycolysis, a decrease in the rate of lactate production from glycolysis relative to the rate of oxidative phosphorylation, an increase in the rate of glutamine uptake, an increase in the fraction of lipids and/or amino acids synthesized using glutamine as a substrate, an increase in the rate at which pyruvate is converted to oxaloacetate by pyruvate carboxylase, an increase in the rate at which adenylosuccinate synthetase produces fumurate, an increase in the rate at which aspartate aminotransferase produces oxaloacetate, and an increase in the rate at which propionyl-CoA carboxylase produces succinyl-CoA (e.g., as compared to control cells provided sufficient 02, energy sources, and essential vitamins).

The rate of lactate production from glycolysis can be measured using any number of methods in the art (see, e.g., Glycolysis Assay (Abcam); Lactate Assay Kit (Sigma-Aldrich); and Glycolysis Cell-Based Assay Kit (Caymen Chemical)). The rate of oxidative phosphorylation can be measured using any number of methods in the art (see, e.g., MitoTox™ Complete OXPHOS Activity Assay Kit (Abcam); MitoTox™ Complete OXPHOS Activity Assay Panel (MitoSciences)). The rate of glutamine uptake can be measured using any number of methods in the art. The fraction of lipids and/or amino acids synthesized using glutamine can be measured using any of the methods known in the art (see, e. as a substrate g., Can et al., Cell. Immunol. Immune Recog. 185:1037-1044, 2010; and McDermott et al., J. Cell. Sci. 104; 51-58, 1993). The rate at which pyruvate is converted to oxaloacetate by pyruvate carboxylase can be measured using any of the methods known in the art (see, e.g., Berndt et al., Anal. Biochem. 86:154=158, 1978; and Kerr et al., Mitochondrial Disorders 837:93-119, 2011). The rate at which adenylosuccinate synthetase produces fumarate can be measured using any of the methods known in the art (see, e.g., Van Der Weyden et al., J. Biol. Chem. 249:7282-7289, 1974; Boitz et al., J. Biol. Chem. 288:8977-8990, 2013). The rate at which aspartate aminotransferase produces oxaloacetate can be measured using any of the methods known in the art (see, e.g., Aspartate Aminotransferase (AST) Activity Assay Kit (Sigma-Aldrich); Aspartate Transaminase Assay Kit (Abnova); Alanine Transaminase Colorimetric Activity Assay Kit (Cayman Chemical)). The rate at which propionyl-CoA carboxylase produces succinyl-CoA can be measured using any of the methods known in the art (see, e.g., Gravel et al., Biochem. Med. 20:1-6, 1978; and Sansaricq et al., Pediatric Res. 18:299A, 1984).

A culture medium sufficient to induce and/or increase anaplerosis can include, e.g., one or more of galactose and no added glucose, 3-bromopyruvate, 2-deoxyglucose, pentavalent arsenic (H3AsO4), lonidamine, imatinib, oxythiamine, pyruvate, odd-chain fatty acids, 5-carbon ketone bodies, and triheptanoin. Additional culture media that are sufficient to induce and/or increase anaplerosis are known in the art.

Conditions that are sufficient to induce and/or increase anaplerosis can be, e.g., conditions that result in one or more of the following in a T cell or an innate immune cell as compared to a control cell: a decrease in the rate of lactate production from glycolysis, a decrease in the rate of lactate production from glycolysis relative to the rate of oxidative phosphorylation, an increase in the rate of glutamine uptake by the cell, an increase in the fraction of lipids and/or amino acids synthesized using glutamine as a substrate, an increase in the rate at which pyruvate is converted to oxaloacetate by pyruvate carboxylase, an increase in the rate at which adenylosuccinate synthetase produces fumurate, an increase in the rate at which aspartate aminotransferase produces oxaloacetate, and an increase in the rate at which propionyl-CoA carboxylase produces succinyl-CoA (e.g., as compared to a control cell, e.g., a similar cell that does not have any detectable analperosis or similar cell not cultured under conditions that would induce and/or increase anaplerosis).

### Cancer

In any of the methods described herein, the mammal can have a cancer. In some examples of any of the methods described herein, the mammal has been identified as having a cancer, or has been diagnosed as having a cancer. Some embodiments of any of the methods described herein include obtaining a T cell or an innate immune cell from a mammal having a cancer, a mammal identified as having a cancer, or a mammal diagnosed as having a cancer.

Non-limiting examples of cancer include: acute lymphoblastic leukemia, acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastric cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, gastric cancer, testicular cancer, throat cancer, thyroid cancer, urethral cancer, uterine cancer, vaginal cancer, and vulvar cancer.

Methods for diagnosing a mammal as having a cancer or identifying a mammal as having a cancer are well known in the art. For example, a medical professional (e.g., a physician, a physician's assistant, or a technician) can diagnose cancer in a mammal by observing one or more symptoms of cancer in a mammal. Non-limiting examples of symptoms of cancer include: fatigue, lump or area of thickening felt under the skin, weight change, jaundice, darkening or redness of the skin, sores that won't heal, changes to existing moles, changes in bowel or bladder habits, persistent cough or trouble breathing, difficulty swallowing, hoarseness, persistent indigestion or discomfort after eating, persistent, unexplained muscle or joint pain, persistent, unexplained fevers or night sweats, and unexplained bleeding or bruising. Methods of diagnosing a mammal as having a cancer or identifying a mammal as having a cancer can further include performing one or more diagnostic tests (e.g., performing one or more diagnostic tests on a biopsy or a blood sample).

In some examples of any of the methods described herein, a mammal can be a mammal having a cancer, a mammal diagnosed as having a cancer, or a mammal identified as having a cancer that has been unresponsive to a previously administered treatment for cancer. In some examples of any of the methods described herein, a T cell or an innate immune cell is obtained from a mammal having a cancer, a mammal diagnosed as having a cancer, or a mammal identified as having a cancer that has been unresponsive to a previously administered treatment for cancer. Diagnostic tests for diagnosing a mammal as having a cancer or identifying a mammal as having a cancer are known in the art.

### Infectious Disease

In any of the methods described herein, the mammal can have an infectious disease. In some examples of any of the methods described herein, the mammal has been identified as having an infectious disease, or has been diagnosed as having an infectious disease. For example, an infectious disease can be caused by a bacterium, virus, fungus, parasite, or a mycobacterium. Some embodiments of any of the methods described herein include obtaining a T cell or an innate immune cell from a mammal having an infectious disease, a mammal identified as having an infectious disease, or a mammal diagnosed as having an infectious disease.

Non-limiting examples of infectious disease include: Acinobacter infection, actinomycosis, African sleeping sickness, acquired immunodeficiency syndrome, amebiasis, anaplasmosis, anthrax, Arcanobacterium haemolyticum infection, Argentine hemorrhagic fever, ascariasis, aspergillosis, astrovirus infection, babesiosis, Bacillus cereus infection, bacterial pneumonia, bacterial vaginosis, Bacteroides infection, balantidiasis, Baylisascaris infection, BK virus infection, black piedra, Blastocystic hominis infection, blastomycosis, Bolivian hemorrhagic fever, botulism, Brazilian hemorrhagic fever, brucellosis, bubonic plaque, Burkholderi infection, Buruli ulcer, Calicivirus infection, camptobacteriosis, candidiasis, cat-scratch disease, cellulitis, Chagas disease, chancroid, chickenpox, chikungunya, chlamydia, Chlamydophila pneumoniae infection, cholera, chromoblastomycosis, clonorchiasis, Clostridium difficile infection, coccidioidomycosis, Colorado tick fever, common cold, Creutzfeldt-Jakob disease, Crimean-Congo hemorrhagic fever, crytococcosis, cryptosporidiosis, cutaneous larva migrans, cyclosporiasis, cysticercosis, cytomegalovirus infection, dengue fever, Desmodesmus infection, deintamoebiasis, diphtheria, diphyllobothriasis, dracunculiasis, ebola hemorrhagic fever, echinococcosis, ehrlichiosis, enterobiasis, Enterococcus infection, Enterovirus infection, epidemic typhus, erythema infection, exanthema subitum, fasciolopsiasis, fasciolosis, fatal familial insomnia, filariasis, food poisoning by Clostridium myonecrosis, free-living amebic infection, Fusobacterium infection, gas gangrene, geotrichosis, Gerstmann-Sträussler-Scheinker syndrome, giardiasis, glanders, gnathostomiasis, gonorrhea, granuloma inguinale, Group A streptococcal infection, Group B streptococcal infection, Haemophilus influenzae infection, hand foot and mouth disease, hantavirus pulmonary syndrome, Heartland virus disease, Heliobacter pylori infection, hemolytic-uremic syndrome, hemorrhagic fever with renal syndrome, hepatitis A, hepatitis B, hepatitis C, hepatitis D, hepatitis E, herpes simplex, histoplasmosis, hookworm infection, human bocavirus infection, human ewingii ehrlichiosis, human granulocyte anaplasmosis, human metapneuomovirus infection, human monocytic ehrlichiosis, human papillomavirus infection, human parainfluenza virus infection, hymenolepiasis, Epstein-Barr virus infectious mononucleosis, influenza, isosporiasis, Kawasaki disease, keratitis, Kingella kingae infection, kuru, lassa fever, Legionnaires' disease, Pontiac fever, leishmaniasis, leprosy, leptospirosis, listeriosis, lyme disease, lymphatic filariasis, lymphocytic choriomeningitis, malaria, Marburg hemorrhagic fever, measles, Middle East respiratory syndrome, melioidosis, meningitis, meningococcal disease, metagonimiasis, microsporidiosis, molluscum contagiosum, monkeypox, mumps, murine typhus, mycoplasma pneumonia, mycetoma, myiasis, neonatal conjunctivitis, variant Creutzfeldt-Jakob disease, nocardiosis, onchocerciasis, paracoccidioidomycosis, paragonimiasis, pasteurellosis, pediculosis capitis, pediculosis corporis, pediculosis pubis, pelvic inflammatory disease, pertussis, plague, pneumonia, poliomyelitis, Prevotella infection, primary amoebic meningoencephalitis, progressive multifocal leukoencephalopathy, psittacosis, Q fever, rabies, relapsing fever, respiratory syncytial virus infection, rhinosporidiosis, rhinovirus infection, rickettsial infection, rickettsialpox, Rift Valley Fever, Rocky Mountain spotted fever, rotavirus infection, rubella, salmonellosis, severe acute respiratory syndrome, scabies, schistosomiasis, sepsis, shigellosis, shingles, smallpox, sporothrichosis, staphylococcal food poisoning, staphylococcal infection, staphylococcal infection, strongyloidiasis, subacute sclerosing panencephalitis, syphilis, taeniasis, tetanus, tinea barabe, tinea capitis, tinea corporis, tinea cruris, tinea manum, tinea nigra, tinea pedis, tinea unguium, tinea versicolor, toxocariasis, trachoma, toxoplasmosis, trichinosis, trichomoniasis, trichuriasis, tuberculosis, tularemia, typhoid fever, Ureaplasma urealyticum infection, valley fever, Venezuelan hemorrhagic fever, viral pneumonia, West Nile fever, white piedra, Yersinia psuedotuberculosis infection, yersiniosis, yellow fever, and zygomycosis.

Methods for diagnosing a mammal as having an infectious disease, or identifying a mammal as having a cancer are well known in the art. For example, a medical professional (e.g., a physician, a physician's assistant, or a technician) can diagnose infectious disease in a mammal by observing one or more symptoms of infectious disease in a mammal. Non-limiting examples of symptoms of infectious disease include: fever, diarrhea, fatigue, and muscle aches. Methods of diagnosing a mammal as having an infectious disease or identifying a mammal as having an infectious disease can further include performing one or more diagnostic tests (e.g., performing one or more diagnostic tests on a biopsy or a blood sample). Diagnostic tests for diagnosing a mammal as having an infectious disease or identifying a mammal as having an infectious disease are known in the art.

### Methods of Increasing Resistance of a T Cell or an Immune Cell to an Immunosuppressive Cytokine

Also provided herein are methods of increasing resistance of a T cell or an innate immune cell to at least one immunosuppressive cytokine (e.g., any one or more of the immunosuppressive cytokines described herein) that include: (a) providing a T cell (e.g., any of the T cells described herein) or an innate immune cell (e.g., any of the innate immune cells described herein), and (b) culturing the T cell or the innate immune cell in a culture medium (e.g., any of the culture media or using any of the culturing methods described herein) including an amount of an NLRP3 activator (e.g., any of the NLRP3 activators described herein) sufficient to increase resistance of a T cell or an innate immune cell to the at least one immunosuppressive cytokine (e.g., as compared to a T cell or an innate immune cell that has not been contacted with an NLRP3 activator). Non-limiting examples of immunosuppressive cytokines include: IL-10, TGF-β, IL-1Ra, IL-18Ra, IL-2, IL-3, IL-4, IL-5, IL-6, IL-7, IL-8, IL-9, IL-11, IL-12, IL-13, IL-14, IL-15, IL-16, IL-17, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, PGE2, SCF, G-CSF, CSF-1R, M-CSF, GM-CSF, IFN-α, IFN-β, IFN-γ, IFN-λ, bFGF, CCL2, CXCL1, CXCL8, CXCL12, CX3CL1, CXCR4, and VEGF.

Exemplary methods for obtaining a T cell (e.g., any of the T cells described herein) or an innate immune cell (e.g., any of the innate immune cells described herein) from a mammal are described herein. Additional methods for obtaining a T cell (e.g., any of the T cells described herein) or an innate immune cell (e.g., any of the innate immune cells described herein) from a mammal are known in the art. The culturing of a T cell or an innate immune cell can be performed using any of the culturing methods, culture conditions, and culture times described herein. Any of the NLRP3 activators described herein can be used in these methods.

Some examples of these methods further include administering the T cell or the innate immune cell back to the mammal (e.g., when the T cell or innate immune cell is obtained from the mammal). In some examples, the mammal has a cancer, is identified as having a cancer, or has been diagnosed as having a cancer. The T cell or the innate immune cell can be administered back to the mammal using intaarterial injection, intravenous injection, or intratumor injection. Any of the total numbers of T cells or innate immune cells, or any of the dosing parameters of T cells or innate immune cells described herein can be used in these methods. The T cells or innate immune cells can be administered in multiple doses to the mammal (e.g., using any of the frequencies of administration described herein). The T cells or innate immune cells can be formulated into any of the exemplary compositions described herein prior to administration.

### Methods of Improving the Anti-Tumor Activity of a T Cell

Also provided herein are methods of improving the anti-tumor activity of a T cell that include: (a) providing a T cell (e.g., any of the T cells described herein); and (b) culturing the T cell (e.g., using any of the culturing methods, culture media, culture conditions, or culture times described herein): in a culture medium sufficient to induce and/or increase anaplerosis (e.g., any of the culture media sufficient to induce and/or increase anaplerosis described herein) and/or comprising a NLRP3 activator (e.g., any of the NLRP3 activators described herein) in an amount sufficient to improve anti-tumor activity of a T cell (e.g., any of the anti-tumor activities of a T cell described herein); and/or under conditions sufficient to induce and/or increase anaplerosis (e.g., any of the conditions sufficient to induce and/or increase anaplerosis described herein), thereby resulting in an improvement in the anti-tumor activity of the T cell.

Any of the culturing methods, culture media, culture conditions, and/or culture time lengths described herein can be used in these methods. Any of the NLRP3 activators described herein, any of the culture media sufficient to induce and/or increase anaplerosis, and/or any of the conditions sufficient to induce and/or increase anaplerosis described herein can be used in these methods.

Non-limiting examples anti-tumor activities of a T cell include: increased differentiation of premature cells into a T cell, increased division and proliferation of a T cell, increased extravasation of a T cell from the blood into a tissue, increased ability of a T cell to infiltrate a solid tumor, increased activation of a T cell, increased ability of a T cell to kill a tumor cell, increased ability of a T cell to recruit other cells of the immune system to kill a tumor cell, and increased ability of T cells to activate factors to kill a tumor cell. Methods for detecting the anti-tumor activities of a T cell in vitro are known in the art. For example, the differentiation of premature cells into T cells can be detected by detecting the level of expression of proteins in the cell that indicate that the cell has differentiated into a T cell (e.g., TCR and CD3). Exemplary assays for detecting the level of extravasation of a T cell include, e.g., the use of Transwell culture systems. Exemplary assays for detecting the level of infiltration of a T cell into a solid tumor are described in Ye et al., J. Clin. Immunol. 26:447-456, 2006. Exemplary assays for detecting the levels of cytokines and/or lymphokines produced by T cells that decrease anti-tumor immune activity or promote anti-tumor immune activity can include, e.g., the use of one or more antibodies that specifically bind to a cytokine or lymphokine produced by a T cell that decrease anti-tumor immune activity or promote anti-tumor immune activity, in e.g., an immunoassay (e.g., an ELISA). These antibodies are commercially available from a number of vendors. Method of detecting the level of T cell activation are known in the art. T-cell activation means the binding of one or more receptors (e.g., a TCR optionally in association with a CD3 dimer) on the surface of a T cell with one or more cognate receptors on the surface of an antigen-presenting cell. The term activate a T cell can include the binding of one or more integrins on the surface of the T cell with their respective ligands (e.g., intercellular adhesion molecule (ICAM), vascular cell adhesion molecule (VCAM), or fibronectin), or the activation of a co-stimulatory receptor on a T cell (e.g., CD28, ICSO, CD40) by binding to its ligand. For example CD28 is activated by CD80 or CD86, ICOS is activated by ICOS-L, and ICOS is activated by ICOS-L. Exemplary methods of detecting activation of a T cell include measuring T cell proliferation, upregulation of activation markers on the T cell surface, differentiation of the T cells into effector cells, induction of cytotoxity or cytokine secretion, and induction of apoptosis. Assays for detecting T cell proliferation are commercially available. Examples of such methods for detecting activation markers on the T cell surface, markers of differentiation of T cells into effector cells, markers of induction of cytotoxicity, cytokines secreted by activated T cells, and markers of apoptosis can be performed using, e.g., one or more antibodies that bind to an activation marker on the T cell surface, a marker of differentiation of T cells into effector cells, a cytokine secreted by activated T cells, or a marker of apoptosis. Such antibodies are commercially available from a variety of different vendors.

Some examples of these methods further include administering the T cell back to the mammal (e.g., when the T cell is obtained from the mammal). In some examples, the mammal has a cancer, is identified as having a cancer, or has been diagnosed as having a cancer. The T cell can be administered back to the mammal using intaarterial injection, intravenous injection, or intratumor injection. Any of the total numbers of T cells, or any of the dosing parameters of T cells described herein can be used in these methods. The T cells can be administered in multiple doses to the mammal (e.g., using any of the frequencies of administration described herein). The T cells can be formulated into any of the exemplary compositions described herein prior to administration.

### T-Cells and Innate Immune Cells

Also provided herein are T cells (e.g., any of the T cells described herein) and innate immune cells (e.g., any of the innate immune cells described herein) produced by any of the methods described herein. For example, provided herein are T cells (e.g., any of the T cells described herein) having increased resistance to at least one immunosuppressive cytokine produced by any of the methods described herein. Also provided are innate immune cells (e.g., any of the innate immune cells described herein) that have increased resistance to at least one immunosuppressive cytokine produced by any of the methods described herein. Also provided are T cells (e.g., any of the T cells described herein) that have improved anti-tumor activity (e.g., any of the exemplary types of improved anti-tumor activity described herein) produced by any of the methods described herein.

### Pharmaceutical Compositions

Also provided herein are pharmaceutical compositions that include any of the T cells and/or innate immune cells described in the above section. The pharmaceutical composition can have a total cell density of about 1×10⁵ cells/mL to about 30×10⁶ cells/mL, about 28×10⁶ cells/mL, about 26×10⁶ cells/mL, about 24×10⁶ cells/mL, about 22×10⁶ cells/mL, about 20×10⁶ cells/mL, about 18×10⁶ cells/mL about 16×10⁶ cells/mL, about 14×10⁶ cells/mL, about 12×10⁶ cells/mL, about 10×10⁶ cells/mL, about 8×10⁶ cells/mL, about 6×10⁶ cells/mL about 4×10⁶ cells/mL, about 2×10⁶ cells/mL, about 1×10⁶ cells/mL, about 0.5×10⁶ cells/mL, or about 0.25×10⁶ cells/mL; about 0.25×10⁶ cells/mL to about 30×10⁶ cells/mL, about 28×10⁶ cells/mL, about 26×10⁶ cells/mL, about 24×10⁶ cells/mL, about 22×10⁶ cells/mL, about 20×10⁶ cells/mL, about 18×10⁶ cells/mL, about 16×10⁶ cells/mL, about 14×10⁶ cells/mL, about 12×10⁶ cells/mL, about 10×10⁶ cells/mL, about 8×10⁶ cells/mL, about 6×10⁶ cells/mL, about 4×10⁶ cells/mL, about 2×10⁶ cells/mL, about 1×10⁶ cells/mL, or about 0.5×10⁶ cells/mL; about 0.5×10⁶ cells/mL to about 30×10⁶ cells/mL, about 28×10⁶ cells/mL, about 26×10⁶ cells/mL, about 24×10⁶ cells/mL, about 22×10⁶ cells/mL, about 20×10⁶ cells/mL, about 18×10⁶ cells/mL, about 16×10⁶ cells/mL, about 14×10⁶ cells/mL, about 12×10⁶ cells/mL, about 10×10⁶ cells/mL, about 8×10⁶ cells/mL, about 6×10⁶ cells/mL, about 4×10⁶ cells/mL, about 2×10⁶ cells/mL, or about 1×10⁶ cells/mL; about 1×10⁶ cells/mL to about 30×10⁶ cells/mL, about 28×10⁶ cells/mL, about 26×10⁶ cells/mL, about 24×10⁶ cells/mL, about 22×10⁶ cells/mL, about 20×10⁶ cells/mL, about 18×10⁶ cells/mL, about 16×10⁶ cells/mL, about 14×10⁶ cells/mL, about 12×10⁶ cells/mL, about 10×10⁶ cells/mL, about 8×10⁶ cells/mL, about 6×10⁶ cells/mL, about 4×10⁶ cells/mL, or about 2×10⁶ cells/mL; about 2×10⁶ cells/mL to about 30×10⁶ cells/mL, about 28×10⁶ cells/mL, about 26×10⁶ cells/mL, about 24×10⁶ cells/mL, about 22×10⁶ cells/mL, about 20×10⁶ cells/mL, about 18×10⁶ cells/mL, about 16×10⁶ cells/mL, about 14×10⁶ cells/mL, about 12×10⁶ cells/mL, about 10×10⁶ cells/mL, about 8×10⁶ cells/mL, about 6×10⁶ cells/mL, or about 4×10⁶ cells/mL; about 4×10⁶ cells/mL to about 30×10⁶ cells/mL, about 28×10⁶ cells/mL, about 26×10⁶ cells/mL, about 24×10⁶ cells/mL, about 22×10⁶ cells/mL, about 20×10⁶ cells/mL, about 18×10⁶ cells/mL, about 16×10⁶ cells/mL, about 14×10⁶ cells/mL, about 12×10⁶ cells/mL, about 10×10⁶ cells/mL, about 8×10⁶ cells/mL, or about 6×10⁶ cells/mL; about 6×10⁶ cells/mL to about 30×10⁶ cells/mL, about 28×10⁶ cells/mL, about 26×10⁶ cells/mL, about 24×10⁶ cells/mL, about 22×10⁶ cells/mL, about 20×10⁶ cells/mL, about 18×10⁶ cells/mL, about 16×10⁶ cells/mL, about 14×10⁶ cells/mL, about 12×10⁶ cells/mL, about 10×10⁶ cells/mL, or about 8×10⁶ cells/mL; about 8×10⁶ cells/mL to about 30×10⁶ cells/mL, about 28×10⁶ cells/mL, about 26×10⁶ cells/mL, about 24×10⁶ cells/mL, about 22×10⁶ cells/mL, about 20×10⁶ cells/mL, about 18×10⁶ cells/mL, about 16×10⁶ cells/mL, about 14×10⁶ cells/mL, about 12×10⁶ cells/mL, or about 10×10⁶ cells/mL; about 10×10⁶ cells/mL to about 30×10⁶ cells/mL, about 28×10⁶ cells/mL, about 26×10⁶ cells/mL, about 24×10⁶ cells/mL, about 22×10⁶ cells/mL, about 20×10⁶ cells/mL, about 18×10⁶ cells/mL, about 16×10⁶ cells/mL, about 14×10⁶ cells/mL, or about 12×10⁶ cells/mL; about 12×10⁶ cells/mL to about 30×10⁶ cells/mL, about 28×10⁶ cells/mL, about 26×10⁶ cells/mL, about 24×10⁶ cells/mL, about 22×10⁶ cells/mL, about 20×10⁶ cells/mL, about 18×10⁶ cells/mL, about 16×10⁶ cells/mL, or about 14×10⁶ cells/mL; about 14×10⁶ cells/mL to about 30×10⁶ cells/mL, about 28×10⁶ cells/mL, about 26×10⁶ cells/mL, about 24×10⁶ cells/mL, about 22×10⁶ cells/mL, about 20×10⁶ cells/mL, about 18×10⁶ cells/mL, or about 16×10⁶ cells/mL; about 16×10⁶ cells/mL to about 30×10⁶ cells/mL, about 28×10⁶ cells/mL, about 26×10⁶ cells/mL, about 24×10⁶ cells/mL, about 22×10⁶ cells/mL, about 20×10⁶ cells/mL, or about 18×10⁶ cells/mL; about 18×10⁶ cells/mL to about 30×10⁶ cells/mL, about 28×10⁶ cells/mL, about 26×10⁶ cells/mL, about 24×10⁶ cells/mL, about 22×10⁶ cells/mL, or about 20×10⁶ cells/mL; about 20×10⁶ cells/mL to about 30×10⁶ cells/mL, about 28×10⁶ cells/mL, about 26×10⁶ cells/mL, about 24×10⁶ cells/mL, or about 22×10⁶ cells/mL; about 22×10⁶ cells/mL to about 30×10⁶ cells/mL, about 28×10⁶ cells/mL, about 26×10⁶ cells/mL, or about 24×10⁶ cells/mL; about 24×10⁶ cells/mL to about 30×10⁶ cells/mL, about 28×10⁶ cells/mL, or about 26×10⁶ cells/mL; about 26×10⁶ cells/mL to about 30×10⁶ cells/mL or about 28×10⁶ cells/mL; or about 28×10⁶ cells/mL to about 30×10⁶ cells/mL.

Each dose of the pharmaceutical composition can be about 0.5×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, about 10.5×10⁶ cells/kg, about 10×10⁶ cells/kg, about 9.5×10⁶ cells/kg, about 9×10⁶ cells/kg, about 8.5×10⁶ cells/kg, about 8×10⁶ cells/kg, about 7.5×10⁶ cells/kg, about 7×10⁶ cells/kg, about 6.5×10⁶ cells/kg, about 6×10⁶ cells/kg, about 5.5×10⁶ cells/kg, about 5×10⁶ cells/kg, about 4.5×10⁶ cells/kg, about 4×10⁶ cells/kg, about 3.5×10⁶ cells/kg, about 3×10⁶ cells/kg, about 2.5×10⁶ cells/kg, about 2×10⁶ cells/kg, about 1.5×10⁶ cells/kg, or about 1×10⁶ cells/kg; about 1×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, about 10.5×10⁶ cells/kg, about 10×10⁶ cells/kg, about 9.5×10⁶ cells/kg, about 9×10⁶ cells/kg, about 8.5×10⁶ cells/kg, about 8×10⁶ cells/kg, about 7.5×10⁶ cells/kg, about 7×10⁶ cells/kg, about 6.5×10⁶ cells/kg, about 6×10⁶ cells/kg, about 5.5×10⁶ cells/kg, about 5×10⁶ cells/kg, about 4.5×10⁶ cells/kg, about 4×10⁶ cells/kg, about 3.5×10⁶ cells/kg, about 3×10⁶ cells/kg, about 2.5×10⁶ cells/kg, about 2×10⁶ cells/kg, or about 1.5×10⁶ cells/kg; 1.5×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, about 10.5×10⁶ cells/kg, about 10×10⁶ cells/kg, about 9.5×10⁶ cells/kg, about 9×10⁶ cells/kg, about 8.5×10⁶ cells/kg, about 8×10⁶ cells/kg, about 7.5×10⁶ cells/kg, about 7×10⁶ cells/kg, about 6.5×10⁶ cells/kg, about 6×10⁶ cells/kg, about 5.5×10⁶ cells/kg, about 5×10⁶ cells/kg, about 4.5×10⁶ cells/kg, about 4×10⁶ cells/kg, about 3.5×10⁶ cells/kg, about 3×10⁶ cells/kg, about 2.5×10⁶ cells/kg, or about 2×10⁶ cells/kg; about 2×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, about 10.5×10⁶ cells/kg, about 10×10⁶ cells/kg, about 9.5×10⁶ cells/kg, about 9×10⁶ cells/kg, about 8.5×10⁶ cells/kg, about 8×10⁶ cells/kg, about 7.5×10⁶ cells/kg, about 7×10⁶ cells/kg, about 6.5×10⁶ cells/kg, about 6×10⁶ cells/kg, about 5.5×10⁶ cells/kg, about 5×10⁶ cells/kg, about 4.5×10⁶ cells/kg, about 4×10⁶ cells/kg, about 3.5×10⁶ cells/kg, about 3×10⁶ cells/kg, or about 2.5×10⁶ cells/kg; 2.5×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, about 10.5×10⁶ cells/kg, about 10×10⁶ cells/kg, about 9.5×10⁶ cells/kg, about 9×10⁶ cells/kg, about 8.5×10⁶ cells/kg, about 8×10⁶ cells/kg, about 7.5×10⁶ cells/kg, about 7×10⁶ cells/kg, about 6.5×10⁶ cells/kg, about 6×10⁶ cells/kg, about 5.5×10⁶ cells/kg, about 5×10⁶ cells/kg, about 4.5×10⁶ cells/kg, about 4×10⁶ cells/kg, about 3.5×10⁶ cells/kg, or about 3×10⁶ cells/kg; about 3×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, about 10.5×10⁶ cells/kg, about 10×10⁶ cells/kg, about 9.5×10⁶ cells/kg, about 9×10⁶ cells/kg, about 8.5×10⁶ cells/kg, about 8×10⁶ cells/kg, about 7.5×10⁶ cells/kg, about 7×10⁶ cells/kg, about 6.5×10⁶ cells/kg, about 6×10⁶ cells/kg, about 5.5×10⁶ cells/kg, about 5×10⁶ cells/kg, about 4.5×10⁶ cells/kg, about 4×10⁶ cells/kg, or about 3.5×10⁶ cells/kg; 3.5×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, about 10.5×10⁶ cells/kg, about 10×10⁶ cells/kg, about 9.5×10⁶ cells/kg, about 9×10⁶ cells/kg, about 8.5×10⁶ cells/kg, about 8×10⁶ cells/kg, about 7.5×10⁶ cells/kg, about 7×10⁶ cells/kg, about 6.5×10⁶ cells/kg, about 6×10⁶ cells/kg, about 5.5×10⁶ cells/kg, about 5×10⁶ cells/kg, about 4.5×10⁶ cells/kg, or about 4×10⁶ cells/kg; about 4×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, about 10.5×10⁶ cells/kg, about 10×10⁶ cells/kg, about 9.5×10⁶ cells/kg, about 9×10⁶ cells/kg, about 8.5×10⁶ cells/kg, about 8×10⁶ cells/kg, about 7.5×10⁶ cells/kg, about 7×10⁶ cells/kg, about 6.5×10⁶ cells/kg, about 6×10⁶ cells/kg, about 5.5×10⁶ cells/kg, about 5×10⁶ cells/kg, or about 4.5×10⁶ cells/kg; 4.5×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, about 10.5×10⁶ cells/kg, about 10×10⁶ cells/kg, about 9.5×10⁶ cells/kg, about 9×10⁶ cells/kg, about 8.5×10⁶ cells/kg, about 8×10⁶ cells/kg, about 7.5×10⁶ cells/kg, about 7×10⁶ cells/kg, about 6.5×10⁶ cells/kg, about 6×10⁶ cells/kg, about 5.5×10⁶ cells/kg, or about 5×10⁶ cells/kg; about 5×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, about 10.5×10⁶ cells/kg, about 10×10⁶ cells/kg, about 9.5×10⁶ cells/kg, about 9×10⁶ cells/kg, about 8.5×10⁶ cells/kg, about 8×10⁶ cells/kg, about 7.5×10⁶ cells/kg, about 7×10⁶ cells/kg, about 6.5×10⁶ cells/kg, about 6×10⁶ cells/kg, or about 5.5×10⁶ cells/kg; about 5.5×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, about 10.5×10⁶ cells/kg, about 10×10⁶ cells/kg, about 9.5×10⁶ cells/kg, about 9×10⁶ cells/kg, about 8.5×10⁶ cells/kg, about 8×10⁶ cells/kg, about 7.5×10⁶ cells/kg, about 7×10⁶ cells/kg, about 6.5×10⁶ cells/kg, or about 6×10⁶ cells/kg; about 6×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, about 10.5×10⁶ cells/kg, about 10×10⁶ cells/kg, about 9.5×10⁶ cells/kg, about 9×10⁶ cells/kg, about 8.5×10⁶ cells/kg, about 8×10⁶ cells/kg, about 7.5×10⁶ cells/kg, about 7×10⁶ cells/kg, or about 6.5×10⁶ cells/kg; about 6.5×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, about 10.5×10⁶ cells/kg, about 10×10⁶ cells/kg, about 9.5×10⁶ cells/kg, about 9×10⁶ cells/kg, about 8.5×10⁶ cells/kg, about 8×10⁶ cells/kg, about 7.5×10⁶ cells/kg, or about 7×10⁶ cells/kg; about 7×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, about 10.5×10⁶ cells/kg, about 10×10⁶ cells/kg, about 9.5×10⁶ cells/kg, about 9×10⁶ cells/kg, about 8.5×10⁶ cells/kg, about 8×10⁶ cells/kg, or about 7.5×10⁶ cells/kg; about 7.5×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, about 10.5×10⁶ cells/kg, about 10×10⁶ cells/kg, about 9.5×10⁶ cells/kg, about 9×10⁶ cells/kg, about 8.5×10⁶ cells/kg, or about 8×10⁶ cells/kg; about 8×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, about 10.5×10⁶ cells/kg, about 10×10⁶ cells/kg, about 9.5×10⁶ cells/kg, about 9×10⁶ cells/kg, or about 8.5×10⁶ cells/kg; about 8.5×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, about 10.5×10⁶ cells/kg, about 10×10⁶ cells/kg, about 9.5×10⁶ cells/kg, or about 9×10⁶ cells/kg; about 9×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, a about 11.5×10⁶ cells/kg, about 11×10⁶ cells/kg, about 10.5×10⁶ cells/kg, about 10×10⁶ cells/kg, or about 9.5×10⁶ cells/kg; about 9.5×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, about 10.5×10⁶ cells/kg, or about 10×10⁶ cells/kg; about 10×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, about 11×10⁶ cells/kg, or about 10.5×10⁶ cells/kg; about 10.5×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, about 11.5×10⁶ cells/kg, or about 11×10⁶ cells/kg; about 11×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, about 12×10⁶ cells/kg, or about 11.5×10⁶ cells/kg; about 11.5×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, about 12.5×10⁶ cells/kg, or about 12×10⁶ cells/kg; about 12×10⁶ cells/kg to about 15×10⁶ cells/kg, about 14.5×10⁶ cells/kg, about 14.0×10⁶ cells/kg, about 13.5×10⁶ cells/kg, about 13.0×10⁶ cells/kg, or about 12.5×10⁶ cells/kg; about 12.5×10⁶ cells/kg to about 15×10⁶ cells/kg, about 14.5×10⁶ cells/kg, about 14.0×10⁶ cells/kg, about 13.5×10⁶ cells/kg, or about 13.0×10⁶ cells/kg; about 13×10⁶ cells/kg to about 15×10⁶ cells/kg, about 14.5×10⁶ cells/kg, about 14.0×10⁶ cells/kg, or about 13.5×10⁶ cells/kg; about 13.5×10⁶ cells/kg to about 15×10⁶ cells/kg, about 14.5×10⁶ cells/kg, or about 14.0×10⁶ cells/kg; about 14×10⁶ cells/kg to about 15×10⁶ cells/kg or about 14.5×10⁶ cells/kg; or about 14.5×10⁶ cells/kg to about 15×10⁶ cells/kg.

Each dose of the pharmaceutical composition can include about 5 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, about 1 billion cells, about 900 million cells, about 800 million cells, about 700 million cells, about 600 million cells, about 500 million cells, about 450 million cells, about 400 million cells, about 350 million cells, about 300 million cells, about 250 million cells, about 200 million cells, about 180 million cells, about 160 million cells, about 140 million cells, about 120 million cells, about 100 million cells, about 80 million cells, about 60 million cells, about 40 million cells, or about 20 million cells; about 20 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 20 million cells, about 1 billion cells, about 900 million cells, about 800 million cells, about 700 million cells, about 600 million cells, about 500 million cells, about 450 million cells, about 400 million cells, about 350 million cells, about 300 million cells, about 250 million cells, about 200 million cells, about 180 million cells, about 160 million cells, about 140 million cells, about 120 million cells, about 100 million cells, about 80 million cells, about 60 million cells, or about 40 million cells; about 40 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, about 1 billion cells, about 900 million cells, about 800 million cells, about 700 million cells, about 600 million cells, about 500 million cells, about 450 million cells, about 400 million cells, about 350 million cells, about 300 million cells, about 250 million cells, about 200 million cells, about 180 million cells, about 160 million cells, about 140 million cells, about 120 million cells, about 100 million cells, about 80 million cells, or about 60 million cells; about 60 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, about 1 billion cells, about 900 million cells, about 800 million cells, about 700 million cells, about 600 million cells, about 500 million cells, about 450 million cells, about 400 million cells, about 350 million cells, about 300 million cells, about 250 million cells, about 200 million cells, about 180 million cells, about 160 million cells, about 140 million cells, about 120 million cells, about 100 million cells, or about 80 million cells; about 80 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, about 1 billion cells, about 900 million cells, about 800 million cells, about 700 million cells, about 600 million cells, about 500 million cells, about 450 million cells, about 400 million cells, about 350 million cells, about 300 million cells, about 250 million cells, about 200 million cells, about 180 million cells, about 160 million cells, about 140 million cells, about 120 million cells, or about 100 million cells; about 100 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, about 1 billion cells. about 900 million cells, about 800 million cells, about 700 million cells, about 600 million cells, about 500 million cells, about 450 million cells, about 400 million cells, about 350 million cells, about 300 million cells, about 250 million cells, about 200 million cells, about 180 million cells, about 160 million cells, about 140 million cells, or about 120 million cells; about 120 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, about 1 billion cells, about 900 million cells, about 800 million cells, about 700 million cells, about 600 million cells, about 500 million cells, a about 450 million cells, about 400 million cells, about 350 million cells, about 300 million cells, about 250 million cells, about 200 million cells, about 180 million cells, about 160 million cells, or about 140 million cells; about 140 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, about 1 billion cells, about 900 million cells, about 800 million cells, about 700 million cells, about 600 million cells, about 500 million cells, about 450 million cells, about 400 million cells, about 350 million cells, about 300 million cells, about 250 million cells, about 200 million cells, about 180 million cells, or about 160 million cells; about 160 millions cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, about 1 billion cells, about 900 million cells, about 800 million cells, about 700 million cells, about 600 million cells, about 500 million cells, about 450 million cells, about 400 million cells, about 350 million cells, about 300 million cells, about 250 million cells, about 200 million cells, or about 180 million cells; about 180 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, about 1 billion cells, about 900 million cells, about 800 million cells, about 700 million cells, about 600 million cells, about 500 million cells, about 450 million cells, about 400 million cells, about 350 million cells, about 300 million cells, about 250 million cells, or about 200 million cells; about 200 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, about 1 billion cells, about 900 million cells, about 800 million cells, about 700 million cells, about 600 million cells, about 500 million cells, about 450 million cells, about 400 million cells, about 350 million cells, about 300 million cells, or about 250 million cells; about 250 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, about 1 billion cells, about 900 million cells, about 800 million cells, about 700 million cells, about 600 million cells, about 500 million cells, about 450 million cells, about 400 million cells, about 350 million cells, or about 300 million cells; about 300 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, about 1 billion cells, about 900 million cells, about 800 million cells, about 700 million cells, about 600 million cells, about 500 million cells, about 450 million cells, about 400 million cells, or about 350 million cells; about 350 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, about 1 billion cells, about 900 million cells, about 800 million cells, about 700 million cells, about 600 million cells, about 500 million cells, about 450 million cell, or about 400 million cells; about 400 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, about 1 billion cells, about 900 million cells, about 800 million cells, about 700 million cells, about 600 million cells, about 500 million cells, or about 450 million cells; about 500 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, about 1 billion cells, about 900 million cells, about 800 million cells, about 700 million cells, or about 600 million cells; about 600 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, about 1 billion cells, about 900 million cells, about 800 million cells, or about 700 million cells; about 700 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, about 1 billion cells, about 900 million cells, or about 800 million cells; about 800 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, about 1 billion cells, or about 900 million cells; about 900 million cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, about 1.1 billion cells, or about 1 billion cells; about 1 billion cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, about 1.2 billion cells, or about 1.1 billion cells; about 1.1 billion cells to about 2.25 billion cells, about 2 billion cells, about 1.9 billion cells, about 1.8 billion cells, about 1.7 billion cells, about 1.6 billion cells, about 1.5 billion cells, about 1.4 billion cells, about 1.3 billion cells, or about 1.2 billion cells; about 1.2 billion cells to about 2.25 billion cells, about 2 billion cells, about 1.9 billion cells, about 1.8 billion cells, about 1.7 billion cells, about 1.6 billion cells, about 1.5 billion cells, about 1.4 billion cells, or about 1.3 billion cells; about 1.3 billion cells to about 2.25 billion cells, about 2 billion cells, about 1.9 billion cells, about 1.8 billion cells, about 1.7 billion cells, about 1.6 billion cells, about 1.5 billion cells, or about 1.4 billion cells; about 1.4 billion cells to about 2.25 billion cells, about 2 billion cells, about 1.9 billion cells, about 1.8 billion cells, about 1.7 billion cells, about 1.6 billion cells, or about 1.5 billion cells; about 1.5 billion cells to about 2.25 billion cells, about 2 billion cells, about 1.9 billion cells, about 1.8 billion cells, about 1.7 billion cells, or about 1.6 billion cells; about 1.6 billion cells to about 2.25 billion cells, about 2 billion cells, about 1.9 billion cells, about 1.8 billion cells, or about 1.7 billion cells; about 1.7 billion cells to about 2.25 billion cells, about 2 billion cells, about 1.9 billion cells, or about 1.8 billion cells; about 1.8 billion cells to about 2.25 billion cells, about 2 billion cells, or about 1.9 billion cells; about 1.9 billion cells to about 2.25 billion cells or about 2 billion cells; or about 2.0 billion cells to about 2.25 billion cells.

A single dose of any of the pharmaceutical compositions described herein can have a volume of about 2.5 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, about 160 mL, about 150 mL, about 140 mL, about 130 mL, about 120 mL, about 100 mL, about 90 mL, about 80 mL, about 70 mL, about 60 mL, about 50 mL, about 40 mL, about 30 mL, about 25 mL, about 20 mL, about 15 mL, about 10 mL, or about 5 mL; about 5.0 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, about 160 mL, about 150 mL, about 140 mL, about 130 mL, about 120 mL, about 110 mL, about 100 mL, about 90 mL, about 80 mL, about 70 mL, about 60 mL, about 50 mL, about 40 mL, about 30 mL, about 25 mL, about 20 mL, about 15 mL, or about 10 mL; about 10 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, about 160 mL, about 150 mL, about 140 mL, about 130 mL, about 120 mL, about 110 mL, about 100 mL, about 90 mL, about 80 mL, about 70 mL, about 60 mL, about 50 mL, about 40 mL, about 30 mL, about 25 mL, about 20 mL, or about 15 mL; about 15 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, about 160 mL, about 150 mL, about 140 mL, about 130 mL, about 120 mL, about 110 mL, about 100 mL, about 90 mL, about 80 mL, about 70 mL, about 60 mL, about 50 mL, about 40 mL, about 30 mL, about 25 mL, or about 20 mL; about 20 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, about 160 mL, about 150 mL, about 140 mL, about 130 mL, about 120 mL, about 110 mL, about 100 mL, about 90 mL, about 80 mL, about 70 mL, about 60 mL, about 50 mL, about 40 mL, about 30 mL, or about 25 mL; about 25 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, about 160 mL, about 150 mL, about 140 mL, about 130 mL, about 120 mL, about 110 mL, about 100 mL, about 90 mL, about 80 mL, about 70 mL, about 60 mL, about 50 mL, about 40 mL, or about 30 mL; about 30 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, about 160 mL, about 150 mL, about 140 mL, about 130 mL about 120 mL, about 110 mL, about 100 mL, about 90 mL, about 80 mL, about 70 mL, about 60 mL, about 50 mL, or about 40 mL; about 40 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, about 160 mL, about 150 mL, about 140 mL, about 130 mL, about 120 mL, about 110 mL, about 100 mL, about 90 mL, about 80 mL, about 70 mL, about 60 mL, or about 50 mL; about 50 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, about 160 mL, about 150 mL, about 140 mL, about 130 mL, about 120 mL, about 110 mL, about 100 mL, about 90 mL, about 80 mL, about 70 mL, or about 60 mL; about 60 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, about 160 mL, about 150 mL, about 140 mL, about 130 mL, about 120 mL, about 110 mL, about 100 mL, about 90 mL, about 80 mL, or about 70 mL; about 70 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, about 160 mL, about 150 mL, about 140 mL, about 130 mL, about 120 mL, about 110 mL, about 100 mL, about 90 mL, or about 80 mL; about 80 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, about 160 mL, about 150 mL, about 140 mL, about 130 mL, about 120 mL, about 110 mL, about 100 mL, or about 90 mL; about 90 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, about 160 mL, about 150 mL, about 140 mL, about 130 mL, about 120 mL, about 110 mL, or about 100 mL; about 100 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, about 160 mL, about 150 mL, about 140 mL, about 130 mL, about 120 mL, or about 110 mL; about 110 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, about 160 mL, about 150 mL, about 140 mL, about 130 mL, or about 120 mL; about 120 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, about 160 mL, about 150 mL, about 140 mL, or about 130 mL; about 130 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, about 160 mL, about 150 mL, or about 140 mL; about 140 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, about 160 mL, or about 150 mL; about 150 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, about 170 mL, or about 160 mL; about 160 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, about 180 mL, or about 170 mL; about 170 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, about 190 mL, or about 180 mL; about 180 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, about 200 mL, or about 190 mL; about 190 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, about 250 mL, or about 200 mL; about 200 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, about 300 mL, or about 250 mL; about 250 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, about 350 mL, or about 300 mL; about 300 mL to about 1 L, about 950 mL, about 900 mL, about 850 mL, about 800 mL, about 750 mL, about 700 mL, about 650 mL, about 600 mL, about 550 mL, about 500 mL, about 450 mL, about 400 mL, or about 350 mL; about 350 mL to about 1 L, about 950 mL, about 900 mL, about 850 mL, about 800 mL, about 750 mL, about 700 mL, about 650 mL, about 600 mL, about 550 mL, about 500 mL, about 450 mL, or about 400 mL; about 400 mL to about 1 L, about 950 mL, about 900 mL, about 850 mL, about 800 mL, about 750 mL, about 700 mL, about 650 mL, about 600 mL, about 550 mL, about 500 mL, or about 450 mL; about 450 mL to about 1 L, about 950 mL, about 900 mL, about 850 mL, about 800 mL, about 750 mL, about 700 mL, about 650 mL, about 600 mL, about 550 mL, or about 500 mL; about 500 mL to about 1 L, about 950 mL, about 900 mL, about 850 mL, about 800 mL, about 750 mL, about 700 mL, about 650 mL, about 600 mL, or about 550 mL; about 550 mL to about 1 L, about 950 mL, about 900 mL, about 850 mL, about 800 mL, about 750 mL, about 700 mL, about 650 mL, or about 600 mL; about 600 mL to about 1 L, about 950 mL, about 900 mL, about 850 mL, about 800 mL, about 750 mL, about 700 mL, or about 650 mL; about 650 mL to about 1 L, about 950 mL, about 900 mL, about 850 mL, about 800 mL, about 750 mL, or about 700 mL; about 700 mL to about 1 L, about 950 mL, about 900 mL, about 850 mL, about 800 mL, or about 750 mL; about 750 mL to about 1 L, about 950 mL, about 900 mL, about 850 mL, or about 800 mL; about 800 mL to about 1 L, about 950 mL, about 900 mL, or about 850 mL; about 850 mL to about 1 L, about 950 mL, or about 900 mL; about 900 mL to about 1 L or about 950 mL; or about 950 mL to about 1 L.

Some embodiments of the pharmaceutical compositions described herein further include a sterile isotonic fluid (e.g., any of the isotonic solutions known in the art, e.g., sterile saline, sterile phosphate buffered solution, sterile Ringer's lactate, sterile 5% albumin solution, sterile D₅W solution, sterile D₅W/1/4 NS solution, sterile ½ NS solution, sterile 25% albumin solution, sterile 5% NaHCO₃, sterile 8.4% NaHCO₃ solution, sterile 3% NaCl solution, and sterile 5% NaCl solution). Some examples of the pharmaceutical compositions further include a mammalian serum albmin (e.g., a human, mouse, rabbit, monkey, chimp, or ape serum albumin) at a final concentration of about 1 mg/mL to about 70 mg/mL, about 65 mg/mL, about 60 mg/mL, about 55 mg/mL, about 50 mg/mL, about 45 mg/mL, about 40 mg/mL, about 35 mg/mL, about 30 mg/mL, about 25 mg/mL, about 20 mg/mL, about 15 mg/mL, about 10 mg/mL, or about 5 mg/mL; about 5 mg/mL to about 70 mg/mL, about 65 mg/mL, about 60 mg/mL, about 55 mg/mL, about 50 mg/mL, about 45 mg/mL, about 40 mg/mL, about 35 mg/mL, about 30 mg/mL, about 25 mg/mL, about 20 mg/mL, about 15 mg/mL, or about 10 mg/mL; about 10 mg/mL to about 70 mg/mL, about 65 mg/mL, about 60 mg/mL, about 55 mg/mL, about 50 mg/mL, about 45 mg/mL, about 40 mg/mL, about 35 mg/mL, about 30 mg/mL, about 25 mg/mL, about 20 mg/mL, or about 15 mg/mL; about 15 mg/mL to about 70 mg/mL, about 65 mg/mL, about 60 mg/mL, about 55 mg/mL, about 50 mg/mL, about 45 mg/mL, about 40 mg/mL, about 35 mg/mL, about 30 mg/mL, about 25 mg/mL, or about 20 mg/mL; about 20 mg/mL to about 70 mg/mL, about 65 mg/mL, about 60 mg/mL, about 55 mg/mL, about 50 mg/mL, about 45 mg/mL, about 40 mg/mL, about 35 mg/mL, about 30 mg/mL, or about 25 mg/mL; about 25 mg/mL to about 70 mg/mL, about 65 mg/mL, about 60 mg/mL, about 55 mg/mL, about 50 mg/mL, about 45 mg/mL, about 40 mg/mL, about 35 mg/mL, about 30 mg/mL; about 30 mg/mL to about 70 mg/mL, about 65 mg/mL, about 60 mg/mL, about 55 mg/mL, about 50 mg/mL, about 45 mg/mL, about 40 mg/mL, or about 35 mg/mL; about 35 mg/mL to about 70 mg/mL, about 65 mg/mL, about 60 mg/mL, about 55 mg/mL, about 50 mg/mL, about 45 mg/mL, or about 40 mg/mL; about 40 mg/mL to about 70 mg/mL, about 65 mg/mL, about 60 mg/mL, about 55 mg/mL, about 50 mg/mL, or about 45 mg/mL; about 45 mg/mL to about 70 mg/mL, about 65 mg/mL, about 60 mg/mL, about 55 mg/mL, or about 50 mg/mL; about 50 mg/mL to about 70 mg/mL, about 65 mg/mL, about 60 mg/mL, or about 55 mg/mL; about 55 mg/mL to about 70 mg/mL, about 65 mg/mL, or about 60 mg/mL; about 60 mg/mL to about 70 mg/mL or about 65 mg/mL; or about 65 ng/mL to about 70 mg/mL. As one skilled in the art can appreciate, a serum albumin should be selected based on the mammal that is to be administered the pharmaceutical agent (e.g., a human should be administered a pharmaceutical composition including a human serum albumin).

Some embodiments of the pharmaceutical compositions further include one or both of: (i) one or more anti-fungal agents (e.g., selected from the group of bifonazole, butoconazole, clotrimazole, econazole, ketoconazole, luliconazole, miconazole, omoconazole, oxiconazole, sertaconazole, sulconazole, tioconazole, albaconazole, efinaconazole, epoziconazole, fluconazole, isavuconazole, itraconazole, posaconazole, propiconazole, ravusconazole, terconazole, voriconazole, abafungin, amorolfin, butenafine, naftifine, terbinafine, anidulafungin, caspofungin, micafungin, benzoic acid, ciclopirox, flucytosine, 5-fluorocytosine, griseofulvin, haloprogin, tolnaflate, undecylenic acid, and balsam of peru) and (ii) one or more antibiotics (e.g., selected from the group of amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem, cilastatin, meropenem, cefadroxil, cefazolin, cefalotin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, penicillin G, temocillin, ticarcillin, amoxicillin, calvulanate, ampicillin, subbactam, piperacillin, tazobactam, ticarcillin, clavulanate, bacitracin, colistin, polymyxin B, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethoxazole, sulfanilimide, sulfasalazine, sulfisoxazole, trimethoprim-sulfamethoxazole, sulfonamideochrysoidine, demeclocycline, minocycline, oytetracycline, tetracycline, clofazimine, dapsone, dapreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin, dalopristin, thiamphenicol, tigecycyline, tinidazole, trimethoprim, and teixobactin) at a final concentration of each anti-fungal and/or each antibiotic of between about 1.0 μg/mL to about 200 μg/mL, about 190 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, about 95 μg/mL, about 90 μg/mL, about 85 μg/mL, about 80 μg/mL, about 75 μg/mL, about 70 μg/mL, about 65 μg/mL, about 60 μg/mL, about 55 μg/mL, about 50 μg/mL, about 45 μg/mL, about 40 μg/mL, about 35 μg/mL, about 30 μg/mL, about 25 μg/mL, about 20 μg/mL, about 15 μg/mL, about 10 μg/mL, or about 5.0 μg/mL; about 5.0 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, about 95 μg/mL, about 90 μg/mL, about 85 μg/mL, about 80 μg/mL, about 75 μg/mL, about 70 μg/mL, about 65 μg/mL, about 60 μg/mL, about 55 μg/mL, about 50 μg/mL, about 45 μg/mL, about 40 μg/mL, about 35 μg/mL, about 30 μg/mL, about 25 μg/mL, about 20 μg/mL, about 15 μg/mL, or about 10 μg/mL; about 10 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, about 95 μg/mL, about 90 μg/mL, about 85 μg/mL, about 80 μg/mL, about 75 μg/mL, about 70 μg/mL, about 65 μg/mL, about 60 μg/mL, about 55 μg/mL, about 50 μg/mL, about 45 μg/mL, about 40 μg/mL, about 35 μg/mL, about 30 μg/mL, about 25 μg/mL, about 20 μg/mL, or about 15 μg/mL; about 15 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, about 95 μg/mL, about 90 μg/mL, about 85 μg/mL, about 80 μg/mL, about 75 μg/mL, about 70 μg/mL, about 65 μg/mL, about 60 μg/mL, about 55 μg/mL, about 50 μg/mL, about 45 μg/mL, about 40 μg/mL, about 35 μg/mL, about 30 μg/mL, about 25 μg/mL, or about 20 μg/mL; about 20 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, about 95 μg/mL, about 90 μg/mL, about 85 μg/mL, about 80 μg/mL, about 75 μg/mL, about 70 μg/mL, about 65 μg/mL, about 60 μg/mL, about 55 μg/mL, about 50 μg/mL, about 45 μg/mL, about 40 μg/mL, about 35 μg/mL, about 30 μg/mL, or about 25 μg/mL; about 25 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, about 95 μg/mL, about 90 μg/mL, about 85 μg/mL, about 80 μg/mL, about 75 μg/mL, about 70 μg/mL, about 65 μg/mL, about 60 μg/mL, about 55 μg/mL, about 50 μg/mL, about 45 μg/mL, about 40 μg/mL, about 35 μg/mL, or about 30 μg/mL; about 30 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, about 95 μg/mL, about 90 μg/mL, about 85 μg/mL, about 80 μg/mL, about 75 μg/mL, about 70 μg/mL, about 65 μg/mL, about 60 μg/mL, about 55 μg/mL, about 50 μg/mL, about 45 μg/mL, about 40 μg/mL, or about 35 μg/mL; about 35 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, about 95 μg/mL, about 90 μg/mL, about 85 μg/mL, about 80 μg/mL, about 75 μg/mL, about 70 μg/mL, about 65 μg/mL, about 60 μg/mL, about 55 μg/mL, about 50 μg/mL, about 45 μg/mL, or about 40 μg/mL; about 40 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, about 95 μg/mL, about 90 μg/mL, about 85 μg/mL, about 80 μg/mL, about 75 μg/mL, about 70 μg/mL, about 65 μg/mL, about 60 μg/mL, about 55 μg/mL, about 50 μg/mL, or about 45 μg/mL; about 45 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, about 95 μg/mL, about 90 μg/mL, about 85 μg/mL, about 80 μg/mL, about 75 μg/mL, about 70 μg/mL, about 65 μg/mL, about 60 μg/mL, about 55 μg/mL, or about 50 μg/mL, about 50 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, about 95 μg/mL, about 90 μg/mL, about 85 μg/mL, about 80 μg/mL, about 75 μg/mL, about 70 μg/mL, about 65 μg/mL, about 60 μg/mL, or about 55 μg/mL; about 55 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, about 95 μg/mL, about 90 μg/mL, about 85 μg/mL, about 80 μg/mL, about 75 μg/mL, about 70 μg/mL, about 65 μg/mL, or about 60 μg/mL; about 60 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, about 95 μg/mL, about 90 μg/mL, about 85 μg/mL, about 80 μg/mL, about 75 μg/mL, about 70 μg/mL, or about 65 μg/mL; about 65 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, about 95 μg/mL, about 90 μg/mL, about 85 μg/mL, about 80 μg/mL, about 75 μg/mL, or about 70 μg/mL; about 70 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, about 95 μg/mL, about 90 μg/mL, about 85 μg/mL, about 80 μg/mL, or about 75 μg/mL; about 75 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, about 95 μg/mL, about 90 μg/mL, about 85 μg/mL, or about 80 μg/mL; about 80 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, about 95 μg/mL, about 90 μg/mL, or about 85 μg/mL; about 85 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, about 95 μg/mL, or about 90 μg/mL; about 90 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, about 100 μg/mL, or about 95 μg/mL; about 95 μg/mL to about 200 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, about 105 μg/mL, or about 100 μg/mL; about 100 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, about 110 μg/mL, or about 105 μg/mL; about 105 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, about 115 μg/mL, or about 110 μg/mL; about 110 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, about 120 μg/mL, or about 115 μg/mL; about 115 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, about 125 μg/mL, or about 120 μg/mL; about 120 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, about 130 μg/mL, or about 125 μg/mL; about 125 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, about 135 μg/mL, or about 130 μg/mL; about 130 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, about 140 μg/mL, or about 135 μg/mL; about 135 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, about 145 μg/mL, or about 140 μg/mL; about 140 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, about 150 μg/mL, or about 145 μg/mL; about 145 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, about 155 μg/mL, or about 150 μg/mL; about 150 μg/mL to about 200 μg/mL, about 195 μg/mL, about 190 μg/mL, about 185 μg/mL, about 180 μg/mL, about 175 μg/mL, about 170 μg/mL, about 165 μg/mL, about 160 μg/mL, or about 155 μg/mL; about 155 μg/mL to about 200 μg/mL, about 195 μg/mL, about 190 μg/mL, about 185 μg/mL, about 180 μg/mL, about 175 μg/mL, about 170 μg/mL, about 165 μg/mL, or about 160 μg/mL; about 160 μg/mL to about 200 μg/mL, about 195 μg/mL, about 190 μg/mL, about 185 μg/mL, about 180 μg/mL, about 175 μg/mL, about 170 μg/mL, or about 165 μg/mL; about 165 μg/mL to about 200 μg/mL, about 195 μg/mL, about 190 μg/mL, about 185 μg/mL, about 180 μg/mL, about 175 μg/mL, or about 170 μg/mL; about 170 μg/mL to about 200 μg/mL, about 195 μg/mL, about 190 μg/mL, about 185 μg/mL, about 180 μg/mL, or about 175 μg/mL; about 175 μg/mL to about 200 μg/mL, about 195 μg/mL, about 190 μg/mL, about 185 μg/mL, or about 180 μg/mL; about 180 μg/mL to about 200 μg/mL, about 195 μg/mL, about 190 μg/mL, or about 185 μg/mL; about 185 μg/mL to about 200 μg/mL, about 195 μg/mL, or about 190 μg/mL; or about 195 μg/mL to about 200 μg/L.

In some embodiments, the pharmaceutical compositions are formulated for intravenously, intraarterial, or intra-tumor administration.

### Kits

Also provided herein are kits including one or more doses of any of the pharmaceutical compositions described herein. In some embodiments, the pharmaceutical compositions are provided in a pre-filled sterile and biocompatible bag. In some embodiments, the pharmaceutical compositions are provided in a pre-filled sterile syringe having a needle having a gauge that would not cause a significant amount of sheer stress on cells (e.g., any of the T cells or innate immune cells described herein) flowed through the needle. In some embodiments, the pharmaceutical compositions are provided in a sterile and biocompatible vial.

The kits can also provide instructions for performing any of the methods described herein.

### Methods of Increasing Anti-Tumor Immunity in a Mammal Having a Cancer

Also provided are methods of increasing anti-tumor immunity in a mammal having a cancer that include: (a) identifying a mammal (e.g., any of the mammals described herein) having a cancer (e.g., any of the cancers described herein); (b) harvesting a T cell from the identified mammal (e.g., performed using any of the techniques described herein); (c) culturing the T cell (e.g., using any of the culturing methods, culture media, culture conditions, or culture times described herein): in a culture medium sufficient to induce and/or increase anaplerosis (e.g., any of the culture media sufficient to induce and/or increase anaplerosis described herein) and/or comprising a NLRP3 activator (e.g., any of the NLRP3 activators described herein) in an amount sufficient to improve anti-tumor activity of a T cell; and/or under conditions sufficient to induce and/or increase anaplerosis (e.g., any of the conditions sufficient to induce and/or increase anaplerosis described herein), thereby resulting in an improvement in the anti-tumor activity of the T cell (e.g., an improvement of any of the one or more anti-tumor activities of a T cell described herein); and (d) administering the T cell to a mammal, thereby the resulting in increasing anti-tumor immunity in the mammal. Any of the methods of culturing or harvesting (isolating) T cells from a mammal described herein can be used in these methods. Harvesting T cells from a mammal may include, e.g., obtaining a blood sample or an apheresis product from a mammal. Any of the NLRP3 activators, any of the culture media sufficient to induce and/or increase anaplerosis, and/or any of the conditions sufficient to induce and/or increase anaplerosis described herein can be used in these methods. The T cells can be administered back to the mammal at any of the doses described herein. The T cells can be administered in any of the formulations described herein. The T cells can be administered to the mammal via intravenous administration, intraarterial administration, or intratumor injection. The T cells can be administered to the mammal at any of the administration frequencies described herein.

Also provided are methods of increasing anti-tumor immunity in a mammal having a mammal that include: (a) identifying a mammal (e.g., any of the mammals described herein) as having a cancer (e.g., any of the cancers described herein); and (b) administering a therapeutically effective amount of a NLRP3 activator (e.g., any of the NLRP3 activators described herein) to the identified mammal. Any of the NLRP3 activators can be used in these methods. The NLRP3 activator can be administered to the mammal at any of the doses described herein. The NLRP3 activator can be administered using any of the routes of administration described herein for NLRP3 activators. The NLRP3 activators can be administered to the mammal at any of the administration frequencies described herein.

In some examples of any of these methods, the mammals are diagnosed as having a cancer. Some embodiments of these methods further include diagnosing a mammal as having a cancer (e.g., using any of the exemplary methods described herein).

In some examples of any of these methods, the increased anti-tumor immunity can be, e.g., one or more of: increased differentiation of premature cells into a T cell in the mammal, increased division and proliferation of a T cells in the mammal, increased extravasation of a T cell from the blood into a tissue in the mammal, increased ability of a T cell to infiltrate a solid tumor in the mammal, increased activation of a T cell in the mammal, a decrease in the ability of Treg cells to decrease the induction and/or proliferation of effector T cells in the mammal, increased production of cytokines and/or lymphokines by the T cells that promote anti-tumor immune activity (e.g., GM-CSF, IL-7, IL-12, IL-15, IL-18, and IL-21) in the mammal, and a decrease in the production of cytokines and/or lymphokines by T cells that decrease anti-tumor activity (e.g., IL-10 and TGF-β) in the mammal. Methods for detecting the anti-tumor activities of a T cell in a mammal are known in the art. For example, the differentiation of premature cells into T cells can be detected by detecting the ratios of premature cells and T cells over time in the mammal (e.g., by analyzing the cells in a blood sample from the mammal, e.g., by FACS analysis). Exemplary assays for detecting the level of extravasation of a T cell in a mammal include, e.g., detecting the level of T cells in a tissue sample from a mammal or by detecting expression of proteins on the surface of a T cell that allow for rolling and extravasation out of the blood stream into a tissue. Exemplary assays for detecting the level of infiltration of a T cell into a solid tumor are described in Ye et al., J. Clin. Immunol. 26:447-456, 2006. The level of infiltration of a T cell into a solid tumor in a mammal can be determined by examining a tumor biopsy sample from the mammal before treatment and a tumor biopsy sample from the mammal after treatment. Such methods can include microscopy techniques to determine the presence of T cells in the tumor biopsy samples. Exemplary assays for detecting the levels of cytokines and/or lymphokines produced by T cells that decrease anti-tumor immune activity or promote anti-tumor immune activity can include, e.g., the use of one or more antibodies that specifically bind to a cytokine or lymphokine produced by a T cell that decrease anti-tumor immune activity or promote anti-tumor immune activity, in e.g., an immunoassay (e.g., an ELISA) performed using a blood or serum sample from the mammal as a sample. These antibodies are commercially available from a number of vendors. Method of detecting the level of T cell activation are known in the art. T-cell activation means the binding of one or more receptors (e.g., a TCR optionally in association with a CD3 dimer) on the surface of a T cell with one or more cognate receptors on the surface of an antigen-presenting cell. The term activate a T cell can include the binding of one or more integrins on the surface of the T cell with their respective ligands (e.g., intercellular adhesion molecule (ICAM), vascular cell adhesion molecule (VCAM), or fibronectin), or the activation of a co-stimulatory receptor on a T cell (e.g., CD28, ICSO, CD40) by binding to its ligand. For example CD28 is activated by CD80 or CD86, ICOS is activated by ICOS-L, and ICOS is activated by ICOS-L. Exemplary methods of detecting activation of a T cell include measuring T cell proliferation, upregulation of activation markers on the T cell surface, differentiation of the T cells into effector cells, induction of cytotoxity or cytokine secretion, and induction of apoptosis. Assays for detecting T cell proliferation in a sample including T cells from a mammal are commercially available. Examples of such methods for detecting activation markers on the T cell surface, markers of differentiation of T cells into effector cells, markers of induction of cytotoxicity, cytokines secreted by activated T cells, and markers of apoptosis can be performed using, e.g., one or more antibodies that bind to an activation marker on the T cell surface, a marker of differentiation of T cells into effector cells, a cytokine secreted by activated T cells, or a marker of apoptosis. Such antibodies are commercially available from a variety of different vendors.

In some examples, the increase in anti-tumor activity in the mammal can be indirectly observed through a decrease in the size of one or more solid tumors, a decrease in the severity or number of symptoms of a cancer in a mammal, an increase in the time of remission of a cancer in a mammal, an increase in the time of survival of a mammal having a cancer, and decrease in the risk of developing a metastasis or developing additional metastases.

### Methods of Increasing Time of Remission of a Cancer in a Mammal

Provided herein are methods of increasing the time of remission of a cancer in a mammal that include (a) identifying a mammal (e.g., any of the mammals described herein, e.g., a human) having a cancer (e.g., any of the cancers described herein); (b) harvesting a T cell (e.g., any of the T cells described herein) from the identified mammal; (c) culturing the T cell: in a culture medium sufficient to induce and/or increase anaplerosis (e.g., any of the culture media sufficient to induce and/or increase anaplerosis described herein) and/or including a NLRP3 activator (e.g., any of the NLRP3 activators described herein) in an amount sufficient to improve anti-tumor activity of a T cell (e.g., any one or more of the anti-tumor activities of a T cell described herein); and/or under conditions sufficient to induce and/or increase anaplerosis, thereby resulting in an improvement in the anti-tumor activity of the T cell; and (c) administering the T cell to the mammal, thereby the resulting in an increase in the time of remission of a cancer in the mammal. The T cells can be cultured using any of the culturing methods described herein. Any of the doses of T cells described herein can be administered to the mammal in these methods. The T cells can be administered by, e.g., intraarterial administration, intravenous administration, or intra-tumor injection. The T cells can be administered once a week, twice a week, three times a week, once every two weeks, once every three weeks, once a month, twice a month, once every two months, once every three months, once every four months, once every five months, once every six months, once every seven months, once every eight months, once every nine months, once every ten months, once every eleven months, or once every twelve months. The T cells can be administered in any of the formulations described herein.

Also provided are methods of increasing the time of remission of a cancer in a mammal that include (a) identifying a mammal (e.g., any of the mammals described herein, e.g., a human) as having a cancer (e.g., any of the cancers described herein); and (b) administering a therapeutically effective amount of a NLRP3 activator (e.g., any of the NLRP3 activators described herein) to the identified mammal (e.g., at any of the dosages of an NLRP3 activator described herein). Any of the doses of an NLRP3 activator described herein can be administered to the mammal in these methods. In some examples, the NLRP3 activator is administered by oral administration, intraperitoneal administration, intramuscular administration, intraarterial administration, or intravenous administration. The NLRP3 activator can be administered once a day, twice a day, three times a day, four times a day, once a week, twice a week, three times a week, once every two weeks, once every three weeks, once a month, twice a month, once every two months, once every three months, once every four months, once every five months, once every six months, once every seven months, once every eight months, once every nine months, once every ten months, once every eleven months, or once every twelve months. In some examples, the mammal has been diagnosed as having a cancer. Some embodiments further include a step of diagnosing the cancer in the mammal.

In some embodiments of any of the methods of increasing the time of remission of cancer in a mammal, the increase in the time of remission is compared to a control mammal (e.g., a mammal or a population of mammals having the same or a similar type of cancer). In some embodiments, the mammal is not yet in remission. In some examples, the mammal is already in remission. In some examples, the increase in remission is a statistically significant increase. In some examples, the increase in the time of remission is about 1 day to about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, about 10 months, about 8 months, about 6 months, about 4 months, about 2 months, about 1 month, or about 2 weeks; about 2 weeks to about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, about 10 months, about 8 months, about 6 months, about 4 months, about 2 months, or about 1 month; about 1 month to about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, about 10 months, about 8 months, about 6 months, about 4 months, or about 2 months; about 2 month to about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, about 10 months, about 8 months, about 6 months, or about 4 months; about 4 month to about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, about 10 months, about 8 months, or about 6 months; about 6 month to about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, about 10 months, or about 8 months; about 8 month to about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, or about 10 months; about 10 month to about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, or about 1 year; about 1 year to about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, or about 1.5 years; about 1.5 years to about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, to about 2 years; about 2 years to about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, or about 2.5 years; about 2.5 years to about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, or about 3 years; about 3 years to about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, or about 3.5 years; about 3.5 years to about 10 years, about 9.5 years, about 9 years, about 8.5 years, about 8 years, about 7.5 years, about 7 years, about 6.5 years, about 6 years, about 5.5 years, about 5 years, about 4.5 years, or about 4 years; about 4 years to about 10 years, about 9.5 years, about 9 years, about 8.5 years, about 8 years, about 7.5 years, about 7 years, about 6.5 years, about 6 years, about 5.5 years, about 5 years, or about 4.5 years; about 4.5 years to about 10 years, about 9.5 years, about 9 years, about 8.5 years, about 8 years, about 7.5 years, about 7 years, about 6.5 years, about 6 years, about 5.5 years, or about 5 years; about 5 years to about 10 years, about 9.5 years, about 9 years, about 8.5 years, about 8 years, about 7.5 years, about 7 years, about 6.5 years, about 6 years, or about 5.5 years; about 5.5 years to about 10 years, about 9.5 years, about 9 years, about 8.5 years, about 8 years, about 7.5 years, about 7 years, about 6.5 years, or about 6 years; about 6 years to about 10 years, about 9.5 years, about 9 years, about 8.5 years, about 8 years, about 7.5 years, about 7 years, or about 6.5 years; about 6.5 years to about 10 years, about 9.5 years, about 9 years, about 8.5 years, about 8 years, about 7.5 years, or about 7 years; about 7 years to about 10 years, about 9.5 years, about 9 years, about 8.5 years, about 8 years, or about 7.5 years; about 7.5 years to about 10 years, about 9.5 years, about 9 years, about 8.5 years, or about 8 years; about 8 years to about 10 years, about 9.5 years, about 9 years, or about 8.5 years; about 8.5 years to about 10 years, about 9.5 years, or about 9 years; about 9 years to about 10 years or about 9.5 years; or about 9.5 years to about 10 years (e.g., compared to a control mammal, e.g., a mammal or a population of mammals having the same or a similar type of cancer).

Methods for determining whether or not a mammal is in remission are known by those skilled in the art. For example, a PET scan, MM, CT scan, ultrasound, and X-ray of the mammal's body may be obtained, and such data can be used to determine whether or not a mammal is in remission. In some examples, diagnostic tests can be performed on samples from a mammal (e.g., a blood sample or a biopsy) to determine whether or not the mammal is still in remission.

### Methods of Treating a Mammal Having a Cancer or an Infectious Disease

Also provided are methods of treating a mammal having a cancer or an infectious disease that include: (a) identifying a mammal (e.g., any of the mammals described herein) having a cancer (e.g., any of the cancers described herein) or an infectious disease (e.g., any of the infectious diseases described herein); (b) harvesting a T cell (e.g., any of the T cells described herein) from the identified mammal; (c) culturing the T cell (e.g., using any of the culturing methods, culture media, culture conditions, or culture times described here): in a culture medium sufficient to induce and/or increase anaplerosis (e.g., any of the culture media sufficient to induce and/or increase anaplerosis described herein) and/or including a NLRP3 activator (e.g., any of the NLRP3 activators described herein) in an amount sufficient to improve anti-tumor or anti-infectious disease activity of a T cell (e.g., any of the anti-tumor activities or anti-infectious disease activities of a T cell described herein); and/or under conditions sufficient to induce and/or increase anaplerosis (e.g., any of the conditions sufficient to induce and/or increase anaplerosis described herein), thereby resulting in an improvement in the anti-tumor or anti-infectious disease activity of the T cell; and (e) administering the T cell to the mammal. Any of the methods of culturing or harvesting (isolating) T cells from a mammal described herein can be used in these methods. Harvesting T cells from a mammal may include, e.g., obtaining a blood sample or an apheresis product from a mammal. Any of the NLRP3 activators, any of the culture media sufficient to induce and/or increase anaplerosis, and/or any of the conditions sufficient to induce and/or increase anaplerosis described herein can be used in these methods. The T cells can be administered back to the mammal at any of the doses described herein. The T cells can be administered in any of the formulations described herein. The T cells can be administered to the mammal via intravenous administration, intraarterial administration, or intratumor injection. The T cells can be administered to the mammal at any of the administration frequencies described herein.

Also provided are methods treating a mammal having a cancer or an infectious disease that include: (a) identifying a mammal having a cancer (e.g., any of the cancers described herein) or an infectious disease (e.g., any of the infectious diseases described herein); and (b) administering a therapeutically effective amount of a NLRP3 activator (e.g., any of the NLRP3 activators described herein) to the identified mammal. Any of the NLRP3 activators can be used in these methods. The NLRP3 activator can be administered to the mammal at any of the doses described herein. The NLRP3 activator can be administered using any of the routes of administration described herein for NLRP3 activators. The NLRP3 activators can be administered to the mammal at any of the administration frequencies described herein.

In some examples of any of these methods, the mammals are diagnosed as having a cancer or an infectious disease. Some embodiments of these methods further include diagnosing a mammal as having a cancer or an infectious disease (e.g., using any of the exemplary methods described herein).

In some embodiments, treatment of the cancer results in one or more of decreasing the severity of a cancer in a mammal, and/or decreasing the number and/or frequency of one or symptoms of a cancer in a mammal. For example, a decrease in the severity of a cancer can include a decrease in the size of one or more solid tumors in a mammal (e.g., as compared to the size of the one or more tumors prior to treatment). A decrease in the severity of a cancer in a mammal can include a decrease in the rate of growth of one or more solid tumors in a mammal over time (e.g., as compared to the rate of growth of the one or more solid tumors prior to treatment). A decrease in the severity of a cancer can also be indicated by an increased time of remission of a cancer in the mammal (e.g., as compared to the average length of remission in a mammal having a similar cancer but receiving a different treatment). A decrease in the severity of a cancer can also, e.g., mean a decrease in the rate of development of metastasis or the rate of development of additional metastasis in a mammal having a cancer (e.g., as compared to a population of mammals having a similar cancer). A decrease in the severity of a cancer can be, e.g., an increase in the time of survival of a mammal having a cancer (e.g., as compared to a population of mammals having a similar cancer). A decrease in the severity of a cancer can, e.g., result in an improved prognosis of a mammal having a cancer.

In some embodiments, treatment of the infectious disease results in one or more of decreasing the severity of an infectious disease in a mammal, and/or decreasing the number and/or frequency of one or symptoms of an infectious disease in a mammal (e.g., any of the symptoms of infectious disease described herein). For example, a decrease in the severity of a an infectious disease can include a decrease in the titers of a pathogen in a mammal or a sample from the mammal (e.g., as compared to the titers of a pathogen in a mammal prior to treatment or a sample from the mammal obtained from the mammal prior to treatment). A decrease in the severity of an infectious disease in a mammal can include a decrease in the rate of propagation of a pathogen in a mammal over time (e.g., as compared to the rate of propagation of the pathogen prior to treatment).

### Methods of Increasing the Time of Survival of a Mammal Having a Cancer

Provided herein are methods of increasing the time of survival of a mammal having a cancer that include (a) identifying a mammal (e.g., any of the mammals described herein, e.g., a human) having a cancer (e.g., any of the cancers described herein); (b) harvesting a T cell (e.g., any of the T cells described herein) from the identified mammal; (c) culturing the T cell: in a culture medium sufficient to induce and/or increase anaplerosis (e.g., any of the culture media sufficient to induce and/or increase anaplerosis described herein) and/or including a NLRP3 activator (e.g., any of the NLRP3 activators described herein) in an amount sufficient to improve anti-tumor activity of a T cell (e.g., any one or more of the anti-tumor activities of a T cell described herein); and/or under conditions sufficient to induce and/or increase anaplerosis, thereby resulting in an improvement in the anti-tumor activity of the T cell; and (c) administering the T cell to the mammal (thereby the resulting in an increase in the time of survival of a mammal having a cancer). The T cells can be cultured using any of the culturing methods described herein. Any of the doses of T cells described herein can be administered to the mammal in these methods. The T cells can be administered by, e.g., intraarterial administration, intravenous administration, or intra-tumor injection. The T cells can be administered once a week, twice a week, three times a week, once every two weeks, once every three weeks, once a month, twice a month, once every two months, once every three months, once every four months, once every five months, once every six months, once every seven months, once every eight months, once every nine months, once every ten months, once every eleven months, or once every twelve months. The T cells can be administered in any of the formulations described herein.

Also provided are methods of increasing the time of survival of a mammal having a cancer that include (a) identifying a mammal (e.g., any of the mammals described herein, e.g., a human) as having a cancer (e.g., any of the cancers described herein); and (b) administering a therapeutically effective amount of a NLRP3 activator (e.g., any of the NLRP3 activators described herein) to the identified mammal (e.g., at any of the dosages of an NLRP3 activator described herein). Any of the doses of an NLRP3 activator described herein can be administered to the mammal in these methods. In some examples, the NLRP3 activator is administered by oral administration, intraperitoneal administration, intramuscular administration, intraarterial administration, or intravenous administration. The NLRP3 activator can be administered once a day, twice a day, three times a day, four times a day, once a week, twice a week, three times a week, once every two weeks, once every three weeks, once a month, twice a month, once every two months, once every three months, once every four months, once every five months, once every six months, once every seven months, once every eight months, once every nine months, once every ten months, once every eleven months, or once every twelve months. In some examples, the mammal has been diagnosed as having a cancer. Some embodiments further include a step of diagnosing the cancer in the mammal.

In some embodiments of any of the methods of increasing the time of remission of cancer in a mammal, the increase in the time of remission is compared to a control mammal (e.g., a mammal or a population of mammals having the same or a similar type of cancer). In some embodiments, the mammal can have an early stage of cancer (e.g., Stage 1 or 2). In some embodiments, the mammal can have a late stage of cancer (e.g., Stage 3 or 4). In some examples, the increase in time of survival is a statistically significant increase. In some examples, the increase in the time of survival is about 1 day to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, about 10 months, about 8 months, about 6 months, about 4 months, about 2 months, about 1 month, or about 2 weeks; about 2 weeks to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, about 10 months, about 8 months, about 6 months, about 4 months, about 2 months, or about 1 month; about 1 month to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, about 10 months, about 8 months, about 6 months, about 4 months, or about 2 months; about 2 months to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, about 10 months, about 8 months, about 6 months, or about 4 months; about 4 months to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, about 10 months, about 8 months, or about 6 months; about 6 months to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, about 10 months, or about 8 months; about 8 months to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, or about 10 months; about 10 months to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, or about 1 year; about 1 year to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, or about 1.5 years; about 1.5 year to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, or about 2 years; about 2 year to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, or about 2.5 years; about 2.5 year to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, or about 3 years; about 3 year to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, or about 3.5 years; about 3.5 years to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, about 6.5 years, about 6 years, about 5.5 years, about 5 years, about 4.5 years, or about 4 years; about 4 years to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, about 6.5 years, about 6 years, about 5.5 years, about 5 years, or about 4.5 years; about 4.5 years to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, about 6.5 years, about 6 years, about 5.5 years, or about 5 years; about 5 years to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, about 6.5 years, about 6 years, or about 5.5 years; about 5.5 years to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, about 6.5 years, or about 6 years; about 6 years to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, about 7 years, or about 6.5 years; about 6.5 years to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, about 7.5 years, or about 7 years, about 7 years to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, about 8 years, or about 7.5 years; about 7.5 years to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, about 8.5 years, or about 8 years; about 8 years to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, about 9 years, or about 8.5 years; about 8.5 years to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, about 9.5 years, or about 9 years; about 9 years to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, about 10 years, or about 9.5 years; about 9.5 years to about 40 years, about 38 years, about 36 years, about 34 years, about 32 years, about 30 years, about 28 years, about 26 years, about 24 years, about 22 years, about 20 years, about 18 years, about 16 years, about 14 years, about 12 years, or about 10 years; about 10 years to about 40 years, about 38 years, about 36 years, about 34 years, about 32 years, about 30 years, about 28 years, about 26 years, about 24 years, about 22 years, about 20 years, about 18 years, about 16 years, about 14 years, or about 12 years; about 12 years to about 40 years, about 38 years, about 36 years, about 34 years, about 32 years, about 30 years, about 28 years, about 26 years, about 24 years, about 22 years, about 20 years, about 18 years, about 16 years, or about 14 years; about 14 years to about 40 years, about 38 years, about 36 years, about 34 years, about 32 years, about 30 years, about 28 years, about 26 years, about 24 years, about 22 years, about 20 years, about 18 years, or about 16 years; about 16 years to about 40 years, about 38 years, about 36 years, about 34 years, about 32 years, about 30 years, about 28 years, about 26 years, about 24 years, about 22 years, about 20 years, or about 18 years; about 18 years to about 40 years, about 38 years, about 36 years, about 34 years, about 32 years, about 30 years, about 28 years, about 26 years, about 24 years, about 22 years, or about 20 years; about 20 years to about 40 years, about 38 years, about 36 years, about 34 years, about 32 years, about 30 years, about 28 years, about 26 years, about 24 years, or about 22 years; about 22 years to about 40 years, about 38 years, about 36 years, about 34 years, about 32 years, about 30 years, about 28 years, about 26 years, or about 24 years; about 24 years to about 40 years, about 38 years, about 36 years, about 34 years, about 32 years, about 30 years, about 28 years, or about 26 years; about 26 years to about 40 years, about 38 years, about 36 years, about 34 years, about 32 years, about 30 years, or about 28 years; about 28 years to about 40 years, about 38 years, about 36 years, about 34 years, about 32 years, or about 30 years; about 30 years to about 40 years, about 38 years, about 36 years, about 34 years, or about 32 years; about 32 years to about 40 years, about 38 years, about 36 years, or about 34 years; about 34 years to about 40 years, about 38 years, or about 36 years; about 36 years to about 40 years or about 38 years; or about 38 years to about 40 years (e.g., compared to a control mammal, e.g., a mammal or a population of mammals having the same or a similar type of cancer).

### Methods of Decreasing the Size of a Solid Tumor in a Mammal Having a Cancer

Also provided are method of decreasing the size of a solid tumor in a mammal having a cancer that include: (a) identifying a mammal (e.g., any of the mammals described herein) having a cancer (e.g., any of the cancers described herein) and a solid tumor; (b) harvesting a T cell from the identified mammal (e.g., using any of the methods described herein); (c) culturing the T cell (e.g., using any of the culturing methods, culture media, culture conditions, and culture times described herein) in a culture medium sufficient to induce and/or increase anaplerosis (e.g., any of the culture media sufficient to induce and/or increase anaplerosis described herein) and/or comprising a NLRP3 activator (e.g., any of the NLRP3 activators described herein) in an amount sufficient to improve anti-tumor activity of a T cell; and/or under conditions sufficient to induce and/or increase anaplerosis (e.g., any of the conditions for inducing and/or increasing anaplerosis described herein), thereby resulting in an improvement in the anti-tumor activity of the T cell; and (d) administering the T cell to the mammal. Any of the methods of culturing or harvesting (isolating) T cells from a mammal described herein can be used in these methods. Harvesting T cells from a mammal may include, e.g., obtaining a blood sample or an apheresis product from a mammal. Any of the NLRP3 activators, any of the culture media sufficient to induce and/or increase anaplerosis, and/or any of the conditions sufficient to induce and/or increase anaplerosis described herein can be used in these methods. The T cells can be administered back to the mammal at any of the doses described herein. The T cells can be administered in any of the formulations described herein. The T cells can be administered to the mammal via intravenous administration, intraarterial administration, or intratumor injection. The T cells can be administered to the mammal at any of the administration frequencies described herein.

Also provided are methods of decreasing the size of a solid tumor in a mammal having a cancer that include: (a) identifying a mammal (e.g., any of the mammals described herein) having a cancer (e.g., any of the cancers described herein) and a solid tumor; and (b) administering a therapeutically effective amount of a NLRP3 activator (e.g., any of the NLRP3 activators described herein to the identified mammal. Any of the NLRP3 activators can be used in these methods. The NLRP3 activator can be administered to the mammal at any of the doses described herein. The NLRP3 activator can be administered using any of the routes of administration described herein for NLRP3 activators. The NLRP3 activators can be administered to the mammal at any of the administration frequencies described herein.

In some examples of any of these methods, the mammals are diagnosed as having a cancer. Some embodiments of these methods further include diagnosing a mammal as having a cancer (e.g., using any of the exemplary methods described herein).

Some examples of these methods result in the decrease in the size of one, two, three, four, five, six, seven, eight, nine, or ten solid tumors in the mammal. In some examples the reduction in the size of the one or more solid tumors (e.g., two, three, four, five, six, seven, eight, nine, or ten solid tumors) is about 1% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, about 38%, about 36%, about 34%, about 32%, about 30%, about 28%, about 26%, about 24%, about 22%, about 20%, about 18%, about 16%, about 14%, about 12%, about 10%, about 8%, about 6%, about 4%, or about 2%; about 2% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, about 38%, about 36%, about 34%, about 32%, about 30%, about 28%, about 26%, about 24%, about 22%, about 20%, about 18%, about 16%, about 14%, about 12%, about 10%, about 8%, about 6%, or about 4%; about 4% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, about 38%, about 36%, about 34%, about 32%, about 30%, about 28%, about 26%, about 24%, about 22%, about 20%, about 18%, about 16%, about 14%, about 12%, about 10%, about 8%, or about 6%; about 6% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, about 38%, about 36%, about 34%, about 32%, about 30%, about 28%, about 26%, about 24%, about 22%, about 20%, about 18%, about 16%, about 14%, about 12%, about 10%, or about 8%; about 8% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, about 38%, about 36%, about 34%, about 32%, about 30%, about 28%, about 26%, about 24%, about 22%, about 20%, about 18%, about 16%, about 14%, about 12%, or about 10%; about 10% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, about 38%, about 36%, about 34%, about 32%, about 30%, about 28%, about 26%, about 24%, about 22%, about 20%, about 18%, about 16%, about 14%, or about 12%; about 12% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, about 38%, about 36%, about 34%, about 32%, about 30%, about 28%, about 26%, about 24%, about 22%, about 20%, about 18%, about 16%, or about 14%; about 14% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, about 38%, about 36%, about 34%, about 32%, about 30%, about 28%, about 26%, about 24%, about 22%, about 20%, about 18%, or about 16%; about 16% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, about 38%, about 36%, about 34%, about 32%, about 30%, about 28%, about 26%, about 24%, about 22%, about 20%, or about 18%; about 18% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, about 38%, about 36%, about 34%, about 32%, about 30%, about 28%, about 26%, about 24%, about 22%, or about 20%; about 20% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, about 38%, about 36%, about 34%, about 32%, about 30%, about 28%, about 26%, about 24%, or about 22%; about 22% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, about 38%, about 36%, about 34%, about 32%, about 30%, about 28%, about 26%, or about 24%; about 24% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, about 38%, about 36%, about 34%, about 32%, about 30%, about 28%, or about 26%; about 26% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, about 38%, about 36%, about 34%, about 32%, about 30%, or about 28%; about 28% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, about 38%, about 36%, about 34%, about 32%, or about 30%; about 30% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, about 38%, about 36%, about 34%, or about 32%; about 32% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, about 38%, about 36%, or about 34%; about 34% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, about 38%, or about 36%; about 36% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, about 40%, or about 38%; about 38% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, about 42%, or about 40%; about 40% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, about 44%, or about 42%; about 42% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, about 46%, or about 44%; about 44% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, about 48%, or about 46%, about 46% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, about 50%, or about 48%; about 48% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, about 52%, or about 50%; about 50% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, about 54%, or about 52%; about 52% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, about 56%, or about 54%; about 54% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, about 58%, or about 56%; about 56% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, about 60%, or about 58%; about 58% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, about 62%, or about 60%; about 60% to about 100%, about 98%, about 96%, about 94%, about 90%, about 88%, about 86%, about 84%, about 82%, about 80%, about 78%, about 76%, about 74%, about 72%, about 70%, about 68%, about 66%, about 64%, or about 62%; about 62% to about 100%, about 98%, about 96%, about 94%, about 90%, about 88%, about 86%, about 84%, about 82%, about 80%, about 78%, about 76%, about 74%, about 72%, about 70%, about 68%, about 66%, or about 64%; about 64% to about 100%, about 98%, about 96%, about 94%, about 90%, about 88%, about 86%, about 84%, about 82%, about 80%, about 78%, about 76%, about 74%, about 72%, about 70%, about 68%, or about 66%; about 66% to about 100%, about 98%, about 96%, about 94%, about 90%, about 88%, about 86%, about 84%, about 82%, about 80%, about 78%, about 76%, about 74%, about 72%, about 70%, or about 68%; about 68% to about 100%, about 98%, about 96%, about 94%, about 90%, about 88%, about 86%, about 84%, about 82%, about 80%, about 78%, about 76%, about 74%, about 72%, or about 70%; about 70% to about 100%, about 98%, about 96%, about 94%, about 90%, about 88%, about 86%, about 84%, about 82%, about 80%, about 78%, about 76%, about 74%, or about 72%; about 72% to about 100%, about 98%, about 96%, about 94%, about 90%, about 88%, about 86%, about 84%, about 82%, about 80%, about 78%, about 76%, or about 74%; about 74% to about 100%, about 98%, about 96%, about 94%, about 90%, about 88%, about 86%, about 84%, about 82%, about 80%, about 78%, or about 76%; about 76% to about 100%, about 98%, about 96%, about 94%, about 90%, about 88%, about 86%, about 84%, about 82%, about 80%, or about 78%; about 78% to about 100%, about 98%, about 96%, about 94%, about 90%, about 88%, about 86%, about 84%, about 82%, or about 80%; about 80% to about 100%, about 98%, about 96%, about 94%, about 90%, about 88%, about 86%, about 84%, or about 82%; about 82% to about 100%, about 98%, about 96%, about 94%, about 90%, about 88%, about 86%, or about 84%; about 84% to about 100%, about 98%, about 96%, about 94%, about 90%, about 88%, or about 86%; about 86% to about 100%, about 98%, about 96%, about 94%, about 90%, or about 88%; about 88% to about 100%, about 98%, about 96%, about 94%, or about 90%; about 90% to about 100%, about 98%, about 96%, or about 94%; about 92% to about 100%, about 98%, or about 96%; about 94% to about 100% or about 98%; about 96% to about 100% or about 98%; or about 98% to about 100%. Methods of detecting the size of a solid tumor in a mammal include, e.g., a PET scan, MRI, CT scan, ultrasound, and X-ray.

In some examples, the decrease in the size of one or more solid tumors and/or the time of treatment occurs over a period of about 1 week to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, about 10 months, about 8 months, about 6 months, about 4 months, about 2 months, or about 1 month; about 1 month to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, about 10 months, about 8 months, about 6 months, about 4 months, or about 2 months; about 2 months to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, about 10 months, about 8 months, about 6 months, or about 4 months; about 4 months to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, about 10 months, about 8 months, or about 6 months; about 6 months to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, about 10 months, or about 8 months; about 8 months to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, about 1 year, or about 10 months; about 10 months to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, about 1.5 years, or about 1 year; about 1 year to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, about 2 years, or about 1.5 years; about 1.5 years to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, about 2.5 years, or about 2 years; about 2 years to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, about 3 years, or about 2.5 years; about 2.5 years to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, about 3.5 years, or about 3 years; about 3 years to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, about 7 years, about 6.5 years, about 6 years, about 5.5 years, about 5 years, about 4.5 years, about 4 years, or about 3.5 years; about 3.5 years to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, about 7 years, about 6.5 years, about 6 years, about 5.5 years, about 5 years, about 4.5 years, or about 4 years; about 4 years to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, about 7 years, about 6.5 years, about 6 years, about 5.5 years, about 5 years, or about 4.5 years; about 4.5 years to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, about 7 years, about 6.5 years, about 6 years, about 5.5 years, or about 5 years; about 5 years to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, about 7 years, about 6.5 years, about 6 years, or about 5.5 years; about 5.5 years to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, about 7 years, about 6.5 years, or about 6 years; about 6 years to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, about 7 years, or about 6.5 years; about 6.5 years to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, about 7.5 years, or about 7 years; about 7 years to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, about 8 years, or about 7.5 years; about 7.5 years to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, about 8.5 years, or about 8 years; about 8 years to about 15 years, about 14.5 years, about 14 years, about 13.5 years, about 13 years, about 12.5 years, about 12 years, about 11.5 years, about 11 years, about 10.5 years, about 10 years, about 9.5 years, about 9 years, or about 8.5 years; about 8.5 years to about 15 years, about 14.5 years, about 14 years, about 13.5 years, about 13 years, about 12.5 years, about 12 years, about 11.5 years, about 11 years, about 10.5 years, about 10 years, about 9.5 years, or about 9 years; about 9 years to about 15 years, about 14.5 years, about 14 years, about 13.5 years, about 13 years, about 12.5 years, about 12 years, about 11.5 years, about 11 years, about 10.5 years, about 10 years, or about 9.5 years; about 9.5 years to about 15 years, about 14.5 years, about 14 years, about 13.5 years, about 13 years, about 12.5 years, about 12 years, about 11.5 years, about 11 years, about 10.5 years, or about 10 years; about 10 years to about 15 years, about 14.5 years, about 14 years, about 13.5 years, about 13 years, about 12.5 years, about 12 years, about 11.5 years, about 11 years, or about 10.5 years; about 10.5 years to about 15 years, about 14.5 years, about 14 years, about 13.5 years, about 13 years, about 12.5 years, about 12 years, about 11.5 years, or about 11 years; about 11 years to about 15 years, about 14.5 years, about 14 years, about 13.5 years, about 13 years, about 12.5 years, about 12 years, or about 11.5 years; about 11.5 years to about 15 years, about 14.5 years, about 14 years, about 13.5 years, about 13 years, about 12.5 years, or about 12 years; about 12 years to about 15 years, about 14.5 years, about 14 years, about 13.5 years, about 13 years, or about 12.5 years; about 12.5 years to about 15 years, about 14.5 years, about 14 years, about 13.5 years, or about 13 years; about 13 years to about 15 years, about 14.5 years, about 14 years, or about 13.5 years; about 13.5 years to about 15 years, about 14.5 years, or about 14 years; about 14 years to about 15 years or about 14.5 years; or about 14.5 years to about 15 years.

### Methods of Improving the Prognosis of a Mammal Having a Cancer

Also provided herein are methods of improving the prognosis of a mammal having a cancer that include: (a) identifying a mammal (e.g., any of the mammals described herein) having a cancer (e.g., any of the cancers described herein); (b) harvesting a T cell (e.g., any of the T cells described herein) from the identified mammal; (c) culturing the T cell (e.g., using any of the culturing methods, culture media, culturing conditions, or culture times described here): in a culture medium sufficient to induce and/or increase anaplerosis (e.g., any of the culture media sufficient to induce and/or increase anaplerosis described herein) and/or comprising a NLRP3 activator (e.g., any of the NLRP3 activators described herein) in an amount sufficient to improve anti-tumor activity of a T cell (e.g., any of the anti-tumor activities of a T cell described herein); and/or under conditions sufficient to induce and/or increase anaplerosis (e.g., any of the conditions sufficient to induce and/or increase anaplerosis described herein), thereby resulting in an improvement in the anti-tumor activity of the T cell; and (d) administering the T cell to the mammal. Any of the NLRP3 activators, any of the culture media sufficient to induce and/or increase anaplerosis, and/or any of the conditions sufficient to induce and/or increase anaplerosis described herein can be used in these methods. The T cells can be administered back to the mammal at any of the doses described herein. The T cells can be administered in any of the formulations described herein. The T cells can be administered to the mammal via intravenous administration, intraarterial administration, or intratumor injection. The T cells can be administered to the mammal at any of the administration frequencies described herein.

Also provided are methods of improving the prognosis of a mammal having a cancer that include: (a) identifying a mammal (e.g., any of the mammals described herein) having a cancer (e.g., any of the cancers described herein); and (b) administering a therapeutically effective amount of a NLRP3 activator (e.g., any of the NLRP3 activators described herein) to the identified mammal. Any of the NLRP3 activators can be used in these methods. The NLRP3 activator can be administered to the mammal at any of the doses described herein. The NLRP3 activator can be administered using any of the routes of administration described herein for NLRP3 activators. The NLRP3 activators can be administered to the mammal at any of the administration frequencies described herein.

In some examples of any of these methods, the mammals are diagnosed as having a cancer. Some embodiments of these methods further include diagnosing a mammal as having a cancer (e.g., using any of the exemplary methods described herein).

In any of these methods, the mammal's prognosis is improved as compared to the mammal's prognosis prior to treatment, or as compared to a mammal or a populations of mammals having the same or a similar cancer and receiving no treatment or a different treatment. In some examples of any of these methods, an improved prognosis of a mammal having a cancer can include one or more of: an increased likelihood of having an increased time of survival, a decreased likelihood of developing a metastasis, a decreased likelihood of developing one or more additional metastases, an increased likelihood of having at least one solid tumor having a decrease in size over time, and an increased likelihood of having an increased time of remission of the cancer in the mammal (e.g., as compared to a mammal or a population of mammals having the same or a similar cancer and receiving no treatment or receiving a different treatment).

### Methods of Decreasing the Risk of Developing a Metastasis or an Additional Metastasis in a Mammal Having a Cancer

Also provided are methods of decreasing the risk of developing a metastasis or an additional metastasis in a mammal having a cancer that include: (a) identifying a mammal (e.g., any of the mammals described herein) having a cancer (e.g., any of the cancers described herein); (b) harvesting a T cell (e.g., any of the T cells described herein) from the identified mammal; (c) culturing the T cell (e.g., using any of the culturing methods, culture media, culture conditions, and/or culture times described herein): in a culture medium sufficient to induce and/or increase anaplerosis (e.g., any of the culture media sufficient to induce and/or increase anaplerosis described herein) and/or comprising a NLRP3 activator (e.g., any of the NLRP3 activators described herein) in an amount sufficient to improve anti-tumor activity of a T cell; and/or under conditions sufficient to induce and/or increase anaplerosis (e.g., any of conditions sufficient to induce and/or increase anaplerosis described herein), thereby resulting in an improvement in the anti-tumor activity of the T cell (e.g., any of the anti-tumor activities of a T cell described herein); and (d) administering the T cell to the mammal. Any of the NLRP3 activators, any of the culture media sufficient to induce and/or increase anaplerosis, and/or any of the conditions sufficient to induce and/or increase anaplerosis described herein can be used in these methods. The T cells can be administered back to the mammal at any of the doses described herein. The T cells can be administered in any of the formulations described herein. The T cells can be administered to the mammal via intravenous administration, intraarterial administration, or intratumor injection. The T cells can be administered to the mammal at any of the administration frequencies described herein.

Also provided are methods of decreasing the risk of developing a metastasis or an additional metastasis in a mammal having a cancer that include: (a) identifying a mammal (e.g., any of the mammals described herein) having a cancer (e.g., any of the cancers described herein); and (b) administering a therapeutically effective amount of a NLRP3 activator (e.g., any of the NLRP3 activators described herein) to the identified mammal. Any of the NLRP3 activators can be used in these methods. The NLRP3 activator can be administered to the mammal at any of the doses described herein. The NLRP3 activator can be administered using any of the routes of administration described herein for NLRP3 activators. The NLRP3 activators can be administered to the mammal at any of the administration frequencies described herein.

In some examples of any of these methods, the mammals are diagnosed as having a cancer. Some embodiments of these methods further include diagnosing a mammal as having a cancer (e.g., using any of the exemplary methods described herein).

The decrease in the risk of developing a metastasis or an additional metastasis in a mammal having a cancer can be as compared to the risk of developing a metastasis or an additional metastasis in the mammal prior to treatment, or as compared to a mammal or a population of mammals having a similar or the same cancer that has received no treatment or a different treatment. The decrease in the risk of developing a metastasis or an additional metastasis can be about 1% to about 99%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%; about 5% to about 99%, about 95%, about 90%, about 85%, about 80%, about 75%, about 70%, about 65%, about 60%, about 55%, about 50%, about 45%, about 40%, about 35%, about 30%, about 25%, about 20%, about 15%, or about 10%; about 10% to about 99%, about 95%, about 90%, about 85%, about 80%, about 75%, about 70%, about 65%, about 60%, about 55%, about 50%, about 45%, about 40%, about 35%, about 30%, about 25%, about 20%, or about 15%; about 15% to about 99%, about 95%, about 90%, about 85%, about 80%, about 75%, about 70%, about 65%, about 60%, about 55%, about 50%, about 45%, about 40%, about 35%, about 30%, about 25%, or about 20%; about 20% to about 99%, about 95%, about 90%, about 85%, about 80%, about 75%, about 70%, about 65%, about 60%, about 55%, about 50%, about 45%, about 40%, about 35%, about 30%, or about 25%; about 25% to about 99%, about 95%, about 90%, about 85%, about 80%, about 75%, about 70%, about 65%, about 60%, about 55%, about 50%, about 45%, about 40%, about 35%, or about 30%; about 30% to about 99%, about 95%, about 90%, about 85%, about 80%, about 75%, about 70%, about 65%, about 60%, about 55%, about 50%, about 45%, about 40%, or about 35%; about 35% to about 99%, about 95%, about 90%, about 85%, about 80%, about 75%, about 70%, about 65%, about 60%, about 55%, about 50%, about 45%, or about 40%; about 40% to about 99%, about 95%, about 90%, about 85%, about 80%, about 75%, about 70%, about 65%, about 60%, about 55%, about 50%, or about 45%; about 45% to about 99%, about 95%, about 90%, about 85%, about 80%, about 75%, about 70%, about 65%, about 60%, about 55%, or about 50%; about 50% to about 99%, about 95%, about 90%, about 85%, about 80%, about 75%, about 70%, about 65%, about 60%, or about 55%; about 55% to about 99%, about 95%, about 90%, about 85%, about 80%, about 75%, about 70%, about 65%, or about 60%; about 60% to about 99%, about 95%, about 90%, about 85%, about 80%, about 75%, about 70%, or about 65%; about 65% to about 99%, about 95%, about 90%, about 85%, about 80%, about 75%, or about 70%; about 70% to about 99%, about 95%, about 90%, about 85%, about 80%, or about 75%; about 75% to about 99%, about 95%, about 90%, about 85%, or about 80%; about 80% to about 99%, about 95%, about 90%, or about 85%; about 85% to about 99%, about 95%, or about 90%; about 90% to about 99% or about 90%; or about 95% to about 99% as compared to the risk of developing a metastasis or an additional metastasis in the mammal prior to treatment, or as compared to a mammal or a population of mammals having a similar or the same cancer that has received no treatment or a different treatment

In some examples, the risk of developing a metastasis or an additional metastasis is over about 2 weeks, 1 month, 1.5 months, 2 months, 2.5 months, 3 months, 3.5 months, 4 months, 4.5 months, 5 months, 5.5 months, 6 months, 6.5 months, 7 months, 7.5 months, 8 months, 8.5 months, 9 months, 9.5 months, 10 months, 10.5 months, 11 months, 11.5 months, 12 months, 1.5 years, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, or 10 years.

Methods of Increasing the Level of an Anti-Tumor Lymphokine and/or an Anti-Tumor Cytokine

Also provided are methods of increasing the level of at least one (e.g., two, three, four, five, six, seven, eight, nine, or ten) anti-tumor lymphokine and/or at least one anti-tumor cytokine in a mammal having a cancer that include: (a) identifying a mammal (e.g., any of the mammals described herein) having a cancer (e.g., any of the cancers described herein); (b) harvesting a T cell (e.g., any of the T cells described herein) from the identified mammal; (c) culturing the T cell (e.g., using any of the culturing methods, culture media, culture conditions, and/or culture times described herein): in a culture medium sufficient to induce and/or increase anaplerosis (e.g., any of the culture media sufficient to induce and/or increase anaplerosis described herein) and/or including a NLRP3 activator (e.g., any of the NLRP3 activators described herein) in an amount sufficient to improve anti-tumor activity of a T cell; and/or under conditions sufficient to induce and/or increase anaplerosis (e.g., any of the conditions sufficient to induce and/or increase anaplerosis described herein), thereby resulting in an improvement in the anti-tumor activity of the T cell (e., any of the anti-tumor activities described herein); and (d) administering the T cell to the mammal. Any of the NLRP3 activators, any of the culture media sufficient to induce and/or increase anaplerosis, and/or any of the conditions sufficient to induce and/or increase anaplerosis described herein can be used in these methods. The T cells can be administered back to the mammal at any of the doses described herein. The T cells can be administered in any of the formulations described herein. The T cells can be administered to the mammal via intravenous administration, intraarterial administration, or intratumor injection. The T cells can be administered to the mammal at any of the administration frequencies described herein.

Also provided are methods of increasing the level of at least one (e.g., two, three, four, five, six, seven, eight, nine, or ten) anti-tumor lymphokine and/or at least one anti-tumor cytokine in a mammal having a cancer that include: (a) identifying a mammal (e.g., any of the mammals described herein) having a cancer (e.g., any of the cancers described herein); and (b) administering a therapeutically effective amount of a NLRP3 activator (e.g., any of the NLRP3 activators described herein) to the identified mammal. Any of the NLRP3 activators can be used in these methods. The NLRP3 activator can be administered to the mammal at any of the doses described herein. The NLRP3 activator can be administered using any of the routes of administration described herein for NLRP3 activators. The NLRP3 activators can be administered to the mammal at any of the administration frequencies described herein.

In some examples of any of these methods, the mammals are diagnosed as having a cancer. Some embodiments of these methods further include diagnosing a mammal as having a cancer (e.g., using any of the exemplary methods described herein).

In some embodiments, the at least one anti-tumor cytokine and/or lymphokine is selected from the group of: GM-CSF, IL-7, IL-12, IL-15, IL-18, and IL-21. The levels of anti-tumor cytokines and/or lymphokines in a sample from a mammal can be detected using one or more antibodies that bind specifically to the anti-tumor cytokine and/or lymphokine (e.g., one or more commercially available antibodies that bin specifically to the anti-tumor cytokine and/or lymphokine). In some examples, the increase in the level of the at least one anti-tumor cytokine and/or lymphokine is as compared to the levels of the at least one anti-tumor cytokine and/or lymphokine in the mammal prior to treatment. In some examples, the increase in the level of the at least one anti-tumor cytokine and/or lymphokine is as compared to the levels of the at least one anti-tumor cytokine and/or lymphokine in a mammal or a population of mammals having the same or a similar cancer, but receiving no treatment or a different treatment.

In some examples, the increase in the level of the at least one anti-tumor cytokine and/or lymphokine can be about 1% to about 200%, about 180%, about 160%, about 140%, about 120%, about 100%, about 90%, about 80%, about 70%, about 60%, about 50%, about 40%, about 30%, about 20%, about 10%, or about 5%; about 5% to about 200%, about 180%, about 160%, about 140%, about 120%, about 100%, about 90%, about 80%, about 70%, about 60%, about 50%, about 40%, about 30%, about 20%, or about 10%; about 10% to about 200%, about 180%, about 160%, about 140%, about 120%, about 100%, about 90%, about 80%, about 70%, about 60%, about 50%, about 40%, about 30%, or about 20%; about 20% to about 200%, about 180%, about 160%, about 140%, about 120%, about 100%, about 90%, about 80%, about 70%, about 60%, about 50%, about 40%, or about 30%; about 30% to about 200%, about 180%, about 160%, about 140%, about 120%, about 100%, about 90%, about 80%, about 70%, about 60%, about 50%, or about 40%; about 40% to about 200%, about 180%, about 160%, about 140%, about 120%, about 100%, about 90%, about 80%, about 70%, about 60%, or about 50%; about 50% to about 200%, about 180%, about 160%, about 140%, about 120%, about 100%, about 90%, about 80%, about 70%, or about 60%; about 60% to about 200%, about 180%, about 160%, about 140%, about 120%, about 100%, about 90%, about 80%, or about 70%; about 70% to about 200%, about 180%, about 160%, about 140%, about 120%, about 100%, about 90%, or about 80%; about 80% to about 200%, about 180%, about 160%, about 140%, about 120%, about 100%, or about 90%; about 90% to about 200%, about 180%, about 160%, about 140%, about 120%, or about 100%; about 100% to about 200%, about 180%, about 160%, about 140%, or about 120%; about 120% to about 200%, about 180%, about 160%, or about 140%; about 140% to about 200%, about 180%, or about 160%; about 160% to about 200% or about 180%; or about 180% to about 200%, as compared to the levels of the at least one anti-tumor cytokine and/or lymphokine in the mammal prior to treatment, or as compared to the levels of the at least one anti-tumor cytokine and/or lymphokine in a mammal or a population of mammals having the same or a similar cancer, but receiving no treatment or a different treatment.

The increase in the level of at least one anti-tumor cytokine and/or lymphokine can be determined after about 1 day to about 2 years, about 22 months, about 20 months, about 18 months, about 16 months, about 14 months, about 12 months, about 10 months, about 8 months, about 6 months, about 4 months, about 2 months, about 1 month, about 2 weeks, or about 1 week; about 1 week to about 2 years, about 22 months, about 20 months, about 18 months, about 16 months, about 14 months, about 12 months, about 10 months, about 8 months, about 6 months, about 4 months, about 2 months, about 1 month, or about 2 weeks; about 2 week to about 2 years, about 22 months, about 20 months, about 18 months, about 16 months, about 14 months, about 12 months, about 10 months, about 8 months, about 6 months, about 4 months, about 2 months, or about 1 month; about 1 month to about 2 years, about 22 months, about 20 months, about 18 months, about 16 months, about 14 months, about 12 months, about 10 months, about 8 months, about 6 months, about 4 months, or about 2 months; about 2 months to about 2 years, about 22 months, about 20 months, about 18 months, about 16 months, about 14 months, about 12 months, about 10 months, about 8 months, about 6 months, or about 4 months; about 4 months to about 2 years, about 22 months, about 20 months, about 18 months, about 16 months, about 14 months, about 12 months, about 10 months, about 8 months, to about 6 months; about 6 months to about 2 years, about 22 months, about 20 months, about 18 months, about 16 months, about 14 months, about 12 months, about 10 months, or about 8 months; about 8 months to about 2 years, about 22 months, about 20 months, about 18 months, about 16 months, about 14 months, about 12 months, or about 10 months; about 10 months to about 2 years, about 22 months, about 20 months, about 18 months, about 16 months, about 14 months, or about 12 months; about 12 months to about 2 years, about 22 months, about 20 months, about 18 months, about 16 months, or about 14 months; about 14 months to about 2 years, about 22 months, about 20 months, about 18 months, or about 16 months; about 16 months to about 2 years, about 22 months, about 20 months, or about 18 months; about 18 months to about 2 years, about 22 months, or about 20 months; about 20 months to about 2 years or about 22 months; or about 22 months to about 24 months of treatment.

Methods of Maintaining a Population of Recombinant T Cells in a Mammal after Administering the Recombinant T Cells to the Mammal

Also provided are methods for maintaining a population of recombinant T cells in a mammal (e.g., any of the mammals described herein, e.g., a human) for at least one month after administering the recombinant T cell to the mammal that include: (a) identifying a mammal having a cancer (e.g., any of the cancers described herein); (b) harvesting a T cell (e.g., any of the T cells described herein) from the identified mammal; (c) introducing a recombinant nucleic acid into the T cell to generate a recombinant T cell (e.g., a T cell including a heterologous TCR or a CAR T cell); (d) culturing the recombinant T cell: in a culture medium sufficient to induce and/or increase anaplerosis (e.g., any of the culture media sufficient to induce and/or increase anaplerosis described herein) and/or comprising a NLRP3 activator (e.g., any of the NLRP3 activators described herein) in an amount sufficient to improve anti-tumor activity of a T cell (e.g., one or more of any of the anti-tumor activities of a T cell described herein); and/or under conditions sufficient to induce and/or increase anaplerosis, thereby resulting in an improvement in the anti-tumor activity of the recombinant T cell; and (d) administering the recombinant T cell to the mammal, where a population of the recombinant T cells persists in the mammal for at least one month after the administering of the recombinant T cell to the mammal. The recombinant T cells can be cultured using any of the culturing methods described herein. Any of the doses of recombinant T cells described herein can be administered to the mammal in these methods. The recombinant T cells can be administered by, e.g., intraarterial administration, intravenous administration, or intra-tumor injection. The recombinant T cells can be administered once a week, twice a week, three times a week, once every two weeks, once every three weeks, once a month, twice a month, once every two months, once every three months, once every four months, once every five months, once every six months, once every seven months, once every eight months, once every nine months, once every ten months, once every eleven months, or once every twelve months. The recombinant T cells can be administered in any of the formulations described herein.

The mammal may by diagnosed as having a cancer. Some embodiments further include an active step of diagnosing a mammal as having a cancer. In some embodiments, the mammal may have already been administered a treatment and the treatment was not effective.

In some examples, the methods result in a population of recombinant T cells being maintained in a mammal for at least one month, at least two months, at least three months, at least four months, at least five months, at least 6 months, at least 7 months, at least 8 months, at least 9 months, at least 10 months, at least 11 months, at least 1 year, at least 13 months, at least 14 months, at least 15 months, at least 16 months, at least 17 months, at least 18 months, at least 19 months, at least 20 months, at least 21 months, at least 22 months, at least 23 months, at least 2 years, at least 25 months, at least 26 months, at least 27 months, at least 28 months, at least 29 months, at least 30 months, at least 31 months, at least 32 months, at least 33 months, at least 34 months, at least 35 months, at least 3 years, at least 37 months, at least 38 months, at least 39 months, at least 40 months, at least 41 months, at least 42 months, at least 43 months, at least 44 months, at least 45 months, at least 46 months, at least 47 months, at least 4 years, at least 49 months, at least 50 months, at least 51 months, at least 52 months, at least 53 months, at least 54 months, at least 55 months, at least 56 months, at least 57 months, at least 58 months, at least 59 months, at least 5 years, at least 61 months, at least 62 months, at least 63 months, at least 64 months, at least 65 months, at least 66 months, at least 67 months, at least 68 months, at least 69 months, at least 70 months, at least 71 months, or at least 6 years after administering the recombinant T cell to the mammal.

In some examples, the methods result in a population of recombinant T cells being maintained in a mammal for about 1 month to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, about 20 months, about 19 months, about 18 months, about 17 months, about 16 months, about 15 months, about 14 months, about 13 months, about 12 months, about 11 months, about 10 months, about 9 months, about 8 months, about 7 months, about 6 months, about 5 months, about 4 months, about 3 months, or about 2 months; about 2 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, about 20 months, about 19 months, about 18 months, about 17 months, about 16 months, about 15 months, about 14 months, about 13 months, about 12 months, about 11 months, about 10 months, about 9 months, about 8 months, about 7 months, about 6 months, about 5 months, about 4 months, or about 3 months; about 3 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, about 20 months, about 19 months, about 18 months, about 17 months, about 16 months, about 15 months, about 14 months, about 13 months, about 12 months, about 11 months, about 10 months, about 9 months, about 8 months, about 7 months, about 6 months, about 5 months, or about 4 months; about 4 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, about 20 months, about 19 months, about 18 months, about 17 months, about 16 months, about 15 months, about 14 months, about 13 months, about 12 months, about 11 months, about 10 months, about 9 months, about 8 months, about 7 months, about 6 months, or about 5 months; about 5 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, about 20 months, about 19 months, about 18 months, about 17 months, about 16 months, about 15 months, about 14 months, about 13 months, about 12 months, about 11 months, about 10 months, about 9 months, about 8 months, about 7 months, or about 6 months; about 6 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, about 20 months, about 19 months, about 18 months, about 17 months, about 16 months, about 15 months, about 14 months, about 13 months, about 12 months, about 11 months, about 10 months, about 9 months, about 8 months, or about 7 months; about 7 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, about 20 months, about 19 months, about 18 months, about 17 months, about 16 months, about 15 months, about 14 months, about 13 months, about 12 months, about 11 months, about 10 months, about 9 months, or about 8 months; about 8 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, about 20 months, about 19 months, about 18 months, about 17 months, about 16 months, about 15 months, about 14 months, about 13 months, about 12 months, about 11 months, about 10 months, or about 9 months; about 9 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, about 20 months, about 19 months, about 18 months, about 17 months, about 16 months, about 15 months, about 14 months, about 13 months, about 12 months, about 11 months, or about 10 months; about 10 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, about 20 months, about 19 months, about 18 months, about 17 months, about 16 months, about 15 months, about 14 months, about 13 months, about 12 months, or about 11 months; about 11 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, about 20 months, about 19 months, about 18 months, about 17 months, about 16 months, about 15 months, about 14 months, about 13 months, or about 12 months; about 12 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, about 20 months, about 19 months, about 18 months, about 17 months, about 16 months, about 15 months, about 14 months, or about 13 months; about 13 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, about 20 months, about 19 months, about 18 months, about 17 months, about 16 months, about 15 months, or about 14 months; about 14 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, about 20 months, about 19 months, about 18 months, about 17 months, about 16 months, or about 15 months; about 15 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, about 20 months, about 19 months, about 18 months, about 17 months, or about 16 months; about 16 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, about 20 months, about 19 months, about 18 months, or about 17 months; about 17 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, about 20 months, about 19 months, or about 18 months; about 18 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, about 20 months, or about 19 months; about 19 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, about 21 months, or about 20 months; about 20 months to about 7 years, about 6.5 years, about 6 years, about 5.5 years, about 5 years, about 4.5 years, about 4 years, about 3.5 years, about 3 years, about 2.5 years, about 2 years, about 23 months, about 22 months, or about 21 months; about 21 months to about 7 years, about 6.5 years, about 6 years, about 5.5 years, about 5 years, about 4.5 years, about 4 years, about 3.5 years, about 3 years, about 2.5 years, about 2 years, about 23 months, or about 22 months; about 22 months to about 7 years, about 6.5 years, about 6 years, about 5.5 years, about 5 years, about 4.5 years, about 4 years, about 3.5 years, about 3 years, about 2.5 years, about 2 years, or about 23 months; about 23 months to about 7 years, about 6.5 years, about 6 years, about 5.5 years, about 5 years, about 4.5 years, about 4 years, about 3.5 years, about 3 years, about 2.5 years, or about 2 years; about 2 years to about 7 years, about 6.5 years, about 6 years, about 5.5 years, about 5 years, about 4.5 years, about 4 years, about 3.5 years, about 3 years, or about 2.5 years; about 2.5 years to about 7 years, about 6.5 years, about 6 years, about 5.5 years, about 5 years, about 4.5 years, about 4 years, about 3.5 years, or about 3 years; about 3 years to about 7 years, about 6.5 years, about 6 years, about 5.5 years, about 5 years, about 4.5 years, about 4 years, or about 3.5 years; about 3.5 years to about 7 years, about 6.5 years, about 6 years, about 5.5 years, about 5 years, about 4.5 years, or about 4 years; about 4 years to about 7 years, about 6.5 years, about 6 years, about 5.5 years, about 5 years, or about 4.5 years; about 4.5 years to about 7 years, about 6.5 years, about 6 years, about 5.5 years, or about 5 years; about 5 years to about 7 years, about 6.5 years, about 6 years, or about 5.5 years; about 5.5 years to about 7 years, about 6.5 years, or about 6 years; about 6 years to about 7 years or about 6.5 years; or about 6.5 years to about 7 years.

### Methods of Identifying a Candidate Agent for Increasing Resistance of a T Cell to at Least One Immunosuppressive Cytokine

Also provided are methods (e.g., in vitro methods) of identifying a candidate agent for increasing resistance of a T cell (e.g., any of the T cells described herein) to at least one immunosuppressive cytokine (e.g., any of the immunosuppressive cytokines described herein) that include: (a) providing a mammalian cell (e.g., an epithelial cell, an endothelial cell, a HEK cell, a CHO cell, or any of the innate immune cells described herein, or any of the T cells described herein) that expresses NLRP3 protein; (b) contacting the mammalian cell with an agent (e.g., a molecule having a mass of less than 5 kDa, a protein, a lipid, a nucleic acid, a saccharide, or any combination thereof); (c) detecting the level of NLRP3 activity in the cell in step (b); and (d) identifying an agent that increases NLRP3 activity or a level of NLRP3 (e.g., NLRP3 protein or a nucleic acid encoding a NLRP3 protein) in the cell as compared to a control level of NLRP3 activity or a control level of NLRP3 as a candidate agent for increasing resistance of a T cell to at least one immunosuppressive cytokine. The control level of NLRP3 activity is the level of NLRP3 activity in the cell (or a similar cell) in the absence of the agent. The control level of NLRP3 is the level of NLRP3 protein or a nucleic acid encoding a NLRP3 protein in the cell (or a similar cell) in the absence of the agent. Exemplary methods for detecting the activity of NLRP3, detecting the level of NLRP3 protein, and detecting the level of a nucleic acid encoding a NLRP3 protein are described herein. In some embodiments, the NLRP3 activity is NLRP3 downstream signaling activity (e.g., the different NLRP3 downstream signaling events described herein).

Some embodiments further include (e) contacting the mammalian cell (e.g., any of the T cells described herein) with the at least one immunosuppressive cytokine (e.g., any of the immunosuppressive cytokines described herein) and the candidate agent; and (f) determining the ability of the candidate agent to block the immunosuppressive activity of the at least one immunosuppressive cytokine on the T cell.

### Example

### Example 1

### An NLRP3 Activator Increases T Cell Resistance to an Immunosuppressive Cytokine

### Objective

An experiment was performed to see if an exemplary NLRP3 activator, IL-1β, is capable of increasing T cell resistance to an exemplary immunosuppressive cytokine, TGF-β.

### The Model

Flank tumors were generated in mice using the OVA-albumin (OA) expressing thymoma cell line, EG7. Splenocytes from transgenic OT-1 mice (whose CD8⁺ T-cells recognize an immunodominant peptide in OA) were activated in vitro with different cytokines and adoptively transferred into mice bearing large tumors. This is a well-established model to evaluate anti-cancer activity of in vitro differentiated cells.

### Materials and Methods

### Cell Isolation and Culture

EG7 is a derivative of a parental murine thymoma cell line (EL4) that was transfected with an OVA cDNA construct. EG7 cells were cultured and maintained in media consisting of RPMI media (Gibco) supplemented with 100 mg/ml G418, 10% fetal bovine serum, 2 mM glutamine, 100 U/ml penicillin, 100 μg/ml streptomycin, 100 mM sodium pyruvate, and 2.5% β-mercaptoethanol.

### In Vitro T Cell Stimulation and Isolation

OT-1 mice were anesthetized and spleens were harvested and strained through a 70-um filter with PBS with 10% FBS. Red blood cells were removed with lysing buffer (BD Pharmingen) for 15 minutes at room temperature. After washing, cells were counted and placed in tissue culture at a final concentration of 1×10⁶ cells/mL. OT-1 splenocytes were activated with media containing the SIINFEKL peptide (1 μg/mL) (Bachem, King of Prussia, Pa.) on Day 0. Cells were treated with TGF-β (10 ng/mL) (R&D Systems, Minneapolis, Minn.) or with TGF-β (10 ng/mL) and IL-1β (10 ng/mL) or not. After 5 days CD8⁺ cells were isolated by negative selection with magnetic beads using a CD8 isolation kit from Mylteni, resuspended in PBS, and used for adoptive transfer and T cell studies.

### Mouse Strain and Housing

Nine-week old C57BL/6J female mice were purchased from the Jackson Laboratory and housed at a temperature ranging from 68 degrees F. to 74 degrees F. with a diurnal 12 hour light cycle in a specific pathogen-free facility in ventilated cages. Food and water was provided ad libitum. Animals were acclimated to local microbiota for 7 days before the beginning of the experiment.

### Animal Tumor Model

Tumors were established with subcutaneous flank injections of 1×10⁶ cells suspended in 100 μL PBS. Tumors were measured three times a week and volumes were estimated using the formula 3.14×[largest diameter×(perpendicular diameter)²]/6. Treatment was administered after 7 days when tumors were palpable and mice followed for tumor growth. Mice were sacrificed when the estimated tumors volume was greater than 2500 mm³ or the mice demonstrated signs of distress.

### Adoptive T Cell Transfer

CD8⁺ cells isolated from splenocytes activated in vitro in the presence of TGF-β, or in the presence of TGF-β and IL-1β, were resuspended in PBS. For each group (no cytokines, TGF-β, TGF-β and IL-1β) 5×10⁶ cells in 200 μL of PBS (vehicle) alone were adoptively transferred via intraperitoneal injection. A control group of mice was injected with vehicle alone to estimate tumor growth in the absence of treatment.

### Calculation of Therapeutic Effect—

The average tumor volumes of the combination treatment group over time was plotted and compared to control treated with vehicle. Each experiment had 10 mice per group. For statistical comparisons, Two-Way Anova with Bonferroni correction was calculated with GaphPrism software.

### CONCLUSIONS

These data show that an NLRP3 activator can be used to increase the resistance of a T cell to an immunosuppressive cytokine, and thereby result in an increase in anti-tumor activity of a T cell.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of increasing resistance of a T cell or an innate immune cell to at least one immunosuppressive cytokine, the method comprising:
(a) providing a T cell or an innate immune cell, and
(b) culturing the T cell or the innate immune cell in a culture medium comprising an amount of an NLRP3 activator sufficient to increase resistance of a T cell or an innate immune cell to the at least one immunosuppressive cytokine.

2. The method of claim 1, wherein the culture medium further comprises one or more agents that activate the T cell or the innate immune cell.

3. The method of claim 2, wherein the one or more agents that activate the T cell or the innate immune cell comprises an isolated tumor antigen, a tumor lysate, necrotic tumor cells, tumor apoptotic bodies, an isolated antigen from a pathogenic organism or virus, or a tumor vaccine.

4. The method of any one of claims 1-3, wherein culturing the T cell or the innate immune cell in step (b) results in an increase of NLRP3 downstream signaling in the T cell or the innate immune cell.

5. The method of any one of claims 1-3, wherein culturing the T cell or the innate immune cell in step (b) results in an increase in the level of NLRP3 protein in the T cell or the innate immune cell.

6. The method of any one of claims 1-5, wherein the innate immune cell is a dendritic cell, a macrophage, or a monocyte.

7. The method of any one of claims 1-6, wherein the method comprises providing an innate immune cell and culturing the innate immune cell in a culture medium comprising an amount of an NLRP3 activator sufficient to increase resistance of an innate immune cell to the at least one immunosuppressive cytokine.

8. The method of any one of claims 1-5, wherein the method comprises providing a T cell and culturing the T cell in a culture medium comprising an amount of an NLRP3 activator sufficient to increase resistance of a T cell to the at least one immunosuppressive cytokine.

9. The method of any one of claims 1-5 and 8, wherein the T cell is a CD4⁺ T cell or a CD8⁺ cell.

10. The method of any one of claims 1-5 and 8, wherein the T cell is selected from the group consisting of: a lymphoid progenitor cell, an immature thymocyte, a peripheral blood lymphocyte, a naïve T cell, a pluripotent T_{H} cell precursor, a T_{reg} cell, a memory T cell, a T_{H}17 cell, a T_{H}22 cell, a T_{H}9 cell, a T_{H}2 cell, a T_{H}1 cell, a T_{H}3 cell, γδ T cell, an αβ T cell, and a tumor-infiltrating T cell.

11. The method of claims 1-5 and 8-10, wherein the T cell is a chimeric antigen receptor (CAR)-T cell.

12. The method of claim 11, wherein the CAR-T cell comprises a nucleic acid encoding a CAR protein comprising:
an antigen-binding domain,
a transmembrane domain, and
a cytoplasmic signaling domain.

13. The method of claim 12, wherein the antigen-binding domain is an antibody, an antigen-binding fragment of an antibody, a Fab fragment, and a scFv.

14. The method of claim 13, wherein the antibody is a human or humanized antibody, or the antigen-binding fragment of an antibody or the Fab is a fragment of a human or humanized antibody.

15. The method of any one of claims 12-14, wherein the cytoplasmic signaling domain comprises a CD3ζ cytoplasmic sequence.

16. The method of claim 15, wherein the cytoplasmic signaling domain further comprises a cytoplasmic sequence of one or more of the following proteins: CD27, CD28, 4-1BB, OX40, CD30, CD40, PD-1, ICOS, LFA-1, CD2, CD7, LIGHT, NKG2C, B7-H3, and a ligand that specifically binds to CD83.

17. The method of claim 12-16, wherein the transmembrane domain comprises a transmembrane sequence from CD3ζ, CD8, or CD28.

18. The method of any one of claims 12-17, wherein the CAR protein further comprises a linker sequence between the antigen-binding domain and the transmembrane domain.

19. The method of any one of claims 1-10, wherein the T cell is harvested from a mammal.

20. The method of any one of claims 1-7, wherein the innate immune cell is harvested from a mammal.

21. The method of claim 19 or 20, wherein the mammal has been identified as having a cancer or an infectious disease.

22. The method of claim 21, wherein the mammal has been identified as having a cancer selected from the group consisting of: acute lymphoblastic leukemia, acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastric cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, gastric cancer, testicular cancer, throat cancer, thyroid cancer, urethral cancer, uterine cancer, vaginal cancer, and vulvar cancer.

23. The method of claim 19 or 20, wherein the mammal has been identified as having an infectious disease selected from the group of: Acinobacter infection, actinomycosis, African sleeping sickness, acquired immunodeficiency syndrome, amebiasis, anaplasmosis, anthrax, Arcanobacterium haemolyticum infection, Argentine hemorrhagic fever, ascariasis, aspergillosis, astrovirus infection, babesiosis, Bacillus cereus infection, bacterial pneumonia, bacterial vaginosis, Bacteroides infection, balantidiasis, Baylisascaris infection, BK virus infection, black piedra, Blastocystic hominis infection, blastomycosis, Bolivian hemorrhagic fever, botulism, Brazilian hemorrhagic fever, brucellosis, bubonic plaque, Burkholderi infection, Buruli ulcer, Calicivirus infection, camptobacteriosis, candidiasis, cat-scratch disease, cellulitis, Chagas disease, chancroid, chickenpox, chikungunya, chlamydia, Chlamydophila pneumoniae infection, cholera, chromoblastomycosis, clonorchiasis, Clostridium difficile infection, coccidioidomycosis, Colorado tick fever, common cold, Creutzfeldt-Jakob disease, Crimean-Congo hemorrhagic fever, crytococcosis, cryptosporidiosis, cutaneous larva migrans, cyclosporiasis, cysticercosis, cytomegalovirus infection, dengue fever, Desmodesmus infection, deintamoebiasis, diphtheria, diphyllobothriasis, dracunculiasis, ebola hemorrhagic fever, echinococcosis, ehrlichiosis, enterobiasis, Enterococcus infection, Enterovirus infection, epidemic typhus, erythema infection, exanthema subitum, fasciolopsiasis, fasciolosis, fatal familial insomnia, filariasis, food poisoning by Clostridium myonecrosis, free-living amebic infection, Fusobacterium infection, gas gangrene, geotrichosis, Gerstmann-Sträussler-Scheinker syndrome, giardiasis, glanders, gnathostomiasis, gonorrhea, granuloma inguinale, Group A streptococcal infection, Group B streptococcal infection, Haemophilus influenzae infection, hand foot and mouth disease, hantavirus pulmonary syndrome, Heartland virus disease, Heliobacter pylori infection, hemolytic-uremic syndrome, hemorrhagic fever with renal syndrome, hepatitis A, hepatitis B, hepatitis C, hepatitis D, hepatitis E, herpes simplex, histoplasmosis, hookworm infection, human bocavirus infection, human ewingii ehrlichiosis, human granulocyte anaplasmosis, human metapneuomovirus infection, human monocytic ehrlichiosis, human papillomavirus infection, human parainfluenza virus infection, hymenolepiasis, Epstein-Barr virus infectious mononucleosis, influenza, isosporiasis, Kawasaki disease, keratitis, Kingella kingae infection, kuru, lassa fever, Legionnaires' disease, Pontiac fever, leishmaniasis, leprosy, leptospirosis, listeriosis, lyme disease, lymphatic filariasis, lymphocytic choriomeningitis, malaria, Marburg hemorrhagic fever, measles, Middle East respiratory syndrome, melioidosis, meningitis, meningococcal disease, metagonimiasis, microsporidiosis, molluscum contagiosum, monkeypox, mumps, murine typhus, mycoplasma pneumonia, mycetoma, myiasis, neonatal conjunctivitis, variant Creutzfeldt-Jakob disease, nocardiosis, onchocerciasis, paracoccidioidomycosis, paragonimiasis, pasteurellosis, pediculosis capitis, pediculosis corporis, pediculosis pubis, pelvic inflammatory disease, pertussis, plague, pneumonia, poliomyelitis, Prevotella infection, primary amoebic meningoencephalitis, progressive multifocal leukoencephalopathy, psittacosis, Q fever, rabies, relapsing fever, respiratory syncytial virus infection, rhinosporidiosis, rhinovirus infection, rickettsial infection, rickettsialpox, Rift Valley Fever, Rocky Mountain spotted fever, rotavirus infection, rubella, salmonellosis, severe acute respiratory syndrome, scabies, schistosomiasis, sepsis, shigellosis, shingles, smallpox, sporothrichosis, staphylococcal food poisoning, staphylococcal infection, staphylococcal infection, strongyloidiasis, subacute sclerosing panencephalitis, syphilis, taeniasis, tetanus, tinea barabe, tinea capitis, tinea corporis, tinea cruris, tinea manum, tinea nigra, tinea pedis, tinea unguium, tinea versicolor, toxocariasis, trachoma, toxoplasmosis, trichinosis, trichomoniasis, trichuriasis, tuberculosis, tularemia, typhoid fever, Ureaplasma urealyticum infection, valley fever, Venezuelan hemorrhagic fever, viral pneumonia, West Nile fever, white piedra, Yersinia psuedotuberculosis infection, yersiniosis, yellow fever, and zygomycosis.

24. The method of any one of claims 19 and 21-23, further comprising:
harvesting the T cell from the mammal.

25. The method of any one of claims any one of claims 20-23, further comprising:
harvesting the innate immune cell from the mammal.

26. The method of any one of claims 1-25, wherein the at least one immunosuppressive cytokine is selected from the group consisting of: IL-10, TGF-β, IL-1Ra, IL-18Ra, IL-2, IL-3, IL-4, IL-5, IL-6, IL-7, IL-8, IL-9, IL-11, IL-12, IL-13, IL-14, IL-15, IL-16, IL-17, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, PGE2, SCF, G-CSF, CSF-1R, M-CSF, GM-CSF, IFN-α, IFN-β, IFN-γ, IFN-λ, bFGF, CCL2, CXCL1, CXCL8, CXCL12, CX3CL1, CXCR4, and VEGF.

27. The method of any one of claim 26, wherein the at least one immunosuppressive cytokine is TGF-β.

28. The method of any one of claims 1-27, wherein the NLRP3 activator is IL-1α or IL-1β.

29. The method of any one of claims 1-27, wherein the NLRP3 activator is a molecule having a molecular weight of less than 5 kDa.

30. The method of claim 29, wherein the NLRP3 activator is imiquimod or resiquimod, or a pharmaceutically acceptable salt thereof.

31. The method of claim 29, wherein the NLRP3 activator is: 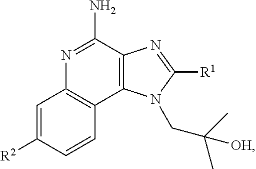
wherein:
(a) R¹ is H, and R² is H;
(b) R¹ is a butyl group and R² is H;
(c) R¹ is H and R² is —CO₂CH₃; or
(d) R¹ is a butyl group and R² is —CO₂CH₃.

32. The method of claim 29, wherein the NLRP3 activator is selected from the group consisting of:
an imadazoquinoline;
an imidazonaphthyridine;
a pyrazolopyridine;
an aryl-substituted imidazoquinoline;
a compound having a 1-alkoxy 1H-imidazo ring system;
an oxazolo [4,5-c]-quinolin-4-amine;
an thiazolo [4,5-c]-quinolin-4-amine;
a selenazolo [4,5-c]-quinolin-4-amine;
an imidazonaphthyridine;
an imidazoquinolinamine;
a 1-substituted, 2-substituted 1H-imidazo[4,5-C]quinolin-4-amine;
a fused cycloalkylimidazopyridine;
a 1H-imidazo[4,5-c]quinolin-4-amine;
a 1-substituted 1H-imidazo-[4,5-c]quinolin-4-amine;
an imidazo-[4,5-C]quinolin-4-amine;
a 2-ethyl 1H-imidazo[4,5-ciquinolin-4-amine;
an olfenic 1H-imidazo[4,5-c]quinolin-4-amine;
a 6,7-dihydro-8-(imidazol-1-yl)-5-methyl-1-oxo-1H,5H-benzo[ij]quinolizine-2-carboxylic acid;
a pyridoquinoxaline-6-carboxylic acid;
a 6,7-dihydro-8-(imidazol-1-yl)-5-methyl-1-oxo-1H,5H-benzo [ij]quinolizine-2-carboxylic acid;
a substituted naphtho[ij]quinolizine;
a substituted pyridoquinoxaline-6-carboxylic acid;
a 7-hydroxy-benzo[ij]quinolizine-2-carboxylic acid derivative;
a substituted benzo[ij]quinolizine-2-carboxylic acid;
a 7-hydroxy-benzo[ij]quinolizine-2-carboxylic acid;
a substituted pyrido[1,2,3,-de]-1,4-benzoxazine; and
a N-methylene malonate of tetrahydroquinoline.

33. A T cell having increased resistance to at least one immunosuppressive cytokine produced by the method of any one of claims 1-5, 8-19, 21-24, and 26-32.

34. A pharmaceutical composition comprising the T cell of claim 33.

35. A kit comprising a pharmaceutical composition of claim 34.

36. An innate immune cell having increased resistance to at least one immunosuppressive cytokine produced by the method of any one of claims 1-7, 20-23, and 26-32.

37. A pharmaceutical composition comprising the innate immune cell of claim 36.

38. A kit comprising a pharmaceutical composition of claim 37.

39. A method of improving the anti-tumor activity of a T cell, the method comprising:
(a) providing a T cell; and
(b) culturing the T cell:
in a culture medium sufficient to induce and/or increase anaplerosis and/or comprising a NLRP3 activator in an amount sufficient to improve anti-tumor activity of a T cell; and/or
under conditions sufficient to induce and/or increase anaplerosis, thereby resulting in an improvement in the anti-tumor activity of the T cell.

40. The method of claim 39, wherein the method comprises culturing the T cell in a culture medium sufficient to induce and/or increase anaplerosis.

41. The method of claim 39, wherein the method comprises culturing the T cell in a culture medium comprising a NLRP3 activator in an amount sufficient to improve anti-tumor activity of a T cell.

42. The method of claim 39, wherein the method comprises culturing the T cell in a culture medium sufficient to induce and/or increase anaplerosis and comprising a NLRP3 activator in an amount sufficient to improve anti-tumor activity of a T cell.

43. The method of any one of claims 40-42, wherein the method further comprises culturing the T cell under conditions sufficient to induce and/or increase anaplerosis.

44. The method of claim 39, wherein the method comprises culturing the T cell under conditions sufficient to induce and/or increase anaplerosis.

45. The method of any one of claims 39-44, wherein the culture medium further comprises one or more agents that activate the T cell.

46. The method of claim 45, wherein the one or more agents that activate the T cell comprises an isolated tumor antigen, tumor lysate, necrotic tumor cells, tumor apoptotic bodies, an isolated antigen from a pathogenic organism or virus, or a tumor vaccine.

47. The method of any one of claims 39, 40, 42, 43, 45, and 46, wherein the culture medium sufficient to induce and/or increase anaplerosis comprises one or more of:
(i) galactose without glucose supplementation;
(ii) culture media that contains galactose and glucose added in any amount that does not prohibit an induce and/or increase in anaplerosis above rates in control cells; and/or
(iii) one or more of 3-bromopyruvate, 2-deoxyglucose, pentavalent arsenic (H₃AsO₄), lonidamine, imatinib, oxythiamine, pyruvate, odd-chain fatty acids, 5-carbon ketone bodies, and triheptanoin.

48. The method of any one of claims 39 and 43-46, wherein the conditions sufficient to induce and/or increase anaplerosis are conditions that result in one or more of the following in the T cell:
a decrease in the rate of lactate production from glycolysis,
a decrease in the rate of lactate production from glycolysis relative to the rate of oxidative phosphorylation,
an increase in the rate of glutamine uptake by the T cell,
an increase in the fraction of lipids and/or amino acids synthesized using glutamine as a substrate,
an increase in the rate at which pyruvate is converted to oxaloacetate by pyruvate carboxylase,
an increase in the rate at which adenylosuccinate synthetase produces fumurate,
an increase in the rate at which aspartate aminotransferase produces oxaloacetate, and
an increase in the rate at which propionyl-CoA carboxylase produces succinyl-CoA.

49. The method of any one of claims 39, 41, 42, 43, 45, and 46, wherein the NLRP3 activator results in an increase in the level of an NLRP3 protein in the T cell.

50. The method of any one of claims 39, 41, 42, 43, 45, and 46, wherein the NLRP3 activator results in an increase NLRP3 downstream signaling in the T cell.

51. The method of any one of claims 39, 41, 42, 43, 45, 46, 49, and 50, wherein the NLRP3 activator is interleukin-1α or interleukin-1β.

52. The method of any one of claims 39, 41, 42, 43, 45, 46, 49, and 50, wherein the NLRP3 activator is a molecule having a molecular weight of less than 5 kDa.

53. The method of claim 52, wherein the NLRP3 activator is imiquimod or resiquimod, or a pharmaceutically acceptable salt thereof.

54. The method of claim 52, wherein the NLRP3 activator is: 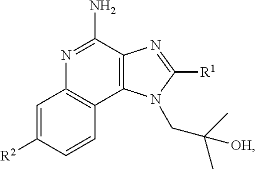
wherein:
(a) R¹ is H, and R² is H;
(b) R¹ is a butyl group and R² is H;
(c) R¹ is H and R² is —CO₂CH₃; or
(d) R¹ is a butyl group and R² is —CO₂CH₃.

55. The method of claim 52, wherein the NLRP3 activator is selected from the group consisting of:
an imadazoquinoline;
an imidazonaphthyridine;
a pyrazolopyridine;
an aryl-substituted imidazoquinoline;
a compound having a 1-alkoxy 1H-imidazo ring system;
an oxazolo [4,5-c]-quinolin-4-amine;
an thiazolo [4,5-c]-quinolin-4-amine;
a selenazolo [4,5-c]-quinolin-4-amine;
an imidazonaphthyridine,
an imidazoquinolinamine;
a 1-substituted, 2-substituted 1H-imidazo[4,5-C]quinolin-4-amine;
a fused cycloalkylimidazopyridine;
a 1H-imidazo[4,5-c]quinolin-4-amine;
a 1-substituted 1H-imidazo-[4,5-c]quinolin-4-amine;
an imidazo-[4,5-C]quinolin-4-amine;
a 2-ethyl 1H-imidazo[4,5-ciquinolin-4-amine;
an olfenic 1H-imidazo[4,5-c]quinolin-4-amine;
a 6,7-dihydro-8-(imidazol-1-yl)-5-methyl-1-oxo-1H,5H-benzo[ij]quinolizine-2-carboxylic acid;
a pyridoquinoxaline-6-carboxylic acid;
a 6,7-dihydro-8-(imidazol-1-yl)-5-methyl-1-oxo-1H,5H-benzo [ij]quinolizine-2-carboxylic acid;
a substituted naphtho[ij]quinolizine;
a substituted pyridoquinoxaline-6-carboxylic acid;
a 7-hydroxy-benzo[ij]quinolizine-2-carboxylic acid derivative;
a substituted benzo[ij]quinolizine-2-carboxylic acid;
a7-hydroxy-benzo[ij]quinolizine-2-carboxylic acid;
a substituted pyrido[1,2,3,-de]-1,4-benzoxazine; and
a N-methylene malonate of tetrahydroquinoline.

56. The method of any one of claims 39-55, wherein the T cell is a CD4⁺ T cell or a CD8⁺ cell.

57. The method of any one of claims 39-55, wherein the T cell is selected from the group consisting of: a lymphoid progenitor cell, an immature thymocyte, a peripheral blood lymphocyte, a naïve T cell, a pluripotent T_{H} cell precursor, a T_{reg} cell, a memory T cell, a T_{H}17 cell, a T_{H}22 cell, a T_{H}9 cell, a T_{H}2 cell, a T_{H}1 cell, a T_{H}3 cell, γδ T cell, an αβ T cell, and a tumor-infiltrating T cell.

58. The method of any one of claims 1-57, wherein the T cell is a chimeric antigen receptor (CAR)-T cell.

59. The method of claim 58, wherein the CAR-T cell comprises a nucleic acid encoding a CAR protein comprising:
an antigen-binding domain,
a transmembrane domain, and
a cytoplasmic signaling domain.

60. The method of claim 59, wherein the antigen-binding domain is an antibody, an antigen-binding fragment of an antibody, a Fab fragment, and a scFv.

61. The method of claim 60, wherein the antibody is a human or humanized antibody, or the antigen-binding fragment of an antibody or the Fab is a fragment of a human or humanized antibody.

62. The method of any one of claims 59-61, wherein the cytoplasmic signaling domain comprises a CD3ζ cytoplasmic sequence.

63. The method of claim 62, wherein the cytoplasmic signaling domain further comprises a cytoplasmic sequence of one or more of the following proteins: CD27, CD28, 4-1BB, OX40, CD30, CD40, PD-1, ICOS, LFA-1, CD2, CD7, LIGHT, NKG2C, B7-H3, and a ligand that specifically binds to CD83.

64. The method of claim 59-63, wherein the transmembrane domain comprises a transmembrane sequence from CD3ζ, CD8, or CD28.

65. The method of any one of claims 59-64, wherein the CAR protein further comprises a linker sequence between the antigen-binding domain and the transmembrane domain.

66. The method of any one of claims 39-57, wherein the T cell is harvested from a mammal.

67. The method of claim 66, wherein the mammal has been identified as having a cancer or an infectious disease.

68. The method of claim 67, wherein the mammal has been identified as having a cancer selected from the group consisting of: acute lymphoblastic leukemia, acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastric cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, gastric cancer, testicular cancer, throat cancer, thyroid cancer, urethral cancer, uterine cancer, vaginal cancer, and vulvar cancer.

69. The method of claim 67, wherein the mammal has been identified as having an infectious disease selected from the group of: Acinobacter infection, actinomycosis, African sleeping sickness, acquired immunodeficiency syndrome, amebiasis, anaplasmosis, anthrax, Arcanobacterium haemolyticum infection, Argentine hemorrhagic fever, ascariasis, aspergillosis, astrovirus infection, babesiosis, Bacillus cereus infection, bacterial pneumonia, bacterial vaginosis, Bacteroides infection, balantidiasis, Baylisascaris infection, BK virus infection, black piedra, Blastocystic hominis infection, blastomycosis, Bolivian hemorrhagic fever, botulism, Brazilian hemorrhagic fever, brucellosis, bubonic plaque, Burkholderi infection, Buruli ulcer, Calicivirus infection, camptobacteriosis, candidiasis, cat-scratch disease, cellulitis, Chagas disease, chancroid, chickenpox, chikungunya, chlamydia, Chlamydophila pneumoniae infection, cholera, chromoblastomycosis, clonorchiasis, Clostridium difficile infection, coccidioidomycosis, Colorado tick fever, common cold, Creutzfeldt-Jakob disease, Crimean-Congo hemorrhagic fever, crytococcosis, cryptosporidiosis, cutaneous larva migrans, cyclosporiasis, cysticercosis, cytomegalovirus infection, dengue fever, Desmodesmus infection, deintamoebiasis, diphtheria, diphyllobothriasis, dracunculiasis, ebola hemorrhagic fever, echinococcosis, ehrlichiosis, enterobiasis, Enterococcus infection, Enterovirus infection, epidemic typhus, erythema infection, exanthema subitum, fasciolopsiasis, fasciolosis, fatal familial insomnia, filariasis, food poisoning by Clostridium myonecrosis, free-living amebic infection, Fusobacterium infection, gas gangrene, geotrichosis, Gerstmann-Sträussler-Scheinker syndrome, giardiasis, glanders, gnathostomiasis, gonorrhea, granuloma inguinale, Group A streptococcal infection, Group B streptococcal infection, Haemophilus influenzae infection, hand foot and mouth disease, hantavirus pulmonary syndrome, Heartland virus disease, Heliobacter pylori infection, hemolytic-uremic syndrome, hemorrhagic fever with renal syndrome, hepatitis A, hepatitis B, hepatitis C, hepatitis D, hepatitis E, herpes simplex, histoplasmosis, hookworm infection, human bocavirus infection, human ewingii ehrlichiosis, human granulocyte anaplasmosis, human metapneuomovirus infection, human monocytic ehrlichiosis, human papillomavirus infection, human parainfluenza virus infection, hymenolepiasis, Epstein-Barr virus infectious mononucleosis, influenza, isosporiasis, Kawasaki disease, keratitis, Kingella kingae infection, kuru, lassa fever, Legionnaires' disease, Pontiac fever, leishmaniasis, leprosy, leptospirosis, listeriosis, lyme disease, lymphatic filariasis, lymphocytic choriomeningitis, malaria, Marburg hemorrhagic fever, measles, Middle East respiratory syndrome, melioidosis, meningitis, meningococcal disease, metagonimiasis, microsporidiosis, molluscum contagiosum, monkeypox, mumps, murine typhus, mycoplasma pneumonia, mycetoma, myiasis, neonatal conjunctivitis, variant Creutzfeldt-Jakob disease, nocardiosis, onchocerciasis, paracoccidioidomycosis, paragonimiasis, pasteurellosis, pediculosis capitis, pediculosis corporis, pediculosis pubis, pelvic inflammatory disease, pertussis, plague, pneumonia, poliomyelitis, Prevotella infection, primary amoebic meningoencephalitis, progressive multifocal leukoencephalopathy, psittacosis, Q fever, rabies, relapsing fever, respiratory syncytial virus infection, rhinosporidiosis, rhinovirus infection, rickettsial infection, rickettsialpox, Rift Valley Fever, Rocky Mountain spotted fever, rotavirus infection, rubella, salmonellosis, severe acute respiratory syndrome, scabies, schistosomiasis, sepsis, shigellosis, shingles, smallpox, sporothrichosis, staphylococcal food poisoning, staphylococcal infection, staphylococcal infection, strongyloidiasis, subacute sclerosing panencephalitis, syphilis, taeniasis, tetanus, tinea barabe, tinea capitis, tinea corporis, tinea cruris, tinea manum, tinea nigra, tinea pedis, tinea unguium, tinea versicolor, toxocariasis, trachoma, toxoplasmosis, trichinosis, trichomoniasis, trichuriasis, tuberculosis, tularemia, typhoid fever, Ureaplasma urealyticum infection, valley fever, Venezuelan hemorrhagic fever, viral pneumonia, West Nile fever, white piedra, Yersinia psuedotuberculosis infection, yersiniosis, yellow fever, and zygomycosis.

70. The method of any one of claims 66-69, further comprising:
harvesting the T cell from the mammal.

71. A T cell having improved resistance to at least one immunosuppressive cytokine produced by the method of any one of claims 39-70.

72. A pharmaceutical composition comprising the T cell of claim 71.

73. A kit comprising a pharmaceutical composition of claim 34.

74. A method of increasing anti-tumor immunity in a mammal having a cancer, the method comprising:
(a) identifying a mammal having a cancer;
(b) harvesting a T cell from the identified mammal;
(c) culturing the T cell:
in a culture medium sufficient to induce and/or increase anaplerosis and/or comprising a NLRP3 activator in an amount sufficient to improve anti-tumor activity of a T cell; and/or
under conditions sufficient to induce and/or increase anaplerosis, thereby resulting in an improvement in the anti-tumor activity of the T cell; and
(d) administering the T cell to a mammal, thereby the resulting in increasing anti-tumor immunity in the mammal.

75. A method of increasing the time of remission of a cancer in a mammal, the method comprising:
(a) identifying a mammal having a cancer;
(b) harvesting a T cell from the identified mammal;
(c) culturing the T cell:
in a culture medium sufficient to induce and/or increase anaplerosis and/or comprising a NLRP3 activator in an amount sufficient to improve anti-tumor activity of a T cell; and/or
under conditions sufficient to induce and/or increase anaplerosis, thereby resulting in an improvement in the anti-tumor activity of the T cell; and
(c) administering the T cell to the mammal, thereby the resulting in an increase in the time of remission of a cancer in the mammal.

76. A method of treating a mammal having a cancer or an infectious disease, the method comprising:
(a) identifying a mammal having a cancer or an infectious disease;
(b) harvesting a T cell from the identified mammal;
(c) culturing the T cell:
in a culture medium sufficient to induce and/or increase anaplerosis and/or comprising a NLRP3 activator in an amount sufficient to improve anti-tumor or anti-infectious disease activity of a T cell; and/or
under conditions sufficient to induce and/or increase anaplerosis, thereby resulting in an improvement in the anti-tumor or anti-infectious disease activity of the T cell; and
(e) administering the T cell to the mammal.

77. A method of increasing the time of survival of a mammal having a cancer, the method comprising:
(a) identifying a mammal having a cancer;
(b) harvesting a T cell from the identified mammal;
(c) culturing the T cell:
in a culture medium sufficient to induce and/or increase anaplerosis and/or comprising a NLRP3 activator in an amount sufficient to improve anti-tumor activity of a T cell; and/or
under conditions sufficient to induce and/or increase anaplerosis, thereby resulting in an improvement in the anti-tumor activity of the T cell; and
(d) administering the T cell to the mammal.

78. A method of decreasing the size of a solid tumor in a mammal having a cancer, the method comprising:
(a) identifying a mammal having a cancer and a solid tumor;
(b) harvesting a T cell from the identified mammal;
(c) culturing the T cell:
in a culture medium sufficient to induce and/or increase anaplerosis and/or comprising a NLRP3 activator in an amount sufficient to improve anti-tumor activity of a T cell; and/or
under conditions sufficient to induce and/or increase anaplerosis, thereby resulting in an improvement in the anti-tumor activity of the T cell; and
(d) administering the T cell to the mammal.

79. A method of improving the prognosis of a mammal having a cancer, the method comprising:
(a) identifying a mammal having a cancer;
(b) harvesting a T cell from the identified mammal;
(c) culturing the T cell:
in a culture medium sufficient to induce and/or increase anaplerosis and/or comprising a NLRP3 activator in an amount sufficient to improve anti-tumor activity of a T cell; and/or
under conditions sufficient to induce and/or increase anaplerosis, thereby resulting in an improvement in the anti-tumor activity of the T cell; and
(d) administering the T cell to the mammal.

80. A method of decreasing the risk of developing a metastasis or an additional metastasis in a mammal having a cancer, the method comprising:
(a) identifying a mammal having a cancer;
(b) harvesting a T cell from the identified mammal;
(c) culturing the T cell:
in a culture medium sufficient to induce and/or increase anaplerosis and/or comprising a NLRP3 activator in an amount sufficient to improve anti-tumor activity of a T cell; and/or
under conditions sufficient to induce and/or increase anaplerosis, thereby resulting in an improvement in the anti-tumor activity of the T cell; and
(d) administering the T cell to the mammal.

81. A method of increasing the level of at least one anti-tumor lymphokine and/or at least one anti-tumor cytokine in a mammal having a cancer, the method comprising:
(a) identifying a mammal having a cancer;
(b) harvesting a T cell from the identified mammal;
(c) culturing the T cell:
in a culture medium sufficient to induce and/or increase anaplerosis and/or comprising a NLRP3 activator in an amount sufficient to improve anti-tumor activity of a T cell; and/or
under conditions sufficient to induce and/or increase anaplerosis, thereby resulting in an improvement in the anti-tumor activity of the T cell; and
(d) administering the T cell to the mammal.

82. A method for maintaining a population of recombinant T cells in a mammal for at least one month after administering the recombinant T cell to the mammal, the method comprising:
(a) identifying a mammal having a cancer;
(b) harvesting a T cell from the identified mammal;
(c) introducing a recombinant nucleic acid into the T cell to generate a recombinant T cell;
(d) culturing the recombinant T cell:
in a culture medium sufficient to induce and/or increase anaplerosis and/or comprising a NLRP3 activator in an amount sufficient to improve anti-tumor activity of a T cell; and/or
under conditions sufficient to induce and/or increase anaplerosis, thereby resulting in an improvement in the anti-tumor activity of the recombinant T cell; and
(d) administering the recombinant T cell to the mammal, whereby a population of the recombinant T cells persists in the mammal for at least one month after the administering of the recombinant T cell to the mammal.

83. The method of any one of claims 74-81, wherein the method comprises culturing the T cell in a culture medium sufficient to induce and/or increase anaplerosis.

84. The method of claim 82, wherein the method comprises culturing the recombinant T cell in a culture medium sufficient to induce and/or increase anaplerosis.

85. The method of any one of claims 74-81, wherein the method comprises culturing the T cell in a culture medium comprising a NLRP3 activator in an amount sufficient to improve anti-tumor activity of a T cell.

86. The method of claim 82, wherein the method comprises culturing the recombinant T cell in a culture medium comprising a NLRP3 activator in an amount sufficient to improve anti-tumor activity of a T cell.

87. The method of any one of claims 74-81, wherein the method comprises culturing the T cell in a culture medium sufficient to induce and/or increase anaplerosis and comprising a NLRP3 activator in an amount sufficient to improve anti-tumor activity of a T cell.

88. The method of claim 82, wherein the method comprises culturing the recombinant T cell in a culture medium sufficient to induce and/or increase anaplerosis and comprising a NLRP3 activator in an amount sufficient to improve anti-tumor activity of a T cell.

89. The method of any one of claims 83, 85, and 87, wherein the method further comprises culturing the T cell under conditions sufficient to induce and/or increase anaplerosis.

90. The method of any one of claims 84, 86, and 88, wherein the method further comprises culturing the recombinant T cell under conditions sufficient to induce and/or increase anaplerosis.

91. The method of claim 74-81, wherein the method comprises culturing the T cell under conditions sufficient to induce and/or increase anaplerosis.

92. The method of claim 82, wherein the method comprises culturing the recombinant T cell under conditions sufficient to induce and/or increase anaplerosis.

93. The method of any one of claims 74-81, 83, 85, 87, 89, and 91, wherein the culture medium further comprises one or more agents that activate the T cell.

94. The method of any one of claims 82, 84, 86, 88, 90, and 92, wherein the culture medium comprises one or more agents that activate the recombinant T cell.

95. The method of claim 93, wherein the one or more agents that activate the T cell comprises an isolated tumor antigen, an isolated antigen from a pathogenic organism or virus, or a tumor vaccine.

96. The method of claim 94, wherein the one or more agents that activate the recombinant T cell comprises an isolated tumor antigen, an isolated antigen from a pathogenic organism or virus, or a tumor vaccine.

97. The method of any one of claims 74-84, 87-90, and 93-96, wherein the culture medium sufficient to induce and/or increase anaplerosis comprises one or more of:
(i) galactose without glucose supplementation;
(ii) culture media that contains galactose and glucose added in any amount that does not prohibit an increase in anaplerosis above rates in control cells; and/or
(iii) one or more of 3-bromopyruvate, 2-deoxyglucose, pentavalent arsenic (H3AsO4), lonidamine, imatinib, oxythiamine, pyruvate, odd-chain fatty acids, 5-carbon ketone bodies, and triheptanoin.

98. The method of any one of claims 74-82 and 89-96, wherein the conditions sufficient to induce and/or increase anaplerosis are conditions that result in one or more of the following in the T cell:
a decrease in the rate of lactate production from glycolysis,
a decrease in the rate of lactate production from glycolysis relative to the rate of oxidative phosphorylation,
an increase in the rate of glutamine uptake by the T cell,
an increase in the fraction of lipids and/or amino acids synthesized using glutamine as a substrate,
an increase in the rate at which pyruvate is converted to oxaloacetate by pyruvate carboxylase,
an increase in the rate at which adenylosuccinate synthetase produces fumurate,
an increase in the rate at which aspartate aminotransferase produces oxaloacetate, and
an increase in the rate at which propionyl-CoA carboxylase produces succinyl-CoA.

99. The method of any one of claims 72-81, 85, 87, 89, 93, and 95, wherein the NLRP3 activator results in an increase in the level of an NLRP3 protein in the T cell.

100. The method of any one of claims 82, 86, 88, 90, 94, and 96, wherein the NLRP3 activator results in an increase in the level of an NLRP3 protein in the recombinant T cell.

101. The method of any one of claims 72-81, 85, 87, 89, 93, and 95, wherein the NLRP3 activator results in an increase NLRP3 downstream signaling in the T cell.

102. The method of any one of claims 82, 86, 88, 90, 94, and 96, wherein the NLRP3 activator results in an increase in NLRP3 downstream signaling in the recombinant T cell.

103. The method of any one of claims 72-82, 85-90, and 93-96, wherein the NLRP3 activator is interleukin-1α or interleukin-1β.

104. The method of any one of claims 72-82, 85-90, and 93-96, wherein the NLRP3 activator is a molecule having a molecular weight of less than 5 kDa.

105. The method of claim 104, wherein the NLRP3 activator is imiquimod or resiquimod, or a pharmaceutically acceptable salt thereof.

106. The method of claim 104 wherein the NLRP3 activator is: 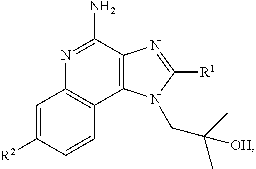
wherein:
(a) R¹ is H, and R² is H;
(b) R¹ is a butyl group and R² is H;
(c) R¹ is H and R² is —CO₂CH₃; or
(d) R¹ is a butyl group and R² is —CO₂CH₃.

107. The method of claim 104, wherein the NLRP3 activator is selected from the group consisting of:
an imadazoquinoline;
an imidazonaphthyridine;
a pyrazolopyridine;
an aryl-substituted imidazoquinoline;
a compound having a 1-alkoxy 1H-imidazo ring system;
an oxazolo [4,5-c]-quinolin-4-amine;
an thiazolo [4,5-c]-quinolin-4-amine;
a selenazolo [4,5-c]-quinolin-4-amine;
an imidazonaphthyridine,
an imidazoquinolinamine,
a 1-substituted, 2-substituted 1H-imidazo[4,5-C]quinolin-4-amine;
a fused cycloalkylimidazopyridine;
a 1H-imidazo[4,5-c]quinolin-4-amine;
a 1-substituted 1H-imidazo-[4,5-c]quinolin-4-amine;
an imidazo-[4,5-C]quinolin-4-amine;
a 2-ethyl 1H-imidazo[4,5-ciquinolin-4-amine;
an olfenic 1H-imidazo[4,5-c]quinolin-4-amine;
a 6,7-dihydro-8-(imidazol-1-yl)-5-methyl-1-oxo-1H,5H-benzo[ij]quinolizine-2-carboxylic acid;
a pyridoquinoxaline-6-carboxylic acid;
a 6,7-dihydro-8-(imidazol-1-yl)-5-methyl-1-oxo-1H,5H-benzo [ij]quinolizine-2-carboxylic acid;
a substituted naphtho[ij]quinolizine;
a substituted pyridoquinoxaline-6-carboxylic acid;
a 7-hydroxy-benzo[ij]quinolizine-2-carboxylic acid derivative;
a substituted benzo[ij]quinolizine-2-carboxylic acid;
a7-hydroxy-benzo[ij]quinolizine-2-carboxylic acid;
a substituted pyrido[1,2,3,-de]-1,4-benzoxazine; and
a N-methylene malonate of tetrahydroquinoline.

108. The method of any one of claims 74-107, wherein the T cell is a CD4⁺ T cell or a CD8⁺ cell.

109. The method of any one of claims 74-107, wherein the T cell is selected from the group consisting of: a lymphoid progenitor cell, an immature thymocyte, a peripheral blood lymphocyte, a naïve T cell, a pluripotent T_{H} cell precursor, a T_{reg} cell, a memory T cell, a T_{H}17 cell, a T_{H}22 cell, a T_{H}9 cell, a T_{H}2 cell, a T_{H}1 cell, a T_{H}3 cell, γδ T cell, an αβ T cell, and a tumor-infiltrating T cell.

110. The method of any one of claims 74-81, 83, 85, 87, 89, 91, 93, 95, 97-99, 101, and 103-107, wherein the T cell is a chimeric antigen receptor (CAR)-T cell.

111. The method of any one of claims 82, 84, 86, 88, 90, 92, 94, 96-98, 100, and 102-109, wherein the recombinant T cell is a CAR-T cell.

112. The method of claim 110 or 111, wherein the CAR-T cell comprises a nucleic acid encoding a CAR protein comprising:
an antigen-binding domain,
a transmembrane domain, and
a cytoplasmic signaling domain.

113. The method of claim 112, wherein the antigen-binding domain is an antibody, an antigen-binding fragment of an antibody, a Fab fragment, and a scFv.

114. The method of claim 113, wherein the antibody is a human or humanized antibody, or the antigen-binding fragment of an antibody or the Fab is a fragment of a human or humanized antibody.

115. The method of any one of claims 112-114, wherein the cytoplasmic signaling domain comprises a CD3ζ cytoplasmic sequence.

116. The method of claim 115, wherein the cytoplasmic signaling domain further comprises a cytoplasmic sequence of one or more of the following proteins: CD27, CD28, 4-1BB, OX40, CD30, CD40, PD-1, ICOS, LFA-1, CD2, CD7, LIGHT, NKG2C, B7-H3, and a ligand that specifically binds to CD83.

117. The method of claim 112-116, wherein the transmembrane domain comprises a transmembrane sequence from CD3ζ, CD8, or CD28.

118. The method of any one of claims 112-117, wherein the CAR protein further comprises a linker sequence between the antigen-binding domain and the transmembrane domain.

119. The method of any one of claims 74-118, wherein the mammal has been identified as having a cancer.

120. The method of claims 76, 83, 85, 87, 89, 91, 93, 95, 97-99, 101, 103-110, and 112-118, wherein the mammal has been identified as having an infectious disease.

121. The method of claim 119, wherein the mammal has been identified as having a cancer selected from the group consisting of: acute lymphoblastic leukemia, acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastric cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, gastric cancer, testicular cancer, throat cancer, thyroid cancer, urethral cancer, uterine cancer, vaginal cancer, and vulvar cancer.

122. The method of claim 120, wherein the mammal has been identified as having an infectious disease selected from the group of: Acinobacter infection, actinomycosis, African sleeping sickness, acquired immunodeficiency syndrome, amebiasis, anaplasmosis, anthrax, Arcanobacterium haemolyticum infection, Argentine hemorrhagic fever, ascariasis, aspergillosis, astrovirus infection, babesiosis, Bacillus cereus infection, bacterial pneumonia, bacterial vaginosis, Bacteroides infection, balantidiasis, Baylisascaris infection, BK virus infection, black piedra, Blastocystic hominis infection, blastomycosis, Bolivian hemorrhagic fever, botulism, Brazilian hemorrhagic fever, brucellosis, bubonic plaque, Burkholderi infection, Buruli ulcer, Calicivirus infection, camptobacteriosis, candidiasis, cat-scratch disease, cellulitis, Chagas disease, chancroid, chickenpox, chikungunya, chlamydia, Chlamydophila pneumoniae infection, cholera, chromoblastomycosis, clonorchiasis, Clostridium difficile infection, coccidioidomycosis, Colorado tick fever, common cold, Creutzfeldt-Jakob disease, Crimean-Congo hemorrhagic fever, crytococcosis, cryptosporidiosis, cutaneous larva migrans, cyclosporiasis, cysticercosis, cytomegalovirus infection, dengue fever, Desmodesmus infection, deintamoebiasis, diphtheria, diphyllobothriasis, dracunculiasis, ebola hemorrhagic fever, echinococcosis, ehrlichiosis, enterobiasis, Enterococcus infection, Enterovirus infection, epidemic typhus, erythema infection, exanthema subitum, fasciolopsiasis, fasciolosis, fatal familial insomnia, filariasis, food poisoning by Clostridium myonecrosis, free-living amebic infection, Fusobacterium infection, gas gangrene, geotrichosis, Gerstmann-Sträussler-Scheinker syndrome, giardiasis, glanders, gnathostomiasis, gonorrhea, granuloma inguinale, Group A streptococcal infection, Group B streptococcal infection, Haemophilus influenzae infection, hand foot and mouth disease, hantavirus pulmonary syndrome, Heartland virus disease, Heliobacter pylori infection, hemolytic-uremic syndrome, hemorrhagic fever with renal syndrome, hepatitis A, hepatitis B, hepatitis C, hepatitis D, hepatitis E, herpes simplex, histoplasmosis, hookworm infection, human bocavirus infection, human ewingii ehrlichiosis, human granulocyte anaplasmosis, human metapneuomovirus infection, human monocytic ehrlichiosis, human papillomavirus infection, human parainfluenza virus infection, hymenolepiasis, Epstein-Barr virus infectious mononucleosis, influenza, isosporiasis, Kawasaki disease, keratitis, Kingella kingae infection, kuru, lassa fever, Legionnaires' disease, Pontiac fever, leishmaniasis, leprosy, leptospirosis, listeriosis, lyme disease, lymphatic filariasis, lymphocytic choriomeningitis, malaria, Marburg hemorrhagic fever, measles, Middle East respiratory syndrome, melioidosis, meningitis, meningococcal disease, metagonimiasis, microsporidiosis, molluscum contagiosum, monkeypox, mumps, murine typhus, mycoplasma pneumonia, mycetoma, myiasis, neonatal conjunctivitis, variant Creutzfeldt-Jakob disease, nocardiosis, onchocerciasis, paracoccidioidomycosis, paragonimiasis, pasteurellosis, pediculosis capitis, pediculosis corporis, pediculosis pubis, pelvic inflammatory disease, pertussis, plague, pneumonia, poliomyelitis, Prevotella infection, primary amoebic meningoencephalitis, progressive multifocal leukoencephalopathy, psittacosis, Q fever, rabies, relapsing fever, respiratory syncytial virus infection, rhinosporidiosis, rhinovirus infection, rickettsial infection, rickettsialpox, Rift Valley Fever, Rocky Mountain spotted fever, rotavirus infection, rubella, salmonellosis, severe acute respiratory syndrome, scabies, schistosomiasis, sepsis, shigellosis, shingles, smallpox, sporothrichosis, staphylococcal food poisoning, staphylococcal infection, staphylococcal infection, strongyloidiasis, subacute sclerosing panencephalitis, syphilis, taeniasis, tetanus, tinea barabe, tinea capitis, tinea corporis, tinea cruris, tinea manum, tinea nigra, tinea pedis, tinea unguium, tinea versicolor, toxocariasis, trachoma, toxoplasmosis, trichinosis, trichomoniasis, trichuriasis, tuberculosis, tularemia, typhoid fever, Ureaplasma urealyticum infection, valley fever, Venezuelan hemorrhagic fever, viral pneumonia, West Nile fever, white piedra, Yersinia psuedotuberculosis infection, yersiniosis, yellow fever, and zygomycosis.

123. The method of any one of claims 74-81, 83, 85, 87, 89, 91, 93, 95, 97-99, 101, 103-109, 110, and 112-118, wherein the T cell is administered by intravenously, intraarterial, or intra-tumor administration.

124. The method of any one of claims 82, 84, 86, 88, 90, 92, 94, 96-98, 100, 102-109, and 111-118, wherein the recombinant T cell is administered by intravenous, intraarterial, or intra-tumor administration.

125. A method of increasing anti-tumor immunity in a mammal having cancer, the method comprising:
(a) identifying a mammal as having a cancer; and
(b) administering a therapeutically effective amount of a NLRP3 activator to the identified mammal.

126. A method of increasing the time of remission of a cancer in a mammal, the method comprising:
(a) identifying a mammal as having a cancer; and
(b) administering a therapeutically effective amount of a NLRP3 activator to the identified mammal.

127. A method of treating a mammal having a cancer or an infectious disease, the method comprising:
(a) identifying a mammal having a cancer or an infectious disease; and
(b) administering a therapeutically effective amount of a NLRP3 activator to the identified mammal.

128. A method of increasing the time of survival of a mammal having a cancer, the method comprising:
(a) identifying a mammal having a cancer; and
(b) administering a therapeutically effective amount of a NLRP3 activator to the identified mammal.

129. A method of decreasing the size of a solid tumor in a mammal having a cancer, the method comprising:
(a) identifying a mammal having a cancer and a solid tumor; and
(b) administering a therapeutically effective amount of a NLRP3 activator to the identified mammal.

130. A method of improving the prognosis of a mammal having a cancer, the method comprising:
(a) identifying a mammal having a cancer; and
(b) administering a therapeutically effective amount of a NLRP3 activator to the identified mammal.

131. A method of decreasing the risk of developing a metastasis or an additional metastasis in a mammal having a cancer, the method comprising:
(a) identifying a mammal having a cancer; and
(b) administering a therapeutically effective amount of a NLRP3 activator to the identified mammal.

132. A method of increasing the level of at least one anti-tumor lymphokine and/or at least one anti-tumor cytokine in a mammal having a cancer, the method comprising:
(a) identifying a mammal having a cancer; and
(b) administering a therapeutically effective amount of a NLRP3 activator to the identified mammal.

133. The method of claim 127, wherein the mammal is identified as having an infectious disease.

134. The method of claim 127, wherein the mammal is identified as having a cancer.

135. The method of any one of claims 125, 126, 129-132, and 134, wherein the mammal is identified as having a cancer selected from the group consisting of: acute lymphoblastic leukemia, acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastric cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, gastric cancer, testicular cancer, throat cancer, thyroid cancer, urethral cancer, uterine cancer, vaginal cancer, and vulvar cancer.

136. The method of claim 133, wherein the mammal is identified as having an infectious disease selected from the group consisting of: Acinobacter infection, actinomycosis, African sleeping sickness, acquired immunodeficiency syndrome, amebiasis, anaplasmosis, anthrax, Arcanobacterium haemolyticum infection, Argentine hemorrhagic fever, ascariasis, aspergillosis, astrovirus infection, babesiosis, Bacillus cereus infection, bacterial pneumonia, bacterial vaginosis, Bacteroides infection, balantidiasis, Baylisascaris infection, BK virus infection, black piedra, Blastocystic hominis infection, blastomycosis, Bolivian hemorrhagic fever, botulism, Brazilian hemorrhagic fever, brucellosis, bubonic plaque, Burkholderi infection, Buruli ulcer, Calicivirus infection, camptobacteriosis, candidiasis, cat-scratch disease, cellulitis, Chagas disease, chancroid, chickenpox, chikungunya, chlamydia, Chlamydophila pneumoniae infection, cholera, chromoblastomycosis, clonorchiasis, Clostridium difficile infection, coccidioidomycosis, Colorado tick fever, common cold, Creutzfeldt-Jakob disease, Crimean-Congo hemorrhagic fever, crytococcosis, cryptosporidiosis, cutaneous larva migrans, cyclosporiasis, cysticercosis, cytomegalovirus infection, dengue fever, Desmodesmus infection, deintamoebiasis, diphtheria, diphyllobothriasis, dracunculiasis, ebola hemorrhagic fever, echinococcosis, ehrlichiosis, enterobiasis, Enterococcus infection, Enterovirus infection, epidemic typhus, erythema infection, exanthema subitum, fasciolopsiasis, fasciolosis, fatal familial insomnia, filariasis, food poisoning by Clostridium myonecrosis, free-living amebic infection, Fusobacterium infection, gas gangrene, geotrichosis, Gerstmann-Sträussler-Scheinker syndrome, giardiasis, glanders, gnathostomiasis, gonorrhea, granuloma inguinale, Group A streptococcal infection, Group B streptococcal infection, Haemophilus influenzae infection, hand foot and mouth disease, hantavirus pulmonary syndrome, Heartland virus disease, Heliobacter pylori infection, hemolytic-uremic syndrome, hemorrhagic fever with renal syndrome, hepatitis A, hepatitis B, hepatitis C, hepatitis D, hepatitis E, herpes simplex, histoplasmosis, hookworm infection, human bocavirus infection, human ewingii ehrlichiosis, human granulocyte anaplasmosis, human metapneuomovirus infection, human monocytic ehrlichiosis, human papillomavirus infection, human parainfluenza virus infection, hymenolepiasis, Epstein-Barr virus infectious mononucleosis, influenza, isosporiasis, Kawasaki disease, keratitis, Kingella kingae infection, kuru, lassa fever, Legionnaires' disease, Pontiac fever, leishmaniasis, leprosy, leptospirosis, listeriosis, lyme disease, lymphatic filariasis, lymphocytic choriomeningitis, malaria, Marburg hemorrhagic fever, measles, Middle East respiratory syndrome, melioidosis, meningitis, meningococcal disease, metagonimiasis, microsporidiosis, molluscum contagiosum, monkeypox, mumps, murine typhus, mycoplasma pneumonia, mycetoma, myiasis, neonatal conjunctivitis, variant Creutzfeldt-Jakob disease, nocardiosis, onchocerciasis, paracoccidioidomycosis, paragonimiasis, pasteurellosis, pediculosis capitis, pediculosis corporis, pediculosis pubis, pelvic inflammatory disease, pertussis, plague, pneumonia, poliomyelitis, Prevotella infection, primary amoebic meningoencephalitis, progressive multifocal leukoencephalopathy, psittacosis, Q fever, rabies, relapsing fever, respiratory syncytial virus infection, rhinosporidiosis, rhinovirus infection, rickettsial infection, rickettsialpox, Rift Valley Fever, Rocky Mountain spotted fever, rotavirus infection, rubella, salmonellosis, severe acute respiratory syndrome, scabies, schistosomiasis, sepsis, shigellosis, shingles, smallpox, sporothrichosis, staphylococcal food poisoning, staphylococcal infection, staphylococcal infection, strongyloidiasis, subacute sclerosing panencephalitis, syphilis, taeniasis, tetanus, tinea barabe, tinea capitis, tinea corporis, tinea cruris, tinea manum, tinea nigra, tinea pedis, tinea unguium, tinea versicolor, toxocariasis, trachoma, toxoplasmosis, trichinosis, trichomoniasis, trichuriasis, tuberculosis, tularemia, typhoid fever, Ureaplasma urealyticum infection, valley fever, Venezuelan hemorrhagic fever, viral pneumonia, West Nile fever, white piedra, Yersinia psuedotuberculosis infection, yersiniosis, yellow fever, and zygomycosis.

137. The method of any one of claims 125-136, wherein the NLRP3 activator is interleukin-1α or interleukin-1β.

138. The method of any one of claims 125-136, wherein the NLRP3 activator is a molecule having a molecular weight of less than 5 kDa.

139. The method of claim 138, wherein the NLRP3 activator is imiquimod or resiquimod, or a pharmaceutically acceptable salt thereof.

140. The method of claim 138 wherein the NLRP3 activator is: 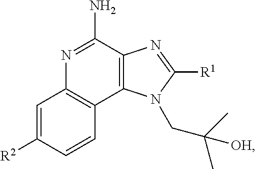
wherein:
(a) R¹ is H, and R² is H;
(b) R¹ is a butyl group and R² is H;
(c) R¹ is H and R² is —CO₂CH₃; or
(d) R¹ is a butyl group and R² is —CO₂CH₃.

141. The method of claim 138, wherein the NLRP3 activator is selected from the group consisting of:
an imadazoquinoline;
an imidazonaphthyridine;
a pyrazolopyridine;
an aryl-substituted imidazoquinoline;
a compound having a 1-alkoxy 1H-imidazo ring system;
an oxazolo [4,5-c]-quinolin-4-amine;
an thiazolo [4,5-c]-quinolin-4-amine;
a selenazolo [4,5-c]-quinolin-4-amine;
an imidazonaphthyridine,
an imidazoquinolinamine;
a 1-substituted, 2-substituted 1H-imidazo[4,5-C]quinolin-4-amine;
a fused cycloalkylimidazopyridine;
a 1H-imidazo[4,5-c]quinolin-4-amine;
a 1-substituted 1H-imidazo-[4,5-c]quinolin-4-amine;
an imidazo-[4,5-C]quinolin-4-amine;
a 2-ethyl 1H-imidazo[4,5-ciquinolin-4-amine;
an olfenic 1H-imidazo[4,5-c]quinolin-4-amine;
a 6,7-dihydro-8-(imidazol-1-yl)-5-methyl-1-oxo-1H,5H-benzo[ij]quinolizine-2-carboxylic acid;
a pyridoquinoxaline-6-carboxylic acid;
a 6,7-dihydro-8-(imidazol-1-yl)-5-methyl-1-oxo-1H,5H-benzo [ij]quinolizine-2-carboxylic acid;
a substituted naphtho[ij]quinolizine;
a substituted pyridoquinoxaline-6-carboxylic acid;
a 7-hydroxy-benzo[ij]quinolizine-2-carboxylic acid derivative;
a substituted benzo[ij]quinolizine-2-carboxylic acid;
a7-hydroxy-benzo[ij]quinolizine-2-carboxylic acid;
a substituted pyrido[1,2,3,-de]-1,4-benzoxazine; and
a N-methylene malonate of tetrahydroquinoline.

142. The method of any one of claims 125-141, wherein the NLRP3 activator is administered by intravenously, intraarterial, or intra-tumor administration.

143. A method of identifying a candidate agent for increasing resistance of a T cell to at least one immunosuppressive cytokine, the method comprising:
(a) providing a mammalian cell that expresses NLRP3 protein;
(b) contacting the mammalian cell with an agent;
(c) detecting the level of NLRP3 activity in the cell in step (b); and
(d) identifying an agent that increases an increased level of NLRP3 activity in the cell as compared to a control level of NLRP3 activity as a candidate agent for increasing resistance of a T cell to at least one immunosuppressive cytokine.

144. The method of claim 143, wherein the control level of NLRP3 activity is the level of NLRP3 activity in the cell in the absence of the agent.

145. The method of claim 143 or 144, wherein the NLRP3 activity is NLRP3 downstream signaling activity.

146. The method of any one of claims 143-145, wherein the mammalian cell is a T cell.

147. The method of any one of claims 143-146, further comprising:
(e) contacting a T cell with the at least one immunosuppressive cytokine and the candidate agent; and
(f) determining the ability of the candidate agent to block the immunosuppressive activity of the at least one immunosuppressive cytokine on the T cell.
